(19) United States
(12) Patent Application Publication
(10) Pub. No.: US 2009/0202995 A1
(43) Pub. Date: Aug. 13, 2009

(54) MOLECULAR CARDIOTOXICOLOGY MODELING

(76) Inventors: Donna L. Mendrick, Gaithersburg, MD (US); Kory R. Johnson, Gaithersburg, MD (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/064,933

(22) PCT Filed: Aug. 28, 2006
(86) PCT No.: PCT/US2006/033712

§ 371 Date: Sep. 30, 2008

Related U.S. Application Data
(60) Provisional application No. 60/711,444, filed on Aug. 26, 2005.

(51) Int. Cl. C12Q 1/68

(52) U.S. Cl. 435/6

(57) ABSTRACT

The present invention includes methods of predicting cardiotoxicity of test agents and methods of generating cardiotoxicity prediction models using algorithms for analyzing quantitative gene expression information. The invention also includes microarrays, computer systems comprising the toxicity prediction models, as well as methods of using the computer systems by remote users for determining the toxicity of test agents.

### RELATED APPLICATIONS

This application is entitled to priority pursuant to 35 U.S.C. §119(e) to U.S. provisional patent application No. 60/711,444, which was filed on Aug. 26, 2005, which is incorporated herein in its entirety. This application is related to, but does not claim priority to PCT/U505/011532, which is herein incorporated by reference in its entirety.

### SEQUENCE LISTING SUBMISSION ON COMPACT DISC

The contents of the submission on compact discs submitted herewith are incorporated herein by reference in their entirety: A compact disc copy of the Sequence Listing (COPY 1) (filename: GENE 127 01WO SeqList.txt, date recorded: Aug. 24, 2006, file size 2,853 kilobytes); a duplicate compact disc copy of the Sequence Listing (COPY 2) (filename: GENE 127 01WO SeqList.txt, date recorded: Aug. 24, 2006, file size 2,853 kilobytes); a duplicate compact disc copy of the Sequence Listing (COPY 3) (filename: GENE 127 01WO SeqList.txt, date recorded: Aug. 24, 2006, file size 2,853 kilobytes); a computer readable format copy of the Sequence Listing (CRF COPY) (filename: GENE 127 01WO SeqList.txt, date recorded: Aug. 24, 2006, file size 2,853 kilobytes).

### BACKGROUND OF THE INVENTION

The need for methods of assessing the toxic impact of a compound, pharmaceutical agent or environmental pollutant on a cell or living organism has led to the development of procedures which utilize living organisms as biological monitors. The simplest and most convenient of these systems utilize unicellular microorganisms such as yeast and bacteria, since they are the most easily maintained and manipulated. In addition, unicellular screening systems often use easily detectable changes in phenotype to monitor the effect of test compounds on the cell. Unicellular organisms, however, are inadequate models for estimating the potential effects of many compounds on complex multicellular animals, as they do not have the ability to carry out biotransformations.

The biotransformation of chemical compounds by multicellular organisms is a significant factor in determining the overall toxicity of agents to which they are exposed. Accordingly, multicellular screening systems may be preferred or required to detect the toxic effects of compounds. The use of multicellular organisms as toxicology screening tools has been significantly hampered, however, by the lack of convenient screening mechanisms or endpoints, such as those available in yeast or bacterial systems. Additionally, certain previous attempts to produce toxicology prediction systems have failed to provide the necessary modeling data and statistical information to accurately predict toxic responses (e.g., WO 00/12760, WO 00/47761, WO 00/63435, WO 01/32928, and WO 01/38579).

The pharmaceutical industry spends significant resources to ensure that therapeutic compounds of interest are not toxic to human beings. This process is lengthy as well as expensive and involves testing in a series of organisms starting with rodents and progressing to dogs or non-human primates. Moreover, modeling methods for designing candidate pharmaceuticals and their synthesis in nucleic acid, peptide or organic compound libraries has increased the need for inexpensive, fast and accurate methods to predict toxic responses. Toxicity modeling methods based on nucleic acid hybridization platforms would allow the use of biological samples from compound-exposed animal tissue or cell samples, such as rat tissues or cells, to detect human organ toxicity much earlier than has been possible to date.

### SUMMARY OF THE INVENTION

The present invention is based, in part, on the elucidation of the global changes in gene expression in tissues or cells exposed to known toxins, in particular cardiotoxins, as compared to unexposed tissues or cells as well as the identification of individual genes that are differentially expressed upon toxin exposure.

The invention includes methods of predicting at least one toxic effect of a compound, comprising: detecting the level of expression in cardiac tissues or cells exposed to the compound of two or more genes from Table 1, 2 or 4 and presenting information related to the detection; wherein differential expression of the genes in Table 1, 2 or 4 is indicative of at least one toxic effect. The invention also includes methods of predicting at least one toxic effect of a test compound, comprising: preparing a gene profile from tissues or cells exposed to the test compound; and comparing the gene expression profile to a database comprising quantitative gene expression information for at least one gene or gene fragment of Table 1, 2 or 4 from cardiac tissues or cells that have been exposed to at least one toxin and quantitative gene expression information for at least one gene or gene fragment of Table 1, 2 or 4 from control tissues or cells exposed to the excipients in the toxin formulation, thereby predicting at least one toxic effect of the test compound.

In various aspects, the invention also includes methods of predicting at least one toxic effect of a test agent by comparing gene expression information from agent-exposed cardiac samples to a database of gene expression information from toxin-exposed and control cardiac samples (vehicle-exposed samples or samples exposed to a non-toxic compound or experimental condition or low levels of a toxic compound). These methods comprise providing or generating quantitative gene expression information from the samples, converting the gene expression information to matrices of logged fold-change values by a robust multi-array (RMA) algorithm, generating a gene regulation score for each gene that is differentially expressed upon exposure to the test agent by a partial least squares (PLS) algorithm, and calculating a sample prediction score for the test agent. This sample prediction score is then compared to a reference prediction score for one or more toxicity models. The sample prediction score can be generated from at least one gene regulation score, or at least about 5, 10, 25, 50, 100, 500 or about 1,000 or more gene regulation scores.

In various aspects, the invention includes methods of creating a toxicity model. These methods comprise providing or generating quantitative nucleic acid hybridization data for a plurality of genes from cardiac tissues or cells exposed to a toxin and tissues or cells exposed to the toxin vehicle, converting the hybridization data from at least one gene to a gene expression measure, such as logged fold-change value, by a robust multi-array (RMA) algorithm, generating a gene regulation score from gene expression measure for the at least one gene by a partial least squares (PLS) algorithm, and generating a toxicity reference prediction score for the toxin, thereby creating a toxicity model.

The invention further provides a set of genes or gene fragments, listed in Tables 1, 2 and 4, from which probes can be made and attached to solid supports. These genes serve as a preferred set of markers of cardiotoxicity and can be used with the methods of the invention to predict or monitor a toxic effect of a compound or to modulate the onset or progression of a toxic response.

In other aspects, the invention includes a computer system comprising a computer readable medium containing a toxicity model for predicting the toxicity of a test agent and software that allows a user to predict at least one toxic effect of a test agent by comparing a sample prediction score for the test agent to a toxicity reference prediction score for the toxicity model.

In further aspects of the invention, the gene expression information from test agent-exposed tissues or cells may be prepared and transmitted via the Internet for analysis and comparisons to the toxicity models stored on a remote, central server. After processing, the user that sent the text files receives a report indicating the toxicity or non-toxicity of the test agent.

### Tables

Table 1: Table 1 provides the GLGC identifier (fragment names from Table 2) in relation to the SEQ ID NO. and GenBank Accession number for each of the gene or gene fragments listed in Table 2 (all of which are herein incorporated by reference and replicated in the attached sequence listing). Also included in the Table are gene names and Unigene cluster ID.

Table 2: Table 2 presents the PLS weight scores (index scores) for each gene from a series of cardiotoxicity models.

Table 3: Table 3 lists the toxins and negative control compounds used to build and train each cardiotoxicity model. The designation “1” for a particular compound in a particular model indicates that the compound (at the dose indicated) was used to train that model on the “Tox” portion of the model. It means that this compound is known to cause general toxicity and/or the pathology(ies) indicated. The designation “−1” for a particular compound in a particular model indicates that the compound (at the dose indicated) was used to train that model on the “Non-Tox” portion of the model. It means that this compound is known not to cause general toxicity and/or the pathology(ies) indicated. The designation “Not Used” indicates that that compound's data (at the dose indicated) was not used in building the particular model.

For the general model, “1” indicates compounds that cause toxicity in humans but may or may not cause toxicity in rats; “−1” indicates compounds that do not cause toxicity in humans, but may or may not cause toxicity in rats.

For the pathology or other compound-grouped models, “1” indicates compounds that cause that pathology or are a part of the compound group being assayed for in humans or, if the pathology or other factor is known to be a rat-specific event, compounds that cause that pathology in rats; “−1” indicates compounds that do not cause that pathology in humans or, if the pathology is known to be a rat-specific event, compounds that do not cause that pathology in rats.

Table 4: Table 4 supplies information concerning the metabolic pathways in which the genes and gene fragments of Tables 1 and 2 function.

### DETAILED DESCRIPTION

Many biological functions are accomplished by altering the expression of various genes through transcriptional (e.g. through control of initiation, provision of RNA precursors, RNA processing, etc.) and/or translational control. For example, fundamental biological processes such as cell cycle, cell differentiation and cell death are often characterized by the variations in the expression levels of groups of genes.

Changes in gene expression are also associated with the effects of various chemicals, drugs, toxins, pharmaceutical agents and pollutants on an organism or cells. For example, the lack of sufficient expression of functional tumor suppressor genes and/or the over expression of oncogene/protooncogenes after exposure to an agent could lead to tumorgenesis or hyperplastic growth of cells (Marshall (1991) Cell 64: 313-326; Weinberg (1991) Science 254:1138-1146). Thus, changes in the expression levels of particular genes (e.g. oncogenes or tumor suppressors) may serve as indicators of the presence and/or progression of toxicity or other cellular responses to exposure to a particular compound.

Monitoring changes in gene expression may also provide certain advantages during drug screening and development. Often drugs are screened for the ability to interact with an intended target with little or no regard to other effects the drugs may have on cells. These cellular effects may cause toxicity in the whole animal, which prevents the development and clinical use of the potential drug.

The present inventors have examined cardiac tissue from animals exposed to known cardiotoxins which induce detrimental heart effects in humans and/or nonclinical species, to identify global changes in gene expression and individual changes in gene expression induced by these compounds. These changes in gene expression, which can be detected by producing or obtaining gene expression profiles (an expression level of one or more genes), provide useful toxicity markers that can be used to monitor toxicity and/or toxicity progression by a test compound. Some of these markers may also be used to monitor or detect various disease or physiological states, disease progression, drug efficacy and drug metabolism.

### DEFINITIONS

As used herein, “nucleic acid hybridization data” refers to any data derived from the hybridization of a sample of nucleic acids to a one or more of a series of reference nucleic acids. Such reference nucleic acids may be in the form of probes on a microarray or may be in the form of primers that are used in polymerization reactions, such as PCR amplification, to detect hybridization of the primers to the sample nucleic acids. Nucleic hybridization data may be in the form of numerical representations of the hybridization and may be derived from quantitative, semi-quantitative or non-quantitative analysis techniques or technology platforms. Nucleic acid hybridization data includes, but is not limited to gene expression data. The data may be in any form, including florescence data or measurements of fluorescence probe intensities from a microarray or other hybridization technology platform. The nucleic acid hybridization data may be raw data or may be normalized to correct for, or take into account, background or raw noise values, including background generated by microarray high/low intensity spots, scratches, high regional or overall background and raw noise generated by scanner electrical noise and sample quality fluctuation.

As used herein, “cell or tissue samples” refers to one or more samples comprising cell or tissue from an animal or other organism, including laboratory animals such as rats or mice. The cell or tissue sample may comprise a mixed population of cells or tissues or may be substantially a single cell or tissue type. Cell or tissue samples as used herein may also be in vitro grown cells or tissue, such as primary cell cultures, immortalized cell cultures, cultured heart tissue, etc. Cells or tissue may be derived from any organ, including but not limited to, liver, kidney, cardiac, muscle (skeletal or cardiac) or brain. Preferred cells or tissues are cardiac cells or tissues, such as rat cardiac cells or tissues.

As used herein, “test agent” refers to an agent, compound, biologic such as an antibody, or composition that is being tested or analyzed in a method of the invention. For instance, a test agent may be a pharmaceutical candidate for which toxicology data is desired.

As used herein, “pathology” refers to an observable endpoint indicative of toxicity as classified by a pathologist or other practitioner with experience in the field. Most models built from expression data are based on compounds that cause common pathology endpoints. However, some models may be based on other factors for which compound commonality can be derived, including structural or mechanistic factors. The term “pathology” is used as the most common embodiment, but generally includes the other factors of compound commonality.

As used herein, “test agent vehicle” refers to the diluent or carrier in which the test agent is dissolved, suspended in or administered in, to an animal, organism or cells.

As used herein, “toxin vehicle” refers to the diluent or carrier in which a toxin is dissolved, suspended in or administered in, to an animal, organism or cells.

As used herein, a “gene expression measure” refers to any numerical representation of the expression level of a gene or gene fragment in a cell or tissue sample. A “gene expression measure” includes, but is not limited to, a fold change value.

As used herein, “at least one gene” refers to a nucleic acid molecule detected by the methods of the invention in a sample. The term “gene” as used herein, includes fully characterized open reading frames and the encoded mRNA as well as fragments of expressed RNA that are detectable by any hybridization method in the cell or tissue samples assayed as described herein. For instance, a “gene” includes any species of nucleic acid that is detectable by hybridization to a probe in a microarray, such as the “genes” of Tables 1, 2 and 4. As used herein, at least one gene includes a “plurality of genes.”

As used herein, “fold change value” refers to a numerical representation of the expression level of a gene, genes or gene fragments between experimental paradigms, such as a test or treated cell or tissue sample, compared to any standard or control. For instance, a fold change value may be presented as microarray-derived florescence or probe intensities for a gene or genes from a test cell or tissue sample compared to a control, such as an unexposed cell or tissue sample or a vehicle-exposed cell or tissue sample. An RMA logged fold change value as described herein is a non-limiting example of a fold change value calculated by methods of the invention.

As used herein, “gene regulation score” refers to a quantitative measure of gene expression for a gene or gene fragment as derived from a weighted index score or PLS score for each gene and the fold change value from treated vs. control samples.

As used herein, “sample prediction score” refers to a numerical score produced via methods of the invention as herein described. For instance, a “sample prediction score” may be calculated using the weighted index score or PLS score for at least one gene in a gene expression profile generated from the sample and the RMA fold change value for that same gene. A “sample prediction score” is derived from summing the individual gene regulation scores calculated for a given sample.

As used herein, “toxicity reference prediction score” refers to a numerical score generated from a toxicity model that can be used as a cut-off score to predict at least one toxic effect of a test agent. For instance, a sample prediction score can be compared to a toxicity reference prediction score to determine if the sample score is above or below the toxicity reference prediction score. Sample prediction scores falling below the value of a toxicity reference prediction score are scored as not exhibiting at least one toxic effect and sample prediction scores above the value if a toxicity reference prediction score are scored as exhibiting at least one toxic effect.

As used herein, a log scale linear additive model includes any log scale linear model such as log scale robust multi-array analysis or RMA (see, for example, Irizarry et al., Nucleic Acids Research 31(4) e15 (2003).

As used herein, “remote connection” refers to a connection to a server by a means other than a direct hard-wired connection. This term includes, but is not limited to, connection to a server through a dial-up line, broadband connection, Wi-Fi connection, or through the Internet.

As used herein, a “CEL file” refers to a file that contains the average probe intensities associated with a coordinate position, cell or feature on a microarray. See the Affymetrix GeneChip® Expression Analysis Technical Manual, which is herein incorporated by reference.

As used herein, a “gene expression profile” comprises any quantitative representation of the expression of at least one mRNA species in a cell sample or population and includes profiles made by various methods such as differential display, PCR, microarray and other hybridization analysis, etc.

As used herein, a “general toxicity model” refers to a model that is not limited to a specific pathology or mechanism. This category classifies compounds by their ability to induce toxicity in one or more species, including humans.

As used herein, an “arrhythmia model” refers to a model wherein the condition of the heart is characterized by a disturbance in the electrical activity that manifests as an abnormality in heart rate or heart rhythm. Patients with a cardiac arrhythmia may experience a wide variety of symptoms ranging from palpitations to fainting.

As used herein, a “myocardial necrosis model” refers to a model wherein an area of necrosis of the heart results from an insufficiency of coronary blood supply.

As used herein, a “heart failure model” refers to a model of an abnormality of cardiac function where the heart does not pump blood at the rate needed for the requirements of metabolizing tissues. The heart failure can be caused by any number of factors, including ischemic, congenital, rheumatic, or idiopathic forms.

As used herein, an “adrenergic agonist model” refers a condition where there is ineffective pumping of the heart leading to an accumulation of fluid in the lungs. Typical symptoms include shortness of breath with exertion, difficulty breathing when lying flat and leg or ankle swelling. Causes include chronic hypertension, cardiomyopathy, and myocardial infarction.

As used herein, “vasculature agents” refers to agents that cause physiological change of the vasculature.

### Methods of Generating Toxicity Models

To evaluate and identify gene expression changes that are predictive of toxicity, studies using selected compounds with well characterized toxicity may be used to build a model or database of the present invention. In the present studies, the following cardiotoxins and non-cardiotoxins were used to build one or more of the models of the invention: acyclovir, adriamycin, amphotericin B, BI compound, carboplatin, CCl4, cisplatin, clenbuterol, cyclophosphamide, dantrolene, dopamine, epinephrine, epirubicin, famotidine, hydralazine, ifosfamide, imatinib, isoproterenol, minoxidil, monocrotaline, norephinephrine, paroxetine, pentamidine, Pfizer compound, phenylpropanolamine, rosiglitazone, and temozolomide. Methods used to prepare the models of the present invention include an RMA/PLS method (analysis of raw gene expression data by the robust multi-array average algorithm, with evaluation of predictive ability by the partial least squares algorithm).

In general, the models of the invention are built using cardiac tissue and cell samples that are analyzed after exposure to compounds known to exhibit at least one toxic effect. Compounds that are known not to exhibit at least one toxic effect may also be used as negative controls. The changes in gene expression levels in samples treated with the compound were considered to represent a specific toxic response, and the genes whose expression was up- or down-regulated upon treatment with the compound were classified as marker genes that may be used as indicators of a specific type of toxic response, i.e., a specific type of heart pathology. These marker genes may also be used to prepare reference gene expression profiles that characterize a specific cardiotoxic response. To train a toxicity model that is initially built from a database of gene expression information classified as showing a toxic response or not showing a toxic response, information from samples treated with some compounds is removed from the model, while information from samples treated with other compounds is retained. If the model with the retained information also retains the ability of the original model to distinguish between a toxic response and the lack of a toxic response in test samples compared to the model, the genes in the training model whose expression is up- or down-regulated are used to build a specific toxicity model. These genes are used on the tox side of the training model.

The toxins and negative control compounds used to build and train each toxicity model are shown in Table 3. The designation “1” for a particular compound in a particular model indicates that the compound was used on the toxicity/pathology (tox) side for training the model. Where a particular compound in a particular model has the designation of “−1”, the gene expression information from samples treated with that compound is considered to represent the absence of a toxic response or pathology. This information was used on the non-tox side, or negative control side, for training a model to produce a specific toxicity model. The genes analyzed in these samples are considered not to be markers of toxicity. Where a particular compound in a particular model has the designation “Not Used,” the compound was not used to train that model.

In the present invention, a toxicity study or “tox study” comprises a set of cardiac tissues or cells that have been exposed to one or more toxins and may include matched samples exposed to the toxin vehicle or a low, non-toxic, dose of the toxin. As described below, the cell or tissue samples may be exposed to the toxin and control treatments in vivo or in vitro. In some studies, toxin and control exposure to the cell or tissue samples may take place by administering an appropriate dose to an animal model, such as a laboratory rat. In some studies, toxin and control exposure to the cell or tissue samples may take place by administering an appropriate dose to a sample of in vitro grown cells or tissue. These samples are typically organized into cohorts by test compound, time (for instance, time from initial test compound dosage to time at which rats are sacrificed or the time at which RNA is harvested from cell or tissue samples), and dose (amount of test compound administered). All cohorts in a tox study typically share the same vehicle control. For example, a cohort may be a set of samples of tissues or cells from laboratory rats that were treated with isoproterenol for 6 hours at a dosage of 0.5 mg/kg. A time-matched vehicle cohort is a set of samples that serve as controls for treated tissues or cells within a tox study, e.g., for 6-hour isoproterenol-treated samples the time-matched vehicle cohort would be the 6-hour vehicle-treated samples with that study.

A toxicity database or “tox database” is a set of tox studies that alone or in combination comprise a reference database. For instance, a reference database may include data from rat cardiac tissue and cell samples from rats that were treated with different test compounds at different dosages and exposed to the test compounds for varying lengths of time. A cardiotoxicity database is a set of cardiotoxicity studies that alone or in combination comprises a reference database.

RMA, or robust multi-array average, is an algorithm that converts raw fluorescence intensities, such as those derived from hybridization of sample nucleic acids to an Affymetrix GeneChip® microarray, into expression values, one value for each gene fragment on a chip (see, for example, Irizarry et al. (2003), Nucleic Acids Res. 31(4):e15, 8 pp.; and Irizarry et al. (2003) “Exploration, normalization, and summaries of high density oligonucleotide array probe level data,” Biostatistics 4(2): 249-264). RMA produces values on a log2 scale, typically between 4 and 12, for genes that are expressed significantly above or below control levels. These RMA values can be positive or negative and are centered around zero for a fold-change of about 1. A matrix of gene expression values generated by RMA can be subjected to PLS to produce a model for prediction of toxic responses, e.g., a model for predicting heart or kidney toxicity. In a preferred embodiment, the model is validated by techniques known to those skilled in the art. Preferably, a cross-validation technique is used. In such a technique, the data is broken into training and test sets several times until an acceptable model success rate is determined. Most preferably, such technique uses a “compound drop” cross-validation, where each compound's set of data is dropped and the data from the remaining compounds are used to rebuild the model. PLS, or Partial Least Squares, is a modeling algorithm that takes as inputs a matrix of predictors and a vector of supervised scores to generate a set of prediction weights for each of the input predictors (see, for example, Nguyen et al. (2002), Bioinformatics 18:39-50). These prediction weights are then used to calculate a gene regulation score to indicate the ability of each analyzed gene to predict a toxic response. As described in the examples, the gene regulation scores may then be used to calculate a toxicity reference prediction score.

From the nucleic acid hybridization data, a gene expression measure is calculated for one or more genes whose level of expression is detected in the nucleic acid hybridization value. As described above, the gene expression measure may comprise an RMA fold change value. The toxicity reference score=ΣwᵢR^{FCi}. “i” is the index number for each gene in a gene expression profile to be evaluated. “wᵢ” is the PLS weight (or PLS score, see Table 2) for each gene. “R^{FCi}” is the RMA fold-change value for the i^{th} gene, as determined from a normalized RMA matrix of gene expression data from the sample (described above). The PLS weight multiplied by the RMA fold-change value gives a gene regulation score for each gene, and the regulation scores for all the individual genes are added to give a toxicity reference prediction score for a sample or cohort of sample. A toxicity reference prediction score can be calculated from at least one gene regulation score, or at least about 5, 10, 25, 50, 100, 500 or about 1,000 or more gene regulation scores, including gene regulation scores calculated for the genes of the attached Tables, in particular Tables 1 and 2 as herein described.

In one embodiment of the invention, a toxicology or toxicity model of the invention is prepared or created by the steps of (a) providing nucleic acid hybridization data for a plurality of genes from tissues or cells exposed to a toxin and tissues or cells exposed to the toxin vehicle; (b) converting the hybridization data from at least one gene to a gene expression measure; (c) generating a gene regulation score from gene expression measure for said at least one gene; and (d) generating a toxicity reference prediction score for the toxin, thereby creating a toxicity model. The gene expression measure may be a gene fold change value calculated by a log scale linear additive model such as RMA and the toxicity reference prediction score may be generated with PLS. The toxicity reference prediction score may then be added to a toxicity model or database and be used to predict at least one toxic effect of an unknown test agent or compound.

In another preferred embodiment, the model is validated by techniques known to those skilled in the art. Preferably, a cross-validation technique is used. In such a technique, the data is broken into training and test sets several times until an acceptable model success rate is determined. Most preferably, such technique uses a “compound drop” cross-validation, where each compound's set of data is dropped and the data from the remaining compounds are used to rebuild the model.

### Methods of Predicting Toxic Effects

The gene regulation scores and toxicity prediction scores derived from cell or tissue samples exposed to toxins may be used to predict at least one toxic effect, including the cardiotoxicity or other tissue toxicity of a test or unknown agent or compound. The gene regulation scores and toxicity prediction scores from heart cell or tissue samples exposed to toxins may also be used to predict the ability of a test agent or compound to induce tissue pathology, such as arrhythmia, in a sample. The toxicology prediction methods of the invention are limited only by the availability of the appropriate toxicity model and toxicology prediction scores. For instance, the prediction methods of a given system, such as a computer system or database of the invention, can be expanded simply by running new toxicology studies and models of the invention using additional toxins or specific tissue pathology inducing agents and the appropriate cell or tissue samples.

As used, herein, at least one toxic effect includes, but is not limited to, a detrimental change in the physiological status of a cell or organism. The response may be, but is not required to be, associated with a particular pathology, such as tissue necrosis. Accordingly, the toxic effect includes effects at the molecular and cellular level. Cardiotoxicity, for instance, is an effect as used herein and includes but is not limited to the pathologies of: myocarditis, arrhythmias, tachycardia, myocardial ischemia, myocardial necrosis, heart failure, angina, hypertension, hypotension, dyspnea, and cardiogenic shock.

In general, assays to predict the toxicity of a test agent (or compound or multi-component composition) comprise the steps of exposing a living animal, such as a laboratory rat, to the test agent or compound, isolating the tissues and cells from the animal, providing nucleic acid hybridization data for at least one gene from the test agent exposed cell or tissue sample(s), by, for instance, assaying or measuring the level of relative or absolute gene expression of one or more of the genes, such as one or more of the genes in Table 1, 2 or 4, calculating a sample prediction score and comparing the sample prediction score to one or more toxicology reference scores (see Example 1).

Sample prediction scores may be calculated as follows: sample prediction score=ΣwⁱR^{FCi}. “i” is the index number for each gene in a gene expression profile to be evaluated. “wᵢ” is the PLS weight (or PLS score) for each gene derived from a toxicity model. “R^{FCi}” is the RMA fold-change value for the i^{th} gene, as determined from a normalized RMA matrix of gene expression data from the sample (described above). The PLS weight from a given model multiplied by the RMA fold-change value gives a gene regulation score for each gene, and the regulation scores for all the individual genes are added to give a prediction score for the sample. A sample prediction score can be calculated from at least one gene regulation score, or at least about 5, 10, 25, 50, 100, 500 or about 1,000 or more gene regulation scores (or see the numbers of genes below), including gene regulation scores calculated for the genes of the attached Tables, in particulare Tables 1 and 2 as herein described.

Nucleic acid hybridization data or methods of the invention may include any measurement of the hybridization of sample nucleic acids to probes or gene expression levels corresponding to about 2, 3, 4, 5, 6, 7, 8, 9, 10, 15, 20, 25, 30, 50, 75, 100, 200, 500, 1000 or more genes, or ranges of these numbers, such as about 2-10, about 10-20, about 20-50, about 50-100, about 100-200, about 200-500 or about 500-1000 genes of Table 1, 2 or 4. In an alternate format, PCR technology may be used to measure gene expression levels for these same numbers of genes from Table 1, 2 or 4. Nucleic acid hybridization data for toxicity prediction may also include the measurement of nearly all the genes in a toxicity model. “Nearly all” the genes may be considered to mean at least about 80% of the genes in any one toxicity model. These same numbers of genes may be used a taught herein in any step of the disclosed methods or a genes in a gene expression database as appropriate.

The methods of the invention to predict at least one toxic effect of a test agent or compound may be practiced by one individual or at one location, or may be practiced by more than one individual or at more than one location. For instance, methods of the invention include steps wherein the exposure of a test agent or compound to a cell or tissue sample(s) is accomplished in one location, nucleic acid processing and the generation of nucleic acid hybridization data takes place at another location and gene regulation and sample prediction scores calculated or generated at another location.

In another embodiment of the invention, cell or tissue samples are exposed to a test agent or compound by administering the agent to laboratory rats or to cultured heart cells and nucleic acids are processed from selected tissues and hybridized to a microarray to produce nucleic acid hybridization data. The nucleic acid hybridization data is then sent to a remote server comprising a toxicology reference database and software that enables generation of individual gene regulation scores and one or more sample prediction scores from the nucleic acid hybridization data. The software may also enable to user to pre-select specific toxicity models and to compare the generated sample prediction scores to one or more toxicology reference scores contained within a database of such scores. The user may then generate or order an appropriate output product(s) that presents or represents the results of the data analysis, generation of gene regulation scores, sample prediction scores and/or comparisons to one or more toxicology reference scores.

Data, including nucleic acid hybridization data, may be transmitted to a server via any means available, including a secure direct dial-up or a secure or unsecured internet connection. Toxicology prediction reports or any result of the methods herein may also be transmitted via these same mechanisms. For instance, a first user may transmit nucleic acid hybridization data to a remote server via a secure password protected internet link and then request transmission of a toxicology report from the server via that same internet link.

Data transmitted by a remote user of a toxicity database or model may be raw, un-normalized data or may be normalized from various background parameters before transmission. For instance, data from a microarray may be normalized for various chip and background parameters such as those described above, before transmission. The data may be in any form, as long as the data can be recognized and properly formatted by available software or the software provided as part of a database or computer system. For instance, microarray data may be provided and transmitted in a CEL file or any other common data files produced from the analysis of microarray based hybridization on commercially available technology platforms (see, for instance, the Affymetrix GeneChip® Expression Analysis Technical Manual available at www.affymetrix.com). Such files may or may not be annotated with various information, for instance, but not limited to, information related to the customer or remote user, cell or tissue sample data or information, hybridization technology or platform on which the data was generated and/or test agent data or information.

Once data is received, the nucleic acid hybridization data may be screened for database compatibility by any available means. In one embodiment, commonly available data quality control metrics can be applied. For instance, outlier analysis methods or techniques may be utilized to identify samples incompatible with the database, for instance, samples exhibiting erroneous florescence values from control probes which are common between the data and the database or toxicity model. In addition, various data QC metrics can be applied, including one or more disclosed in PCT/US03/24160, filed Aug. 1, 2003, which claims priority to U.S. provisional application 60/399,727.

### Cell or Tissue Sample Preparation

As described above, the cell population that is exposed to the test agent, compound or composition may be exposed in vitro or in vivo. For instance, cultured or freshly isolated heart cells, in particular rat heart cells, may be exposed to the agent under standard laboratory and cell culture conditions. In another assay format, in vivo exposure may be accomplished by administration of the agent to a living animal, for instance a laboratory rat.

Procedures for designing and conducting toxicity tests in in vitro and in vivo systems are well known, and are described in many texts on the subject, such as Loomis et al., Loomis's Essentials of Toxicology, 4th Ed., Academic Press, New York, 1996; Echobichon, The Basics of Toxicity Testing, CRC Press, Boca Raton, 1992; Frazier, editor, In Vitro Toxicity Testing, Marcel Dekker, New York, 1992; and the like.

In in vivo toxicity testing, two groups of test organisms are usually employed. One group serves as a control, and the other group receives the test compound in a single dose (for acute toxicity tests) or a regimen of doses (for prolonged or chronic toxicity tests). Because, in some cases, the extraction of tissue as called for in the methods of the invention requires sacrificing the test animal, both the control group and the group receiving compound must be large enough to permit removal of animals for sampling tissues, if it is desired to observe the dynamics of gene expression through the duration of an experiment.

In setting up a toxicity study, extensive guidance is provided in the literature for selecting the appropriate test organism for the compound being tested, route of administration, dose ranges, and the like. Water or physiological saline (0.9% NaCl in water) is the solute of choice for the test compound since these solvents permit administration by a variety of routes. When this is not possible because of solubility limitations, vegetable oils such as corn oil or organic solvents such as propylene glycol may be used.

Regardless of the route of administration, the volume required to administer a given dose is limited by the size of the animal that is used. It is desirable to keep the volume of each dose uniform within and between groups of animals. When rats or mice are used, the volume administered by the oral route generally should not exceed about 0.005 ml per gram of animal. Even when aqueous or physiological saline solutions are used for parenteral injection the volumes that are tolerated are limited, although such solutions are ordinarily thought of as being innocuous. The intravenous LD₅₀ of distilled water in the mouse is approximately 0.044 ml per gram and that of isotonic saline is 0.068 ml per gram of mouse. In some instances, the route of administration to the test animal should be the same as, or as similar as possible to, the route of administration of the compound to humans for therapeutic purposes.

When a compound is to be administered by inhalation, special techniques for generating test atmospheres are necessary. The methods usually involve aerosolization or nebulization of fluids containing the compound. If the agent to be tested is a fluid that has an appreciable vapor pressure, it may be administered by passing air through the solution under controlled temperature conditions. Under these conditions, dose is estimated from the volume of air inhaled per unit time, the temperature of the solution, and the vapor pressure of the agent involved. Gases are metered from reservoirs. When particles of a solution are to be administered, unless the particle size is less than about 2 μm the particles will not reach the terminal alveolar sacs in the lungs. A variety of apparati and chambers are available to perform studies for detecting effects of irritant or other toxic endpoints when they are administered by inhalation. The preferred method of administering an agent to animals is via the oral route, either by intubation or by incorporating the agent in the feed.

When the agent is exposed to cells in vitro or in cell culture, the cell population to be exposed to the agent may be divided into two or more subpopulations, for instance, by dividing the population into two or more identical aliquots. In some preferred embodiments of the methods of the invention, the cells to be exposed to the agent are derived from heart tissue. For instance, cultured or freshly isolated rat heart cells may be used.

The methods of the invention may be used generally to predict at least one toxic response, and, as described in the Examples, may be used to predict the likelihood that a compound or test agent will induce various specific pathologies, such as arrhythmias, myocardial necrosis, heart failure, or other pathologies associated with at least one known toxin. The methods of the invention may also be used to determine the similarity of a toxic response to one or more individual compounds. In addition, the methods of the invention may be used to predict or elucidate the potential cellular pathways influenced, induced or modulated by the compound or test agent.

### Databases and Computer Systems

Databases and computer systems of the present invention typically comprise one or more data structures, saved to a computer readable medium, comprising toxicity or toxicology models as described herein, including models comprising individual gene or toxicology marker weighted index scores or PLS scores (See Table 2), gene regulation scores, sample prediction scores and/or toxicity reference prediction scores. Such databases and computer systems may also comprise software that allows a user to manipulate the database content or to calculate or generate scores as described herein, including individual gene regulation scores and sample prediction scores from nucleic acid hybridization data. The software may also allow the user to compare one or more sample prediction scores to one or more toxicity reference paradigm scores in at least one toxicity model.

As discussed above, the databases and computer systems of the invention may comprise equipment and software that allow access directly or through a remote link, such as direct dial-up access or access via a password protected Internet link. Any available hardware may be used to create computer systems of the invention. Any appropriate computer platform, user interface, etc. may be used to perform the necessary comparisons between sequence information, gene or toxicology marker information and any other information in the database or information provided as an input. For example, a large number of computer workstations are available from a variety of manufacturers. Client/server environments, database servers and networks are also widely available and appropriate platforms for the databases of the invention.

The databases may be designed to include different parts, for instance a sequence database and a toxicology reference database. Methods for the configuration and construction of such databases and computer-readable media containing such databases are widely available, for instance, see U.S. Publication No. 2003/0171876 (Ser. No. 10/090,144), filed Mar. 5, 2002, PCT Publication No. WO 02/095659, published Nov. 23, 2002, and U.S. Pat. No. 5,953,727, which are herein incorporated by reference in their entirety. In a preferred embodiment, the database is a ToxExpress® or BioExpress database marketed by Gene Logic Inc., Gaithersburg, Md. A toxicology database of the invention may include gene expression information for about or at least about 2, 3, 4, 5, 6, 7, 8, 9, 10, 15, 20, 25, 30, 50, 75, 100, 200, 500, 1000 or more genes from Table 2 (or Table 1), wherein the gene expression information is from cardiac tissues or cells exposed in vivo or in vitro to one or more of the toxins or controls as described herein.

The databases of the invention may be linked to an outside or external database such as GenBank (www.ncbi.nlm.nih.gov/entrez.index.html); KEGG (www.genome.ad.jp/kegg); SPAD (www.grt.kyushu-u.ac.jp/spad/index.html); HUGO (www.gene.ucl.ac.uk/hugo); Swiss-Prot (www.expasy.ch.sprot); Prosite (www.expasy.ch/tools/scnpsitl.html); OMIM (www.ncbi.nlm.nih.gov/omim); and GDB (www.gdb.org). In a preferred embodiment, the external database is GenBank and the associated databases maintained by the National Center for Biotechnology Information (NCBI) (www.ncbi.nlm.nih.gov).

Any appropriate computer platform, user interface, etc. may be used to perform the necessary comparisons between sequence information, gene expression information and any other information in the database or information provided as an input. For example, a large number of computer workstations are available from a variety of manufacturers, such has those available from Silicon Graphics. Client/server environments, database servers and networks are also widely available and appropriate platforms for the databases of the invention.

The databases of the invention may be used to produce, among other things, eNortherns™ reports (Gene Logic, Inc) that allow the user to determine the cell type or tissue in which a given gene is expressed and to allow determination of the abundance or expression level of a given gene in a particular tissue or cell.

### Toxicity or Toxicology Reports

As described above, the methods, databases and computer systems of the invention can be used to produce, deliver and/or send a toxicity, cardiotoxicity or toxicology report. As consistent with the use of the terms “toxicity” and “toxicology” as used herein, a “toxicity report” and a “toxicology report” are interchangeable.

The toxicity report of the invention typically comprises information or data related to the results of the practice of a method of the invention. For instance, the practice of a method of identifying at least one toxic effect of a test agent or compound as herein described may result in the preparation or production of a report describing the results of the method. The report may comprise information related to the toxic effects predicted by the comparison of at least one sample prediction score to at least one toxicity reference prediction score from the database. The report may also present information concerning the nucleic acid hybridization data, such as the integrity of the data as well as information inputted by the user of the database and methods of the invention, such as information used to annotate the nucleic acid hybridization data.

As an exemplary, non-limiting example, a toxicity report of the invention may be in a form such as the reports disclosed in PCT/US02/22701, filed Jul. 18, 2002, which is herein incorporated by reference in its entirety. As described elsewhere in this specification, the report may be generated by a server or computer system to which is loaded nucleic acid hybridization data by a user. The report related to that nucleic acid data may be generated and delivered to the user via remote means such as a password secured environment available over the internet or via available computer communication means such as email.

### Generating Nucleic Acid Hybridization Data

Any assay format to detect gene expression may be used to produce nucleic acid hybridization data. For example, traditional Northern blotting, dot or slot blot, nuclease protection, primer directed amplification, RT-PCR, semi- or quantitative PCR, branched-chain DNA and differential display methods may be used for detecting gene expression levels or producing nucleic acid hybridization data. Those methods are useful for some embodiments of the invention. In cases where smaller numbers of genes are detected, amplification based assays may be most efficient. Methods and assays of the invention, however, may be most efficiently designed with high-throughput hybridization-based methods for detecting the expression of a large number of genes.

To produce nucleic acid hybridization data, any hybridization assay format may be used, including solution-based and solid support-based assay formats. Solid supports containing oligonucleotide probes for differentially expressed genes of the invention can be filters, polyvinyl chloride dishes, particles, beads, microparticles or silicon or glass based chips, etc. Such chips, wafers and hybridization methods are widely available, for example, those disclosed by Beattie (WO 95/11755).

Any solid surface to which oligonucleotides can be bound, either directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 or more of such features on a single solid support. The solid support, or the area within which the probes are attached may be on the order of about a square centimeter. Probes corresponding to the genes or gene fragments of Table 1, 2 or 4 may be attached to single or multiple solid support structures, e.g., the probes may be attached to a single chip or to multiple chips to comprise a chip set. The genes or gene fragments described in the related applications mentioned above may also be attached to these solid supports.

Oligonucleotide probe arrays for expression monitoring can be made and used according to any techniques known in the art (see for example, Lockhart et al. (1996), Nat Biotechnol 14:1675-1680; McGall et al. (1996), Proc Nat Acad Sci USA 93: 13555-13460). Such probe arrays may contain at least two or more oligonucleotides that are complementary to or hybridize to two or more of the genes or gene fragments described in Table 1, 2 or 4. For instance, such arrays may contain oligonucleotides that are complementary to or hybridize to at least about 2, 3, 4, 5, 6, 7, 8, 9, 10, 20, 30, 50, 70, 100, 500 or 1,000 or more of the genes described herein. Preferred arrays contain all, or substantially all, of the genes or gene fragments listed in Table 1, 2 or 4. As used herein, “substantially all” of the genes in Table 1, 2 or 4 refers to a set of genes or gene fragments containing at least 80% of the genes or gene fragments in Table 1, 2 or 4. In another preferred embodiment, arrays are constructed that contain oligonucleotides to detect all or nearly all of the genes in Table 1, 2 or 4, or a single model of Table 1, 2 or 4, on a single solid support substrate, such as a chip.

The sequences of the genes and gene fragments of Table 1, 2 or 4 are in the public databases. Table 1 provides the SEQ ID NO: and GenBank Accession Number (NCBI RefSeq ID) for each of the sequences (see www.ncbi.nlm.nih.gov/), as well as the title for the cluster of which gene is part. The sequences of the genes in GenBank are expressly herein incorporated by reference in their entirety as of the filing date of this application, as are related sequences, for instance, sequences from the same gene of different lengths, variant sequences, polymorphic sequences, genomic sequences of the genes and related sequences from different species, including the human counterparts, where appropriate. As described above, in addition to the sequences of the GenBank Accession Numbers disclosed in the Table 1, 2 or 4, sequences such as naturally occurring variant or polymorphic sequences may be used in the methods and compositions of the invention. For instance, expression levels of various allelic or homologous forms of a gene or gene fragment disclosed in Table 1, 2 or 4 may be assayed. Any and all nucleotide variations that do not alter the functional activity of a gene or gene fragment listed in Table 1, 2 or 4, including all naturally occurring allelic variants of the genes herein disclosed, may be used in the methods and to make the compositions (e.g., arrays) of the invention.

Probes based on the sequences of the genes described above may be prepared by any commonly available method. Oligonucleotide probes for screening or assaying a tissue or cell sample are preferably of sufficient length to specifically hybridize only to appropriate, complementary genes or transcripts. Typically the oligonucleotide probes will be at least about 10, 12, 14, 16, 18, 20 or 25 nucleotides in length. In some cases, longer probes of at least 30, 40, or 50 nucleotides will be desirable.

As used herein, oligonucleotide sequences that are complementary to one or more of the genes or gene fragments described in Table 1, 2 or 4 refer to oligonucleotides that are capable of hybridizing under stringent conditions to at least part of the nucleotide sequences of said genes. Such hybridizable oligonucleotides will typically exhibit at least about 75% sequence identity at the nucleotide level to said genes, preferably about 80% or 85% sequence identity or more preferably about 90% or 95% or more sequence identity to said genes (see GeneChip® Expression Analysis Manual, Affymetrix, Rev. 3, which is herein incorporated by reference in its entirety).

### Probe Design

One of skill in the art will appreciate that an enormous number of array designs are suitable for the practice of this invention. The high density array will typically include a number of test probes that specifically hybridize to the sequences of interest. Probes may be produced from any region of the genes or gene fragments identified in Table 1, 2 or 4 and the attached representative sequence listing. In instances where the gene reference in the Tables is a gene fragment, probes may be designed from that sequence or from other regions of the corresponding full-length transcript that may be available in any of the sequence databases, such as those herein described. See WO 99/32660 for methods of producing probes for a given gene or genes. In addition, any available software may be used to produce specific probe sequences, including, for instance, software available from Molecular Biology Insights, Olympus Optical Co. and Biosoft International. In a preferred embodiment, the array will also include one or more control probes.

High density array chips of the invention include “test probes.” Test probes may be oligonucleotides that range from about 5 to about 500, or about 7 to about 50 nucleotides, more preferably from about 10 to about 40 nucleotides and most preferably from about 15 to about 35 nucleotides in length. In other particularly preferred embodiments, the probes are about 20 or 25 nucleotides in length. In another preferred embodiment, test probes are double or single strand DNA sequences. DNA sequences are isolated or cloned from natural sources or amplified from natural sources using native nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes may fall into three categories referred to herein as 1) normalization controls; 2) expression level controls; and 3) mismatch controls.

Normalization controls are oligonucleotide or other nucleic acid probes that are complementary to labeled reference oligonucleotides or other nucleic acid sequences that are added to the nucleic acid sample to be screened. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like. Examples of expression level control probes may be found in U.S. application Ser. Nos. 10/479,866, 10/483,889, 10/620,765 and 10/629,618. Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20 mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. For example, if the target is present the perfect match probes should be consistently brighter than the mismatch probes. In addition, if all central mismatches are present, the mismatch probes can be used to detect a mutation, for instance, a mutation of a gene or gene fragment in Table 1, 2 or 4. The difference in intensity between the perfect match and the mismatch probe provides a good measure of the concentration of the hybridized material.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” or “specifically hybridizes” refers to the binding, duplexing, or hybridizing of a molecule substantially to or only to a particular nucleotide sequence or sequences under stringent conditions when that sequence is present in a complex mixture (e.g., total cellular) DNA or RNA.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

### Forming High Density Arrays

Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling (see Pirrung, U.S. Pat. No. 5,143,854).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO 93/09668 and WO 01/23614. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

### Nucleic Acid Samples

Cell or tissue samples may be exposed to the test agent in vitro or in vivo. When cultured cells or tissues are used, appropriate mammalian cell extracts, such as liver extracts, may also be added with the test agent to evaluate agents that may require biotransformation to exhibit toxicity. In a preferred format, primary isolates, cultured cell lines or freshly isolated or frozen animal or human heart cells may be used.

The genes which are assayed according to the present invention are typically in the form of mRNA or reverse transcribed mRNA. The genes may or may not be cloned. The genes may or may not be amplified. The cloning and/or amplification do not appear to bias the representation of genes within a population. In some assays, it may be preferable, however, to use polyA+ RNA as a source, as it can be used with less processing steps.

As is apparent to one of ordinary skill in the art, nucleic acid samples used in the methods and assays of the invention may be prepared by any available method or process. Methods of isolating total mRNA are well known to those of skill in the art. For example, methods of isolation and purification of nucleic acids are described in detail in Chapter 3 of Laboratory Techniques in Biochemistry and Molecular Biology. Vol. 24, Hybridization With Nucleic Acid Probes: Theory and Nucleic Acid Probes, P. Tijssen, Ed., Elsevier Press, New York, 1993. Such samples include RNA samples, but also include cDNA synthesized from a mRNA sample isolated from a cell or tissue of interest. Such samples also include DNA amplified from the cDNA, and RNA transcribed from the amplified DNA. One of skill in the art would appreciate that it is desirable to inhibit or destroy RNase present in homogenates before homogenates are used.

Biological samples may be of any biological tissue or fluid or cells from any organism as well as cells raised in vitro, such as cell lines and tissue culture cells. Frequently the sample will be a tissue or cell sample that has been exposed to a compound, agent, drug, pharmaceutical composition, potential environmental pollutant or other composition. In some formats, the sample will be a “clinical sample” which is a sample derived from a patient. Typical clinical samples include, but are not limited to, sputum, blood, blood-cells (e.g., white cells), tissue or fine needle biopsy samples, urine, peritoneal fluid, and pleural fluid, or cells therefrom. Biological samples may also include sections of tissues, such as frozen sections or formalin fixed sections taken for histological purposes.

### Hybridization

Nucleic acid hybridization simply involves contacting a probe and target nucleic acid under conditions where the probe and its complementary target can form stable hybrid duplexes through complementary base pairing. See WO 99/32660. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency, in this case in 6×SSPET at 37° C. (0.005% Triton X-100), to ensure hybridization and then subsequent washes are performed at higher stringency (e.g., 1×SSPET at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

### Signal Detection

The hybridized nucleic acids are typically detected by detecting one or more labels attached to the sample nucleic acids. The labels may be incorporated by any of a number of means well known to those of skill in the art. See WO 99/32660.

### Kits

The invention further includes kits combining, in different combinations, high-density oligonucleotide arrays, reagents for use with the arrays, signal detection and array-processing instruments, toxicology databases and analysis and database management software described above. The kits may be used, for example, to predict or model the toxic response of a test compound.

The databases that may be packaged with the kits are described above. In particular, the database software and packaged information may contain the databases saved to a computer-readable medium, or transferred to a user's local server. In another format, database and software information may be provided in a remote electronic format, such as a website, the address of which may be packaged in the kit.

Databases and software designed for use with microarrays are discussed in Balaban et al., U.S. Pat. Nos. 6,229,911, a computer-implemented method for managing information collected from small or large numbers of microarrays, and 6,185,561, a computer-based method with data mining capability for collecting gene expression level data, adding additional attributes and reformatting the data to produce answers to various queries. Chee et al., U.S. Pat. No. 5,974,164, disclose a software-based method for identifying mutations in a nucleic acid sequence based on differences in probe fluorescence intensities between wild type and mutant sequences that hybridize to reference sequences.

### Diagnostic Uses for the Toxicity Markers

As described above, the genes and gene expression information or portfolios of the genes with their expression information as provided in the accompanying Tables may be used as diagnostic markers for the prediction or identification of the physiological state of tissue or cell sample that has been exposed to a compound or to identify or predict the toxic effects of a compound or agent. For instance, a tissue sample such as a sample of peripheral blood cells or some other easily obtainable tissue sample may be assayed by any of the methods described above, and the expression levels from a gene or gene fragment of Table 1, 2 or 4 may be compared to the expression levels found in tissues or cells exposed to the toxins described herein. These methods may result in the diagnosis of a physiological state in the cell or may be used to identify the potential toxicity of a compound, for instance a new or unknown compound or agent. The comparison of expression data, as well as available sequence or other information may be done by researcher or diagnostician or may be done with the aid of a computer and databases as described below.

### Use of the Markers for Monitoring Toxicity Progression

As described above, the genes and gene expression information provided in Table 1, 2 or 4 may also be used as markers for the monitoring of toxicity progression, such as that found after initial exposure to a drug, drug candidate, toxin, pollutant, etc. For instance, a tissue or cell sample may be assayed by any of the methods described above, and the expression levels from a gene or gene fragment of Table 1, 2 or 4 may be compared to the expression levels found in tissue or cells exposed to the cardiotoxins described herein. The comparison of the expression data, as well as available sequence or other information may be done by researcher or diagnostician or may be done with the aid of a computer and databases.

### Use of the Toxicity Markers for Drug Screening

According to the present invention, the genes and gene fragments identified in Table 1, 2 or 4 may be used as markers or drug targets to evaluate the effects of a candidate drug, chemical compound or other agent on a cell or tissue sample. The genes may also be used as drug targets to screen for agents that modulate their expression and/or activity. In various formats, a candidate drug or agent can be screened for the ability to stimulate the transcription or expression of a given marker or markers or to down-regulate or counteract the transcription or expression of a marker or markers. According to the present invention, one can also compare the specificity of a drug's effects by looking at the number of markers which the drug induces and comparing them. More specific drugs will have less transcriptional targets. Similar sets of markers identified for two drugs may indicate a similarity of effects.

Assays to monitor the expression of a marker or markers as defined in Table 1, 2 or 4 may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, gene chips containing probes to one, two or more genes or gene fragments from Table 1, 2 or 4 may be used to directly monitor or detect changes in gene expression in the treated or exposed cell. Cell lines, tissues or other samples are first exposed to a test agent and in some instances, a known toxin, and the detected expression levels of one or more, or preferably 2 or more of the genes or gene fragments of Table 1, 2 or 4 are compared to the expression levels of those same genes exposed to a known toxin alone. Compounds that modulate the expression patterns of the known toxin(s) would be expected to modulate potential toxic physiological effects in vivo. The genes and gene fragments in Table 1, 2 or 4 are particularly appropriate markers in these assays as they are differentially expressed in cells upon exposure to a known cardiotoxin.

In another format, cell lines that contain reporter gene fusions between the open reading frame and/or the transcriptional regulatory regions of a gene or gene fragment in Table 1, 2 or 4 and any assayable fusion partner may be prepared. Numerous assayable fusion partners are known and readily available including the firefly luciferase gene and the gene encoding chloramphenicol acetyltransferase (Alam et al. (1990) Anal Biochem 188:245-254). Cell lines containing the reporter gene fusions are then exposed to the agent to be tested under appropriate conditions and time. Differential expression of the reporter gene between samples exposed to the agent and control samples identifies agents which modulate the expression of the nucleic acid.

Additional assay formats may be used to monitor the ability of the agent to modulate the expression of a gene identified in Table 1, 2 or 4. For instance, as described above, mRNA expression may be monitored directly by hybridization of probes to the nucleic acids of the invention. Cell lines are exposed to the agent to be tested under appropriate conditions and time and total RNA or mRNA is isolated by standard procedures such those disclosed in Sambrook et al. (Molecular Cloning: A Laboratory Manual, Third Ed Cold Spring Harbor Laboratory Press, Cold Spring Harbor, N.Y., 2001).

Agents that are assayed in the above methods can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see G. A. Grant in: Molecular Biology and Biotechnology, Meyers, ed., pp. 659-664, VCH Publishers, New York, 1995). A skilled artisan can readily recognize that there is no limit as to the structural nature of the agents of the present invention.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Generation of Toxicity Models using RMA and PLS

The cardiotoxins and control compositions including, but not limited to, acyclovir, adriamycin, amphotericin B, BI compound, carboplatin, CCl4, cisplatin, clenbuterol, cyclophosphamide, dantrolene, dopamine, epinephrine, epirubicin, famotidine, hydralazine, ifosfamide, imatinib, isoproterenol, minoxidil, monocrotaline, norephinephrine, paroxetine, pentamidine, Pfizer compound, phenylpropanolamine, rosiglitazone, and temozolomide were administered to male Sprague-Dawley rats at various time points using administration diluents, protocols and dosing regimes described above as well as previously described in the art and in the related applications discussed above.

After administration, the dosed animals were observed and tissues were collected as described below, although heart tissues were used in the cardiotoxicity models described herein.

### Observation of Animals

1. Clinical Observations—Twice daily: mortality and moribundity check. Cage Side Observations—skin and fur, eyes and mucous membrane, respiratory system, circulatory system, autonomic and central nervous system, somatomotor pattern, and behavior pattern. Potential signs of toxicity, including tremors, convulsions, salivation, diarrhea, lethargy, coma or other atypical behavior or appearance, were recorded as they occurred and included a time of onset, degree, and duration.

2. Physical Examinations—Prior to randomization, prior to initial treatment, and prior to sacrifice.

3. Body Weights—Prior to randomization, prior to initial treatment, and prior to sacrifice.

Clinical Pathology

| | | |
|---|---|---|
| 1. | Frequency | Prior to necropsy. |
| 2. | Number of animals | All surviving animals. |
| 3. | Bleeding Procedure | Blood was obtained by puncture of |
| | | the orbital sinus while under 70% |
| | | CO₂/30% O₂ anesthesia. |
| 4. | Collection of Blood Samples | Approximately 0.5 mL of blood was |
| | | collected into EDTA tubes for |
| | | evaluation of hematology parameters. |
| | | Approximately 1 mL of blood was |
| | | collected into serum separator tubes |
| | | clinical chemistry analysis. |
| | | Approximately 200 μL of plasma was |
| | | obtained and frozen at ~−80° C. for |
| | | test compound/metabolite estimation. |
| | | An additional ~2 mL of blood was |
| | | collected into a 15 mL conical poly- |
| | | propylene vial to which ~3 mL of |
| | | Trizol was immediately added. The |
| | | contents were immediately mixed |
| | | with a vortex and by repeated in- |
| | | version. The tubes were frozen in |
| | | liquid nitrogen and stored at ~−80° C. |

### Termination Procedures

Terminal Sacrifice

At the sampling times indicated in Table 3 for each cardiotoxin, and as previously described in the related applications mentioned above, rats were weighed, physically examined, sacrificed by decapitation, and exsanguinated. The animals were necropsied within approximately five minutes of sacrifice. Separate sterile, disposable instruments were used for each animal, with the exception of bone cutters, which were used to open the skull cap. The bone cutters were dipped in disinfectant solution between animals.

Necropsies were conducted on each animal following procedures approved by board-certified pathologists.

Animals not surviving until terminal sacrifice were discarded without necropsy (following euthanasia by carbon dioxide asphyxiation, if moribund). The approximate time of death for moribund or found dead animals was recorded.

Postmortem Procedures

Fresh and sterile disposable instruments were used to collect tissues. Gloves were worn at all times when handling tissues or vials. All tissues were collected and frozen within approximately 5 minutes of the animal's death. The liver sections, kidneys and hearts were frozen within approximately 3-5 minutes of the animal's death. The time of euthanasia, an interim time point at freezing of liver sections and kidneys, and time at completion of necropsy were recorded. Tissues were stored at approximately −80° C. or preserved in 10% neutral buffered formalin.

Tissue Collection and Processing

Liver—

1. Right medial lobe—snap frozen in liquid nitrogen and stored at −80° C.
2. Left medial lobe—Preserved in 10% neutral-buffered formalin (NBF) and evaluated for gross and microscopic pathology.
3. Left lateral lobe—snap frozen in liquid nitrogen and stored at ˜−80° C.

Heart—

A sagittal cross-section containing portions of the two atria and of the two ventricles was preserved in 10% NBF. The remaining heart was frozen in liquid nitrogen and stored at ˜−80° C.

Kidneys (both)—

1. Left—Hemi-dissected; half was preserved in 10% NBF and the remaining half was frozen in liquid nitrogen and stored at ˜−80° C.
2. Right—Hemi-dissected; half was preserved in 10% NBF and the remaining half was frozen in liquid nitrogen and stored at ˜−80° C.

Testes (both)—

A sagittal cross-section of each testis was preserved in 10% NBF. The remaining testes were frozen together in liquid nitrogen and stored at ˜−80° C.

Brain (whole)—

A cross-section of the cerebral hemispheres and of the diencephalon was preserved in 10% NBF, and the rest of the brain was frozen in liquid nitrogen and stored at ˜−80° C.

RNA Collection from Tissues or Cells and Processing

Microarray sample preparation is conducted with minor modifications, following the protocols set forth in the Affymetrix GeneChip® Expression Technical Analysis Manual (Affymetrix, Inc. Santa Clara, Calif.). Frozen cardiac cells are ground to a powder using a Spex Certiprep 6800 Freezer Mill. Total RNA is extracted with Trizol (Invitrogen, Carlsbad Calif.) utilizing the manufacturer's protocol. The total RNA yield for each sample is typically 200-500 μg per 300 mg cells. mRNA is isolated using the Oligotex mRNA Midi kit (Qiagen) followed by ethanol precipitation. Double stranded cDNA is generated from mRNA using the SuperScript Choice system (Invitrogen, Carlsbad Calif.). First strand cDNA synthesis is primed with a T7-(dT24) oligonucleotide. The cDNA is phenol-chloroform extracted and ethanol precipitated to a final concentration of 1 μg/ml. From 2 μg of cDNA, cRNA is synthesized using Ambion's T7 MegaScript in vitro Transcription Kit.

To biotin label the cRNA, nucleotides Bio-11-CTP and Bio-16-UTP (Enzo Diagnostics) are added to the reaction. Following a 37° C. incubation for six hours, impurities are removed from the labeled cRNA following the RNeasy Mini kit protocol (Qiagen). cRNA is fragmented (fragmentation buffer consisting of 200 mM Tris-acetate, pH 8.1, 500 mM KOAc, 150 mM MgOAc) for thirty-five minutes at 94° C. Following the Affymetrix protocol, 55 μg of fragmented cRNA is hybridized on the Affymetrix rat array set for twenty-four hours at 60 rpm in a 45° C. hybridization oven. The chips are washed and stained with Streptavidin Phycoerythrin (SAPE) (Molecular Probes) in Affymetrix fluidics stations. To amplify staining, SAPE solution is added twice with an anti-streptavidin biotinylated antibody (Vector Laboratories) staining step in between. Hybridization to the probe arrays is detected by fluorometric scanning (Hewlett Packard Gene Array Scanner). Data is analyzed using Affymetrix GeneChip®0 and Expression Data Mining (EDMT) software, the GeneExpress® database, and S-Plus® statistical analysis software (Insightful Corp.).

### Identification of Toxicity Markers and Model Building Using RMA and PLS Algorithms

RMA/PLS models are built as follows. From DNA microarray data from one or more studies, a matrix of RMA fold-change expression values is generated (in this study, nucleic acid hybridization from heart tissue exposed to various cardiotoxins or control compounds in used). These values are generated, for example, according to the method of Irizarry et al. (Nucl Acids Res 31 (4):e 15, 2003, which is herein incorporated by reference in its entirety), which uses the following equation to produce a log scale linear additive model: T(PMᵢⱼ)=eᵢ+aⱼ+εᵢⱼ. T represents the transformation that corrects for background and normalizes and converts the PM (perfect match) intensities to a log scale. eᵢ represents the log2 scale expression values found on arrays i=1−I, aⱼ represents the log scale affinity effects for probes j=1−J, and εᵢⱼ represents error (to correct for the differences in variances when using probes that bind with different intensities).

In RMA fold-change matrices, the rows represent individual fragments, and the columns are individual samples. A vehicle cohort median matrix is then calculated, in which the rows represent fragments and the columns represent vehicle cohorts, one cohort for each study/time-point combination. The values in this matrix are the median RMA expression values across the samples within those cohorts. Next, a matrix of normalized RMA expression values is generated, in which the rows represent individual fragments and the columns are individual samples. The normalized RMA values are the RMA values minus the value from the vehicle cohort median matrix corresponding to the time-matched vehicle cohort. Next, the absolute value of the mean of these differences is calculated. These absolute mean difference values serve as the base data on which both fragment selection and PLS modeling is calculated.

Fragment selection is achieved through several successive steps. Step 1, a “Control Cohort” matrix is created using the absolute mean difference values, where the rows represent fragments and the columns represent vehicle and/or non-cardiotoxin absolute mean difference values for each cohort. Step 2, a “Toxin Cohort” matrix is created using the absolute mean difference values, where the rows represent fragments and the columns represent cardiotoxin absolute mean difference values for each cohort. Step 3, remove fragments from the “Control Cohort” matrix that are uniquely regulated for any single cohort within that matrix. This is done by removing those fragments where the highest absolute mean difference value is 1.25 times greater than the next highest absolute mean difference value. This step is done to reduce the incidence of false-positives due to aberrant unique regulation in the “Control” class. These same fragments are also removed from the “Toxin Cohort” matrix. Step 4, the “Toxin Cohort” matrix is converted to a binary coding based on whether the cardiotoxin absolute mean difference value is 1.25 times greater than or equal to the maximum observed absolute mean difference value in the “Control Cohort” matrix. For each fragment and cohort that meets this criteria, a value of “1” is assigned; otherwise, a value of “0” is assigned. This binary coding is done for each cell of the “Toxin Cohort” matrix. Step 5, a new matrix, the “Toxin Compound” matrix, is created by taking the maximum binary assigned code over each cardiotoxin's cohorts. Therefore, each compound is represented for each fragment with a “1” where any of its treatment cohorts contains a “1” in the “Toxin Cohort” binary matrix, or with a “0” where all of its treatment cohorts contain a “0.” Step 6, each row of the “Toxin Compound” matrix is summed, yielding the number of cardiotoxins that a fragment is regulated by relative to vehicles and non-cardiotoxicants.

PLS modeling is then applied to the absolute mean difference values (a subset by taking certain fragments as described below), using a −1=non-tox, +1=tox supervised score vector as the dependant variable and the rows of normalized RMA matrix as the independent variables. PLS works by computing a series of PLS components, where each component is a weighted linear combination of fragment values. In this case, the nonlinear iterative partial least squares method is used to compute the PLS components.

PLS modeling and compound drop cross-validation are then performed based on taking the top N fragments according to the frequency of regulation observed in the “Toxin Compound” matrix, varying N and the number of PLS components, and recording the model success rate for each combination. N is chosen to be the point at which the cross-validated error rate is minimized. In the PLS model, each of those N fragments receives a PLS weight (PLS score) corresponding to the fragment's utility, or predictive ability, in the model (see Table 2 for lists of PLS weight scores for individual genes and gene fragments in the various cardiotoxicity models). Table 2 presents several cardiotoxicity models and includes the gene or gene fragment name for each marker and the corresponding PLS weight or index score for each gene or gene fragment in each model. The models are as follows: general toxicity, adrenergic agonist, arrhythmia, heart failure, myocardial necrosis, and vasculature agent.

To establish a toxicity prediction score cut-off value for a toxicity model, the true-positive and false positive rates for each possible score cut-off value are computed, using the scores from all tox and non-tox samples in the training set. This generates an ROC curve, which is used to set the cut-off score at the point on the ROC curve corresponding to −5% false positive rate.

The model can be trained by setting a score of −1 for each gene that cannot predict a toxic response and by setting a score of +1 for each gene that can predict a toxic response. Cross-validation of RMA/PLS models may be performed by the compound-drop method and by the 2/3:1/3 method. In the compound-drop method, sample data from animals treated with one particular test compound are removed from a model, and the ability of this model to predict toxicity is compared to that of a model containing a full data set. In the 2/3:1/3 method, gene expression information from a random third of the genes in the model is removed, and the ability of this subset model to predict toxicity is compared to that of a model containing a full data set.

| Model | Cut-off score |
|---|---|
| general | 1.41 |
| adrenergic agonist | 0.97 |
| arrhythmia | 1.25 |
| heart failure | 1.29 |
| myocardial necrosis | 0.87 |
| vasculature agent | 0.80 |

### Example 2

### Methods of Predicting at Least One Toxic Effect of a Test Agent

To determine whether or not a cardiac cell or tissue sample such as tissues or cells treated with a test agent or compound exhibits at least one toxic effect or response, RNA is prepared from tissues or cells exposed to the agent and hybridized to a DNA microarray, as described in Example 1 above. From the nucleic acid hybridization data, a prediction score is calculated for that sample and compared to a reference score from a toxicity reference database according to the following equation. The sample prediction score=ΣwᵢR^{FCi}. “i” is the index number for each gene in a gene expression profile to be evaluated. “wᵢ” is the PLS weight score (or PLS index score, see Table 2 for the lists of PLS scores for each cardiotoxicity model) for each gene. “R^{FCi}” is the RMA fold-change value for the i^{th} gene, as determined from a normalized RMA matrix of gene expression data from the sample (described above). The PLS weight multiplied by the RMA fold-change value gives a gene regulation score for each gene, and the regulation scores for all the individual genes are added to give a prediction score for the sample.

As a quality control (QC) check, for each incoming study, an average correlation assessment may be performed. After the RMA matrix is generated (genes by samples), a Pearson correlation matrix is calculated of the samples to each other. This matrix is samples by samples. For each sample row of the matrix, the mean of all correlation values in that row of the matrix, excluding the diagonal is calculated (which is always 1). This mean is the average correlation for that sample. If the average correlation is less than a threshold (for instance 0.90), the sample is flagged as a potential outlier. This process is repeated for each row (sample) in the study. Outliers flagged by the average correlation QC check are dropped out of any downstream normalization, prediction or compound similarity steps in the process.

In the cardiotoxicity models of Table 2, the cut-off prediction scores range from about 0.80 to about 1.41, as indicated above. If a sample score, when compared to a particular cardiotoxicity model, e.g. the arrhythmia pathology model, is about 1.25 or above, it can be predicted that the sample shows a toxic response after exposure to the test compound. If the sample score is below 1.25, it can be predicted that the sample does not show a toxic response.

Compound similarity is assessed in the following way. In the same manner as described above, a cohort fold change vector for each study/time-point/compound/dose combination is calculated. This vector is reduced to only the fragments used in the PLS predictive models. We then calculate Pearson correlations of that cohort fold change vector to each subsetted cohort vector in our reference database. Finally, Pearson correlations are calculated ranked from highest to lowest and the results are stored in the toxicity model and reported.

A report may be generated comprising information or data related to the results of the methods of predicting at least one toxic effect. The report may comprise information related to the toxic effects predicted by the comparison of at least one sample prediction score to at least one toxicity reference prediction score from the database. The report may also present information concerning the nucleic acid hybridization data, such as the integrity of the data as well as information inputted by the user of the database and methods of the invention, such as information used to annotate the nucleic acid hybridization data. See PCT US02/22701 for a non-limiting example of a toxicity report that may be generated.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

TABLE 1

| SEQ ID NO. | GLGC ID No. | Fragment Name | GenBank Acc or RefSeq ID No. | Entrez Gene ID | Gene Name | Unigene ID |
|---|---|---|---|---|---|---|
| 1 | 77058 | A03913cds_s_at | A03913 | | | |
| 2 | 77023 | AA684963_at | AA684963 | 293702 | similar to binding protein | Rn.65866 |
| 3 | 77024 | AA685112_at | AA685112 | 293652 | NADH dehydrogenase (ubiquinone) Fe—S protein 8 (predicted) | Rn.3373 |
| 4 | 77026 | AA685376_f_at | AA685376 | | | Rn.25165 |
| 5 | 77028 | AA685903_at | NM_001012197 | 362862 | tumor rejection antigen gp96 (predicted) | Rn.101146 |
| 6 | 77029 | AA686031_at | NM_001005550 | 301458 | NADH dehydrogenase (ubiquinone) Fe—S protein 1, 75 kDa | Rn.1467 |
| 7 | 77030 | AA686579_at | NM_001009672 | 301442 | SMT3 suppressor of mif two 3 homolog 1 (yeast) (predicted) | Rn.1221 |
| 8 | 77032 | AA686870_f_at | AA686870 | | | Rn.25165 |
| 9 | 82173 | AA799389_at | NM_031091 | 81755 | RAB3B, member RAS oncogene family | Rn.3788 |
| 9 | 82174 | AA799389_g_at | NM_031091 | 81755 | RAB3B, member RAS oncogene family | Rn.3788 |
| 10 | 82240 | AA801441_at | NM_031763 | 83572 | platelet-activating factor acetylhydrolase, isoform Ib, alpha subunit 45 kDa | Rn.5827 |
| 11 | 77033 | AA819943_at | AA819943 | | | |
| 12 | 77036 | AA848421_at | AA848421 | 304577 | uracil-DNA glycosylase (predicted) | Rn.104379 |
| 13 | 77037 | AA848546_at | NM_001009542 | 494345 | similar to programmed cell death 10 | Rn.1176 |
| 14 | 76160 | rc_AA818604_s_at | NM_212504 | 294254 | heat shock 70 kD protein 1B | Rn.1950 |
| 14 | 76161 | AA848563_s_at | NM_212504 | 294254 | heat shock 70 kD protein 1B | Rn.1950 |
| 14 | 76534 | L16764_s_at | NM_212504 | 294254 | heat shock 70 kD protein 1B | Rn.1950 |
| 14 | 76766 | Z75029_s_at | NM_212504 | 294254 | heat shock 70 kD protein 1B | Rn.1950 |
| 14 | 79333 | Z27118cds_s_at | NM_212504 | 294254 | heat shock 70 kD protein 1B | Rn.1950 |
| 15 | 77039 | AA875509_at | AA875509 | 314856 | transformed mouse 3T3 cell double minute 2 (predicted) | Rn.91829 |
| 16 | 80380 | AB000098_at | NM_022607 | 64539 | MIPP65 protein | Rn.6452 |
| 17 | 80197 | AB000113_at | NM_017217 | 29485 | solute carrier family 7 (cationic amino acid transporter, y+ system), member 3 | Rn.9804 |
| 18 | 82747 | AB000216_at | NM_134403 | 171440 | ankyrin repeat and BTB (POZ) domain containing 2 | Rn.11149 |
| 19 | 80386 | AB000928_at | NM_053509 | 85271 | zona pellucida glycoprotein 1 | Rn.10890 |
| 20 | 77074 | AB001982_g_at | NM_032075 | 84022 | growth hormone secretagogue receptor | Rn.74241 |
| 21 | 77078 | AB002561_at | NM_021867 | 60464 | fibroblast growth factor 16 | Rn.44348 |
| 22 | 77080 | AB003357_at | NM_019349 | 54308 | serine/threonine kinase 2 | Rn.33275 |
| 23 | 77082 | AB003478_g_at | NM_133553 | 171079 | UDP-Gal:betaGlcNAc beta 1,3-galactosyltransferase, polypeptide 4 | Rn.24575 |
| 24 | 82743 | AB003515_at | NM_022706 | 64670 | GABA(A) receptor-associated protein like 2 | Rn.64537 |
| 25 | 77204 | AB017140_at | NM_031707 | 29546 | homer homolog 1 (Drosophila) | Rn.37500 |
| 25 | 77607 | AF093268_s_at | NM_031707 | 29546 | homer homolog 1 (Drosophila) | Rn.37500 |
| 25 | 80343 | AB003726_at | NM_031707 | 29546 | homer homolog 1 (Drosophila) | Rn.37500 |
| 26 | 77083 | AB003753cds#1_at | AB003753 | | | |
| 27 | 77099 | AB005541_at | AB005541 | | | |
| 28 | 82881 | AB006138_at | NM_031635 | 58924 | fucosyltransferase 2 (secretor status included) | Rn.10678 |
| 29 | 82878 | AB006450_at | NM_019351 | 54311 | translocator of inner mitochondrial membrane 17a (yeast) | Rn.22514 |
| 30 | 82879 | AB006451_at | NM_019352 | 54312 | translocase of inner mitochondrial membrane 23 homolog (yeast) | Rn.2230 |
| 31 | 77104 | AB006710_s_at | NM_057135 | 117276 | 6-phosphofructo-2-kinase/fructose-2,6-biphosphatase 3 | Rn.10791 |
| 32 | 77105 | AB006802_at | AB006802 | | | |
| 33 | 77107 | AB006881mRNA_at | NM_147207 | 259227 | ischemia related factor vof-16 | Rn.38750 |
| 34 | 77111 | AB007689_at | NM_053309 | 29547 | homer homolog 2 (Drosophila) | Rn.30014 |
| 35 | 82920 | AB008538_at | NM_031753 | 79559 | activated leukocyte cell adhesion molecule | Rn.5789 |
| 36 | 77135 | AB010154_at | NM_147135 | 113907 | SH3-binding kinase | Rn.42909 |
| 37 | 77141 | AB010963_s_at | NM_019273 | 29747 | potassium large conductance calcium-activated channel, subfamily M, beta | Rn.10820 |
| | | | | | member 1 | |
| 38 | 77155 | AB011679_at | NM_173102 | 29214 | tubulin, beta 5 | Rn.2458 |
| 38 | 83224 | rc_AA860030_s_at | NM_173102 | 29214 | tubulin, beta 5 | Rn.2458 |
| 39 | 80155 | AB012233_at | AB012233 | 29228 | nuclear factor I/C | Rn.86582 |
| 40 | 80157 | AB012234_g_at | AB012234 | 81524 | nuclear factor I/X | Rn.56776 |
| 41 | 77161 | AB012600_s_at | NM_024362 | 29657 | aryl hydrocarbon receptor nuclear translocator-like | Rn.14532 |
| 41 | 82819 | AF015953_at | NM_024362 | 29657 | aryl hydrocarbon receptor nuclear translocator-like | Rn.14532 |
| 42 | 77162 | AB012933_at | NM_053607 | 94340 | acyl-CoA synthetase long-chain family member 5 | Rn.105862 |
| 43 | 77171 | AB013453_at | NM_013030 | 25548 | solute carrier family 34 (sodium phosphate), member 1 | Rn.92022 |
| 43 | 77173 | AB013455_at | NM_013030 | 25548 | solute carrier family 34 (sodium phosphate), member 1 | Rn.92022 |
| 44 | 77172 | AB013454_at | AB013454 | 360649 | similar to Ac2-210 | Rn.110865 |
| 45 | 77178 | AB015191_at | NM_022505 | 60414 | Rhesus blood group CE and D | Rn.1943 |
| 45 | 77179 | AB015191_g_at | NM_022505 | 60414 | Rhesus blood group CE and D | Rn.1943 |
| 46 | 77190 | AB015946_s_at | NM_145778 | 252921 | tubulin, gamma 1 | Rn.8218 |
| 47 | 77200 | AB016536_s_at | NM_031330 | 83498 | heterogeneous nuclear ribonucleoprotein A/B | Rn.64591 |
| 47 | 81530 | rc_AI104524_s_at | NM_031330 | 83498 | heterogeneous nuclear ribonucleoprotein A/B | Rn.1427 |
| 48 | 77202 | AB017044exon_at | AB017044 | | | |
| 49 | 77206 | AB017170_s_at | NM_022953 | 65047 | slit homolog 1 (Drosophila) | Rn.30002 |
| 50 | 77215 | AB017711_at | NM_031335 | 83503 | polymerase II | Rn.28212 |
| 51 | 77228 | AB020504_g_at | AB020504 | 171453 | PMF32 protein | Rn.13083 |
| 52 | 77235 | AF000900_at | NM_139091 | 245922 | nucleoporin like 1 | Rn.11099 |
| 52 | 81017 | rc_AA891045_at | NM_139091 | 245922 | nucleoporin like 1 | Rn.11099 |
| 53 | 77239 | AF000943_at | NM_022208 | 83830 | general transcription factor 2a, 1 | Rn.44436 |
| 54 | 76391 | AF003523_s_at | NM_022698 | 64639 | bcl2-associated death promoter | Rn.36696 |
| 55 | 77247 | AF004218_s_at | NM_030996 | 29336 | opioid receptor, sigma 1 | Rn.1129 |
| 56 | 82857 | AF004811_at | NM_030863 | 81521 | moesin | Rn.2762 |
| 57 | 77252 | AF008912_at | AF008912 | | | Rn.114289 |
| 58 | 77253 | AF009656mRNA_s_at | NM_012583 | 24465 | hypoxanthine guanine phosphoribosyl transferase | Rn.47 |
| 59 | 82942 | AF014503_at | NM_053611 | 113900 | nuclear protein 1 | Rn.11182 |
| 60 | 80323 | AF016179_at | NM_173315 | 286981 | putative pheromone receptor (Go-VN2) | Rn.10807 |
| 61 | 80326 | AF016182_at | NM_173130 | 286914 | putative pheromone receptor (Go-VN5) | Rn.10810 |
| 62 | 77270 | AF018261_at | NM_057136 | 117277 | Epsin 1 | Rn.30007 |
| 63 | 77271 | AF019624_at | NM_019151 | 29152 | growth differentiation factor 8 | Rn.44460 |
| 64 | 77275 | AF020046_s_at | NM_031768 | 83577 | integrin, alpha E, epithelial-associated | Rn.29975 |
| 65 | 77276 | AF020210_s_at | NM_053655 | 114114 | dynamin 1-like | Rn.10830 |
| 65 | 77277 | AF020211_s_at | NM_053655 | 114114 | dynamin 1-like | Rn.10830 |
| 66 | 78927 | U88324_at | NM_030987 | 24400 | guanine nucleotide binding protein, beta 1 | Rn.126047 |
| 66 | 82918 | AF022083_s_at | NM_030987 | 24400 | guanine nucleotide binding protein, beta 1 | Rn.126047 |
| 67 | 76834 | AF022729_at | NM_080397 | 140568 | carbohydrate sulfotransferase 10 | Rn.11224 |
| 68 | 77284 | AF022742cds_at | NM_031771 | 83580 | thrombomodulin | Rn.88295 |
| 69 | 82949 | AF022774_g_at | NM_133591 | 171123 | rabphilin 3A-like (without C2 domains) | Rn.10986 |
| 70 | 77286 | AF022952_at | AF022952 | 89811 | vascular endothelial growth factor B | Rn.70676 |
| 71 | 77287 | AF023087_s_at | NM_012551 | 24330 | early growth response 1 | Rn.9096 |
| 71 | 78865 | U75397UTR#1_s_at | NM_012551 | 24330 | early growth response 1 | Rn.9096 |
| 72 | 77289 | AF023621_at | AF023621 | 83576 | sortilin 1 | Rn.11286 |
| 73 | 77291 | AF025671_s_at | NM_022522 | 64314 | caspase 2 | Rn.1438 |
| 74 | 80347 | AF026476_at | NM_031777 | 83586 | upstream transcription factor 1 | Rn.10845 |
| 75 | 80348 | AF026529_s_at | AF026529 | 79423 | stathmin-like 4 | Rn.5658 |
| 76 | 77293 | AF027188UTR#1_at | AF027188 | 309037 | similar to RIKEN cDNA 4933424N09 (predicted) | Rn.88644 |
| 77 | 77295 | AF027571_s_at | NM_024353 | 25031 | phospholipase C, beta 4 | Rn.6155 |
| 78 | 77298 | AF028784mRNA#1_s_at | AF028784 | | | |
| 79 | 77300 | AF029357cds_at | NM_001000583 | 366456 | olfactory receptor 857 (predicted) | |
| 80 | 77309 | AF030091UTR#1_g_at | NM_053662 | 114121 | cyclin L1 | Rn.12962 |
| 81 | 77312 | AF030358_g_at | NM_134455 | 89808 | chemokine (C—X3—C motif) ligand 1 | Rn.107266 |
| 82 | 82963 | AF032668_at | NM_019277 | 50556 | SEC15-like 1 (S. cerevisiae) | Rn.1188 |
| 83 | 82964 | AF032669_at | NM_139043 | 245709 | exocyst complex component 8 | Rn.10901 |
| 84 | 77331 | AF034896_f_at | NM_206850 | 294158 | olfactory receptor 1696 | Rn.107045 |
| 85 | 77336 | AF035632_s_at | NM_022939 | 65033 | syntaxin 12 | Rn.54570 |
| 86 | 77339 | AF035952_at | AF035952 | 171291 | kinesin family member 6 | Rn.82703 |
| 87 | 77349 | AF036959_at | NM_031787 | 83617 | homeodomain interacting protein kinase 3 | Rn.8099 |
| 87 | 83287 | rc_AA866455_at | NM_031787 | 83617 | homeodomain interacting protein kinase 3 | Rn.8099 |
| 88 | 82705 | AF037071_at | NM_138922 | 192363 | C-terminal PDZ domain ligand of neuronal nitric oxide synthase | Rn.9903 |
| 89 | 80689 | rc_AI639215_at | NM_131913 | 170751 | X-prolyl aminopeptidase (aminopeptidase P) 1, soluble | Rn.25763 |
| 89 | 82946 | AF038591_at | NM_131913 | 170751 | X-prolyl aminopeptidase (aminopeptidase P) 1, soluble | Rn.25763 |
| 90 | 80379 | AF039085_at | NM_053553 | 89815 | synaptogyrin 2 | Rn.34912 |
| 91 | 77354 | AF039308_at | AF039308 | 83621 | glutaminyl-peptide cyclotransferase (glutaminyl cyclase) | |
| 92 | 82951 | AF040954_at | NM_022951 | 65045 | protein phosphatase 1, regulatory subunit 10 | Rn.37758 |
| 93 | 80384 | AF041107_g_at | AF041107 | 56785 | GTPase activating RANGAP domain-like 1 | Rn.86984 |
| 94 | 77374 | AF044574_at | NM_171996 | 64461 | 2-4-dienoyl-Coenzyme A reductase 2, peroxisomal | Rn.6965 |
| 95 | 82965 | AF044910_at | NM_022509 | 64301 | survival of motor neuron 1, telomeric | Rn.1119 |
| 96 | 77380 | AF048828_at | NM_031353 | 83529 | voltage-dependent anion channel 1 | Rn.54594 |
| 96 | 77381 | AF048828_g_at | NM_031353 | 83529 | voltage-dependent anion channel 1 | Rn.54594 |
| 97 | 77385 | AF050214_at | AF050214 | | | |
| 98 | 77389 | AF050662UTR#1_at | NM_031593 | 29643 | synaptic vesicle glycoprotein 2c | Rn.24210 |
| 98 | 77428 | AF060174_at | NM_031593 | 29643 | synaptic vesicle glycoprotein 2c | Rn.24210 |
| 99 | 77395 | AF051895_at | NM_013132 | 25673 | annexin A5 | Rn.3318 |
| 100 | 77406 | AF053938_s_at | NM_181092 | 192117 | synaptic Ras GTPase activating protein 1 homolog (rat) | Rn.9908 |
| 101 | 80160 | AF053987_at | AF053987 | 297590 | putative pheromone receptor V2R1 | Rn.107049 |
| 102 | 80164 | AF053991_at | NM_173318 | 286984 | putative pheromone receptor (Go-VN4) | Rn.10809 |
| 103 | 77414 | AF055667mRNA_at | AF055667 | | | |
| 104 | 77420 | AF056324_at | NM_022394 | 64196 | scaffold attachment factor B | Rn.88640 |
| 104 | 77421 | AF056324_g_at | NM_022394 | 64196 | scaffold attachment factor B | Rn.88640 |
| 105 | 80256 | AF059258_at | NM_031744 | 65200 | solute carrier family 16, member 8 | Rn.14526 |
| 106 | 82798 | AF059530_at | NM_053557 | 89820 | heterogeneous nuclear ribonucleoprotein methyltransferase-like 3 (S. cerevisiae) | Rn.33389 |
| 107 | 77438 | AF061947_g_at | NM_053575 | 94165 | calcineurin binding protein 1 | Rn.4 |
| 108 | 77441 | AF062594_g_at | NM_053561 | 89825 | nucleosome assembly protein 1-like 1 | Rn.18480 |
| 109 | 77445 | AF062741_g_at | NM_145091 | 246311 | pyruvate dehydrogenase phosphatase isoenzyme 2 | Rn.30021 |
| 110 | 77450 | AF063249_at | NM_022925 | 360417 | protein tyrosine phosphatase, receptor type, Q | Rn.30011 |
| 111 | 82806 | AF063447_at | NM_053563 | 89827 | nuclear RNA helicase, DECD variant of DEAD box family | Rn.14550 |
| 112 | 76776 | U69109_s_at | NM_017318 | 50646 | protein tyrosine kinase 2 beta | Rn.11025 |
| 112 | 77453 | AF063890_s_at | NM_017318 | 50646 | protein tyrosine kinase 2 beta | Rn.11025 |
| 113 | 77456 | AF064868_g_at | NM_024163 | 79146 | brain-enriched guanylate kinase-associated | Rn.30036 |
| 114 | 77460 | AF065387_at | NM_031756 | 81716 | gamma-glutamyl carboxylase | Rn.22410 |
| 115 | 77473 | AF071014mRNA_s_at | NM_024483 | 29413 | adrenergic receptor, alpha 1d | Rn.11314 |
| 116 | 77480 | AF072892_s_at | AF072892 | 114122 | chondroitin sulfate proteoglycan 2 | Rn.35666 |
| 117 | 77481 | AF072935_at | NM_022692 | 64633 | RAB5A, member RAS oncogene family | Rn.44477 |
| 118 | 77483 | AF073891_at | NM_133610 | 171146 | potassium voltage-gated channel, subfamily H (eag-related), member 5 | Rn.48753 |
| 119 | 77491 | AF076183_at | NM_134406 | 171444 | phosphofurin acidic cluster sorting protein 1 | Rn.105884 |
| 119 | 77493 | AF076184_at | NM_134406 | 171444 | phosphofurin acidic cluster sorting protein 1 | Rn.105884 |
| 120 | 77494 | AF076619_at | NM_031623 | 58844 | growth factor receptor bound protein 14 | Rn.30028 |
| 121 | 77496 | AF077000_at | NM_057204 | 117552 | protein tyrosine phosphatase, non-receptor type 23 | Rn.54442 |
| 121 | 77497 | AF077000_g_at | NM_057204 | 117552 | protein tyrosine phosphatase, non-receptor type 23 | Rn.54442 |
| 122 | 77507 | AF079864_at | NM_173293 | 170816 | olfactory receptor 59 | Rn.12541 |
| 123 | 77518 | AF081196_at | NM_019211 | 29434 | RAS guanyl releasing protein 1 | Rn.32156 |
| 124 | 77530 | AF083269_at | NM_019289 | 54227 | actin related protein 2/3 complex, subunit 1B | Rn.2090 |
| 124 | 77531 | AF083269_g_at | NM_019289 | 54227 | actin related protein 2/3 complex, subunit 1B | Rn.2090 |
| 125 | 77537 | AF084576_at | NM_053666 | 114125 | delta-like 3 (Drosophila) | Rn.23105 |
| 126 | 77546 | AF087037_g_at | NM_019290 | 54230 | B-cell translocation gene 3 | Rn.8897 |
| 127 | 77548 | AF087431_g_at | NM_031749 | 78947 | glucosidase 1 | Rn.22161 |
| 128 | 77551 | AF087454_at | NM_031597 | 29682 | potassium voltage-gated channel, subfamily Q, member 3 | Rn.55340 |
| 129 | 77565 | AF090113_at | NM_138535 | 171571 | glutamate receptor interacting protein 2 | Rn.15680 |
| 130 | 77572 | AF090306_s_at | NM_031816 | 83712 | retinoblastoma binding protein 7 | Rn.3600 |
| 131 | 77576 | AF091561_at | NM_001000504 | 308853 | olfactory receptor 37 | Rn.110414 |
| 132 | 77577 | AF091562_s_at | NM_001000507 | 309225 | olfactory receptor 337 | Rn.110431 |
| 133 | 77582 | AF091566_f_at | NM_001006598 | 363546 | olfactory receptor 1362 | Rn.113183 |
| 134 | 77583 | AF091567_s_at | NM_001000724 | 404977 | olfactory receptor 1468 | Rn.129488 |
| 135 | 77591 | AF091577_at | NM_001000979 | 405334 | olfactory receptor 1370 (predicted) | Rn.124616 |
| 136 | 77595 | AF091580_s_at | AF091580 | 296156 | carboxypeptidase X 1 (M14 family) (predicted) | Rn.19382 |
| 137 | 77603 | AF092523_s_at | NM_053665 | 114124 | A kinase (PRKA) anchor protein 1 | Rn.91372 |
| 138 | 77625 | AF099093_g_at | NM_022690 | 64631 | ubiquitin-conjugating enzyme E2G 1 (UBC7 homolog, C. elegans) | Rn.1854 |
| 139 | 77643 | AJ000347_g_at | NM_171990 | 64473 | 3′(2′),5′-bisphosphate nucleotidase | Rn.8453 |
| 140 | 80387 | AJ000485_at | NM_021997 | 29264 | cytoplasmic linker 2 | Rn.10893 |
| 141 | 77645 | AJ000557cds_s_at | NM_031514 | 24514 | Janus kinase 2 | Rn.18909 |
| 141 | 78719 | U13396_g_at | NM_031514 | 24514 | Janus kinase 2 | Rn.18909 |
| 142 | 77648 | AJ001290cds_at | NM_053715 | 114507 | solute carrier family 5 (inositol transporters), member 3 | Rn.79242 |
| 143 | 77649 | AJ001517cds_at | NM_053301 | 29199 | hemochromatosis | Rn.42931 |
| 144 | 77651 | AJ001637cds_at | NM_013004 | 25512 | phosphate regulating gene with homologies to endopeptidases on the X | Rn.37861 |
| | | | | | chromosome | |
| 145 | 76426 | AJ002942cds_at | AJ002942 | 24706 | retinoic acid receptor, beta | Rn.38896 |
| 146 | 77660 | AJ004858_at | NM_053349 | 84046 | SRY-box containing gene 11 | Rn.12111 |
| 147 | 77666 | AJ005394_at | NM_134452 | 85490 | collagen, type V, alpha 1 | Rn.117 |
| 147 | 83143 | rc_AA859757_g_at | NM_134452 | 85490 | collagen, type V, alpha 1 | Rn.117 |
| 148 | 77670 | AJ005984_at | NM_021842 | 60334 | endosulfine alpha | Rn.27154 |
| 149 | 76430 | AJ006070_at | AJ006070 | 84600 | recombination activating gene 1 | Rn.92344 |
| 150 | 77676 | AJ006519_at | NM_024154 | 79123 | amiloride-sensitive cation channel 2, neuronal | Rn.37385 |
| 151 | 77684 | AJ007291_at | NM_057143 | 117287 | fertility protein SP22 | Rn.30105 |
| 151 | 77685 | AJ007291_g_at | NM_057143 | 117287 | fertility protein SP22 | Rn.30105 |
| 152 | 77686 | AJ007422_s_at | NM_133567 | 171097 | centaurin, alpha 1 | Rn.10539 |
| 153 | 77690 | AJ007632_s_at | NM_145095 | 246325 | potassium voltage-gated channel, subfamily H, member 8 | Rn.30029 |
| 154 | 77699 | AJ011115_g_at | NM_021838 | 24600 | nitric oxide synthase 3, endothelial cell | Rn.44265 |
| 154 | 77700 | AJ011116_at | NM_021838 | 24600 | nitric oxide synthase 3, endothelial cell | Rn.44265 |
| 155 | 77704 | AJ011606_at | NM_053480 | 85242 | polymerase (DNA directed), alpha 2 | Rn.91557 |
| 156 | 77708 | AJ011656cds_s_at | NM_031700 | 65130 | claudin 3 | Rn.4513 |
| 156 | 81868 | M74067_at | NM_031700 | 65130 | claudin 3 | Rn.4513 |
| 157 | 77709 | AJ011811_at | AJ011811 | 65132 | claudin 7 | Rn.32259 |
| 158 | 77713 | AJ012603cds_at | NM_020306 | 57027 | a disintegrin and metalloproteinase domain 17 (tumor necrosis factor, alpha, | Rn.24102 |
| | | | | | converting enzyme) | |
| 158 | 77714 | AJ012603UTR#1_at | NM_020306 | 57027 | a disintegrin and metalloproteinase domain 17 (tumor necrosis factor, alpha, | Rn.24102 |
| | | | | | converting enzyme) | |
| 158 | 77715 | AJ012603UTR#1_g_at | NM_020306 | 57027 | a disintegrin and metalloproteinase domain 17 (tumor necrosis factor, alpha, | Rn.24102 |
| | | | | | converting enzyme) | |
| 159 | 76433 | AJ223083_at | AJ223083 | 83574 | retinoid X receptor gamma | Rn.40816 |
| 160 | 77725 | AJ223355_at | NM_133418 | 170943 | solute carrier family 25 (mitochondrial carrier; dicarboxylate transporter), | Rn.3631 |
| | | | | | member 10 | |
| 161 | 76815 | AJ224120_at | NM_053487 | 85249 | peroxisomal biogenesis factor 11A | Rn.14519 |
| 162 | 77728 | D00092_s_at | D00092 | 290937 | similar to Dihydrolipoamide acetyltransferase component of pyruvate | Rn.122656 |
| | | | | | dehydrogenase complex (E2) (PDC-E2) (70 kDa mitochondrial autoantigen of | |
| | | | | | primary biliary cirrhosis) (PBC) | |
| 163 | 76780 | D00569_at | NM_057197 | 117543 | 2,4-dienoyl CoA reductase 1, mitochondrial | Rn.2854 |
| 163 | 76781 | D00569_g_at | NM_057197 | 117543 | 2,4-dienoyl CoA reductase 1, mitochondrial | Rn.2854 |
| 164 | 79889 | D00634_at | NM_012701 | 24925 | adrenergic receptor, beta 1 | Rn.87064 |
| 165 | 76114 | rc_AI229440_s_at | NM_138877 | 25035 | diaphorase 1 | Rn.35994 |
| 165 | 77732 | D00636cds_s_at | NM_138877 | 25035 | diaphorase 1 | Rn.35994 |
| 166 | 76441 | D00688_s_at | D00688 | 29253 | monoamine oxidase A | Rn.16867 |
| 167 | 79598 | D00729_at | NM_017306 | 29740 | dodecenoyl-coenzyme A delta isomerase | Rn.80835 |
| 168 | 81799 | D00753_at | NM_031531 | 24795 | Serine protease inhibitor | Rn.128 |
| 169 | 77738 | D01046_at | NM_032617 | 79434 | RAB11B, member RAS oncogene family | Rn.3903 |
| 170 | 82782 | D10233_g_at | NM_031095 | 81759 | renin binding protein | Rn.11116 |
| 171 | 82255 | D10262_at | NM_017127 | 29194 | choline kinase alpha | Rn.10985 |
| 172 | 77749 | D10693_at | NM_031044 | 81676 | histamine N-methyltransferase | Rn.13145 |
| 173 | 77751 | D10699_g_at | NM_017237 | 29545 | ubiquitin carboxy-terminal hydrolase L1 | Rn.107213 |
| 174 | 77759 | D10755_s_at | NM_017283 | 29673 | proteasome (prosome, macropain) subunit, alpha type 6 | Rn.107278 |
| 175 | 77765 | D10853_at | D10853 | 117544 | phosphoribosyl pyrophosphate amidotransferase | Rn.18690 |
| 176 | 77769 | D10952_i_at | NM_053586 | 94194 | cytochrome c oxidase subunit Vb | Rn.6686 |
| 177 | 77771 | D11445exon#1-4_s_at | NM_030845 | 81503 | chemokine (C—X—C motif) ligand 1 | Rn.10907 |
| 178 | 77781 | D12771_s_at | NM_057102 | 25176 | solute carrier family 25 (mitochondrial carrier; adenine nucleotide translocator), | Rn.102263 |
| | | | | | member 5 | |
| 179 | 77785 | D12978cds_s_at | D12978 | 171068 | POU domain, class 2, transcription factor 1 | Rn.46306 |
| 180 | 77786 | D13120_s_at | NM_019383 | 56080 | ATP synthase, H+ transporting, mitochondrial F0 complex, subunit d | Rn.80 |
| 181 | 77787 | D13122_i_at | NM_012915 | 25392 | ATPase inhibitor | Rn.1658 |
| 182 | 77790 | D13124_s_at | NM_133556 | 171082 | ATP synthase, H+ transporting, mitochondrial F0 complex, subunit c (subunit | Rn.127811 |
| | | | | | 9), isoform 2 | |
| 183 | 77796 | D13309_s_at | NM_031563 | 29206 | nuclease sensitive element binding protein 1 | Rn.110976 |
| 184 | 82850 | D13376_at | NM_024349 | 24183 | adenylate kinase 1 | Rn.79537 |
| 185 | 77798 | D13417_g_at | NM_024360 | 29577 | hairy and enhancer of split 1 (Drosophila) | Rn.19727 |
| 186 | 82035 | D13518_at | NM_012764 | 25172 | GATA binding protein 1 | Rn.10024 |
| 187 | 77800 | D13556exon_at | D13556 | | | |
| 188 | 77801 | D13623_at | NM_001008280 | 287633 | similar to ribosome-binding protein p34 - rat (predicted) | Rn.2783 |
| 189 | 82985 | D13907_g_at | NM_022395 | 64198 | peptidase (mitochondrial processing) beta | Rn.841 |
| 190 | 77807 | D13927_at | NM_053327 | 79425 | chloride channel K1 | Rn.88871 |
| 190 | 77808 | D13927_g_at | NM_053327 | 79425 | chloride channel K1 | Rn.88871 |
| 191 | 77810 | D13962_g_at | NM_017102 | 25551 | solute carrier family 2 (facilitated glucose transporter), member 3 | Rn.95055 |
| 192 | 77811 | D13963_at | NM_022920 | 24419 | glutamate receptor, metabotropic 6 | Rn.44615 |
| 193 | 77816 | D13985_g_at | NM_031719 | 65160 | chloride channel, nucleotide-sensitive, 1A | Rn.4089 |
| 193 | 81481 | rc_AI169005_at | NM_031719 | 65160 | chloride channel, nucleotide-sensitive, 1A | Rn.4089 |
| 194 | 76448 | D14014_g_at | NM_171992 | 58919 | cyclin D1 | Rn.22279 |
| 195 | 77817 | D14029_at | NM_019236 | 29567 | hairy and enhancer of split 2 (Drosophila) | Rn.44655 |
| 196 | 77819 | D14048_g_at | NM_057139 | 117280 | heterogeneous nuclear ribonucleoprotein U | Rn.4328 |
| 197 | 82979 | D14076_at | NM_138538 | 171574 | testicular dynamin | Rn.11191 |
| 198 | 77828 | D14568_at | NM_017309 | 29748 | protein phospatase 3, regulatory subunit B, alpha isoform, type 1 | Rn.42903 |
| 199 | 82030 | D16302_at | NM_030861 | 81519 | Alpha-1,3-mannosyl-glycoprotein 2-beta-N-acetylglucosaminyltransferase | Rn.2712 |
| 200 | 77839 | D16443_at | NM_012704 | 24929 | prostaglandin E receptor 3 (subtype EP3) | Rn.10361 |
| 200 | 77840 | D16443_g_at | NM_012704 | 24929 | prostaglandin E receptor 3 (subtype EP3) | Rn.10361 |
| 201 | 77842 | D16817_g_at | NM_031040 | 81672 | glutamate receptor, metabotropic 7 | Rn.10409 |
| 202 | 82056 | D17614_at | NM_013053 | 25577 | tyrosine 3-monooxygenase/tryptophan 5-monooxygenase activation protein, | Rn.2502 |
| | | | | | theta polypeptide | |
| 203 | 76462 | D21799_g_at | NM_017284 | 29675 | proteasome (prosome, macropain) subunit, beta type 2 | Rn.1981 |
| 204 | 77855 | D21869_s_at | NM_031715 | 65152 | phosphofructokinase, muscle | Rn.11004 |
| 205 | 76463 | D25233UTR#1_at | D25233 | 24708 | retinoblastoma 1 | Rn.55115 |
| 206 | 77861 | D26111_s_at | NM_173103 | 79430 | chloride channel Kb | Rn.10339 |
| 207 | 77865 | D26393exon_s_at | D26393 | | | |
| 208 | 82419 | D26439_at | NM_017079 | 25109 | CD1d1 antigen | Rn.11120 |
| 209 | 77881 | D28111_g_at | NM_012720 | 25037 | myelin-associated oligodendrocytic basic protein | Rn.91185 |
| 210 | 77883 | D28512_at | NM_019122 | 25731 | synaptotagmin 3 | Rn.48884 |
| 211 | 76467 | D28754_s_at | NM_199501 | 362817 | cyclin dependent kinase 2 | Rn.104460 |
| 212 | 82751 | D29646_at | NM_013127 | 25668 | CD38 antigen | Rn.11414 |
| 213 | 82152 | D29683_at | NM_053596 | 94204 | endothelin converting enzyme 1 | Rn.7000 |
| 214 | 77893 | D29766cds#1_s_at | NM_012931 | 25414 | breast cancer anti-estrogen resistance 1 | Rn.40101 |
| 215 | 77896 | D30649mRNA_s_at | NM_019370 | 54410 | ectonucleotide pyrophosphatase/phosphodiesterase 3 | Rn.44 |
| 216 | 82555 | D30735_at | NM_013222 | 27100 | growth factor, erv1-like | Rn.11039 |
| 217 | 82531 | D37920_at | NM_017136 | 29230 | squalene epoxidase | Rn.33239 |
| 218 | 79730 | D37934_at | D37934 | | | |
| 219 | 77914 | D38036_at | D38036 | | | |
| 220 | 77916 | D38061exon_s_at | NM_057105 | 113992 | UDP glycosyltransferase 1 family, polypeptide A6 | Rn.26489 |
| 220 | 78466 | S56937_s_at | NM_057105 | 113992 | UDP glycosyltransferase 1 family, polypeptide A6 | Rn.26489 |
| 221 | 77918 | D38063exon_at | NM_175846 | 301595 | UDP glycosyltransferase 1 family, polypeptide A8 | Rn.26489 |
| 222 | 77921 | D38067exon_at | D38067 | | | |
| 223 | 82871 | D38468_s_at | NM_013016 | 25528 | protein tyrosine phosphatase, non-receptor type substrate 1 | Rn.53971 |
| 224 | 77926 | D38494_at | NM_017292 | 29695 | gamma-aminobutyric acid A receptor, rho 2 | Rn.48659 |
| 225 | 77929 | D42116_s_at | NM_001005330 | 54243 | complement receptor related protein | Rn.5825 |
| 225 | 82202 | L36532_s_at | NM_001005330 | 54243 | complement receptor related protein | Rn.5825 |
| 226 | 77931 | D42148_at | NM_057100 | 58935 | growth arrest specific 6 | Rn.52228 |
| 227 | 77938 | D45247_at | D45247 | 29425 | proteasome (prosome, macropain) subunit, beta type 5 | Rn.2 |
| 227 | 77939 | D45247_g_at | D45247 | 29425 | proteasome (prosome, macropain) subunit, beta type 5 | Rn.2 |
| 228 | 77945 | D45254_g_at | NM_022598 | 64530 | cellular nucleic acid binding protein 1 | Rn.6187 |
| 229 | 79794 | D45413_at | D45413 | 171125 | protein tyrosine phosphatase, receptor type, H | Rn.10285 |
| 230 | 77948 | D45862_g_at | NM_013076 | 25608 | leptin | Rn.44444 |
| 231 | 82654 | D49434_at | D49434 | 25227 | arylsulfatase B | Rn.94004 |
| 232 | 80069 | D50093_s_at | NM_012631 | 24686 | prion protein | Rn.3936 |
| 233 | 81357 | rc_AI044488_at | NM_017126 | 29189 | ferredoxin 1 | Rn.6946 |
| 233 | 82653 | D50436_at | NM_017126 | 29189 | ferredoxin 1 | Rn.6946 |
| 234 | 82514 | D50694_at | NM_033236 | 25581 | proteasome (prosome, macropain) 26S subunit, ATPase 2 | Rn.1202 |
| 235 | 82516 | D50695_at | NM_057122 | 117262 | proteasome (prosome, macropain) 26S subunit, ATPase 4 | Rn.11341 |
| 236 | 82515 | D50696_at | NM_057123 | 117263 | peptidase (prosome, macropain) 26S subunit, ATPase 1 | Rn.10526 |
| 237 | 76786 | D63673_at | NM_057125 | 117265 | peroxisomal biogenesis factor 6 | Rn.10675 |
| 238 | 76837 | D63761_g_at | NM_024153 | 79122 | ferredoxin reductase | Rn.10860 |
| 239 | 82703 | D63772_at | NM_013032 | 25550 | solute carrier family 1 (neuronal/epithelial high affinity glutamate transporter, | Rn.6384 |
| | | | | | system Xag), member 1 | |
| 240 | 80296 | D64046_g_at | NM_022185 | 29741 | phosphatidylinositol 3-kinase, regulatory subunit, polypeptide 2 | Rn.22497 |
| 241 | 82225 | D64050_at | NM_053594 | 94202 | protein tyrosine phosphatase, receptor type, R | Rn.6277 |
| 242 | 77970 | D64085_g_at | NM_022211 | 60662 | fibroblast growth factor 5 | Rn.44445 |
| 243 | 80129 | D82868_at | NM_019326 | 54276 | neurogenic differentiation 2 | Rn.10724 |
| 244 | 82508 | D83349_at | NM_019161 | 29182 | cadherin 22 | Rn.108785 |
| 245 | 79979 | D83598_at | NM_013040 | 25560 | ATP-binding cassette, sub-family C (CFTR/MRP), member 9 | Rn.10528 |
| 246 | 77984 | D84485_at | D84485 | 362600 | ADP-ribosylhydrolase like 2 (predicted) | Rn.1834 |
| 247 | 82892 | D85189_at | NM_053623 | 113976 | acyl-CoA synthetase long-chain family member 4 | Rn.87821 |
| 248 | 77988 | D85435_at | NM_134449 | 85332 | protein kinase C, delta binding protein | Rn.12281 |
| 248 | 77989 | D85435_g_at | NM_134449 | 85332 | protein kinase C, delta binding protein | Rn.12281 |
| 249 | 76759 | D86215_at | NM_012985 | 25488 | NADH dehydrogenase (ubiquinone) 1 alpha subcomplex 5 | Rn.100240 |
| 250 | 82830 | D86711_at | D86711 | 362156 | similar to mKIAA1748 protein | Rn.4240 |
| 251 | 77993 | D86745cds_s_at | NM_057133 | 117274 | nuclear receptor subfamily 0, group B, member 2 | Rn.10712 |
| 252 | 82643 | D87515_at | NM_031097 | 81761 | arginyl aminopeptidase (aminopeptidase B) | Rn.10979 |
| 253 | 80119 | D87748_at | NM_017179 | 29375 | Unc4.1 homeobox (C. elegans) | Rn.10710 |
| 254 | 77996 | D87922exon_at | D87922 | | | |
| 255 | 77997 | D87991_at | NM_199081 | 287642 | galactose transporter | Rn.124916 |
| 255 | 77998 | D87991_g_at | NM_199081 | 287642 | galactose transporter | Rn.124916 |
| 256 | 82666 | D88666_at | NM_138882 | 85311 | phosphatidylserine-specific phospholipase A1 | Rn.10696 |
| 257 | 80060 | D88751_at | NM_013100 | 25637 | prostaglandin E receptor 1 | Rn.11423 |
| 258 | 76489 | D89069_f_at | NM_019170 | 29224 | carbonyl reductase 1 | Rn.3425 |
| 258 | 82598 | X95986mRNA#1_f_at | NM_019170 | 29224 | carbonyl reductase 1 | Rn.3425 |
| 259 | 76490 | D89070cds_s_at | D89070 | | | |
| 260 | 82923 | D89983_at | NM_022585 | 58961 | ornithine decarboxylase antizyme inhibitor | Rn.6290 |
| 261 | 81175 | rc_AA946532_at | NM_012804 | 25270 | ATP-binding cassette, sub-family D (ALD), member 3 | Rn.7024 |
| 261 | 82757 | D90038_at | NM_012804 | 25270 | ATP-binding cassette, sub-family D (ALD), member 3 | Rn.7024 |
| 262 | 78004 | D90258_s_at | NM_017280 | 29670 | proteasome (prosome, macropain) subunit, alpha type 3 | Rn.3997 |
| 263 | 78005 | D90265_s_at | NM_017278 | 29668 | proteasome (prosome, macropain) subunit, alpha type 1 | Rn.2668 |
| 264 | 78009 | D90404_g_at | NM_017097 | 25423 | cathepsin C | Rn.122504 |
| 265 | 79398 | E00988mRNA_s_at | NM_031511 | 24483 | insulin-like growth factor 2 | Rn.118681 |
| 266 | 76108 | rc_AI137856_s_at | NM_031576 | 29441 | P450 (cytochrome) oxidoreductase | Rn.11359 |
| 266 | 76549 | M10068mRNA_s_at | NM_031576 | 29441 | P450 (cytochrome) oxidoreductase | Rn.11359 |
| 266 | 79394 | E01524cds_s_at | NM_031576 | 29441 | P450 (cytochrome) oxidoreductase | Rn.11359 |
| 267 | 79390 | E01884cds_s_at | NM_031512 | 24494 | interleukin 1 beta | Rn.9869 |
| 268 | 79388 | E02223cds_f_at | NM_012548 | 24323 | endothelin 1 | Rn.10918 |
| 269 | 76498 | E03344cds_s_at | NM_017234 | 29534 | peroxisomal membrane protein 3 | Rn.4065 |
| 270 | 79380 | E03424cds_s_at | NM_024356 | 29244 | GTP cyclohydrolase 1 | Rn.28195 |
| 271 | 79366 | E12275cds_s_at | NM_031049 | 81681 | lanosterol synthase | Rn.10211 |
| 272 | 79363 | E12625cds_at | NM_080886 | 140910 | sterol-C4-methyl oxidase-like | Rn.7167 |
| 273 | 79358 | E13541cds_s_at | NM_019284 | 50568 | chondroitin sulfate proteoglycan 5 | Rn.10146 |
| 274 | 79356 | E13573cds_s_at | NM_057130 | 117271 | BH3 interacting (with BCL2 family) domain, apoptosis agonist | Rn.89639 |
| 275 | 79354 | E13732cds_at | NM_020542 | 57301 | macrophage inflammatory protein-1 alpha receptor gene | Rn.34673 |
| 276 | 76501 | J00728cds_f_at | J00728 | | | |
| 277 | 78012 | J00735_g_at | NM_012559 | 24367 | fibrinogen, gamma polypeptide | Rn.1702 |
| 277 | 78973 | X05861exon#1-6_s_at | NM_012559 | 24367 | fibrinogen, gamma polypeptide | Rn.1702 |
| 278 | 78017 | J00776cds_s_at | NM_207593 | 293731 | prostatic steroid-binding protein C2 | Rn.10394 |
| 279 | 78025 | J02596cds_g_at | NM_012501 | 24207 | apolipoprotein C-III | Rn.36813 |
| 280 | 79531 | J02675_at | NM_013024 | 25540 | spermine binding protein | Rn.9914 |
| 281 | 76104 | J02679_s_at | NM_017000 | 24314 | NAD(P)H dehydrogenase, quinone 1 | Rn.11234 |
| 282 | 82783 | J02962_at | NM_031832 | 83781 | lectin, galactose binding, soluble 3 | Rn.764 |
| 283 | 78027 | J02998_at | NM_031090 | 81754 | RAB1, member RAS oncogene family | Rn.106743 |
| 284 | 78998 | X08056cds_s_at | NM_012793 | 25257 | guanidinoacetate methyltransferase | Rn.33890 |
| 284 | 83017 | J03588_at | NM_012793 | 25257 | guanidinoacetate methyltransferase | Rn.33890 |
| 285 | 82029 | J03819_at | NM_012672 | 24831 | thyroid hormone receptor beta | Rn.88692 |
| 286 | 82766 | J04024_at | NM_017290 | 29693 | ATPase, Ca++ transporting, cardiac muscle, slow twitch 2 | Rn.2305 |
| 287 | 82122 | J04197_i_at | NM_012621 | 24638 | 6-phosphofructo-2-kinase/fructose-2,6-biphosphatase 1 | Rn.10115 |
| 288 | 79491 | J04503_at | NM_017038 | 24666 | protein phosphatase 1A, magnesium dependent, alpha isoform | Rn.37403 |
| 289 | 81903 | J04532_at | NM_022507 | 25522 | protein kinase C, zeta | Rn.1109 |
| 290 | 78277 | M25350_s_at | NM_017031 | 24626 | phosphodiesterase 4B | Rn.37733 |
| 290 | 82065 | J04563_at | NM_017031 | 24626 | phosphodiesterase 4B | Rn.37733 |
| 290 | 84558 | rc_AA799729_at | NM_017031 | 24626 | phosphodiesterase 4B | Rn.37733 |
| 290 | 84559 | rc_AA799729_g_at | NM_017031 | 24626 | phosphodiesterase 4B | Rn.37733 |
| 291 | 82978 | J04591_at | NM_012789 | 25253 | dipeptidylpeptidase 4 | Rn.91364 |
| 292 | 80223 | J04731_at | NM_012970 | 25468 | potassium voltage-gated channel, shaker-related subfamily, member 2 | Rn.10298 |
| 293 | 82069 | J04792 at | NM_012615 | 24609 | ornithine decarboxylase 1 | Rn.874 |
| 294 | 78032 | J04807mRNA_i_at | NM_019130 | 24506 | insulin 2 | Rn.989 |
| 295 | 82769 | J04943_at | NM_012992 | 25498 | nucleophosmin 1 | Rn.54537 |
| 296 | 76874 | J05181_at | NM_012815 | 25283 | glutamate-cysteine ligase, catalytic subunit | Rn.8365 |
| 297 | 76521 | J05425cds_s_at | NM_017202 | 29445 | cytochrome c oxidase subunit IV isoform 1 | Rn.2528 |
| 298 | 79529 | J05571_s_at | NM_134351 | 171347 | methionine adenosyltransferase II, alpha | Rn.41420 |
| 299 | 81901 | J05592_at | NM_022676 | 58977 | protein phosphatase 1, regulatory (inhibitor) subunit 1A | Rn.9756 |
| 299 | 81902 | J05592_g_at | NM_022676 | 58977 | protein phosphatase 1, regulatory (inhibitor) subunit 1A | Rn.9756 |
| 300 | 81324 | rc_AI013194_at | NM_020075 | 56783 | eukaryotic translation initiation factor 5 | Rn.40123 |
| 300 | 81643 | rc_AI177986_at | NM_020075 | 56783 | eukaryotic translation initiation factor 5 | Rn.40123 |
| 300 | 81949 | K01677_at | NM_020075 | 56783 | eukaryotic translation initiation factor 5 | Rn.40123 |
| 301 | 80428 | K01933_at | NM_012582 | 24464 | haptoglobin | Rn.10950 |
| 302 | 81866 | K02816_g_at | NM_001009618 | 192269 | RNA polymerase II transcriptional coactivator | Rn.8706 |
| 303 | 81837 | K03242_at | NM_017027 | 24564 | myelin protein zero | Rn.8997 |
| 304 | 78051 | K03468_s_at | NM_012604 | 24583 | myosin, heavy polypeptide 3, skeletal muscle, embryonic | Rn.98847 |
| 305 | 79785 | L00124_at | NM_012553 | 24332 | elastase 2 | Rn.10272 |
| 306 | 78053 | L00131cds_s_at | NM_012729 | 25052 | protease, serine, 2 | Rn.1584 |
| 307 | 79481 | L01507_at | NM_013008 | 25517 | POU domain, class 1, transcription factor 1 | Rn.10445 |
| 308 | 81912 | L01702_at | NM_012763 | 25167 | protein tyrosine phosphatase, receptor type, A | Rn.18043 |
| 309 | 78057 | L01793_at | NM_031043 | 81675 | glycogenin 1 | Rn.3661 |
| 309 | 82733 | U96130_at | NM_031043 | 81675 | glycogenin 1 | Rn.3661 |
| 310 | 80221 | L02315_at | L02315 | 58942 | calcium channel, voltage-dependent, beta 4 subunit | Rn.9863 |
| 311 | 82997 | L02529_at | NM_021266 | 58868 | frizzled homolog 1 (Drosophila) | Rn.6575 |
| 312 | 76530 | L02926_s_at | NM_012854 | 25325 | interleukin 10 | Rn.9868 |
| 312 | 79984 | X60675_at | NM_012854 | 25325 | interleukin 10 | Rn.9868 |
| 313 | 76623 | L03294_at | NM_012598 | 24539 | lipoprotein lipase | Rn.3834 |
| 313 | 81788 | rc_AI237731_s_at | NM_012598 | 24539 | lipoprotein lipase | Rn.3834 |
| 314 | 81888 | L03382_at | NM_022707 | 64672 | phospholamban | Rn.9740 |
| 315 | 82146 | L05489_at | NM_012945 | 25433 | diphtheria toxin receptor | Rn.10148 |
| 316 | 79422 | L06804_at | L06804 | 296706 | LIM homeobox protein 2 (predicted) | Rn.81063 |
| 317 | 82020 | L07073_at | NM_133593 | 171126 | adaptor-related protein complex 3, mu 1 subunit | Rn.10959 |
| 318 | 82021 | L07074_at | NM_133305 | 140667 | adaptor-related protein complex 3, mu 2 subunit | Rn.11007 |
| 319 | 78074 | L07380_g_at | NM_012850 | 25321 | growth hormone releasing hormone recepor | Rn.10936 |
| 320 | 78080 | L07402_f_at | L07402 | | | Rn.119903 |
| 321 | 78082 | L07408_at | L07408 | | | Rn.117930 |
| 322 | 78084 | L07410_at | L07410 | | | Rn.117938 |
| 323 | 78085 | L07925_at | NM_019250 | 29622 | ral guanine nucleotide dissociation stimulator | Rn.40174 |
| 323 | 78086 | L07925_g_at | NM_019250 | 29622 | ral guanine nucleotide dissociation stimulator | Rn.40174 |
| 324 | 78087 | L08228exon#22_s_at | NM_017010 | 24408 | glutamate receptor, ionotropic, N-methyl D-aspartate 1 | Rn.9840 |
| 324 | 80281 | U11418_s_at | NM_017010 | 24408 | glutamate receptor, ionotropic, N-methyl D-aspartate 1 | Rn.9840 |
| 325 | 78093 | L08495cds_s_at | NM_021841 | 29708 | gamma-aminobutyric acid A receptor, alpha 6 | Rn.96305 |
| 326 | 78094 | L08496cds_s_at | NM_017289 | 29689 | gamma-aminobutyric acid A receptor, delta | Rn.10927 |
| 327 | 82058 | L11319_at | NM_031723 | 65166 | signal peptidase complex 18 kD | Rn.13070 |
| 328 | 79571 | L11694_at | NM_017033 | 24645 | phosphoglucomutase 1 | Rn.9970 |
| 328 | 84381 | rc_AA894296_at | NM_017033 | 24645 | phosphoglucomutase 1 | Rn.9970 |
| 329 | 82641 | L12025_at | NM_017076 | 25066 | poliovirus receptor | Rn.10677 |
| 330 | 82726 | L12383_at | NM_024151 | 79120 | ADP-ribosylation factor 4 | Rn.35935 |
| 331 | 78109 | L13039_s_at | NM_019905 | 56611 | annexin A2 | Rn.90546 |
| 332 | 78122 | L13407_i_at | NM_012519 | 24246 | calcium/calmodulin-dependent protein kinase II, delta | Rn.87208 |
| 333 | 78124 | L13619_at | NM_022392 | 64194 | insulin induced gene 1 | Rn.772 |
| 334 | 78129 | L14003UTR#1_f_at | NM_012723 | 25046 | polymeric immunoglobulin receptor | Rn.54456 |
| 335 | 78133 | L14462_at | NM_019220 | 29466 | amino-terminal enhancer of split | Rn.11495 |
| 336 | 78136 | L14937cds#1_s_at | NM_133559 | 171085 | proprotein convertase subtilisin/kexin type 4 | Rn.2899 |
| 336 | 78137 | L14937cds#2_s_at | NM_133559 | 171085 | proprotein convertase subtilisin/kexin type 4 | Rn.2899 |
| 337 | 81165 | rc_AA946044_s_at | NM_030857 | 81515 | Yamaguchi sarcoma viral (v-yes-1) oncogene homolog | Rn.4338 |
| 337 | 81831 | L14951_at | NM_030857 | 81515 | Yamaguchi sarcoma viral (v-yes-1) oncogene homolog | Rn.4338 |
| 338 | 82053 | L16532_at | NM_012809 | 25275 | cyclic nucleotide phosphodiesterase 1 | Rn.31762 |
| 339 | 79985 | L17138_at | NM_017265 | 29632 | hydroxy-delta-5-steroid dehydrogenase, 3 beta- and steroid delta-isomerase 1 | Rn.109394 |
| 340 | 76217 | rc_AA893328_at | NM_172008 | 29144 | calnexin | Rn.1762 |
| 340 | 76535 | L18889_at | NM_172008 | 29144 | calnexin | Rn.1762 |
| 341 | 81921 | L19698_at | NM_031093 | 81757 | v-ral simian leukemia viral oncogene homolog A (ras related) | Rn.100380 |
| 342 | 80288 | L20468_at | NM_138511 | 171517 | glypican 2 (cerebroglycan) | Rn.88131 |
| 343 | 76875 | M32167_at | NM_031836 | 83785 | vascular endothelial growth factor A | Rn.1923 |
| 343 | 78149 | L20913_s_at | NM_031836 | 83785 | vascular endothelial growth factor A | Rn.1923 |
| 344 | 82209 | L22760_at | NM_019185 | 29300 | GATA binding protein 6 | Rn.8701 |
| 344 | 84783 | rc_AA800786_at | NM_019185 | 29300 | GATA binding protein 6 | Rn.8701 |
| 345 | 81900 | L22761_at | NM_144730 | 54254 | GATA binding protein 4 | Rn.26251 |
| 346 | 81867 | L24051_at | NM_053820 | 116543 | early B-cell factor 1 | Rn.11257 |
| 347 | 79745 | L24389_at | L24389 | | | |
| 348 | 79563 | L24776_at | NM_057208 | 117557 | tropomyosin 3, gamma | Rn.37575 |
| 349 | 76634 | L24896_s_at | NM_017165 | 29328 | glutathione peroxidase 4 | Rn.3647 |
| 350 | 79650 | L24897_s_at | L24897 | 29605 | myosin, heavy polypeptide 13, skeletal muscle | Rn.97830 |
| 351 | 82256 | L24907_at | NM_134468 | 171503 | calcium/calmodulin-dependent protein kinase I | Rn.11018 |
| 352 | 82405 | L25785_at | L25785 | 25564 | transforming growth factor beta 1 induced transcript 4 | Rn.3545 |
| 353 | 79890 | L26293_at | L26293 | | | Rn.114012 |
| 354 | 79592 | L27075_at | NM_016987 | 24159 | ATP citrate lyase | Rn.29771 |
| 355 | 80226 | L27663_at | L27663 | 29588 | POU domain, class 3, transcription factor 2 | Rn.82733 |
| 356 | 82033 | L29281_at | NM_019335 | 54287 | Protein kinase, interferon-inducible double stranded RNA dependent | Rn.10022 |
| 357 | 78173 | L29573_s_at | NM_031343 | 83511 | solute carrier family 6 (neurotransmitter transporter, noradrenalin), member 2 | Rn.14577 |
| 358 | 81857 | L31619_g_at | NM_012832 | 25302 | cholinergic receptor, nicotinic, alpha polypeptide 7 | Rn.9698 |
| 359 | 78177 | L31622_at | NM_019297 | 54239 | cholinergic receptor, nicotinic, beta polypeptide 2 (neuronal) | Rn.53978 |
| 360 | 76093 | rc_AI070295_at | NM_024127 | 25112 | growth arrest and DNA-damage-inducible 45 alpha | Rn.10250 |
| 360 | 76094 | rc_AI070295_g_at | NM_024127 | 25112 | growth arrest and DNA-damage-inducible 45 alpha | Rn.10250 |
| 360 | 76542 | L32591mRNA_g_at | NM_024127 | 25112 | growth arrest and DNA-damage-inducible 45 alpha | Rn.10250 |
| 361 | 78178 | L34074cds_at | NM_021860 | 60451 | olfactory receptor 1654 (predicted) | |
| 362 | 82025 | L34262_at | NM_022502 | 29411 | palmitoyl-protein thioesterase | Rn.1574 |
| 363 | 78179 | L34542cds_at | NM_021859 | 60450 | megakaryocyte-associated tyrosine kinase | Rn.44303 |
| 364 | 79631 | L36029_at | NM_024380 | 79222 | guanylyl cyclase 2e | Rn.10060 |
| 365 | 79632 | L36030_at | NM_053831 | 116556 | guanylate cyclase 2f | Rn.10061 |
| 366 | 78181 | L36664_s_at | NM_012544 | 24310 | angiotensin 1 converting enzyme | Rn.10149 |
| 367 | 76686 | L38437_at | NM_019223 | 29478 | NADH dehydrogenase (ubiquinone) Fe—S protein 6 | Rn.127777 |
| 368 | 82063 | L39991_at | L39991 | 81738 | nucleoporin 98 | Rn.11324 |
| 369 | 78187 | L41684mRNA_at | NM_031819 | 83720 | fat tumor suppressor homolog (Drosophila) | Rn.8633 |
| 370 | 78193 | L47281_at | L47281 | 116559 | collagen, type IV, alpha 3 | Rn.121139 |
| 371 | 76548 | L48209_s_at | L48209 | 171335 | cytochrome c oxidase, subunit VIIIa | Rn.2270 |
| 372 | 79441 | M10094_g_at | NM_001008830 | 309627 | RT1 class I, A3 | Rn.128901 |
| 373 | 81871 | M11266_at | NM_013078 | 25611 | ornithine transcarbamylase | Rn.2391 |
| 374 | 76552 | M11942_s_at | NM_024351 | 24468 | heat shock protein 8 | Rn.120392 |
| 375 | 83025 | M12156_at | NM_017248 | 29578 | heterogeneous nuclear ribonucleoprotein A1 | Rn.25771 |
| 375 | 84619 | rc_AA799893_g_at | NM_017248 | 29578 | heterogeneous nuclear ribonucleoprotein A1 | Rn.25771 |
| 376 | 78205 | M12335expandedCDS_s_at | M12335 | | | |
| 377 | 80420 | M12579_at | NM_012767 | 25194 | gonadotropin-releasing hormone 1 | Rn.59459 |
| 378 | 81895 | M13750_at | NM_017036 | 24656 | prolactin-like protein A | Rn.11230 |
| 379 | 83014 | M13979_at | NM_138827 | 24778 | solute carrier family 2 (facilitated glucose transporter), member 1 | Rn.3205 |
| 380 | 81869 | M14656_at | NM_012881 | 25353 | secreted phosphoprotein 1 | Rn.8871 |
| 381 | 76117 | rc_AA945573_f_at | NM_017158 | 29298 | cytochrome P450, family 2, subfamily c, polypeptide 7 | Rn.1247 |
| 381 | 78216 | M14775_s_at | NM_017158 | 29298 | cytochrome P450, family 2, subfamily c, polypeptide 7 | Rn.1247 |
| 382 | 82685 | M15327_at | NM_019286 | 24172 | alcohol dehydrogenase 1 | Rn.40222 |
| 383 | 80435 | M15481_g_at | NM_178866 | 24482 | insulin-like growth factor 1 | Rn.6282 |
| 384 | 78224 | M15528mRNA_at | M15528 | | | Rn.37410 |
| 385 | 80267 | M15882_at | NM_031974 | 83800 | clathrin, light polypeptide (Lca) | Rn.112599 |
| 386 | 78226 | M16406cds_s_at | NM_080773 | 25229 | cholinergic receptor, muscarinic 1 | |
| 387 | 80086 | M16409_at | M16409 | 25111 | cholinergic receptor, muscarinic 4 | Rn.10676 |
| 388 | 79464 | M16410_at | NM_019162 | 29191 | tachykinin 2 | Rn.9708 |
| 389 | 78228 | M17523mRNA_at | M17523 | | | |
| 390 | 76560 | M19533mRNA_i_at | NM_017101 | 25518 | peptidylprolyl isomerase A | Rn.1463 |
| 391 | 78244 | M19648cds_f_at | NM_001005382 | 292855 | glandular kallikrein 12, submandibular/renal | |
| 391 | 78259 | M22922cds_f_at | NM_001005382 | 292855 | glandular kallikrein 12, submandibular/renal | |
| 392 | 82995 | M19651_at | NM_012953 | 25445 | fos-like antigen 1 | Rn.11306 |
| 393 | 78248 | M20722_s_at | M20722 | 287750 | proline-rich protein | Rn.81196 |
| 394 | 78249 | M20724_s_at | M20724 | 287750 | proline-rich protein | Rn.81196 |
| 395 | 78252 | M21770_at | NM_012503 | 24210 | asialoglycoprotein receptor 1 | Rn.44300 |
| 396 | 79690 | M22253_at | NM_030875 | 81574 | sodium channel, voltage-gated, type 1, alpha polypeptide | Rn.32079 |
| 397 | 78261 | M22993cds_s_at | NM_023103 | 65279 | Murinoglobulin 1 homolog (mouse) | Rn.109457 |
| 398 | 78262 | M23566exon_s_at | NM_012488 | 24153 | alpha-2-macroglobulin | Rn.780 |
| 399 | 79040 | X16044cds_s_at | NM_017040 | 24673 | protein phosphatase 2a, catalytic subunit, beta isoform | Rn.977 |
| 399 | 81898 | M23591mRNA#2_at | NM_017040 | 24673 | protein phosphatase 2a, catalytic subunit, beta isoform | Rn.977 |
| 400 | 78263 | M23643cds_s_at | NM_013046 | 25569 | thyrotropin releasing hormone | Rn.22 |
| 400 | 78307 | M36317_s_at | NM_013046 | 25569 | thyrotropin releasing hormone | Rn.22 |
| 401 | 81881 | M23697_at | NM_013151 | 25692 | plasminogen activator, tissue | Rn.107102 |
| 402 | 82017 | M24067_at | NM_012620 | 24617 | serine (or cysteine) proteinase inhibitor, clade E, member 1 | Rn.29367 |
| 403 | 76565 | M24239cds#2_f_at | M24239 | | | |
| 404 | 81820 | M25073_at | NM_031012 | 81641 | alanyl (membrane) aminopeptidase | Rn.11132 |
| 405 | 78276 | M25348_s_at | NM_013101 | 25638 | phosphodiesterase 4A | Rn.91357 |
| 406 | 79667 | M25804_g_at | NM_145775 | 252917 | nuclear receptor subfamily 1, group D, member 1 | Rn.29848 |
| 407 | 78279 | M25823_s_at | NM_138507 | 24699 | protein tyrosine phosphatase, receptor type, C | Rn.90166 |
| 408 | 76660 | M26125_at | NM_012844 | 25315 | epoxide hydrolase 1 | Rn.3603 |
| 409 | 76569 | M26127_s_at | NM_012541 | 24297 | cytochrome P450, family 1, subfamily a, polypeptide 2 | Rn.5563 |
| 410 | 78324 | M61211cds_s_at | NM_012978 | 25477 | luteinizing hormone/choriogonadotropin receptor | Rn.11216 |
| 410 | 79423 | M26199_s_at | NM_012978 | 25477 | luteinizing hormone/choriogonadotropin receptor | Rn.11216 |
| 410 | 81781 | rc_AI236945_s_at | NM_012978 | 25477 | luteinizing hormone/choriogonadotropin receptor | Rn.11216 |
| 411 | 79522 | M26534_f_at | M26534 | | | |
| 412 | 79457 | M26643_at | NM_013178 | 25722 | sodium channel, voltage-gated, type IV, alpha palypeptide | Rn.9700 |
| 413 | 82027 | M26686_at | M26686 | | | |
| 414 | 79451 | M27223_at | NM_019266 | 29710 | sodium channel, voltage-gated, type 8, alpha polypeptide | Rn.91216 |
| 415 | 78285 | M27434_s_at | NM_147214 | 259246 | alpha-2u globulin PGCL1 | Rn.86422 |
| 416 | 76685 | M27466_at | NM_019360 | 54322 | cytochrome c oxidase, subunit VIc | Rn.846 |
| 417 | 81803 | M29014_at | NM_017071 | 24954 | insulin receptor | Rn.9876 |
| 418 | 81965 | M29293_at | NM_031117 | 81781 | small nuclear ribonucleoprotein N | Rn.11169 |
| 419 | 81947 | M29358_at | NM_017160 | 29304 | ribosomal protein S6 | Rn.34341 |
| 420 | 81828 | M30689_at | NM_139257 | 246138 | lymphocyte antigen 6 complex, locus B | Rn.39703 |
| 421 | 78296 | M31229mRNA_s_at | NM_133285 | 201097 | H1 histone family, member 4 | Rn.37879 |
| 422 | 81826 | M32016_at | NM_017068 | 24944 | lysosomal membrane glycoprotein 2 | Rn.1722 |
| 423 | 79474 | M33025_s_at | NM_031975 | 83801 | parathymosin | Rn.3609 |
| 423 | 82271 | X16481_r_at | NM_031975 | 83801 | parathymosin | Rn.3609 |
| 424 | 81915 | M33962_g_at | NM_012637 | 24697 | protein tyrosine phosphatase, non-receptor type 1 | Rn.11317 |
| 425 | 81969 | M35270complete_seq_i_at | NM_030656 | 24792 | alanine-glyoxylate aminotransferase | Rn.9931 |
| 426 | 79525 | M35297_at | NM_153722 | 266762 | MAS-related GPR, member F | Rn.9848 |
| 427 | 78308 | M36418_s_at | NM_031608 | 50592 | glutamate receptor, ionotropic, AMPA1 (alpha 1) | Rn.29971 |
| 428 | 78312 | M36804_s_at | NM_001007597 | 25447 | follicle - stimulating hormone subunit beta | Rn.92303 |
| 429 | 78315 | M37568cds_at | M37568 | | | |
| 430 | 76583 | M38566mRNA_s_at | NM_178847 | 301517 | cytochrome P450, family 27, subfamily a, polypeptide 1 | Rn.94956 |
| 431 | 82043 | M38759_at | NM_017016 | 24443 | histidine decarboxylase | Rn.48653 |
| 432 | 78319 | M55015cds_s_at | NM_012749 | 25135 | nucleolin | Rn.127771 |
| 433 | 80414 | M57507_at | NM_012770 | 25206 | guanylate cyclase 1, soluble, beta 2 | Rn.10933 |
| 434 | 82839 | M57664_g_at | NM_012529 | 24264 | creatine kinase, brain | Rn.1472 |
| 435 | 79560 | M57705_g_at | NM_019353 | 54314 | thyroid peroxidase | Rn.91199 |
| 436 | 81907 | M58758_g_at | NM_031604 | 29757 | ATPase, H+ transporting, lysosomal V0 subunit a isoform 1 | Rn.6015 |
| 437 | 82095 | M59861_at | NM_022547 | 64392 | formyltetrahydrofolate dehydrogenase | Rn.2328 |
| 438 | 76805 | M60322_at | NM_012498 | 24192 | Aldehyde reductase 1 (low Km aldose reductase) (5.8 kb Pstl fragment, | Rn.107801 |
| | | | | | probably the functional gene) | |
| 439 | 82045 | M60655_at | NM_016991 | 24173 | adrenergic receptor, alpha 1b | Rn.10032 |
| 440 | 81141 | rc_AA944156_s_at | NM_017259 | 29619 | B-cell translocation gene 2, anti-proliferative | Rn.27923 |
| 440 | 82679 | M60921_at | NM_017259 | 29619 | B-cell translocation gene 2, anti-proliferative | Rn.27923 |
| 440 | 82680 | M60921_g_at | NM_017259 | 29619 | B-cell translocation gene 2, anti-proliferative | Rn.27923 |
| 441 | 78325 | M61725_s_at | M61725 | 25574 | upstream binding transcription factor, RNA polymerase I | Rn.22469 |
| 442 | 76661 | M61875_s_at | NM_012924 | 25406 | CD44 antigen | Rn.1120 |
| 443 | 76591 | M62388_at | NM_031138 | 81816 | ubiquitin conjugating enzyme | Rn.20766 |
| 444 | 81860 | M62891_at | NM_001010964 | 362443 | killer cell lectin-like receptor subfamily B member 1A | Rn.101826 |
| 445 | 81878 | M63006_at | NM_016989 | 24166 | adenylate cyclase activating polypeptide 1 | Rn.37400 |
| 446 | 76654 | M63122_at | NM_013091 | 25625 | tumor necrosis factor receptor superfamily, member 1a | Rn.11119 |
| 447 | 79660 | M64299_at | NM_017155 | 29290 | adenosine A1 receptor | Rn.32078 |
| 448 | 79622 | M64488_at | NM_012665 | 24805 | synaptotagmin 2 | Rn.88574 |
| 449 | 79518 | M64793_at | M64793 | | | |
| 450 | 79798 | M64795_i_at | M64795 | | | |
| 451 | 79431 | M64862_at | NM_022667 | 24546 | solute carrier organic anion transporter family, member 2a1 | Rn.9671 |
| 452 | 80431 | M64867_at | NM_017254 | 29595 | 5-hydroxytryptamine (serotonin) receptor 2A | Rn.127646 |
| 453 | 82760 | M64986_g_at | NM_012963 | 25459 | high mobility group box 1 | Rn.4121 |
| 454 | 79554 | M65253_at | NM_133514 | 117061 | matrix metallopeptidase 10 | Rn.9946 |
| 455 | 78964 | X02322Poly_A_Site#1_s_at | NM_031577 | 29446 | growth hormone releasing hormone | Rn.10693 |
| 455 | 83008 | M73486_at | NM_031577 | 29446 | growth hormone releasing hormone | Rn.10693 |
| 456 | 81833 | M73714_at | NM_031731 | 65183 | aldehyde dehydrogenase family 3, subfamily A2 | Rn.9113 |
| 457 | 79454 | M74494_g_at | NM_012504 | 24211 | ATPase, Na+/K+ transporting, alpha 1 polypeptide | Rn.2992 |
| 458 | 76598 | M74776_at | M74776 | | | |
| 459 | 81816 | M75148_at | M75148 | 171041 | kinesin 2 | Rn.106230 |
| 460 | 76602 | M76535cds_g_at | NM_019280 | 50563 | gap junction membrane channel protein alpha 5 | Rn.88300 |
| 461 | 76603 | M76704_s_at | NM_012861 | 25332 | O-6-methylguanine-DNA methyltransferase | Rn.9836 |
| 462 | 82775 | M77245_at | NM_017277 | 29663 | adaptor protein complex AP-1, beta 1 subunit | Rn.37383 |
| 463 | 82689 | M80550_at | NM_031007 | 81636 | adenylate cyclase 2 | Rn.10731 |
| 464 | 76605 | M80784_s_at | NM_017256 | 29610 | transforming growth factor, beta receptor 3 | Rn.9953 |
| 465 | 78354 | M81183Exon_UTR_g_at | M81183 | | | |
| 466 | 83033 | M81687_at | NM_013082 | 25615 | syndecan 2 | Rn.11127 |
| 467 | 76606 | M81766_at | NM_206849 | 361801 | retinoid X receptor beta | Rn.49295 |
| 468 | 82099 | M84210_at | NM_053997 | 117101 | potassium voltage gated channel, Shaw-related subfamily, member 3 | Rn.9885 |
| 469 | 80423 | M84361_at | NM_023981 | 78965 | colony stimulating factor 1 (macrophage) | Rn.83632 |
| 470 | 78367 | M86341_at | NM_183325 | 25371 | ADP-ribosylarginine hydrolase | Rn.13315 |
| 471 | 76283 | rc_AA998683_g_at | NM_031970 | 24471 | heat shock 27 kDa protein 1 | Rn.3841 |
| 471 | 76285 | rc_AI176658_s_at | NM_031970 | 24471 | heat shock 27 kDa protein 1 | Rn.3841 |
| 471 | 76607 | M86389cds_s_at | NM_031970 | 24471 | heat shock 27 kDa protein 1 | Rn.3841 |
| 472 | 79498 | M86514_at | M86514 | 287750 | proline-rich protein | Rn.81196 |
| 473 | 82762 | M86564_at | NM_021740 | 29222 | prothymosin alpha | Rn.817 |
| 474 | 76864 | M86758_at | NM_012883 | 25355 | sulfotransferase, estrogen preferring | Rn.32104 |
| 475 | 80222 | M87634_at | NM_012560 | 24370 | forkhead box G1 | Rn.9864 |
| 476 | 78372 | M87786_at | M87786 | | | Rn.117928 |
| 477 | 79425 | M88111_at | NM_053996 | 117100 | solute carrier family 6 (neurotransmitter transporter, L-proline) member 7 | Rn.9663 |
| 478 | 78375 | M88488exon_at | M88488 | | | |
| 479 | 78376 | M89646_at | NM_031112 | 81776 | ribosomal protein S24 | Rn.979 |
| 480 | 78382 | M89953cds_at | NM_012852 | 25323 | 5-hydroxytryptamine (serotonin) receptor 1D | Rn.34834 |
| 481 | 79424 | M91234_f_at | M91234 | | | |
| 482 | 82415 | M91466_at | NM_017161 | 29316 | adenosine A2B receptor | Rn.89544 |
| 483 | 78390 | M91595exon_s_at | NM_013122 | 25662 | insulin-like growth factor binding protein 2 | Rn.6813 |
| 484 | 81988 | M92074_at | NM_017144 | 29248 | troponin I, cardiac | Rn.64141 |
| 485 | 78397 | M93257_s_at | NM_012531 | 24267 | catechol-O-methyltransferase | Rn.220 |
| 486 | 81337 | rc_AI030089_at | NM_022869 | 64896 | nucleolar and coiled-body phosphoprotein 1 | Rn.9517 |
| 486 | 81862 | M94287_at | NM_022869 | 64896 | nucleolar and coiled-body phosphoprotein 1 | Rn.9517 |
| 487 | 76849 | M94548_at | NM_019623 | 56266 | cytochrome P450, family 4, subfamily F, polypeptide 2 | Rn.5722 |
| 488 | 78404 | M95791cds_f_at | M95791 | | | |
| 489 | 82552 | M96548_at | NM_052798 | 24522 | zinc finger protein 354A | Rn.11049 |
| 490 | 78410 | M98819mRNA_s_at | NM_138876 | 25028 | adenosine monophosphate deaminase 1 (isoform M) | Rn.9794 |
| 491 | 78411 | M98826mRNA_at | M98826 | | | |
| 492 | 78412 | M98826mRNA_g_at | NM_031573 | 29353 | phosphorylase kinase gamma 1 | Rn.10399 |
| 493 | 78414 | M99222_s_at | NM_012918 | 25398 | calcium channel, voltage-dependent, P/Q type, alpha 1A subunit | Rn.87769 |
| 494 | 83628 | rc_AA684919_at | AA684919 | | | Rn.14682 |
| 495 | 84403 | rc_AA799323_at | AA799323 | 364206 | pleckstrin (predicted) | Rn.6178 |
| 496 | 84404 | rc_AA799328_at | AA799328 | | | Rn.112856 |
| 497 | 84405 | rc_AA799330_at | NM_001007634 | 294754 | pelota homolog | Rn.3842 |
| 498 | 84416 | rc_AA799412_g_at | NM_001008511 | 293701 | estrogen related receptor, alpha | Rn.2190 |
| 499 | 84420 | rc_AA799427_at | AA799427 | 361194 | similar to RIKEN cDNA 1110018J18 (predicted) | Rn.24906 |
| 500 | 84426 | rc_AA799442_g_at | AA799442 | 293114 | hypothetical LOC293114 (predicted) | Rn.3826 |
| 501 | 84440 | rc_AA799471_at | AA799471 | | | Rn.128444 |
| 502 | 84445 | rc_AA799478_at | NM_022700 | 64664 | ADP-ribosylation factor-like 3 | Rn.9538 |
| 503 | 76270 | rc_AA799479_g_at | AA799479 | 293652 | NADH dehydrogenase (ubiquinone) Fe—S protein 8 (predicted) | Rn.3373 |
| 504 | 84446 | rc_AA799480_at | AA799480 | | | |
| 505 | 84450 | rc_AA799488_at | AA799488 | 316685 | similar to D330021B20 protein (predicted) | Rn.98546 |
| 506 | 84453 | rc_AA799495_at | AA799495 | | | Rn.61074 |
| 507 | 81065 | rc_AA799498_at | NM_031545 | 25105 | natriuretic peptide precursor type B | Rn.3835 |
| 508 | 84474 | rc_AA799534_at | AA799534 | | | Rn.94764 |
| 509 | 84482 | rc_AA799544_at | AA799544 | 313325 | ladinin (predicted) | |
| 510 | 84488 | rc_AA799554_at | AA799554 | | | Rn.6202 |
| 511 | 84491 | rc_AA799566_g_at | AA799566 | 171124 | incisor protein | Rn.128967 |
| 512 | 84521 | rc_AA799637_at | AA799637 | 309798 | LIM and senescent cell antigen-like domains 1 (predicted) | Rn.100399 |
| 513 | 84523 | rc_AA799641_at | NM_001008312 | 296350 | tumor differentially expressed 1 (predicted) | Rn.3775 |
| 513 | 84524 | rc_AA799641_g_at | NM_001008312 | 296350 | tumor differentially expressed 1 (predicted) | Rn.3775 |
| 514 | 84529 | rc_AA799656_at | AA799656 | 290850 | mitochondrial ribosomal protein S31 (predicted) | Rn.22173 |
| 514 | 84530 | rc_AA799656_g_at | AA799656 | 290850 | mitochondrial ribosomal protein S31 (predicted) | Rn.22173 |
| 515 | 84556 | rc_AA799724_g_at | AA799724 | 360762 | similar to RNA polymerase 1-3 | Rn.6228 |
| 516 | 84576 | rc_AA799766_at | AA799766 | 288480 | similar to Jtv1-pending protein | Rn.3333 |
| 517 | 84577 | rc_AA799771_at | AA799771 | | | Rn.17586 |
| 518 | 84578 | rc_AA799773_at | AA799773 | 362332 | filamin C, gamma (actin binding protein 280) (predicted) | Rn.22352 |
| 518 | 84579 | rc_AA799773_g_at | AA799773 | 362332 | filamin C, gamma (actin binding protein 280) (predicted) | Rn.22352 |
| 519 | 84585 | rc_AA799791_at | NM_184049 | 373542 | serine/threonine kinase 25 (STE20 homolog, yeast) | Rn.22352 |
| 520 | 84593 | rc_AA799812_at | AA799812 | | | Rn.22271 |
| 521 | 84603 | rc_AA799847_at | AA799847 | | | Rn.21429 |
| 522 | 84613 | rc_AA799887_s_at | NM_031331 | 83499 | proteasome (prosome, macropain) 26S subunit, non-ATPase, 4 | Rn.3830 |
| 523 | 84630 | rc_AA799992_g_at | AA799992 | 361624 | similar to C11orf17 protein (predicted) | Rn.106344 |
| 524 | 84638 | rc_AA800015_at | AA800015 | 293345 | TAF10 RNA polymerase II, TATA box binding protein (TBP)-associated factor | Rn.104887 |
| | | | | | (predicted) | |
| 525 | 84652 | rc_AA800053_at | NM_001007618 | 289508 | ring finger and CHY zinc finger domain containing 1 | Rn.2007 |
| 526 | 84663 | rc_AA800175_at | AA800175 | 298696 | protein (peptidyl-prolyl cis/trans isomerase) NIMA-interacting 1 (predicted) | Rn.6291 |
| 527 | 84685 | rc_AA800222_at | AA800222 | 361367 | autocrine motility factor receptor (predicted) | Rn.107561 |
| 528 | 84696 | rc_AA800272_at | AA800272 | 300974 | mitochondrial ribosomal protein L3 (predicted) | Rn.6950 |
| 529 | 76706 | U53922_at | NM_022934 | 65028 | DnaJ-like protein | Rn.64562 |
| 529 | 84719 | rc_AA800551_at | NM_022934 | 65028 | DnaJ-like protein | Rn.64562 |
| 530 | 84723 | rc_AA800572_at | AA800572 | 294981 | sprouty homolog 1 (Drosophila) (predicted) | Rn.22787 |
| 531 | 84738 | rc_AA800651_s_at | AA800651 | 312754 | protein phosphatase 2, regulatory subunit B (B56), alpha isoform (predicted) | Rn.104461 |
| 532 | 84740 | rc_AA800665_at | NM_023955 | 65168 | secretory carrier membrane protein 2 | Rn.3752 |
| 533 | 84745 | rc_AA800678_g_at | AA800678 | 363328 | similar to AW046014 protein | Rn.21528 |
| 534 | 84747 | rc_AA800680_at | AA800680 | 366002 | Notch-regulated ankyrin repeat protein (predicted) | Rn.22790 |
| 535 | 84749 | rc_AA800686_at | AA800686 | | | Rn.69085 |
| 536 | 84761 | rc_AA800719_at | AA800719 | | | Rn.98517 |
| 537 | 84773 | rc_AA800750_f_at | AA800750 | | | |
| 538 | 84782 | rc_AA800784_at | NM_031327 | 83476 | cysteine rich protein 61 | Rn.22129 |
| 539 | 84799 | rc_AA800849_f_at | AA800849 | | | |
| 540 | 84804 | rc_AA800853_at | NM_001002830 | 305302 | RAS-like family 11 member B | Rn.36357 |
| 541 | 84816 | rc_AA800912_g_at | NM_001001504 | 246770 | general transcription factor II I repeat domain-containing 1 | Rn.84872 |
| 542 | 81086 | rc_AA801286_at | NM_032057 | 83523 | Inositol (myo)-1(or 4)-monophosphatase 1 | Rn.3975 |
| 543 | 81489 | rc_AA817846_at | NM_053995 | 117099 | 3-hydroxybutyrate dehydrogenase (heart, mitochondrial) | Rn.36635 |
| 544 | 81490 | rc_AA817887_at | NM_022511 | 64303 | profilin 1 | Rn.1152 |
| 545 | 81511 | rc_AA818025_g_at | AA818025 | | | |
| 546 | 81524 | rc_AA818114_at | NM_017355 | 50866 | RAB4B, member RAS oncogene family | Rn.7181 |
| 547 | 81534 | rc_AA818144_at | NM_017096 | 25419 | C-reactive protein, petaxin related | Rn.16463 |
| 548 | 81600 | rc_AA818970_s_at | NM_017333 | 50672 | endothelin receptor type B | Rn.11412 |
| 549 | 81601 | rc_AA818982_at | NM_012887 | 25359 | thymopoietin | Rn.3364 |
| 549 | 81602 | rc_AA818982_g_at | NM_012887 | 25359 | thymopoietin | Rn.3364 |
| 550 | 81090 | rc_AA849036_at | NM_017090 | 25201 | guanylate cyclase 1, soluble, alpha 3 | Rn.1974 |
| 551 | 81092 | rc_AA849648_at | NM_053330 | 79449 | ribosomal protein L21 | Rn.83200 |
| 552 | 81098 | rc_AA850669_at | NM_053891 | 116671 | cyclin-dependent kinase 5, regulatory subunit 1 (p35) | Rn.11213 |
| 552 | 82494 | U50707_at | NM_053891 | 116671 | cyclin-dependent kinase 5, regulatory subunit 1 (p35) | Rn.11213 |
| 553 | 81100 | rc_AA850756_at | NM_012857 | 25328 | lysosomal membrane glycoprotein 1 | Rn.40177 |
| 554 | 76272 | rc_AA851403_at | AA851403 | 293991 | NADH dehydrogenase (ubiquinone) 1 beta subcomplex 8 (predicted) | Rn.3383 |
| 554 | 76273 | rc_AA851403_g_at | AA851403 | 293991 | NADH dehydrogenase (ubiquinone) 1 beta subcomplex 8 (predicted) | Rn.3383 |
| 555 | 81797 | rc_AA858520_at | NM_012561 | 24373 | follistatin | Rn.2743 |
| 556 | 83046 | rc_AA858586_at | AA858586 | | | |
| 557 | 83050 | rc_AA858600_at | AA858600 | 360745 | leucine-zipper-like transcriptional regulator, 1 (predicted) | Rn.15759 |
| 558 | 83052 | rc_AA858603_g_at | AA858603 | 362824 | similar to hypothetical protein MGC20700 (predicted) | Rn.93047 |
| 559 | 78660 | S82383_s_at | NM_173111 | 286890 | tropomyosin isoform 6 | Rn.37575 |
| 559 | 81795 | rc_AA859305_s_at | NM_173111 | 286890 | tropomyosin isoform 6 | Rn.37575 |
| 560 | 76323 | rc_AA859372_s_at | AA859372 | | | |
| 561 | 83066 | rc_AA859473_at | NM_053555 | 89818 | vesicle-associated membrane protein 5 | Rn.22616 |
| 562 | 83069 | rc_AA859483_at | AA859483 | | | Rn.45705 |
| 563 | 83082 | rc_AA859533_at | AA859533 | 309900 | tubby like protein 1 (predicted) | Rn.254 |
| 564 | 83094 | rc_AA859581_at | NM_138518 | 171547 | late gestation lung protein 1 | Rn.4346 |
| 565 | 83099 | rc_AA859597_at | AA859597 | | | Rn.118218 |
| 566 | 77049 | rc_AA859648_at | AA859648 | 361384 | DnaJ (Hsp40) homolog, subfamily B, member 1 (predicted) | Rn.7896 |
| 567 | 83115 | rc_AA859663_at | AA859663 | 301114 | similar to RIKEN cDNA 4933406J04 | Rn.41 |
| 568 | 83122 | rc_AA859680_at | AA859680 | 304649 | similar to CG2662-PA (predicted) | Rn.20 |
| 569 | 83134 | rc_AA859718_at | AA859718 | 361119 | similar to RIKEN cDNA A230063L24 gene | Rn.40562 |
| 570 | 83135 | rc_AA859719_at | AA859719 | 289143 | mitochondrial ribosomal protein S14 (predicted) | Rn.67 |
| 571 | 83138 | rc_AA859740_at | AA859740 | | | Rn.4264 |
| 572 | 83140 | rc_AA859750_at | AA859750 | | | Rn.7937 |
| 573 | 83144 | rc_AA859760_at | AA859760 | | | Rn.120041 |
| 574 | 83150 | rc_AA859804_at | AA859804 | 363275 | eukaryotic translation initiation factor 4E like 3 (predicted) | Rn.11732 |
| 575 | 83151 | rc_AA859805_at | NM_001012125 | 315714 | lysyl oxidase-like 1 (predicted) | Rn.770 |
| 576 | 83152 | rc_AA859806_at | NM_024161 | 79130 | cysteine string protein | Rn.100120 |
| 577 | 83154 | rc_AA859827_at | AA859827 | 171570 | uridine-cytidine kinase 2 | Rn.24811 |
| 578 | 81002 | rc_AA859830_s_at | NM_017087 | 25181 | biglycan | Rn.783 |
| 579 | 78820 | U57050_g_at | NM_031978 | 83806 | proteasome (prosome, macropain) 26S subunit, non-ATPase, 1 | Rn.2891 |
| 579 | 83165 | rc_AA859869_s_at | NM_031978 | 83806 | proteasome (prosome, macropain) 26S subunit, non-ATPase, 1 | Rn.2891 |
| 580 | 77050 | rc_AA900380_at | NM_130426 | 156767 | tumor necrosis factor receptor superfamily, member 1b | Rn.83633 |
| 580 | 83172 | rc_AA859889_at | NM_130426 | 156767 | tumor necrosis factor receptor superfamily, member 1b | Rn.96673 |
| 581 | 83185 | rc_AA859922_at | AA859922 | 305291 | similar to SRD5A2L (predicted) | Rn.819 |
| 582 | 83199 | rc_AA859954_at | NM_138839 | 192129 | vacuole Membrane Protein 1 | Rn.109048 |
| 583 | 83210 | rc_AA859983_at | AA859983 | 363216 | cyclin M4 (predicted) | Rn.857 |
| 584 | 83214 | rc_AA859994_at | AA859994 | | | Rn.40435 |
| 585 | 83215 | rc_AA859996_at | AA859996 | | | Rn.105661 |
| 586 | 83222 | rc_AA860024_at | AA860024 | 293725 | eukaryotic translation elongation factor 1 gamma | Rn.128130 |
| 587 | 81155 | rc_AA945321_at | NM_134326 | 24186 | albumin | Rn.34353 |
| 587 | 83234 | rc_AA860062_g_at | NM_134326 | 24186 | albumin | Rn.34353 |
| 587 | 83240 | rc_AA866237_s_at | NM_134326 | 24186 | albumin | Rn.34353 |
| 588 | 83235 | rc_AA866221_at | AA866221 | | | |
| 589 | 83250 | rc_AA866272_at | AA866272 | 311299 | apoptosis, caspase activation inhibitor (predicted) | Rn.24294 |
| 590 | 83265 | rc_AA866362_at | AA866362 | 308336 | zinc finger protein 580 (predicted) | Rn.6984 |
| 591 | 83274 | rc_AA866414_at | NM_012651 | 24779 | solute carrier family 4, member 1 | Rn.32202 |
| 592 | 83277 | rc_AA866426_at | AA866426 | | | Rn.3101 |
| 593 | 83281 | rc_AA866439_g_at | AA866439 | | | Rn.3109 |
| 594 | 83283 | rc_AA866444_s_at | AA866444 | 307787 | Rho GTPase activating protein 10 (predicted) | Rn.40465 |
| 595 | 83302 | rc_AA874803_g_at | AA874803 | | | Rn.3130 |
| 596 | 83305 | rc_AA874815_at | AA874815 | | | |
| 597 | 83308 | rc_AA874832_at | AA874832 | 288671 | anaphase-promoting complex subunit 5 (predicted) | Rn.101004 |
| 598 | 83322 | rc_AA874874_at | NM_017270 | 29646 | alcohol dehydrogenase 4 (class II), pi polypeptide | Rn.98159 |
| 599 | 83346 | rc_AA874952_at | AA874952 | 313598 | zinc finger protein 262 (predicted) | Rn.3185 |
| 600 | 83354 | rc_AA874995_at | NM_001007703 | 313771 | similar to BC002216 protein | Rn.3197 |
| 601 | 83355 | rc_AA874999_at | AA874999 | 298068 | Sec61 beta subunit (predicted) | Rn.103030 |
| 602 | 83357 | rc_AA875002_at | AA875002 | 311846 | leucine-rich repeat-containing 8 (predicted) | Rn.33512 |
| 603 | 83359 | rc_AA875010_at | NM_053589 | 94197 | RAB14, member RAS oncogene family | Rn.6008 |
| 604 | 83360 | rc_AA875017_at | AA875017 | | | |
| 605 | 83374 | rc_AA875042_at | AA875042 | 361774 | LOC361774 (predicted) | Rn.17036 |
| 606 | 83385 | rc_AA875089_at | NM_053295 | 25403 | calpastatin | Rn.17481 |
| 607 | 83388 | rc_AA875098_at | AA875098 | 300211 | FK506 binding protein 11 (predicted) | Rn.100569 |
| 608 | 83392 | rc_AA875121_at | NM_012866 | 25337 | nuclear transcription factor-Y gamma | Rn.1457 |
| 609 | 83397 | rc_AA875126_g_at | AA875126 | | | Rn.2799 |
| 610 | 83413 | rc_AA875165_at | AA875165 | 368168 | similar to Gamma-tubulin complex component 4 (GCP-4) (hGCP4) (h76p) | Rn.2813 |
| | | | | | (Hgrip76) | |
| 611 | 83415 | rc_AA875171_at | AA875171 | 287134 | N-acetylglucosamine-1-phosphotransferase, gamma subunit (predicted) | Rn.2814 |
| 611 | 83416 | rc_AA875171_g_at | AA875171 | 287134 | N-acetylglucosamine-1-phosphotransferase, gamma subunit (predicted) | Rn.2814 |
| 612 | 83418 | rc_AA875182_at | AA875182 | 361809 | zinc finger protein 523 (predicted) | Rn.13446 |
| 613 | 83423 | rc_AA875197_at | AA875197 | 300806 | similar to 3300001A09Rik protein (predicted) | Rn.102780 |
| 614 | 83424 | rc_AA875198_at | NM_001008386 | 493574 | notch1-induced protein | Rn.119606 |
| 615 | 83428 | rc_AA875205_g_at | AA875205 | 288516 | eukaryotic translation initiation factor 3, subunit 9 (eta) (predicted) | Rn.2829 |
| 616 | 83429 | rc_AA875206_at | NM_053747 | 114590 | ubiquilin 1 | Rn.94864 |
| 617 | 83443 | rc_AA875255_at | AA875255 | | | |
| 618 | 83446 | rc_AA875263_at | AA875263 | 300222 | microspherule protein 1 (predicted) | Rn.129193 |
| 619 | 83450 | rc_AA875275_at | AA875275 | | | Rn.35732 |
| 620 | 76248 | rc_AA875327_at | NM_001006957 | 288599 | eukaryotic translation initiation factor 4H | Rn.79423 |
| 621 | 83459 | rc_AA875348_at | AA875348 | | | |
| 622 | 83463 | rc_AA875405_at | NM_001012091 | 311547 | forkhead-like 18 (Drosophila) (predicted) | Rn.3227 |
| 623 | 83472 | rc_AA875428_at | AA875428 | 306587 | similar to RIKEN cDNA 9130410M22 | Rn.2916 |
| 624 | 83476 | rc_AA875444_at | AA875444 | 25416 | dihydropyrimidinase-like 2 | Rn.2889 |
| 625 | 83480 | rc_AA875468_at | AA875468 | | | Rn.18917 |
| 626 | 81401 | rc_AI070967_g_at | NM_012903 | 25379 | acidic (leucine-rich) nuclear phosphoprotein 32 family, member A | Rn.10123 |
| 626 | 83483 | rc_AA875495_at | NM_012903 | 25379 | acidic (leucine-rich) nuclear phosphoprotein 32 family, member A | Rn.10123 |
| 626 | 84218 | rc_AA893496_at | NM_012903 | 25379 | acidic (leucine-rich) nuclear phosphoprotein 32 family, member A | Rn.10123 |
| 627 | 83486 | rc_AA875506_at | NM_053824 | 116549 | casein kinase II, alpha 1 polypeptide | Rn.4231 |
| 628 | 83505 | rc_AA875598_at | AA875598 | 361258 | cullin 2 (predicted) | Rn.2970 |
| 629 | 83509 | rc_AA875617_at | NM_031083 | 81747 | phosphatidylinositol 4-kinase, catalytic, beta polypeptide | Rn.14991 |
| 630 | 83516 | rc_AA875641_at | AA875641 | | | |
| 631 | 83522 | rc_AA875665_g_at | NM_001008694 | 494125 | reticulocalbin 3, EF-hand calcium binding domain | Rn.2997 |
| 632 | 83635 | rc_AA891032_at | AA891032 | | | |
| 633 | 83642 | rc_AA891058_at | AA891058 | | | Rn.19936 |
| 634 | 83644 | rc_AA891069_at | AA891069 | 296753 | serine/arginine-rich protein specific kinase 2 (predicted) | Rn.13768 |
| 635 | 83649 | rc_AA891140_at | AA891140 | 361408 | similar to CDNA sequence BC027246 (predicted) | Rn.17087 |
| 635 | 83650 | rc_AA891140_g_at | AA891140 | 361408 | similar to CDNA sequence BC027246 (predicted) | Rn.17087 |
| 636 | 83655 | rc_AA891196_at | AA891196 | 300783 | similar to Butyrate-induced transcript 1 | Rn.48852 |
| 637 | 83660 | rc_AA891220_at | NM_177928 | 297508 | pre-B-cell colony enhancing factor 1 | Rn.7264 |
| 638 | 83661 | rc_AA891221_at | AA891221 | 293103 | similar to RIKEN cDNA 0610007P06 | Rn.96145 |
| 639 | 78837 | U63923_at | NM_031614 | 58819 | thioredoxin reductase 1 | Rn.67581 |
| 639 | 83671 | rc_AA891286_at | NM_031614 | 58819 | thioredoxin reductase 1 | Rn.67581 |
| 640 | 83681 | rc_AA891314_at | AA891314 | 363133 | poly(rC) binding protein 4 (predicted) | Rn.40405 |
| 641 | 83684 | rc_AA891356_at | AA891356 | 304650 | hypothetical LOC304650 (predicted) | Rn.40355 |
| 642 | 83685 | rc_AA891362_at | NM_057186 | 113965 | L-3-hydroxyacyl-Coenzyme A dehydrogenase, short chain | Rn.92789 |
| 643 | 83555 | rc_H31665_at | NM_080902 | 140937 | hypoxia induced gene 1 | Rn.2084 |
| 643 | 83688 | rc_AA891422_at | NM_080902 | 140937 | hypoxia induced gene 1 | Rn.2084 |
| 644 | 83689 | rc_AA891423_at | AA891423 | 361184 | similar to hypothetical protein FLJ12118 (predicted) | Rn.54352 |
| 645 | 83698 | rc_AA891476_at | AA891476 | | | Rn.24387 |
| 646 | 83702 | rc_AA891524_at | NM_001008367 | 361501 | similar to RIKEN cDNA 0610042E07 (predicted) | Rn.1804 |
| 647 | 83705 | rc_AA891542_at | AA891542 | 313811 | DnaJ (Hsp40) homolog, subfamily B, member 5 (predicted) | Rn.92865 |
| 648 | 83708 | rc_AA891553_at | NM_001004283 | 362952 | eukaryotic translation initiation factor 3 subunit 7 | Rn.3463 |
| 649 | 83716 | rc_AA891588_at | AA891588 | 301709 | similar to hypothetical protein E130310N06 | Rn.14693 |
| 650 | 83718 | rc_AA891591_at | NM_031356 | 83533 | programmed cell death 8 | Rn.8124 |
| 651 | 83720 | rc_AA891596_at | AA891596 | | | Rn.14694 |
| 652 | 83722 | rc_AA891614_at | NM_139332 | 246215 | two pore channel 1 | Rn.24484 |
| 653 | 83723 | rc_AA891631_at | AA891631 | | | Rn.14698 |
| 654 | 83729 | rc_AA891666_g_at | NM_001007804 | 287645 | similar to EAP30 subunit of ELL complex (predicted) | Rn.128189 |
| 655 | 83743 | rc_AA891713_at | NM_173340 | 317646 | ribosomal protein L13A | Rn.92211 |
| 656 | 83747 | rc_AA891719_at | AA891719 | 306460 | ectonucleotide pyrophosphatase/phosphodiesterase 6 (predicted) | Rn.8484 |
| 657 | 83748 | rc_AA891721_at | NM_001009647 | 293754 | mitochondrial ribosomal protein L16 | Rn.101972 |
| 658 | 83749 | rc_AA891724_at | NM_198765 | 306809 | bicaudal D homolog 2 (Drosophila) | Rn.103443 |
| 659 | 83752 | rc_AA891727_g_at | AA891727 | 361805 | similar to RIKEN cDNA 2900010M23 (predicted) | Rn.128924 |
| 660 | 83757 | rc_AA891737_at | AA891737 | | | Rn.3650 |
| 661 | 83761 | rc_AA891746_at | AA891746 | 296570 | endothelial differentiation-related factor 1 (predicted) | Rn.17092 |
| 662 | 81025 | rc_AA891764_s_at | NM_030827 | 29216 | low density lipoprotein receptor-related protein 2 | Rn.26430 |
| 663 | 83781 | rc_AA891810_at | AA891810 | | | Rn.17620 |
| 663 | 83782 | rc_AA891810_g_at | AA891810 | | | Rn.17620 |
| 664 | 83797 | rc_AA891838_at | AA891838 | 298586 | similar to ribosomal protein P0-like protein; 60S acidic ribosomal protein PO; | Rn.102515 |
| | | | | | ribosomal protein, large, P0-like (predicted) | |
| 665 | 83800 | rc_AA891842_g_at | AA891842 | 316256 | tumor necrosis factor receptor superfamily, member 21 (predicted) | Rn.98293 |
| 666 | 83803 | rc_AA891851_at | AA891851 | | | Rn.52727 |
| 667 | 83813 | rc_AA891877_at | AA891877 | | | Rn.114607 |
| 668 | 83815 | rc_AA891880_g_at | NM_022948 | 65042 | sideroflexin 3 | Rn.37545 |
| 669 | 83822 | rc_AA891903_at | AA891903 | | | Rn.14719 |
| 670 | 83824 | rc_AA891914_at | NM_001005383 | 300981 | aminoacylase 1 | Rn.3679 |
| 671 | 83825 | rc_AA891916_at | NM_032615 | 60418 | membrane interacting protein of RGS16 | Rn.3731 |
| 671 | 83826 | rc_AA891916_g_at | NM_032615 | 60418 | membrane interacting protein of RGS16 | Rn.3731 |
| 672 | 83827 | rc_AA891917_at | AA891917 | 310758 | capping protein (actin filament) muscle Z-line, alpha 1 (predicted) | Rn.13838 |
| 673 | 83838 | rc_AA891940_at | AA891940 | 310756 | similar to mKIAA1631 protein | Rn.3508 |
| 674 | 83839 | rc_AA891943_at | AA891943 | | | Rn.3564 |
| 675 | 83846 | rc_AA891969_at | AA891969 | 289810 | similar to small unique nuclear receptor co-repressor | Rn.102095 |
| 676 | 83851 | rc_AA892006_g_at | AA892006 | 360716 | ATPase, H+ transporting, V1 subunit A, isoform 1 (predicted) | Rn.1431 |
| 677 | 83855 | rc_AA892012_g_at | NM_013177 | 25721 | glutamate oxaloacetate transaminase 2 | Rn.98650 |
| 678 | 83857 | rc_AA892014_r_at | NM_139101 | 245960 | potassium channel regulator 1 | Rn.9968 |
| 679 | 83861 | rc_AA892042_at | AA892042 | 317335 | DEAD (Asp-Glu-Ala-Asp) box polypeptide 3, X-linked | Rn.95841 |
| 680 | 83862 | rc_AA892049_at | AA892049 | 364721 | histone 1, H4j (predicted) | Rn.15656 |
| 681 | 83871 | rc_AA892112_g_at | AA892112 | 361538 | proline dehydrogenase (oxidase) 2 (predicted) | Rn.4247 |
| 682 | 83886 | rc_AA892204_at | AA892204 | | | Rn.132398 |
| 683 | 83888 | rc_AA892228_at | AA892228 | 362160 | CUG triplet repeat, RNA binding protein 1 (predicted) | Rn.105713 |
| 684 | 76221 | rc_AA892248_at | AA892248 | | | |
| 684 | 76222 | rc_AA892248_g_at | AA892248 | | | |
| 685 | 83895 | rc_AA892258_at | NM_053524 | 85431 | NADPH oxidase 4 | Rn.14744 |
| 686 | 83912 | rc_AA892298_at | NM_175707 | 301432 | peptidylprolyl isomerase (cyclophilin)-like 3 | Rn.14747 |
| 687 | 83917 | rc_AA892303_g_at | NM_001009666 | 300078 | dynein, axonemal, light chain 4 (predicted) | Rn.3257 |
| 688 | 83919 | rc_AA892305_at | NM_001007707 | 315094 | brain protein 16 | Rn.17102 |
| 689 | 83922 | rc_AA892310_at | AA892310 | 306332 | adaptor-related protein complex AP-1, mu subunit 1 (predicted) | Rn.7484 |
| 690 | 83928 | rc_AA892318_g_at | AA892318 | 288656 | ADP-ribosylation factor-like 6 interacting protein 4 (predicted) | Rn.105729 |
| 691 | 83947 | rc_AA89237_g_at | AA892378 | 288584 | tetratricopeptide repeat domain 11 (predicted) | Rn.1298 |
| 692 | 83948 | rc_AA892380_at | AA892380 | 306780 | serine palmitoyltransferase, long chain base subunit 1 (predicted) | Rn.127885 |
| 693 | 83958 | rc_AA892400_at | NM_198736 | 293497 | potassium channel tetramerisation domain containing 13 | Rn.103144 |
| 694 | 83965 | rc_AA892462_at | AA892462 | | | Rn.3254 |
| 695 | 83969 | rc_AA892470_at | AA892470 | 289784 | H2A histone family, member V (predicted) | Rn.100938 |
| 696 | 83977 | rc_AA892498_at | NM_001005547 | 300733 | transmembrane 4 superfamily member 8 | Rn.998 |
| 697 | 83985 | rc_AA892520_at | AA892520 | 287721 | vesicle amine transport protein 1 homolog (T californica) (predicted) | Rn.9118 |
| 697 | 83986 | rc_AA892520_g_at | AA892520 | 287721 | vesicle amine transport protein 1 homolog (T californica) (predicted) | Rn.9118 |
| 698 | 83988 | rc_AA892526_at | AA892526 | | | Rn.137471 |
| 699 | 76243 | rc_AA892532_at | NM_001004442 | 286906 | thioredoxin domain containing 7 | Rn.2685 |
| 700 | 83999 | rc_AA892551_i_at | AA892551 | | | |
| 701 | 84004 | rc_AA892557_at | NM_001007802 | 316035 | chemokine-like factor super family 6 (predicted) | Rn.98431 |
| 702 | 84014 | rc_AA892578_at | AA892578 | 360757 | similar to RIKEN cDNA 6330406I15 (predicted) | Rn.14770 |
| 703 | 84015 | rc_AA892582_s_at | AA892582 | | | Rn.9810 |
| 704 | 84016 | rc_AA892593_at | AA892593 | | | |
| 705 | 84017 | rc_AA892598_at | NM_175580 | 290556 | guanine nucleotide binding protein-like 3 (nucleolar) | Rn.4110 |
| 705 | 84018 | rc_AA892598_g_at | NM_175580 | 290556 | guanine nucleotide binding protein-like 3 (nucleolar) | Rn.4110 |
| 706 | 84022 | rc_AA892630_at | AA892630 | 290027 | ADP-ribosyltransferase (NAD+, poly(ADP-ribose) polymerase)-like 2 | Rn.22730 |
| | | | | | (predicted) | |
| 707 | 84025 | rc_AA892635_g_at | AA892635 | | | Rn.129172 |
| 708 | 84031 | rc_AA892647_at | AA892647 | 306955 | histone 1, H4h (predicted) | Rn.44120 |
| 709 | 81037 | rc_AA892775_at | NM_012771 | 25211 | lysozyme | Rn.2283 |
| 710 | 84052 | rc_AA892779_at | AA892779 | 361351 | similar to RIKEN cDNA 3110052N05 (predicted) | Rn.7319 |
| 711 | 84069 | rc_AA892820_at | AA892820 | 306204 | filamin, beta (predicted) | Rn.1761 |
| 712 | 84072 | rc_AA892828_at | NM_001007620 | 289950 | pyruvate dehydrogenase (lipoamide) beta | Rn.102424 |
| 713 | 84076 | rc_AA892832_at | NM_134382 | 171400 | ELOVL family member 5, elongation of long chain fatty acids (yeast) | Rn.4243 |
| 714 | 84079 | rc_AA892843_at | NM_001007637 | 295224 | mitochondrial ribosomal protein L24 | Rn.3728 |
| 715 | 84083 | rc_AA892851_g_at | NM_001008521 | 315265 | protein tyrosine kinase 9 (predicted) | Rn.64467 |
| 716 | 84085 | rc_AA892855_at | NM_001009690 | 309201 | rod outer segment membrane protein 1 (predicted) | Rn.2058 |
| 717 | 84089 | rc_AA892860_g_at | AA892860 | 313845 | Son of sevenless homolog 1 (Drosophila) (predicted) | Rn.91844 |
| 718 | 84090 | rc_AA892861_at | AA892861 | 362576 | similar to RIKEN cDNA 2610528J11 (predicted) | Rn.1992 |
| 719 | 84106 | rc_AA892940_g_at | AA892940 | 287739 | similar to 116 kDa U5 small nuclear ribonucleoprotein component (U5 snRNP- | Rn.62495 |
| | | | | | specific protein, 116 kDa) (U5-116 kDa) | |
| 720 | 84115 | rc_AA892987_at | AA892987 | 361421 | TATA box binding protein (Tbp)-associated factor, RNA polymerase I, C | Rn.13462 |
| | | | | | (predicted) | |
| 721 | 84118 | rc_AA892997_at | AA892997 | | | Rn.23962 |
| 722 | 84128 | rc_AA893035_s_at | NM_134330 | 171179 | kidney expressed gene 1 | Rn.22745 |
| 723 | 84132 | rc_AA893043_at | AA893043 | | | Rn.9073 |
| 724 | 84153 | rc_AA893172_at | AA893172 | | | |
| 725 | 84158 | rc_AA893184_at | AA893184 | 311254 | similar to Ac1164 | Rn.2260 |
| 726 | 84181 | rc_AA893237_at | AA893237 | 300441 | hypothetical LOC300441 | Rn.13486 |
| 727 | 84182 | rc_AA893239_at | NM_053493 | 85255 | 2-hydroxyphytanoyl-Coenzyme A lyase | Rn.21425 |
| 728 | 84183 | rc_AA893242_at | NM_012820 | 25288 | acyl-CoA synthetase long-chain family member 1 | Rn.6215 |
| 728 | 84184 | rc_AA893242_g_at | NM_012820 | 25288 | acyl-CoA synthetase long-chain family member 1 | Rn.6215 |
| 729 | 84185 | rc_AA893244_at | AA893244 | 294103 | 3′-phosphoadenosine 5′-phosphosulfate synthase 2 (predicted) | Rn.79315 |
| 730 | 84186 | rc_AA893246_at | NM_199386 | 299159 | ATPase, H+ transporting, lysosomal 34 kDa, V1 subunit D | Rn.106041 |
| 731 | 84192 | rc_AA893280_at | NM_001007144 | 298199 | adipose differentiation-related protein | Rn.101967 |
| 732 | 84216 | rc_AA893493_i_at | AA893493 | | | Rn.107056 |
| 733 | 84224 | rc_AA893534_at | AA893534 | | | Rn.23024 |
| 734 | 84230 | rc_AA893584_at | AA893584 | 293938 | biogenesis of lysosome-related organelles complex-1, subunit 2 (predicted) | Rn.19119 |
| 735 | 84232 | rc_AA893592_at | AA893592 | 362182 | reticulocalbin (predicted) | Rn.103345 |
| 736 | 84250 | rc_AA893659_at | AA893659 | 362778 | similar to RIKEN cDNA 4933433P14 gene (predicted) | Rn.3701 |
| 737 | 84279 | rc_AA893770_g_at | NM_139104 | 245963 | EGF-like domain 7 | Rn.55456 |
| 738 | 84281 | rc_AA893781_at | AA893781 | 287443 | centaurin, beta 1 (predicted) | Rn.2165 |
| 739 | 84287 | rc_AA893821_at | NM_134398 | 171435 | p34 protein | Rn.12544 |
| 740 | 84291 | rc_AA893857_at | AA893857 | | | Rn.3104 |
| 741 | 84313 | rc_AA893984_at | AA893984 | 303073 | cytoplasmic FMR1 interacting protein 2 (predicted) | Rn.44008 |
| 742 | 84316 | rc_AA894004_at | AA894004 | 297339 | capping protein (actin filament), gelsolin-like (predicted) | Rn.8945 |
| 743 | 84317 | rc_AA894008_at | AA894008 | 313052 | similar to erythrocyte membrane protein band 4.1 (elliptocytosis 1, RH-linked) | Rn.99632 |
| | | | | | isoform 1 | |
| 744 | 84320 | rc_AA894016_at | AA894016 | 362251 | RNA-binding region (RNP1, RRM) containing 2 (predicted) | Rn.8555 |
| 745 | 84322 | rc_AA894027_at | AA894027 | | | |
| 746 | 84326 | rc_AA894045_at | NM_181822 | 294251 | natural cytotoxicity triggering receptor 3 | Rn.14846 |
| 747 | 84338 | rc_AA894092_at | AA894092 | 361945 | periostin, osteoblast specific factor (predicted) | Rn.30516 |
| 748 | 81056 | rc_AA894174_at | NM_001009668 | 300726 | electron transferring flavoprotein, alpha polypeptide | Rn.32496 |
| 749 | 84360 | rc_AA894200_g_at | AA894200 | 362816 | myosin, light polypeptide 6, alkali, smooth muscle and non-muscle (predicted) | Rn.969 |
| 750 | 84374 | rc_AA894259_at | AA894259 | 363852 | similar to RIKEN cDNA 2010110M21 | Rn.13525 |
| 751 | 84380 | rc_AA894292_at | AA894292 | 84420 | adenylate cyclase 7 | Rn.19450 |
| 752 | 84383 | rc_AA894304_at | AA894304 | | | Rn.112831 |
| 753 | 81059 | rc_AA894317_s_at | NM_032083 | 84030 | chimerin (chimaerin) 1 | Rn.11166 |
| 754 | 84387 | rc_AA894318_at | AA894318 | 298201 | similar to RIKEN cDNA B230312A22 (predicted) | Rn.63818 |
| 755 | 84393 | rc_AA894345_at | AA894345 | 364052 | phosphoprotein enriched in astrocytes 15 (predicted) | Rn.2945 |
| 756 | 81110 | rc_AA899106_at | NM_022267 | 64033 | cyclin D2 | Rn.96083 |
| 757 | 81116 | rc_AA900503_at | NM_019147 | 29146 | jagged 1 | Rn.88804 |
| 758 | 81126 | rc_AA900900_s_at | NM_032067 | 84014 | ralA binding protein 1 | Rn.7107 |
| 759 | 77051 | rc_AA925300_at | AA925300 | 303604 | mitogen activated protein kinase kinase kinase 3 (predicted) | Rn.72680 |
| 760 | 81191 | rc_AA925752_at | NM_031561 | 29184 | cd36 antigen | Rn.102418 |
| 761 | 77054 | rc_AA926129_at | AA926129 | 303380 | schlafen 2 (predicted) | Rn.105052 |
| 762 | 76254 | rc_AA926149_g_at | NM_012520 | 24248 | catalase | Rn.3001 |
| 763 | 81152 | rc_AA945169_at | NM_012681 | 24856 | transthyretin | Rn.1404 |
| 764 | 81154 | rc_AA945171_at | AA945171 | 83418 | apolipoprotein C-IV | Rn.33157 |
| 765 | 76346 | rc_AA945704_at | AA945704 | 361384 | DnaJ (Hsp40) homolog, subfamily B, member 1 (predicted) | Rn.7896 |
| 766 | 81162 | rc_AA945737_at | NM_022205 | 60628 | chemokine (C—X—C motif) receptor 4 | Rn.44431 |
| 767 | 76312 | rc_AA946040_at | AA946040 | | | Rn.6009 |
| 768 | 81166 | rc_AA946108_at | AA946108 | 286966 | laminin, alpha 3 | Rn.10597 |
| 769 | 81167 | rc_AA946251_at | NM_030829 | 59075 | G protein-coupled receptor kinase 5 | Rn.6500 |
| 770 | 81172 | rc_AA946439_at | AA946439 | | | Rn.10465 |
| 771 | 81226 | rc_AA957930_s_at | NM_017212 | 29477 | microtubule-associated protein tau | Rn.2455 |
| 772 | 81227 | rc_AA957961_at | NM_054006 | 117180 | unr protein | Rn.3562 |
| 772 | 81395 | rc_AI070521_s_at | NM_054006 | 117180 | unr protein | Rn.3562 |
| 772 | 82424 | X52311_at | NM_054006 | 117180 | unr protein | Rn.3562 |
| 773 | 76318 | rc_AA963449_s_at | NM_012941 | 25427 | cytochrome P450, subfamily 51 | Rn.107152 |
| 773 | 76319 | rc_AA997614_s_at | NM_012941 | 25427 | cytochrome P450, subfamily 51 | Rn.107152 |
| 773 | 76902 | U17697_s_at | NM_012941 | 25427 | cytochrome P450, subfamily 51 | Rn.107152 |
| 774 | 81235 | rc_AA964530_at | NM_024150 | 79119 | ADP-ribosylation factor 2 | Rn.62175 |
| 775 | 81238 | rc_AA965119_at | NM_031534 | 24883 | Wilms tumor 1 | Rn.92531 |
| 776 | 76343 | rc_AI007820_s_at | NM_001004082 | 301252 | heat shock 90 kDa protein 1, beta | Rn.98667 |
| 776 | 76344 | rc_AI008074_s_at | NM_001004082 | 301252 | heat shock 90 kDa protein 1, beta | Rn.98667 |
| 777 | 81262 | rc_AI007824_g_at | AI007824 | | | |
| 778 | 81263 | rc_AI007835_at | NM_053343 | 83825 | double cortin and calcium/calmodulin-dependent protein kinase-like 1 | Rn.80575 |
| 779 | 76186 | rc_AI009141_at | AI009141 | | | Rn.29900 |
| 780 | 76276 | rc_AI009390_at | AI009390 | 362588 | NADH dehydrogenase (ubiquinone) Fe—S protein 5b, 15 kDa (NADH-coenzyme | Rn.3392 |
| | | | | | Q reductase) (predicted) | |
| 781 | 81286 | rc_AI009605_at | NM_013216 | 26954 | Ras homolog enriched in brain | Rn.859 |
| 782 | 81292 | rc_AI010083_at | NM_057114 | 117254 | peroxiredoxin 1 | Rn.2845 |
| 783 | 81295 | rc_AI010453_at | NM_022519 | 24648 | serine (or cysteine) proteinase inhibitor, clade A (alpha-1 antiproteinase, | Rn.1419 |
| | | | | | antitrypsin), member 1 | |
| 784 | 76191 | rc_AI011706_at | AI011706 | 361814 | splicing factor, arginine/serine-rich 3 (SRp20) (predicted) | Rn.9002 |
| 785 | 81309 | rc_AI012595_at | NM_017039 | 24672 | protein phosphatase 2a, catalytic subunit, alpha isoform | Rn.1271 |
| 786 | 81325 | rc_AI013389_at | NM_012521 | 24249 | calbindin 3, (vitamin D-dependent calcium binding protein) | Rn.9974 |
| 787 | 81326 | rc_AI013472_at | NM_138905 | 192270 | ER transmembrane protein Dri 42 | Rn.12038 |
| 788 | 81329 | rc_AI013795_at | NM_022713 | 64679 | transglutaminase 4 (prostate) | Rn.9964 |
| 789 | 76170 | rc_AI013834_s_at | NM_024392 | 79244 | hydroxysteroid (17-beta) dehydrogenase4 | Rn.2082 |
| 790 | 81318 | rc_AI014135_at | AI014135 | | | |
| 791 | 81320 | rc_AI014163_at | NM_019242 | 29596 | interferon-related developmental regulator 1 | Rn.3723 |
| 792 | 81333 | rc_AI029183_s_at | NM_012567 | 24392 | gap junction membrane channel protein alpha 1 | Rn.10346 |
| 793 | 81334 | rc_AI029279_at | NM_053868 | 116647 | neuroligin 1 | Rn.10173 |
| 794 | 81342 | rc_AI031019_at | NM_172029 | 64514 | eukaryotic translation initiation factor 2B, subunit 1 alpha | Rn.9181 |
| 795 | 81359 | rc_AI044517_at | NM_017215 | 29482 | solute carrier family 1 (glial high affinity glutamate transporter), member 2 | Rn.10240 |
| 795 | 82215 | U15098_at | NM_017215 | 29482 | solute carrier family 1 (glial high affinity glutamate transporter), member 2 | Rn.10240 |
| 796 | 81361 | rc_AI044716_at | NM_153735 | 266777 | neuronal pentraxin 1 | Rn.54707 |
| 797 | 81362 | rc_AI044739_at | NM_133423 | 170956 | splicing factor YT521-B | Rn.48752 |
| 798 | 81366 | rc_AI044985_g_at | NM_019241 | 29586 | gap junction membrane channel protein beta 5 | Rn.16074 |
| 799 | 81392 | rc_AI070142_at | NM_017030 | 24624 | propionyl coenzyme A carboxylase, beta polypeptide | Rn.9732 |
| 800 | 81396 | rc_AI070577_at | NM_012817 | 25285 | insulin-like growth factor binding protein 5 | Rn.1593 |
| 801 | 81398 | rc_AI070783_at | NM_017313 | 29885 | RAB3A interacting protein | Rn.31889 |
| 802 | 81403 | rc_AI071104_at | NM_012774 | 25236 | glypican 3 | Rn.9717 |
| 803 | 81405 | rc_AI071299_at | NM_031135 | 81813 | TGFB inducible early growth response | Rn.2398 |
| 804 | 81412 | rc_AI071866_s_at | AI071866 | | | |
| 805 | 81426 | rc_AI073232_s_at | NM_019246 | 29606 | proprotein convertase subtilisin/kexin type 7 | Rn.10653 |
| 806 | 81471 | rc_AI101320_at | AI101320 | 29147 | jagged 2 | Rn.22459 |
| 807 | 81494 | rc_AI102044_at | AI102044 | | | |
| 808 | 81498 | rc_AI102205_s_at | NM_024000 | 79011 | vesicle-associated calmodulin-binding protein | Rn.9958 |
| 809 | 81513 | rc_AI103671_at | NM_053311 | 29598 | ATPase, Ca++ transporting, plasma membrane 1 | Rn.7208 |
| 810 | 81517 | rc_AI103911_at | NM_001008888 | 291103 | ubiquinol-cytochrome c reductase, Rieske iron-sulfur polypeptide 1 | Rn.2603 |
| 811 | 81518 | rc_AI103957_at | NM_013087 | 25621 | CD 81 antigen | Rn.1975 |
| 812 | 76313 | rc_AI104035_s_at | AI104035 | | | Rn.6009 |
| 813 | 76204 | rc_AI104077_at | AI104077 | | | Rn.30301 |
| 814 | 81527 | rc_AI104399_at | NM_022922 | 24849 | triosephosphate isomerase 1 | Rn.37838 |
| 815 | 81529 | rc_AI104513_at | NM_145783 | 252934 | cytochrome c oxidase, subunit Va | Rn.11077 |
| 816 | 81536 | rc_AI104781_at | NM_017260 | 29624 | arachidonate 5-lipoxygenase activating protein | Rn.18399 |
| 817 | 81539 | rc_AI105348_i_at | NM_017147 | 29271 | cofilin 1 | Rn.11675 |
| 818 | 81429 | rc_AI112173_at | NM_013113 | 25650 | ATPase, Na+/K+ transporting, beta 1 polypeptide | Rn.8925 |
| 819 | 81436 | rc_AI136396_at | NM_172034 | 64511 | farnesyltransferase, CAAX box, beta | Rn.8873 |
| 820 | 76228 | rc_AI136977_at | AI136977 | 260321 | FK506 binding protein 4 | Rn.23741 |
| 821 | 81447 | rc_AI137583_at | NM_013060 | 25587 | Inhibitor of DNA binding 2, dominant negative helix-loop-helix protein | Rn.3272 |
| 822 | 81451 | rc_AI138143_at | NM_012796 | 29487 | glutathione S-transferase, theta 2 | Rn.87212 |
| 823 | 81459 | rc_AI145367_at | NM_053874 | 116653 | CAP, adenylate cyclase-associated protein, 2 (yeast) | Rn.10229 |
| 824 | 81465 | rc_AI145680_s_at | NM_012716 | 25027 | solute carrier family 16 (monocarboxylic acid transporters), member 1 | Rn.6085 |
| 825 | 76347 | rc_AI169265_at | AI169265 | | | Rn.793 |
| 826 | 81483 | rc_AI169327_at | NM_053819 | 116510 | tissue inhibitor of metalloproteinase 1 | Rn.25754 |
| 826 | 81484 | rc_AI169327_g_at | NM_053819 | 116510 | tissue inhibitor of metalloproteinase 1 | Rn.25754 |
| 827 | 81544 | rc_AI169612_at | NM_053365 | 79451 | fatty acid binding protein 4, adipocyte | Rn.4258 |
| 828 | 81548 | rc_AI169756_s_at | AI169756 | | | Rn.100336 |
| 829 | 76133 | rc_AI170379_at | AI170379 | | | Rn.101657 |
| 830 | 81557 | rc_AI170411_s_at | NM_053591 | 94199 | dipeptidase 1 (renal) | Rn.6051 |
| 831 | 81559 | rc_AI170608_at | NM_031616 | 58821 | zinc finger protein 265 | Rn.11361 |
| 832 | 76288 | rc_AI170685_at | NM_032079 | 84026 | DnaJ (Hsp40) homolog, subfamily A, member 2 | Rn.3904 |
| 832 | 76289 | rc_AI170685_g_at | NM_032079 | 84026 | DnaJ (Hsp40) homolog, subfamily A, member 2 | Rn.3904 |
| 833 | 79331 | Z18878cds_at | NM_053994 | 117098 | pyruvate dehydrogenase E1 alpha 2 | Rn.11126 |
| 833 | 81563 | rc_AI170911_at | NM_053994 | 117098 | pyruvate dehydrogenase E1 alpha 2 | Rn.11126 |
| 834 | 81565 | rc_AI171067_at | NM_012600 | 24552 | malic enzyme 1 | Rn.3519 |
| 835 | 81577 | rc_AI171844_at | NM_139099 | 245958 | ATP synthase, H+ transporting, mitochondrial F1 complex, epsilon subunit | Rn.3454 |
| 836 | 81584 | rc_AI172064_at | NM_019904 | 56646 | lectin, galactose binding, soluble 1 | Rn.57 |
| 837 | 76357 | rc_AI172452_at | AI172452 | 298762 | cytochrome c oxidase subunit VIIa polypeptide 2 like (predicted) | Rn.3907 |
| 838 | 81595 | rc_AI175486_at | AI175486 | 298661 | hypothetical LOC298661 | Rn.119388 |
| 839 | 81606 | rc_AI175935_at | NM_173101 | 25484 | myosin IE | Rn.8737 |
| 840 | 81611 | rc_AI176351_s_at | NM_031137 | 81815 | tripeptidyl peptidase II | Rn.28991 |
| 840 | 81644 | rc_AI178007_at | NM_031137 | 81815 | tripeptidyl peptidase II | Rn.28991 |
| 841 | 81613 | rc_AI176460_s_at | AI176460 | | | |
| 842 | 81614 | rc_AI176461_s_at | NM_017211 | 29476 | golgi apparatus protein 1 | Rn.89086 |
| 842 | 82500 | U08136_at | NM_017211 | 29476 | golgi apparatus protein 1 | Rn.89086 |
| 843 | 81620 | rc_AI176595_s_at | NM_013156 | 25697 | cathepsin L | Rn.1294 |
| 844 | 81624 | rc_AI176710_at | NM_031628 | 58853 | nuclear receptor subfamily 4, group A, member 3 | Rn.62694 |
| 845 | 76293 | rc_AI176726_at | AI176726 | | | |
| 846 | 81629 | rc_AI177052_at | AI177052 | 25281 | nucleoporin 153 kD | Rn.1347 |
| 847 | 81632 | rc_AI177161_at | NM_031789 | 83619 | nuclear factor, erythroid derived 2, like 2 | Rn.10867 |
| 847 | 81633 | rc_AI177161_g_at | NM_031789 | 83619 | nuclear factor, erythroid derived 2, like 2 | Rn.10867 |
| 848 | 81637 | rc_AI177503_at | NM_053985 | 117056 | H3 histone, family 3B | Rn.29857 |
| 849 | 81654 | rc_AI179012_s_at | NM_031144 | 81822 | actin, beta | Rn.94978 |
| 850 | 81660 | rc_AI179610_at | NM_012580 | 24451 | heme oxygenase (decycling) 1 | Rn.3160 |
| 851 | 81663 | rc_AI180013_at | NM_033351 | 29558 | Fc receptor, IgG, alpha chain transporter | Rn.15845 |
| 852 | 81668 | rc_AI180410_i_at | NM_020079 | 24658 | prolactin-like protein C 1 | Rn.40117 |
| 853 | 81671 | rc_AI180424_at | NM_013011 | 25578 | tyrosine 3-monooxygenase/tryptophan 5-monooxygenase activation protein, | Rn.1292 |
| | | | | | zeta polypeptide | |
| 854 | 81672 | rc_AI180442_at | NM_031840 | 83791 | farensyl diphosphate synthase | Rn.2848 |
| 855 | 81678 | rc_AI228247_at | NM_013106 | 25643 | guanine nucleotide binding protein, alpha inhibiting 3 | Rn.4368 |
| 856 | 81696 | rc_AI230211_s_at | NM_031739 | 65195 | potassium voltage gated channel, Shal-related family, member 3 | Rn.10540 |
| 857 | 81700 | rc_AI230354_at | NM_022538 | 64369 | phosphatidic acid phosphatase 2a | Rn.61687 |
| 858 | 81705 | rc_AI230712_at | NM_012999 | 25507 | Subtilisin - like endoprotease | Rn.950 |
| 859 | 81706 | rc_AI230748_at | NM_053867 | 116646 | tumor protein, translationally-controlled 1 | Rn.36610 |
| 860 | 81722 | rc_AI231445_at | AI231445 | | | Rn.121327 |
| 861 | 81726 | rc_AI231807_at | NM_022500 | 29292 | ferritin light chain 1 | Rn.1905 |
| 862 | 81729 | rc_AI232078_at | NM_021587 | 59107 | latent transforming growth factor beta binding protein 1 | Rn.40942 |
| 863 | 81731 | rc_AI232096_at | NM_031672 | 60577 | solute carrier family 15 (H+/peptide transporter), member 2 | Rn.89268 |
| 864 | 81732 | rc_AI232194_at | NM_032084 | 84031 | chimerin (chimaerin) 2 | Rn.10521 |
| 865 | 81733 | rc_AI232256_at | NM_030586 | 80773 | cytochrome b5, outer mitochondrial membrane isoform | Rn.10249 |
| 866 | 81734 | rc_AI232268_at | AI232268 | 116565 | low density lipoprotein receptor-related protein associated protein 1 | Rn.10293 |
| 867 | 81755 | rc_AI234351_at | AI234351 | 363828 | similar to immunoglobulin light chain variable region | Rn.129 |
| 868 | 81759 | rc_AI234915_at | AI234915 | 303907 | WD repeat domain 5B (predicted) | Rn.38769 |
| 869 | 81778 | rc_AI236484_at | NM_198132 | 362152 | heterogeneous nuclear ribonucleoprotein A3 | Rn.99947 |
| 870 | 76224 | rc_AI236601_at | NM_001011901 | 288444 | heat shock protein 105 (predicted) | Rn.37805 |
| 871 | 81784 | rc_AI237535_s_at | AI237535 | 65161 | LPS-induced TNF-alpha factor | Rn.6940 |
| 872 | 81786 | rc_AI237592_at | AI237592 | 117060 | progestin induced protein | Rn.54812 |
| 873 | 81789 | rc_AI237825_at | NM_012533 | 24271 | carboxypeptidase B1 | Rn.12780 |
| 874 | 80438 | rc_AI638939_at | AI638939 | 303970 | bobby sox homolog (Drosophila) (predicted) | Rn.113000 |
| 875 | 80439 | rc_AI638940_at | AI638940 | | | |
| 876 | 80450 | rc_AI638951_at | AI638951 | | | |
| 877 | 80451 | rc_AI638952_at | AI638952 | | | |
| 878 | 80465 | rc_AI638969_at | NM_133605 | 171140 | calcium/calmodulin-dependent protein kinase II gamma | Rn.10961 |
| 879 | 80489 | rc_AI638996_at | AI638996 | | | |
| 880 | 80497 | rc_AI639007_at | AI639007 | | | |
| 881 | 80523 | rc_AI639035_at | AI639035 | | | Rn.28819 |
| 882 | 80545 | rc_AI639058_s_at | AI639058 | 311676 | transmembrane, prostate androgen induced RNA (predicted) | Rn.108034 |
| 883 | 80546 | rc_AI639060_at | AI639060 | | | Rn.135652 |
| 884 | 80551 | rc_AI639065_at | AI639065 | | | |
| 885 | 80556 | rc_AI639071_at | AI639071 | | | |
| 886 | 80589 | rc_AI639107_at | AI639107 | 364396 | ADP-ribosylation factor-like 11 (predicted) | Rn.23090 |
| 887 | 80596 | rc_AI639115_at | NM_198743 | 296466 | BWK-1 | Rn.28608 |
| 888 | 80610 | rc_AI639132_s_at | AI639132 | 297903 | similar to RIKEN cDNA 6720467C03 (predicted) | Rn.92601 |
| 889 | 80629 | rc_AI639152_at | AI639152 | 314406 | similar to Alpha-1-antitrypsin-related protein precursor (predicted) | Rn.99752 |
| 890 | 80651 | rc_AI639175_at | AI639175 | | | |
| 891 | 80655 | rc_AI639179_at | AI639179 | | | |
| 892 | 80656 | rc_AI639181_at | AI639181 | | | Rn.131203 |
| 893 | 80664 | rc_AI639189_at | AI639189 | 303572 | similar to nuclear protein with a coiled coil-4 domain of bilaterial origin like | Rn.136691 |
| | | | | | (3L720) | |
| 894 | 80665 | rc_AI639190_at | AI639190 | | | |
| 895 | 80667 | rc_AI639192_at | AI639192 | | | |
| 896 | 80684 | rc_AI639209_at | AI639209 | | | |
| 897 | 80685 | rc_AI639210_at | AI639210 | 313352 | doublesex and mab-3 related transcription factor like family A1 (predicted) | Rn.43064 |
| 898 | 80686 | rc_AI639212_at | NM_017298 | 29716 | calcium channel, voltage-dependent, L type, alpha 1D subunit | Rn.89671 |
| 899 | 80691 | rc_AI639217_at | AI639217 | | | |
| 900 | 80692 | rc_AI639218_at | NM_001009268 | 289820 | ARP2 actin-related protein 2 homolog (yeast) (predicted) | Rn.102249 |
| 901 | 80715 | rc_AI639246_at | AI639246 | 293186 | extra cellular link domain-containing 1 (predicted) | Rn.123582 |
| 902 | 80723 | rc_AI639252_at | AI639252 | | | |
| 903 | 80728 | rc_AI639256_at | NM_181376 | 301255 | spermatogenesis associated, serine-rich 1 | Rn.40672 |
| 904 | 80733 | rc_AI639263_at | AI639263 | | | |
| 905 | 80734 | rc_AI639264_at | AI639264 | | | |
| 906 | 80755 | rc_AI639293_at | AI639293 | | | |
| 907 | 80788 | rc_AI639324_g_at | AI639324 | 307390 | F-box protein 38 (predicted) | Rn.13347 |
| 908 | 80792 | rc_AI639328_at | AI639328 | | | |
| 909 | 80793 | rc_AI639329_at | AI639329 | 307075 | WD repeat domain 37 (predicted) | Rn.43138 |
| 910 | 80805 | rc_AI639343_at | AI639343 | | | Rn.13858 |
| 911 | 80831 | rc_AI639370_at | NM_001007725 | 360647 | intercellular adhesion molecule 2 | Rn.112767 |
| 912 | 80857 | rc_AI639400_at | AI639400 | | | Rn.135073 |
| 913 | 80861 | rc_AI639404_at | AI639404 | | | |
| 914 | 80862 | rc_AI639405_at | NM_138831 | 170566 | solute carrier family 16 (monocarboxylic acid transporters), member 10 | Rn.21992 |
| 915 | 80868 | rc_AI639411_at | NM_207614 | 362855 | P55 | Rn.11875 |
| 916 | 80870 | rc_AI639412_at | AI639412 | 306805 | similar to asporin precursor | Rn.45067 |
| 917 | 80888 | rc_AI639432_at | AI639432 | | | |
| 918 | 80018 | U38253_g_at | NM_133609 | 171145 | eukaryotic translation initiation factor 2B, subunit 3 gamma | Rn.10577 |
| 918 | 80897 | rc_AI639441_at | NM_133609 | 171145 | eukaryotic translation initiation factor 2B, subunit 3 gamma | Rn.10577 |
| 919 | 80901 | rc_AI639444_g_at | NM_057191 | 117537 | kelch repeat and BTB (POZ) domain containing 10 | Rn.28875 |
| 920 | 80920 | rc_AI639461_at | AI639461 | | | Rn.120244 |
| 921 | 80927 | rc_AI639467_at | AI639467 | 25716 | synaptotagmin 1 | Rn.7596 |
| 922 | 80937 | rc_AI639476_s_at | AI639476 | 304582 | squamous cell carcinoma antigen recognized by T-cells 3 (predicted) | Rn.101480 |
| 923 | 80954 | rc_AI639495_at | AI639495 | | | |
| 924 | 80957 | rc_AI639498_i_at | AI639498 | | | |
| 925 | 80964 | rc_AI639505_at | AI639505 | | | |
| 926 | 80975 | rc_AI639518_at | AI639518 | 287988 | polymerase (RNA) II (DNA directed) polypeptide H (predicted) | Rn.9160 |
| 927 | 80998 | rc_AI639536_at | AI639536 | | | Rn.64791 |
| 928 | 83529 | rc_H31232_at | NM_053472 | 84683 | cytochrome c oxidase subunit IV isoform 2 | Rn.7214 |
| 929 | 83544 | rc_H31535_at | H31535 | 303564 | similar to RIKEN cDNA 2610511E22 (predicted) | Rn.94772 |
| 930 | 83545 | rc_H31550_at | H31550 | | | Rn.14572 |
| 931 | 83546 | rc_H31554_at | H31554 | | | Rn.14573 |
| 932 | 83549 | rc_H31597_at | NM_153294 | 259224 | prepro-Neuropeptide W polypeptide | Rn.14575 |
| 933 | 83556 | rc_H31692_at | NM_021597 | 59117 | GERp95 | Rn.35512 |
| 934 | 83572 | rc_H31887_at | H31887 | 311428 | similar to RIKEN cDNA 1700037H04 (predicted) | Rn.28249 |
| 935 | 83573 | rc_H31897_at | H31897 | | | Rn.21418 |
| 936 | 83578 | rc_H31964_at | H31964 | | | Rn.23171 |
| 937 | 83581 | rc_H31990_at | H31990 | | | |
| 938 | 83587 | rc_H33084_at | H33084 | | | |
| 939 | 83593 | rc_H33149_at | H33149 | 361151 | similar to RIKEN cDNA 1810047C23 (predicted) | Rn.14630 |
| 940 | 83611 | rc_H33528_at | H33528 | | | Rn.14645 |
| 941 | 83614 | rc_H33566_at | H33566 | | | Rn.92617 |
| 942 | 83615 | rc_H33577_at | H33577 | | | Rn.131034 |
| 943 | 83621 | rc_H33660_at | H33660 | 299153 | similar to chromosome 14 open reading frame 50 (predicted) | Rn.3331 |
| 944 | 78424 | S42358_s_at | NM_024372 | 79213 | solute carrier family 6 (neurotransmitter transporter, GABA), member 11 | Rn.10545 |
| 945 | 78426 | S43408_g_at | NM_013143 | 25684 | meprin 1 alpha | Rn.16265 |
| 946 | 78428 | S45663_at | NM_138549 | 191576 | glycoprotein, synaptic 2 | Rn.107358 |
| 947 | 78435 | S46798cds#1_s_at | S46798 | | | |
| 948 | 78445 | S50461_s_at | NM_031034 | 81663 | guanine nucleotide binding protein, alpha 12 | Rn.10497 |
| 949 | 78452 | S53987_at | S53987 | | | |
| 950 | 78457 | S55427_s_at | NM_017037 | 24660 | peripheral myelin protein 22 | Rn.1476 |
| 951 | 78460 | S56464mRNA_at | S56464 | | | |
| 951 | 78461 | S56464mRNA_g_at | S56464 | | | |
| 952 | 78470 | S58528_at | S58528 | | | |
| 953 | 78478 | S60054_s_at | NM_012642 | 24715 | renin 1 | Rn.9831 |
| 954 | 78481 | S61868_at | NM_012649 | 24771 | syndecan 4 | Rn.2029 |
| 954 | 78482 | S61868_g_at | NM_012649 | 24771 | syndecan 4 | Rn.2029 |
| 955 | 78485 | S61960_s_at | S61960 | 311844 | cysteine conjugate-beta lyase (predicted) | Rn.110564 |
| 956 | 78486 | S61973_at | NM_153308 | 266668 | glutamate receptor, ionotropic, N-methyl D-asparate-associated protein 1 | Rn.5898 |
| | | | | | (glutamate binding) | |
| 957 | 78489 | S62096_s_at | NM_031654 | 58983 | Rab geranylgeranyl transferase, a subunit | Rn.29434 |
| 958 | 78495 | S63233_g_at | NM_053290 | 24642 | phosphoglycerate mutase 1 | Rn.1383 |
| 959 | 78505 | S65091_at | S65091 | 25519 | protein phosphatase 1, regulatory subunit 1C | |
| 960 | 78509 | S65555_at | NM_017305 | 29739 | glutamate cysteine ligase, modifier subunit | Rn.2460 |
| 960 | 78510 | S65555_g_at | NM_017305 | 29739 | glutamate cysteine ligase, modifier subunit | Rn.2460 |
| 961 | 78511 | S66024_at | NM_017334 | 25620 | cAMP responsive element modulator | Rn.10251 |
| 961 | 78512 | S66024_g_at | NM_017334 | 25620 | cAMP responsive element modulator | Rn.10251 |
| 962 | 78513 | S66184_s_at | NM_017061 | 24914 | lysyl oxidase | Rn.11372 |
| 962 | 78601 | S77494_s_at | NM_017061 | 24914 | lysyl oxidase | Rn.11372 |
| 963 | 78516 | S67435_s_at | NM_022852 | 29535 | pancreatic and duodenal homeobox gene 1 | Rn.54603 |
| 964 | 78521 | S67900_s_at | NM_080477 | 24640 | 6-phosphofructo-2-kinase/fructose-2,6-biphosphatase 2 | Rn.44844 |
| 965 | 78529 | S68809_s_at | NM_001007636 | 295214 | S100 calcium binding protein A1 | Rn.11091 |
| 966 | 78532 | S69160_s_at | NM_013047 | 25570 | thyrotropin releasing hormone receptor | Rn.9962 |
| 967 | 76994 | X78848cds_f_at | NM_031509 | 24421 | glutathione S-transferase A5 | Rn.40574 |
| 967 | 78555 | S72505_f_at | NM_031509 | 24421 | glutathione S-transferase A5 | Rn.10460 |
| 968 | 78557 | S72594_s_at | NM_021989 | 29543 | tissue inhibitor of metalloproteinase 2 | Rn.10161 |
| 969 | 78563 | S73424_s_at | NM_031051 | 81683 | macrophage migration inhibitory factor | Rn.2661 |
| 970 | 78569 | S74257_g_at | S74257 | | | |
| 971 | 78572 | S74393_s_at | NM_013001 | 25509 | paired box gene 6 | Rn.89724 |
| 972 | 78574 | S74572_g_at | NM_033096 | 24667 | protein phosphatase 1B, magnesium dependent, beta isoform | Rn.4143 |
| 973 | 78595 | S76466_at | NM_199230 | 29381 | activin A receptor, type 1B | Rn.103436 |
| 974 | 78602 | S77528cds_s_at | NM_024125 | 24253 | CCAAT/enhancer binding protein (C/EBP), beta | Rn.6479 |
| 974 | 82411 | X60769mRNA_at | NM_024125 | 24253 | CCAAT/enhancer binding protein (C/EBP), beta | Rn.6479 |
| 975 | 78603 | S77532_at | S77532 | | | |
| 976 | 78604 | S77556_s_at | NM_013137 | 25678 | discoidin domain receptor family, member 1 | Rn.7807 |
| 977 | 78606 | S77858_s_at | S77858 | | | |
| 978 | 78616 | S78556_at | S78556 | | | |
| 979 | 78623 | S79304_s_at | S79304 | | | |
| 980 | 78629 | S79730_s_at | NM_013007 | 25516 | prepronociceptin | Rn.87935 |
| 981 | 78634 | S79820_g_at | S79820 | | | |
| 982 | 78638 | S80118_s_at | S80118 | | | |
| 983 | 78639 | S80127_s_at | NM_017044 | 24694 | parathyroid hormone | Rn.9768 |
| 984 | 78642 | S80379_s_at | S80379 | | | |
| 985 | 78644 | S80456_s_at | NM_012613 | 24603 | natriuretic peptide receptor 1 | Rn.10463 |
| 986 | 78653 | S81497_s_at | NM_012732 | 25055 | lipase A, lysosomal acid | Rn.48656 |
| 987 | 78670 | S83320_g_at | S83320 | | | |
| 988 | 76872 | S83436_i_at | NM_181371 | 297029 | glutathione S-transferase, mitochondrial | Rn.109452 |
| 989 | 78672 | S85184_at | S85184 | | | |
| 989 | 78673 | S85184_g_at | S85184 | | | |
| 990 | 78679 | S94371_at | S94371 | | | |
| 991 | 79818 | U02316_s_at | NM_031588 | 112400 | neuregulin 1 | Rn.37438 |
| 992 | 82149 | U02522_at | NM_022588 | 6452 | metastasis associated 1 | Rn.5840 |
| 993 | 79758 | U03120_at | NM_013033 | 25552 | solute carrier family 5 (sodium/glucose cotransporter), member 1 | Rn.10224 |
| 994 | 79594 | U03414_s_at | NM_053573 | 93667 | olfactomedin 1 | Rn.11005 |
| 995 | 78694 | U03763cds_at | NM_017174 | 29354 | phospholipase A2, group V | Rn.20244 |
| 996 | 79330 | Z15158mRNA_at | NM_013086 | 25620 | cAMP responsive element modulator | Rn.10251 |
| 996 | 79773 | U04835_at | NM_013086 | 25620 | cAMP responsive element modulator | Rn.10251 |
| 997 | 79725 | U04998_at | NM_013080 | 25613 | protein tyrosine phosphatase, receptor-type, Z polypeptide 1 | Rn.10088 |
| 998 | 76888 | U05014_at | NM_053857 | 116636 | eukaryotic translation initiation factor 4E binding protein 1 | Rn.11161 |
| 999 | 82060 | U05675_at | NM_020071 | 24366 | fibrinogen, B beta polypeptide | Rn.11416 |
| 1000 | 76715 | U06099_at | NM_017169 | 29338 | peroxiredoxin 2 | Rn.2511 |
| 1001 | 78701 | U06752_at | U06752 | 171578 | mucin 4 | Rn.24930 |
| 1002 | 82002 | U07683_at | NM_019276 | 50555 | UDP-glucuronosyltransferase 8 | Rn.9744 |
| 1003 | 79624 | U08256_at | NM_024379 | 79220 | glutamate receptor, ionotropic, delta 2 | Rn.10046 |
| 1004 | 82439 | U09228_at | NM_053369 | 84382 | transcription factor 4 | Rn.23354 |
| 1005 | 79921 | U09229_at | U09229 | 116639 | cut-like 1 (Drosophila) | Rn.10455 |
| 1006 | 78709 | U09401_s_at | U09401 | 116640 | tenascin C | Rn.12723 |
| 1007 | 82163 | U10188_at | NM_017100 | 25515 | polo-like kinase 1 (Drosophila) | Rn.11034 |
| 1008 | 79623 | U11031_at | NM_019329 | 54279 | contactin 3 | Rn.10043 |
| 1009 | 82032 | U11760_at | NM_053864 | 116643 | valosin-containing protein | Rn.98891 |
| 1010 | 79754 | U12184_at | NM_030831 | 80840 | G-protein coupled receptor 12 | Rn.88417 |
| 1011 | 79888 | U12514_at | NM_012982 | 25483 | msh homeo box homolog 2 (Drosophila) | Rn.10414 |
| 1012 | 82031 | U14007_at | NM_012825 | 25293 | aquaporin 4 | Rn.90091 |
| 1013 | 82176 | U14746_at | NM_052801 | 24874 | von Hippel-Lindau syndrome homolog | Rn.11059 |
| 1014 | 79659 | U17253_at | NM_022856 | 64824 | Ngfi-A binding protein 1 | Rn.10099 |
| 1015 | 76900 | U17260_s_at | NM_053853 | 116631 | N-acetyltransferase 1 (arylamine N-acetyltransferase) | Rn.37420 |
| 1016 | 79070 | X52817cds_s_at | NM_053865 | 116644 | reticulon 1 | Rn.55126 |
| 1016 | 82662 | U17604_at | NM_053865 | 116644 | reticulon 1 | Rn.55126 |
| 1017 | 82094 | U18771_at | NM_133580 | 171111 | RAB26, member RAS oncogene family | Rn.10975 |
| 1018 | 76903 | U18982_s_at | NM_012954 | 25446 | fos-like antigen 2 | Rn.10962 |
| 1019 | 82090 | U19614_at | NM_145092 | 246314 | lamina-associated polypeptide 1B | Rn.11373 |
| 1020 | 78731 | U20110cds_at | NM_053325 | 60566 | synaptotagmin 8 | Rn.46519 |
| 1021 | 79688 | U20181_at | NM_022863 | 64831 | iron responsive element binding protein 2 | Rn.10132 |
| 1022 | 79684 | U20283_at | NM_031126 | 81804 | syntaxin binding protein 2 | Rn.10121 |
| 1023 | 76904 | U20643mRNA#2_f_at | NM_012495 | 24189 | aldolase A | Rn.1774 |
| 1024 | 82039 | U20796_at | NM_147210 | 259241 | nuclear receptor subfamily 1, group D, member 2 | Rn.10055 |
| 1025 | 78735 | U21719mRNA_s_at | U21719 | 361847 | DEAD (Asp-Glu-Ala-Asp) box polypeptide 21b (predicted) | Rn.27170 |
| 1026 | 79694 | U22414_at | NM_013025 | 25542 | chemokine (C-C motif) ligand 3 | Rn.10139 |
| 1027 | 78739 | U23146cds_s_at | NM_057103 | 83425 | A kinase (PRKA) anchor protein (gravin) 12 | Rn.122094 |
| 1027 | 78779 | U41453_at | NM_057103 | 83425 | A kinase (PRKA) anchor protein (gravin) 12 | Rn.122094 |
| 1028 | 82134 | U23407_at | NM_017244 | 29563 | cellular retinoic acid binding protein 2 | Rn.11333 |
| 1029 | 76905 | U23438_at | NM_022199 | 60587 | dual specificity phosphatase 4 | Rn.44407 |
| 1030 | 82207 | U23769_at | NM_017365 | 54133 | PDZ and LIM domain 1 (elfin) | Rn.11170 |
| 1031 | 79787 | U25137_at | NM_017064 | 24918 | signal transducer and activator of transcription 5A | Rn.11355 |
| 1032 | 78740 | U25148_at | U25148 | 299509 | myosin IA | Rn.96429 |
| 1033 | 76731 | X05137_at | NM_012610 | 24596 | nerve growth factor receptor (TNFR superfamily, member 16) | Rn.10980 |
| 1033 | 76906 | U25650_f_at | NM_012610 | 24596 | nerve growth factor receptor (TNFR superfamily, member 16) | Rn.10980 |
| 1034 | 82148 | U26033_at | NM_031987 | 83842 | carnitine O-octanoyltransferase | Rn.4896 |
| 1035 | 82480 | U26397_at | NM_031002 | 80849 | inositol polyphosphate-4-phosphatase, type 1 | Rn.11215 |
| 1036 | 82848 | U30381_at | NM_031615 | 58820 | zinc finger protein 148 | Rn.64671 |
| 1037 | 82167 | U30831_at | NM_138849 | 192189 | B/K protein | Rn.31973 |
| 1038 | 78749 | U31160mRNA_s_at | NM_147211 | 259242 | SH3 domain binding protein CR16 | Rn.11272 |
| 1039 | 82136 | U31777_at | NM_017228 | 29515 | dentatorubral pallidoluysian atrophy | Rn.11305 |
| 1040 | 78759 | U33441mRNA_s_at | NM_133513 | 65182 | mucin 10, submandibular gland salivary mucin | Rn.10113 |
| 1041 | 79726 | U35774_at | NM_017253 | 29592 | branched chain aminotransferase 1, cytosolic | Rn.8273 |
| 1042 | 82130 | U35775_g_at | NM_031552 | 25230 | adducin 3 (gamma) | Rn.76589 |
| 1043 | 78762 | U36444cds#1_g_at | NM_031077 | 81741 | PCTAIRE-motif protein kinase 1 | Rn.16871 |
| 1044 | 78765 | U36771_g_at | NM_017274 | 29653 | glycerol-3-phosphate acyltransferase, mitochondrial | Rn.44456 |
| 1044 | 78767 | U36773_g_at | NM_017274 | 29653 | glycerol-3-phosphate acyltransferase, mitochondrial | Rn.44456 |
| 1045 | 76691 | U38376_s_at | NM_133551 | 24653 | phospholipase A2, group IVA (cytosolic, calcium-dependent) | Rn.10162 |
| 1046 | 82601 | U38481_at | NM_013022 | 25537 | Rho-associated coiled-coil forming kinase 2 | Rn.88642 |
| 1047 | 78773 | U39571_at | U39571 | | | |
| 1048 | 78775 | U39608_at | U39608 | | | |
| 1049 | 82520 | U39875_at | NM_024139 | 64152 | calcium binding protein p22 | Rn.11041 |
| 1050 | 78778 | U40819_at | NM_019142 | 65248 | protein kinase, AMP-activated, alpha 1 catalytic subunit | Rn.87789 |
| 1051 | 76916 | U40836mRNA_s_at | NM_012786 | 25250 | Cytochrom C oxidase subunit VIII-H (heart/muscle) | Rn.10325 |
| 1052 | 82715 | U41845_at | NM_012991 | 25497 | nuclear pore associated protein | Rn.3242 |
| 1053 | 76765 | U41853_at | NM_138867 | 192235 | hypoxia up-regulated 1 | Rn.10542 |
| 1054 | 82236 | U43534_at | NM_012894 | 25367 | adenosine deaminase, RNA-specific, B1 | Rn.89675 |
| 1055 | 78781 | U47014_at | U47014 | 116548 | proprotein convertase subtilisin/kexin type 5 | Rn.127948 |
| 1056 | 80045 | U47110_at | NM_022184 | 29647 | calcium/calmodulin-dependent serine protein kinase | Rn.72627 |
| 1057 | 76919 | U47313_at | U47313 | | | Rn.1712 |
| 1058 | 76921 | U47315_s_at | NM_133561 | 171087 | brain protein 44-like | Rn.3718 |
| 1059 | 76101 | U48220_at | NM_138515 | 171522 | cytochrome P450, family 2, subfamily d, polypeptide 22 | Rn.26060 |
| 1060 | 76754 | U48596_at | NM_053887 | 116667 | mitogen activated protein kinase kinase kinase 1 | Rn.11081 |
| 1061 | 82488 | U49099_at | U49099 | | | |
| 1062 | 76767 | U49930_at | NM_012922 | 25402 | caspase 3 | Rn.10562 |
| 1063 | 80029 | U50412_at | NM_013005 | 25513 | phosphatidylinositol 3-kinase, regulatory subunit, polypeptide 1 | Rn.133464 |
| 1064 | 78801 | U52034cds_i_at | NM_022175 | 24631 | placentae and embryos oncofetal gene | Rn.44483 |
| 1065 | 78807 | U52950_g_at | U52950 | 29456 | microtubule-associated protein 1b | Rn.98152 |
| 1066 | 82243 | U53184_at | U53184 | 65161 | LPS-induced TNF-alpha factor | Rn.6940 |
| 1067 | 82483 | U53211_at | NM_012892 | 25364 | amiloride-sensitive cation channel 1, neuronal (degenerin) | Rn.37523 |
| 1068 | 78810 | U53486mRNA_s_at | NM_030999 | 58959 | corticotropin releasing hormone receptor 1 | Rn.10499 |
| 1069 | 82264 | U53513_at | U53513 | 246295 | glycine-, glutamate-, thienylcyclohexylpiperidine-binding protein | |
| 1070 | 82267 | U53706_at | NM_031062 | 81726 | mevalonate (diphospho) decarboxylase | Rn.10288 |
| 1071 | 78811 | U53873cds_at | U53873 | | | |
| 1072 | 78812 | U53927_at | NM_022619 | 64554 | solute carrier family 7 (cationic amino acid transporter, y+ system), member 2 | Rn.48707 |
| 1073 | 80031 | U55836_at | NM_031089 | 81753 | parathyroid hormone receptor 2 | Rn.10601 |
| 1074 | 82684 | U55938_at | NM_013029 | 25547 | sialyltransferase 8 C | Rn.90977 |
| 1075 | 79994 | U56815_at | NM_031665 | 60562 | syntaxin 6 | Rn.100217 |
| 1076 | 82484 | U56839_at | NM_017255 | 29597 | purinergic receptor P2Y, G-protein coupled 2 | Rn.11102 |
| 1077 | 79952 | U57049_at | U57049 | 81724 | 5,10-methylenetetrahydrofolate reductase | Rn.10494 |
| 1078 | 82524 | U57062_at | NM_134332 | 171290 | granzyme C | Rn.88335 |
| 1079 | 79982 | U57063_at | NM_153466 | 266704 | natural killer cell protease 7 | Rn.10534 |
| 1080 | 82577 | U59241_at | NM_013044 | 25566 | tropomodulin 1 | Rn.1646 |
| 1081 | 79961 | U59672_at | NM_024394 | 79246 | 5-hydroxytryptamine (serotonin) receptor 3a | Rn.55109 |
| 1082 | 78834 | U62635_s_at | NM_022529 | 64360 | mitochondrial ribosomal protein L23 | Rn.1608 |
| 1083 | 80063 | U62667_at | NM_031123 | 81801 | stanniocalcin 1 | Rn.10647 |
| 1084 | 76779 | U62940_at | NM_024487 | 79563 | GrpE-like 1, mitochondrial | Rn.10630 |
| 1085 | 78836 | U63318_s_at | NM_139090 | 245921 | activin receptor-like kinase 7 | Rn.10580 |
| 1086 | 76927 | U64451_at | NM_013084 | 25618 | acyl-Coenzyme A dehydrogenase, short/branched chain | Rn.44423 |
| 1087 | 78838 | U64689_at | NM_053600 | 94269 | fasciculation and elongation protein zeta 2 (zygin II) | Rn.8714 |
| 1088 | 78839 | U64705cds_i_at | NM_001008335 | 303831 | eukaryotic translation initiation factor 4A2 (predicted) | Rn.104579 |
| 1089 | 82644 | U65656_at | NM_031054 | 81686 | matrix metallopeptidase 2 | Rn.6422 |
| 1090 | 78843 | U66292_at | NM_021677 | 59313 | pregnancy-specific beta 1-glycoprotein | Rn.42878 |
| 1091 | 78844 | U66293_at | U66293 | 292668 | pregnancy-specific beta 1-glycoprotein | Rn.44241 |
| 1092 | 78845 | U66298_at | NM_013107 | 25644 | bone morphogenetic protein 6 | Rn.40476 |
| 1093 | 80068 | U66322_at | NM_138863 | 192227 | leukotriene B4 12-hydroxydehydrogenase | Rn.10656 |
| 1094 | 80054 | U66478_at | NM_013130 | 25671 | SMAD, mothers against DPP homolog 1 (Drosophila) | Rn.10635 |
| 1095 | 80074 | U67082_at | NM_019620 | 25165 | zinc finger protein 386 (Kruppel-like) | Rn.10663 |
| 1096 | 78848 | U67207_s_at | NM_012596 | 24536 | leptin receptor | Rn.9891 |
| 1097 | 76789 | U69272_at | NM_013129 | 25670 | interleukin 15 | Rn.2490 |
| 1097 | 76790 | U69272_g_at | NM_013129 | 25670 | interleukin 15 | Rn.2490 |
| 1098 | 80122 | U69279_at | NM_053903 | 116683 | ephrin A5 | Rn.10714 |
| 1099 | 82847 | U70050_at | U70050 | 29147 | jagged 2 | Rn.22459 |
| 1100 | 76931 | U71089cds_at | NM_019310 | 54258 | interleukin 8 receptor, alpha | |
| 1101 | 82581 | U72660_at | NM_012867 | 25338 | ninjurin 1 | Rn.11245 |
| 1102 | 80319 | U72741_at | NM_012977 | 25476 | lectin, galactose binding, soluble 9 | Rn.10706 |
| 1102 | 80320 | U72741_g_at | NM_012977 | 25476 | lectin, galactose binding, soluble 9 | Rn.10706 |
| 1103 | 80181 | U72995_at | NM_053585 | 94193 | MAP-kinase activating death domain | Rn.90117 |
| 1104 | 78872 | U75412cds_at | U75412 | | | |
| 1105 | 76936 | U75899mRNA_at | NM_130431 | 161476 | heat shock 27 kD protein 2 | Rn.22486 |
| 1106 | 82650 | U75916_at | U75916 | 115769 | tight junction protein 2 | Rn.10965 |
| 1107 | 82651 | U75917_at | NM_022952 | 65046 | adaptor-related protein complex 2, sigma 1 subunit | Rn.37866 |
| 1108 | 78873 | U75920_at | NM_138509 | 114764 | microtubule-associated protein, RP/EB family, member 1 | Rn.7652 |
| 1109 | 78874 | U75921UTR#1_at | U75921 | | | |
| 1110 | 78875 | U75923cds_at | U75923 | 171557 | isoleucine-tRNA synthetase | |
| 1111 | 80287 | U77583_at | NM_053615 | 113927 | casein kinase 1, alpha 1 | Rn.23810 |
| 1112 | 78894 | U77829mRNA_i_at | U77829 | 81714 | growth arrest specific 5 | Rn.14733 |
| 1113 | 82606 | U77918_at | NM_031595 | 29677 | proteasome (prosome, macropain) 26S subunit, ATPase 3 | Rn.11173 |
| 1114 | 78895 | U77931_at | NM_147136 | 257642 | rRNA promoter binding protein | |
| 1115 | 78902 | U79568_s_at | NM_133289 | 78956 | sodium channel, voltage-gated, type 9, alpha polypeptide | Rn.88082 |
| 1116 | 78903 | U79661_at | NM_022203 | 60591 | calcium-activated potassium channel beta subunit | Rn.127785 |
| 1117 | 76822 | U80054_at | NM_031053 | 81685 | mutL homolog 1 (E. coli) | Rn.20391 |
| 1118 | 78905 | U81035_s_at | NM_053909 | 116690 | neurofascin | Rn.3048 |
| 1118 | 78906 | U81036_s_at | NM_053909 | 116690 | neurofascin | Rn.3048 |
| 1119 | 78907 | U81186_at | NM_032066 | 84013 | smooth muscle-specific 17 beta-hydroxysteroid dehydrogenase type 3 | Rn.2230 |
| 1120 | 82945 | U82591_at | NM_133525 | 171047 | chromosome 6 open reading frame 108 | Rn.6997 |
| 1121 | 82913 | U83230_at | NM_013217 | 26955 | afadin | Rn.58 |
| 1122 | 78914 | U83666_at | U83666 | 171576 | budding uninhibited by benzimidazoles 1 homolog, beta (S. cerevisiae) | Rn.49845 |
| 1123 | 78916 | U83880UTR#1_at | NM_012736 | 25062 | glycerol-3-phosphate dehydrogenase 2 | Rn.89705 |
| 1124 | 82640 | U83896_at | NM_053911 | 116692 | pleckstrin homology, Sec7 and coiled/coil domains 2 | Rn.3732 |
| 1125 | 78919 | U84727_at | NM_022398 | 64201 | 2-oxoglutarate carrier | Rn.853 |
| 1126 | 78921 | U85513_at | NM_031666 | 60567 | synaptotagmin X | Rn.74259 |
| 1127 | 78922 | U87305_at | NM_022206 | 60629 | unc-5 homolog A (C. elegans) | Rn.44433 |
| 1128 | 78923 | U87306_at | NM_022207 | 60630 | unc-5 homolog B (C. elegans) | Rn.34617 |
| 1129 | 82660 | U87983_at | NM_012964 | 25460 | hyaluronan mediated motility receptor | Rn.92304 |
| 1130 | 78933 | U90261UTR#1_g_at | NM_053360 | 84357 | SH3-domain kinase binding protein 1 | Rn.107226 |
| 1131 | 78936 | U90725_s_at | NM_172039 | 64474 | high density lipoprotein binding protein | Rn.8515 |
| 1132 | 78938 | U90829_g_at | NM_032072 | 84019 | amyloid beta precursor protein binding protein 1 | Rn.4279 |
| 1133 | 78942 | U92081mRNA_s_at | NM_019358 | 54320 | glycoprotein 38 | Rn.794 |
| 1134 | 80203 | U92564_g_at | NM_053583 | 94188 | Olf-1/EBF associated Zn finger protein Roaz | Rn.9981 |
| 1135 | 80300 | U92803_at | NM_078621 | 140473 | chemokine binding protein 2 | Rn.10771 |
| 1136 | 78945 | U93052_s_at | NM_031828 | 83731 | potassium large conductance calcium-activated channel, subfamily M, alpha | Rn.30616 |
| | | | | | member 1 | |
| 1137 | 80188 | U94330_at | NM_012870 | 25341 | tumor necrosis factor receptor superfamily, member 11b (osteoprotegerin) | Rn.9792 |
| 1138 | 82933 | U94904_s_at | NM_173143 | 286928 | abl-interactor 2 | Rn.11316 |
| 1139 | 78949 | U95001UTR#1_s_at | NM_053598 | 94267 | nudix (nucleoside diphosphate linked moiety X)-type motif 4 | Rn.8426 |
| 1140 | 80253 | U95178_s_at | NM_024159 | 79128 | disabled homolog 2 (Drosophila) | Rn.14763 |
| 1141 | 82570 | X02361_at | NM_012493 | 24177 | alpha-fetoprotein | Rn.9174 |
| 1142 | 76948 | X02904cds_s_at | NM_138974 | 29438 | glutathione S-transferase, pi 2 | Rn.87063 |
| 1143 | 82357 | X02918_at | NM_012998 | 25506 | prolyl 4-hydroxylase, beta polypeptide | Rn.4234 |
| 1144 | 78968 | X03914mergedCDS_UTR_at | X03914 | | | |
| 1145 | 82436 | X04070_at | NM_017251 | 29584 | gap junction membrane channel protein beta 1 | Rn.10444 |
| 1146 | 82384 | X05300_at | NM_013067 | 25596 | ribophorin I | Rn.4224 |
| 1147 | 78974 | X06150cds_at | NM_017084 | 25134 | glycine N-methyltransferase | Rn.11142 |
| 1148 | 76736 | X06827_at | NM_013168 | 25709 | hydroxymethylbilane synthase | Rn.11080 |
| 1149 | 78981 | X06832cds#2_s_at | NM_021655 | 24258 | chromogranin A | Rn.41024 |
| 1150 | 78982 | X06889cds_at | NM_013018 | 25531 | RAB3A, member RAS oncogene family | Rn.44409 |
| 1151 | 82354 | X06916_at | NM_012618 | 24615 | S100 calcium-binding protein A4 | Rn.504 |
| 1152 | 78984 | X06984cds_s_at | NM_012497 | 24191 | aldolase C, fructose-biphosphate | Rn.11211 |
| 1153 | 78986 | X07259cds_s_at | NM_153307 | 50549 | cytochrome P450, family 4, subfamily a, polypeptide 10 | Rn.92401 |
| 1154 | 79012 | X13905cds_g_at | NM_001008370 | 361706 | similar to Ras-related protein Rab-1B | Rn.105979 |
| 1155 | 76958 | X13933_s_at | NM_031969 | 24242 | calmodulin 1 | Rn.4166 |
| 1156 | 79025 | X14848cds#12_at | X14848 | | | |
| 1157 | 82350 | X15679_at | NM_173301 | 286960 | preprotrypsinogen IV | Rn.10387 |
| 1158 | 76963 | X15705cds_at | NM_021863 | 60460 | heat shock protein 2 | Rn.112579 |
| 1159 | 76722 | X16072_at | NM_012937 | 25422 | crystallin, beta B2 | Rn.10350 |
| 1160 | 79044 | X16623cds_s_at | X16623 | | | |
| 1161 | 79048 | X17053cds s_at | NM_031530 | 24770 | chemokine (C-C motif) ligand 2 | Rn.4772 |
| 1161 | 79049 | X17053mRNA_s_at | NM_031530 | 24770 | chemokine (C-C motif) ligand 2 | Rn.4772 |
| 1162 | 79051 | X17607cds_s_at | NM_012492 | 24176 | adrenergic receptor, beta 2 | Rn.10206 |
| 1163 | 79060 | X51706cds_at | NM_001007598 | 29257 | ribosomal protein L9 | Rn.107914 |
| 1164 | 79063 | X51781cds_s_at | NM_013023 | 25539 | retinal S-antigen | Rn.9856 |
| 1165 | 76971 | X53428cds_s_at | NM_032080 | 84027 | glycogen synthase kinase 3 beta | Rn.10426 |
| 1166 | 79080 | X53504cds_g_at | X53504 | | | |
| 1167 | 82282 | X53773_at | NM_031008 | 81637 | adaptor protein complex AP-2, alpha 2 subunit | Rn.34928 |
| 1168 | 82301 | X54510_at | NM_053602 | 94271 | ATP synthase, H+ transporting, mitochondrial F0 complex, subunit F6 | Rn.5790 |
| 1169 | 82312 | X54531mRNA_at | NM_080689 | 140694 | dynamin 1 | Rn.10354 |
| 1170 | 79087 | X54617mRNA_s_at | X54617 | | | |
| 1171 | 82349 | X54640_at | NM_012783 | 25246 | basigin | Rn.2269 |
| 1172 | 76974 | X54686cds_at | NM_021836 | 24517 | Jun-B oncogene | Rn.15806 |
| 1173 | 76750 | X54793_at | NM_022229 | 63868 | heat shock protein 1 (chaperonin) | Rn.102058 |
| 1174 | 79088 | X54798cds_at | X54798 | | | |
| 1175 | 76748 | X55286_at | NM_013134 | 25675 | 3-hydroxy-3-methylglutaryl-Coenzyme A reductase | Rn.9437 |
| 1176 | 82332 | X55660_at | NM_019331 | 54281 | proprotein convertase subtilisin/kexin type 3 | Rn.3220 |
| 1177 | 79929 | X55955_at | NM_012742 | 25098 | forkhead box A1 | Rn.10470 |
| 1178 | 79090 | X55986mRNA_s_at | NM_017281 | 29671 | proteasome (prosome, macropain) subunit, alpha type 4 | Rn.11076 |
| 1179 | 79095 | X56448mRNA_at | X56448 | 305103 | cell surface glycoprotein gp42 | Rn.73740 |
| 1180 | 82430 | X56546_at | NM_013103 | 25640 | transcription factor 2 | Rn.11342 |
| 1181 | 79104 | X57432cds_s_at | NM_031838 | 83789 | ribosomal protein S2 | Rn.2115 |
| 1182 | 79113 | X58465mRNA_at | X58465 | 25538 | ribosomal protein S5 | Rn.2100 |
| 1183 | 79115 | X58550mRNA_s_at | NM_012490 | 24163 | acrosin | Rn.10423 |
| 1184 | 82525 | X58828_at | NM_053990 | 117063 | protein tyrosine phosphatase, non-receptor type 2 | Rn.33497 |
| 1185 | 82361 | X58865mRNA_at | NM_013190 | 25741 | phosphofructokinase, liver, B-type | Rn.4212 |
| 1186 | 79118 | X59375mRNA_at | X59375 | | | |
| 1187 | 79123 | X59737mRNA_at | X59737 | 29593 | creatine kinase, mitochondrial 1, ubiquitous | Rn.24682 |
| 1187 | 79124 | X59737mRNA_g_at | X59737 | 29593 | creatine kinase, mitochondrial 1, ubiquitous | Rn.24682 |
| 1188 | 79127 | X59949cds_s_at | NM_052799 | 24598 | nitric oxide synthase 1, neuronal | Rn.10573 |
| 1189 | 82393 | X60212_i_at | NM_201415 | 291434 | ribosomal protein L17 | Rn.34429 |
| 1190 | 79132 | X60469mRNA_s_at | NM_080478 | 29722 | amyloid beta (A4) precursor protein-binding, family B, member 1 | Rn.19953 |
| 1191 | 79133 | X60651mRNA_s_at | NM_013026 | 25216 | syndecan 1 | Rn.11176 |
| 1192 | 79145 | X62145cds_at | X62145 | 26962 | ribosomal protein L8 | Rn.127774 |
| 1193 | 79149 | X62166cds_s_at | NM_198753 | 300079 | ribosomal protein L3 | Rn.107726 |
| 1194 | 79153 | X62325cds_f_at | X62325 | | | |
| 1195 | 79166 | X62950mRNA_f_at | X62950 | | | |
| 1196 | 79167 | X62951mRNA_s_at | X62951 | | | |
| 1197 | 79170 | X63369cds_at | NM_133290 | 79426 | zinc finger protein 36 | Rn.82737 |
| 1198 | 79824 | X66974_at | NM_053988 | 117059 | calbindin 2 | Rn.10321 |
| 1199 | 82287 | X67859_at | NM_031119 | 81783 | Sjogren syndrome antigen B | Rn.24494 |
| 1200 | 79893 | X68191_at | NM_019268 | 29715 | solute carrier family 8 (sodium/calcium exchanger), member 1 | Rn.118972 |
| 1201 | 76730 | X68199_at | NM_053986 | 117057 | myosin Ib | Rn.36339 |
| 1202 | 79182 | X70667cds_at | X70667 | 29310 | melanocortin 3 receptor | |
| 1203 | 82425 | X71898_at | NM_134352 | 50692 | plasminogen activator, urokinase receptor | Rn.82711 |
| 1204 | 76720 | X72757_g_at | X72757 | | | |
| 1205 | 82327 | X73371_at | NM_175756 | 289211 | Fc receptor, IgG, low affinity IIb | Rn.33323 |
| 1206 | 79185 | X73411mRNA_s_at | NM_130738 | 113938 | SNRPN upstream reading frame | Rn.11169 |
| 1207 | 79186 | X74227cds_at | NM_019312 | 54260 | inositol 1,4,5-trisphosphate 3-kinase B | Rn.44632 |
| 1208 | 82545 | X74549_at | NM_024382 | 79224 | serine (or cysteine) proteinase inhibitor, dade D, member 1 | Rn.10553 |
| 1209 | 76990 | X74565cds_at | NM_022516 | 29497 | polypyrimidine tract binding protein 1 | Rn.64440 |
| 1209 | 76991 | X74565cds_g_at | NM_022516 | 29497 | polypyrimidine tract binding protein 1 | Rn.64440 |
| 1210 | 82406 | X74593_at | NM_017052 | 24788 | sorbitol dehydrogenase | Rn.11334 |
| 1211 | 79189 | X74833cds_at | NM_012528 | 24261 | cholinergic receptor, nicotinic, beta polypeptide 1 (muscle) | Rn.44611 |
| 1212 | 79190 | X74834cds_s_at | NM_019145 | 25753 | cholinergic receptor, nicotinic, gamma polypeptide | Rn.44634 |
| 1213 | 79192 | X74836cds_s_at | NM_017194 | 29422 | cholinergic receptor, nicotinic, epsilon polypeptide | Rn.10301 |
| 1214 | 79193 | X74978exon_at | X74978 | | | |
| 1215 | 82414 | X75856_at | X75856 | 24822 | testis enhanced gene transcript | Rn.107678 |
| 1216 | 79196 | X76452cds_s_at | NM_001005871 | 29600 | ATPase, Ca++ transporting, plasma membrane 4 | Rn.9986 |
| 1217 | 79198 | X76489cds_at | X76489 | 24936 | CD9 antigen | Rn.2091 |
| 1217 | 79199 | X76489cds_g_at | X76489 | 24936 | CD9 antigen | Rn.2091 |
| 1218 | 80285 | X76724_at | NM_017304 | 29738 | potassium voltage-gated channel, shaker-related subfamily, beta member 2 | Rn.10757 |
| 1219 | 82466 | X76985_at | NM_031655 | 59073 | latexin | Rn.11404 |
| 1220 | 76791 | X77209_at | NM_212546 | 24963 | heat shock 70 kD protein 1-like | Rn.124580 |
| 1221 | 80024 | X77818_at | X77818 | | | |
| 1222 | 82548 | X78606_at | NM_053978 | 117049 | RAB28, member RAS oncogene family | Rn.4023 |
| 1223 | 76995 | X79081mRNA_f_at | NM_019184 | 29277 | Cytochrome P450, subfamily IIC (mephenytoin 4-hydroxylase) | Rn.10870 |
| 1224 | 82210 | X81193_at | NM_057144 | 117505 | cysteine-rich protein 3 | Rn.11345 |
| 1225 | 82331 | X82152_at | NM_080698 | 64507 | fibromodulin | Rn.8778 |
| 1226 | 82554 | X82396_at | NM_022597 | 64529 | cathepsin B | Rn.100909 |
| 1227 | 82392 | X82445_at | NM_017271 | 29648 | nuclear distribution gene C homolog (Aspergillus) | Rn.10413 |
| 1228 | 82223 | X83399_at | NM_053974 | 117045 | eukaryotic translation initiation factor 4E | Rn.11275 |
| 1229 | 79218 | X83585cds_s_at | NM_031602 | 29718 | potassium inwardly-rectifying channel, subfamily J, member 10 | Rn.10196 |
| 1230 | 79221 | X83735exon#3_at | NM_001008889 | 363982 | HIV-induced protein-7-like protease | Rn.60058 |
| 1231 | 79993 | X84210complete_seq_s_at | NM_012988 | 25492 | nuclear factor I/A | Rn.129702 |
| 1232 | 79223 | X86178mRNA_at | X86178 | | | |
| 1233 | 80097 | X86561cds#2_at | NM_001008724 | 361969 | fibrinogen, alpha polypeptide | Rn.98846 |
| 1234 | 79228 | X89695cds_at | NM_214821 | 252887 | olfactory receptor 1413 | Rn.92442 |
| 1235 | 79229 | X89696cds_at | NM_001000363 | 295917 | olfactory receptor 728 (predicted) | Rn.109351 |
| 1236 | 79239 | X89705cds_at | NM_001000502 | 305711 | olfactory receptor 1606 | Rn.115051 |
| 1237 | 82504 | X91234_at | NM_024391 | 79243 | hydroxysteroid (17-beta) dehydrogenase 2 | Rn.10515 |
| 1238 | 82239 | X92069_at | NM_080780 | 113995 | purinergic receptor P2X, ligand-gated ion channel, 5 | Rn.10257 |
| 1239 | 82558 | X92097_at | NM_031722 | 65165 | coated vesicle membrane protein | Rn.1022 |
| 1240 | 79250 | X93219cds_s_at | NM_012896 | 25370 | adenosine A3 receptor | Rn.10457 |
| 1241 | 79988 | X95188_at | NM_053339 | 83522 | acyl-Coenzyme A oxidase 3, pristanoyl | Rn.10546 |
| 1242 | 79256 | X95990exon_s_at | NM_053619 | 113959 | complement component 5, receptor 1 | Rn.10680 |
| 1243 | 79257 | X96437mRNA_at | NM_212505 | 294235 | immediate early response 3 | Rn.23638 |
| 1244 | 77005 | X98490cds_at | NM_021582 | 59102 | replication protein A2 | Rn.40389 |
| 1245 | 79264 | X98564cds_at | NM_021697 | 60326 | potassium channel, subfamily V, member 1 | Rn.42920 |
| 1246 | 79265 | X98746cds_at | NM_134329 | 171178 | alcohol dehydrogenase 7 (class IV), mu or sigma polypeptide | Rn.42935 |
| 1247 | 77006 | Y00156cds_f_at | NM_153314 | 266685 | UDP-glucuronosyltransferase 2B3 precursor, microsomal | Rn.24945 |
| 1248 | 77007 | Y00396mRNA_at | NM_012603 | 24577 | myelocytomatosis viral oncogene homolog (avian) | Rn.12072 |
| 1248 | 77008 | Y00396mRNA_g_at | NM_012603 | 24577 | myelocytomatosis viral oncogene homolog (avian) | Rn.12072 |
| 1249 | 79894 | Y00766_at | NM_013119 | 25657 | sodium channel, voltage-gated, type III, alpha polypeptide | Rn.87394 |
| 1250 | 79281 | Y08138_at | NM_022696 | 64637 | heart and neural crest derivatives expressed transcript 2 | Rn.41057 |
| 1251 | 79288 | Y09453cds_at | NM_019255 | 29658 | calcium channel, voltage-dependent, gamma subunit 1 | Rn.24079 |
| 1252 | 79291 | Y10823cds_s_at | NM_053762 | 114639 | zona pellucida glycoprotein 3 | Rn.10892 |
| 1253 | 79294 | Y11489cds_at | Y11489 | 57023 | a disintegrin and metalloprotease domain 5 | Rn.92321 |
| 1254 | 82731 | Y12635_at | NM_057213 | 117596 | ATPase, H+ transporting, V1 subunit B, isoform 2 | Rn.8109 |
| 1255 | 82853 | Y13714_at | NM_012656 | 24791 | secreted acidic cysteine rich glycoprotein | Rn.98989 |
| 1256 | 76378 | Y15068_at | NM_138911 | 192277 | stress-induced-phosphoprotein 1 (Hsp70/Hsp90-organizing protein) | Rn.22614 |
| 1257 | 79309 | Y17048_g_at | NM_133529 | 171051 | calcium binding protein 1 | Rn.23560 |
| 1258 | 79319 | Z11932cds_g_at | NM_019136 | 25108 | arginine vasopressin receptor 2 | Rn.44344 |
| 1259 | 79325 | Z14118cds_at | Z14118 | 25267 | platelet derived growth factor receptor, alpha polypeptide | Rn.55127 |
| 1259 | 79326 | Z14118cds_g_at | Z14118 | 25267 | platelet derived growth factor receptor, alpha polypeptide | Rn.55127 |
| 1260 | 79327 | Z14119cds_at | NM_031525 | 24629 | platelet derived growth factor receptor, beta polypeptide | Rn.98311 |
| 1261 | 82221 | Z36980_at | NM_024131 | 29318 | D-dopachrome tautomerase | Rn.3464 |
| 1262 | 79349 | Z83757mRNA_at | NM_017094 | 25235 | growth hormone receptor | Rn.2178 |
| 1263 | 79351 | Z83869cds_at | NM_021699 | 60328 | serine/threonine kinase | Rn.42926 |

TABLE 2

| | | | GenBank Acc or | General | Adrenergic Agonist | | Heart Failure | Myocardial Necrosis | Vasculature Agent |
|---|---|---|---|---|---|---|---|---|---|
| SEQ ID NO. | GLGC ID No. | Fragment Name | RefSeq ID No. | Model Weighting | Weighting | Arrhythmia Weighting | Weighting | Weighting | Weighting |
| 1 | 77058 | A03913cds_s_at | A03913 | | | | −0.018538216 | | |
| 2 | 77023 | AA684963_at | AA684963 | | | | | | 0.001491754 |
| 3 | 77024 | AA685112_at | AA685112 | | | | | | −0.023599712 |
| 4 | 77026 | AA685376_f_at | AA685376 | 0.008058474 | | | 0.071586746 | | |
| 5 | 77028 | AA685903_at | NM_001012197 | −0.006684589 | | | | | |
| 6 | 77029 | AA686031_at | NM_001005550 | 0.001231342 | | | | | |
| 7 | 77030 | AA686579_at | NM_001009672 | 0.006230062 | | | | | |
| 8 | 77032 | AA686870_f_at | AA686870 | | | | | | 0.003168987 |
| 9 | 82173 | AA799389_at | NM_031091 | 0.004974244 | | | | | |
| 9 | 82174 | AA799389_g_at | NM_031091 | 0.004582303 | | | | | |
| 10 | 82240 | AA801441_at | NM_031763 | −0.002660145 | | | | | |
| 11 | 77033 | AA819943_at | AA819943 | | | | | | −0.020429244 |
| 12 | 77036 | AA848421_at | AA848421 | −0.001585115 | | | | | |
| 13 | 77037 | AA848546_at | NM_001009542 | 0.013310136 | | | | | |
| 14 | 76160 | rc_AA818604_s_at | NM_212504 | −0.00156337 | 0.011510999 | 0.002767663 | | | |
| 14 | 76161 | AA848563_s_at | NM_212504 | −0.001433022 | | −0.011982835 | | | |
| 14 | 76534 | L16764_s_at | NM_212504 | 0.001445792 | | −0.014449423 | | −0.001452264 | |
| 14 | 76766 | Z75029_s_at | NM_212504 | | | −0.010560855 | | 0.000952824 | |
| 14 | 79333 | Z27118cds_s_at | NM_212504 | | | | | −0.002029624 | |
| 15 | 77039 | AA875509_at | AA875509 | 0.009278474 | | | 0.112712422 | | |
| 16 | 80380 | AB000098_at | NM_022607 | 0.005050612 | | | | | |
| 17 | 80197 | AB000113_at | NM_017217 | 0.004698301 | | | | | |
| 18 | 82747 | AB000216_at | NM_134403 | | | | | | 0.0023802 |
| 19 | 80386 | AB000928_at | NM_053509 | 0.00145853 | | | | | |
| 20 | 77074 | AB001982_g_at | NM_032075 | −0.000167872 | | | | | |
| 21 | 77078 | AB002561_at | NM_021867 | 0.007770658 | | | | | |
| 22 | 77080 | AB003357_at | NM_019349 | −0.006052063 | | | | | |
| 23 | 77082 | AB003478_g_at | NM_133553 | 0.00043827 | | | | | |
| 24 | 82743 | AB003515_at | NM_022706 | 0.005752919 | | | | | |
| 25 | 77204 | AB017140_at | NM_031707 | −0.001689151 | | | −0.026640117 | | |
| 25 | 77607 | AF093268_s_at | NM_031707 | 0.000491799 | | | | | |
| 25 | 80343 | AB003726_at | NM_031707 | | 0.114024028 | | | | |
| 26 | 77083 | AB003753cds#1_at | AB003753 | 0.012466003 | | | | | |
| 27 | 77099 | AB005541_at | AB005541 | −0.005360927 | | | | | |
| 28 | 82881 | AB006138_at | NM_031635 | −0.002661354 | | | | | |
| 29 | 82878 | AB006450_at | NM_019351 | −0.008442076 | | | | | |
| 30 | 82879 | AB006451_at | NM_019352 | −0.004663793 | | | | | |
| 31 | 77104 | AB006710_s_at | NM_057135 | −0.004059713 | | | | | |
| 32 | 77105 | AB006802_at | AB006802 | 0.00247942 | | | | | |
| 33 | 77107 | AB006881mRNA_at | NM_147207 | 0.004993242 | | | | | |
| 34 | 77111 | AB007689_at | NM_053309 | 0.003245644 | | | | | |
| 35 | 82920 | AB008538_at | NM_031753 | 0.012199748 | | | | | |
| 36 | 77135 | AB010154_at | NM_147135 | 0.00478207 | | | | | |
| 37 | 77141 | AB010963_s_at | NM_019273 | 0.004754986 | | | | | |
| 38 | 77155 | AB011679_at | NM_173102 | −0.004758425 | 0.102641129 | | | | |
| 38 | 83224 | rc_AA860030_s_at | NM_173102 | | −0.041422868 | | | 0.011681688 | |
| 39 | 80155 | AB012233_at | AB012233 | 0.001086748 | | | | | |
| 40 | 80157 | AB012234_g_at | AB012234 | | | | | 0.015986643 | |
| 41 | 77161 | AB012600_s_at | NM_024362 | 0.010201774 | | | | | |
| 41 | 82819 | AF015953_at | NM_024362 | 0.008783736 | | | | | |
| 42 | 77162 | AB012933_at | NM_053607 | −0.00303289 | | | | | |
| 43 | 77171 | AB013453_at | NM_013030 | −0.006240329 | | | | | |
| 43 | 77173 | AB013455_at | NM_013030 | 0.004830261 | | | | | |
| 44 | 77172 | AB013454_at | AB013454 | 0.007417977 | | | | | |
| 45 | 77178 | AB015191_at | NM_022505 | 0.003243161 | | | | | |
| 45 | 77179 | AB015191_g_at | NM_022505 | −0.00132699 | | | | | |
| 46 | 77190 | AB015946_s_at | NM_145778 | | | | | | −0.037279404 |
| 47 | 77200 | AB016536_s_at | NM_031330 | −0.010500753 | | | | | |
| 47 | 81530 | rc_AI104524_s_at | NM_031330 | −0.006228102 | | | | | |
| 48 | 77202 | AB017044exon_at | AB017044 | 0.000776987 | | | | | |
| 49 | 77206 | AB017170_s_at | NM_022953 | 0.004949339 | | | | | |
| 50 | 77215 | AB017711_at | NM_031335 | 0.00542016 | | | | | |
| 51 | 77228 | AB020504_g_at | AB020504 | | | | | | 0.020335337 |
| 52 | 77235 | AF000900_at | NM_139091 | | 0.03862251 | | | | |
| 52 | 81017 | rc_AA891045_at | NM_139091 | −0.005187068 | | 0.006437078 | | | |
| 53 | 77239 | AF000943_at | NM_022208 | 6.23E−05 | | | | | |
| 54 | 76391 | AF003523_s_at | NM_022698 | −0.000863097 | | | | | |
| 55 | 77247 | AF004218_s_at | NM_030996 | −0.006820798 | | | | | |
| 56 | 82857 | AF004811_at | NM_030863 | | 0.035814293 | | | | |
| 57 | 77252 | AF008912_at | AF008912 | 0.002589073 | | | | | |
| 58 | 77253 | AF009656mRNA_s_at | NM_012583 | −0.001211495 | | | | | |
| 59 | 82942 | AF014503_at | NM_053611 | | | | | | −0.032310481 |
| 60 | 80323 | AF016179_at | NM_173315 | 0.010186836 | | | | | |
| 61 | 80326 | AF016182_at | NM_173130 | 0.002029218 | | | | | |
| 62 | 77270 | AF018261_at | NM_057136 | | −0.016044391 | | | | |
| 63 | 77271 | AF019624_at | NM_019151 | −0.003928311 | | | | | |
| 64 | 77275 | AF020046_s_at | NM_031768 | 0.00346581 | | | | | |
| 65 | 77276 | AF020210_s_at | NM_053655 | 0.004687364 | | | | | |
| 65 | 77277 | AF020211_s_at | NM_053655 | −0.004012326 | | | | | |
| 66 | 78927 | U88324_at | NM_030987 | 0.004373243 | | | | | |
| 66 | 82918 | AF022083_s_at | NM_030987 | −0.000577858 | | | | | |
| 67 | 76834 | AF022729_at | NM_080397 | −0.000303281 | | | | | |
| 68 | 77284 | AF022742cds_at | NM_031771 | 0.007048985 | | | | | |
| 69 | 82949 | AF022774_g_at | NM_133591 | 0.003323409 | | | | | |
| 70 | 77286 | AF022952_at | AF022952 | | 0.044178164 | | | | |
| 71 | 77287 | AF023087_s_at | NM_012551 | | 0.045546088 | | | | |
| 71 | 78865 | U75397UTR#1_s_at | NM_012551 | | 0.005987095 | | | −0.002966072 | |
| 72 | 77289 | AF023621_at | AF023621 | 0.007952494 | | | | | |
| 73 | 77291 | AF025671_s_at | NM_022522 | 0.007926221 | 0.016222954 | | | | |
| 74 | 80347 | AF026476_at | NM_031777 | | | | | 0.021557864 | |
| 75 | 80348 | AF026529_s_at | AF026529 | | | | | | −0.010439504 |
| 76 | 77293 | AF027188UTR#1_at | AF027188 | | | | | | 0.000302847 |
| 77 | 77295 | AF027571_s_at | NM_024353 | −0.002132984 | | | | | |
| 78 | 77298 | AF028784mRNA#1_s_at | AF028784 | −0.004307178 | | −0.028755557 | | | |
| 79 | 77300 | AF029357cds_at | NM_001000583 | | | | | −0.005808993 | |
| 80 | 77309 | AF030091UTR#1_g_at | NM_053662 | −0.006896238 | | | | | |
| 81 | 77312 | AF030358_g_at | NM_134455 | | | | | | −0.013858595 |
| 82 | 82963 | AF032668_at | NM_019277 | 0.004905557 | | | | | −0.013052396 |
| 83 | 82964 | AF032669_at | NM_139043 | 0.00862072 | | | | | |
| 84 | 77331 | AF034896_f_at | NM_206850 | 0.002824944 | | | | | |
| 85 | 77336 | AF035632_s_at | NM_022939 | 0.004864385 | | | | | |
| 86 | 77339 | AF035952_at | AF035952 | −0.00633573 | | | | | |
| 87 | 77349 | AF036959_at | NM_031787 | −0.000127287 | | | | | |
| 87 | 83287 | rc_AA866455_at | NM_031787 | −0.007503903 | | | | | |
| 88 | 82705 | AF037071_at | NM_138922 | | | | | 0.001848547 | |
| 89 | 80689 | rc_AI639215_at | NM_131913 | −0.00680676 | | −0.030878931 | | | |
| 89 | 82946 | AF038591_at | NM_131913 | 0.002973849 | | | | | 0.05089182 |
| 90 | 80379 | AF039085_at | NM_053553 | | | | 0.003234098 | | |
| 91 | 77354 | AF039308_at | AF039308 | 0.004817531 | | −0.008588927 | | | |
| 92 | 82951 | AF040954_at | NM_022951 | 0.005034157 | | | | | |
| 93 | 80384 | AF041107_g_at | AF041107 | −0.001433491 | | | | | |
| 94 | 77374 | AF044574_at | NM_171996 | −5.25E−05 | | | | | |
| 95 | 82965 | AF044910_at | NM_022509 | | | | | −0.004745544 | |
| 96 | 77380 | AF048828_at | NM_031353 | −0.000179311 | | | | | |
| 96 | 77381 | AF048828_g_at | NM_031353 | | | | −0.055135779 | | |
| 97 | 77385 | AF050214_at | AF050214 | −0.009972814 | | | | | |
| 98 | 77389 | AF050662UTR#1_at | NM_031593 | 0.004343287 | | | | | |
| 98 | 77428 | AF060174_at | NM_031593 | −6.98E−05 | | | | | |
| 99 | 77395 | AF051895_at | NM_013132 | −0.000999555 | | | | | |
| 100 | 77406 | AF053938_s_at | NM_181092 | −0.000529235 | | | | | |
| 101 | 80160 | AF053987_at | AF053987 | −0.007180778 | | | | | |
| 102 | 80164 | AF053991_at | NM_173318 | −0.006955284 | | | | | |
| 103 | 77414 | AF055667mRNA_at | AF055667 | −0.005961157 | | | | | |
| 104 | 77420 | AF056324_at | NM_022394 | −0.005866951 | | | | | |
| 104 | 77421 | AF056324_g_at | NM_022394 | 7.70E−06 | | | | | |
| 105 | 80256 | AF059258_at | NM_031744 | | | | | 0.006168868 | |
| 106 | 82798 | AF059530_at | NM_053557 | | | | −0.019917649 | | |
| 107 | 77438 | AF061947_g_at | NM_053575 | 3.08E−06 | | | | | |
| 108 | 77441 | AF062594_g_at | NM_053561 | −0.002522543 | | | | | |
| 109 | 77445 | AF062741_g_at | NM_145091 | 0.007851302 | 0.010269134 | | | | |
| 110 | 77450 | AF063249_at | NM_022925 | −0.002775662 | | | | | |
| 111 | 82806 | AF063447_at | NM_053563 | | | | | 0.00536819 | |
| 112 | 76776 | U69109_s_at | NM_017318 | −0.003541072 | | | | | |
| 112 | 77453 | AF063890_s_at | NM_017318 | 0.007270182 | | | | | |
| 113 | 77456 | AF064868_g_at | NM_024163 | 0.003108745 | | | | | |
| 114 | 77460 | AF065387_at | NM_031756 | 0.001651406 | | | | 0.021964531 | |
| 115 | 77473 | AF071014mRNA_s_at | NM_024483 | | −0.020616989 | | | | |
| 116 | 77480 | AF072892_s_at | AF072892 | 0.00026861 | | | | | |
| 117 | 77481 | AF072935_at | NM_022692 | 0.00245029 | | | | | |
| 118 | 77483 | AF073891_at | NM_133610 | 0.000238637 | | | | | |
| 119 | 77491 | AF076183_at | NM_134406 | 0.016132894 | | | | | |
| 119 | 77493 | AF076184_at | NM_134406 | −0.004070203 | | | | | |
| 120 | 77494 | AF076619_at | NM_031623 | −0.005929911 | | | | | |
| 121 | 77496 | AF077000_at | NM_057204 | 0.014938977 | | | | | |
| 121 | 77497 | AF077000_g_at | NM_057204 | 0.002639806 | | | | | |
| 122 | 77507 | AF079864_at | NM_173293 | | 0.002053728 | | | | |
| 123 | 77518 | AF081196_at | NM_019211 | −0.005127961 | | | | | |
| 124 | 77530 | AF083269_at | NM_019289 | −0.003163393 | | | | 0.019143438 | |
| 124 | 77531 | AF083269_g_at | M_019289 | −0.003452801 | | | | | |
| 125 | 77537 | AF084576_at | NM_053666 | 0.003383415 | | | | | |
| 126 | 77546 | AF087037_g_at | NM_019290 | 0.001495773 | | | | | |
| 127 | 77548 | AF087431_g_at | NM_031749 | 0.002710741 | | | | | |
| 128 | 77551 | AF087454_at | NM_031597 | | 0.008709922 | | | | |
| 129 | 77565 | AF090113_at | NM_138535 | −0.003243871 | | | | | |
| 130 | 77572 | AF090306_s_at | NM_031816 | −0.007956647 | | | | | |
| 131 | 77576 | AF091561_at | NM_001000504 | | 0.033958369 | | | | |
| 132 | 77577 | AF091562_s_at | NM_001000507 | 0.0018365 | | | | | |
| 133 | 77582 | AF091566_f_at | NM_001006598 | | | | | 0.002484912 | |
| 134 | 77583 | AF091567_s_at | NM_001000724 | 0.003349821 | | | | | |
| 135 | 77591 | AF091577_at | NM_001000979 | 0.014847884 | | | | | |
| 136 | 77595 | AF091580_s_at | AF091580 | 0.000102834 | | | | | |
| 137 | 77603 | AF092523_s_at | NM_053665 | 0.000802342 | | | | | |
| 138 | 77625 | AF099093_g_at | NM_022690 | −0.003399134 | | | | | |
| 139 | 77643 | AJ000347_g_at | NM_171990 | −0.00259151 | | | | | |
| 140 | 80387 | AJ000485_at | NM_021997 | −0.003017347 | | | | | |
| 141 | 77645 | AJ000557cds_s_at | NM_031514 | −0.003608013 | | | | | |
| 141 | 78719 | U13396_g_at | NM_031514 | −0.008191915 | | | | | |
| 142 | 77648 | AJ001290cds_at | NM_053715 | 0.010299894 | | | | | |
| 143 | 77649 | AJ001517cds_at | NM_053301 | 0.007574458 | | | | | |
| 144 | 77651 | AJ001637cds_at | NM_013004 | 0.003912607 | | | | | |
| 145 | 76426 | AJ002942cds_at | AJ002942 | 0.001515852 | | | | | |
| 146 | 77660 | AJ004858_at | NM_053349 | 0.000717469 | | | | | |
| 147 | 77666 | AJ005394_at | NM_134452 | | | | | | 0.034821389 |
| 147 | 83143 | rc_AA859757_g_at | NM_134452 | 0.000952327 | | | | | |
| 148 | 77670 | AJ005984_at | NM_021842 | 0.002768801 | | | | | |
| 149 | 76430 | AJ006070_at | AJ006070 | 0.003561314 | | | | | |
| 150 | 77676 | AJ006519_at | NM_024154 | | −0.021337478 | | | | |
| 151 | 77684 | AJ007291_at | NM_057143 | 0.002229149 | | | | | |
| 151 | 77685 | AJ007291_g_at | NM_057143 | −0.004291246 | | | 0.020686076 | | |
| 152 | 77686 | AJ007422_s_at | NM_133567 | 0.006043464 | | | | | |
| 153 | 77690 | AJ007632_s_at | NM_145095 | 0.001364274 | | | | | |
| 154 | 77699 | AJ011115_g_at | NM_021838 | −0.001564234 | | | | | |
| 154 | 77700 | AJ011116_at | NM_021838 | | 0.06143741 | | | | |
| 155 | 77704 | AJ011606_at | NM_053480 | 0.007626935 | | | | | |
| 156 | 77708 | AJ011656cds_s_at | NM_031700 | | −0.04170253 | | | | |
| 156 | 81868 | M74067_at | NM_031700 | 0.000654705 | | | | | |
| 157 | 77709 | AJ011811_at | AJ011811 | | 0.012905176 | | | | 0.022722704 |
| 158 | 77713 | AJ012603cds_at | NM_020306 | 0.000355205 | | | | | |
| 158 | 77714 | AJ012603UTR#1_at | NM_020306 | 0.003621376 | | | | | |
| 158 | 77715 | AJ012603UTR#1_g_at | NM_020306 | 0.000758627 | | | | | |
| 159 | 76433 | AJ223083_at | AJ223083 | 0.00750333 | | | | | |
| 160 | 77725 | AJ223355_at | NM_133418 | −0.010217123 | | | | | |
| 161 | 76815 | AJ224120_at | NM_053487 | −0.003491567 | | | | | |
| 162 | 77728 | D00092_s_at | D00092 | −0.007939132 | | | | | |
| 163 | 76780 | D00569_at | NM_057197 | 0.001631629 | | | | | |
| 163 | 76781 | D00569_g_at | NM_057197 | 0.010701378 | | | | | |
| 164 | 79889 | D00634_at | NM_012701 | 0.004258233 | | | | | |
| 165 | 76114 | rc_AI229440_s_at | NM_138877 | −0.013972483 | | | | | |
| 165 | 77732 | D00636cds_s_at | NM_138877 | −0.00237716 | | | | | |
| 166 | 76441 | D00688_s_at | D00688 | 0.001134684 | | | | | |
| 167 | 79598 | D00729_at | NM_017306 | −0.001382011 | | | | | |
| 168 | 81799 | D00753_at | NM_031531 | 0.000473488 | | | | | |
| 169 | 77738 | D01046_at | NM_032617 | | | | 0.018439179 | | |
| 170 | 82782 | D10233_g_at | NM_031095 | 0.004545741 | | | | | |
| 171 | 82255 | D10262_at | NM_017127 | | | | −0.00133593 | | |
| 172 | 77749 | D10693_at | NM_031044 | −0.001281149 | | −0.002414368 | | | |
| 173 | 77751 | D10699_g_at | NM_017237 | 0.000713608 | | | | | |
| 174 | 77759 | D10755_s_at | NM_017283 | 0.00460428 | | | | | |
| 175 | 77765 | D10853_at | D10853 | 0.003515636 | | | | | |
| 176 | 77769 | D10952_i_at | NM_053586 | | | | | | 0.042560982 |
| 177 | 77771 | D11445exon#1-4_s_at | NM_030845 | −0.001220281 | | | | | |
| 178 | 77781 | D12771_s_at | NM_057102 | | | | | | −0.011525699 |
| 179 | 77785 | D12978cds_s_at | D12978 | 0.012786587 | | | | | |
| 180 | 77786 | D13120_s_at | NM_019383 | | | | | | −0.037057941 |
| 181 | 77787 | D13122_i_at | NM_012915 | | | | | | −0.019450928 |
| 182 | 77790 | D13124_s_at | NM_133556 | | | | | | 0.005764352 |
| 183 | 77796 | D13309_s_at | NM_031563 | 0.007794571 | | | | 0.003285658 | |
| 184 | 82850 | D13376_at | NM_024349 | | | | 0.09076439 | | |
| 185 | 77798 | D13417_g_at | NM_024360 | 0.00053338 | | | | | 0.002419642 |
| 186 | 82035 | D13518_at | NM_012764 | 0.011310205 | | | | | |
| 187 | 77800 | D13556exon_at | D13556 | −0.004844821 | | | | | |
| 188 | 77801 | D13623_at | NM_001008280 | | −0.047655043 | | | 0.001217751 | |
| 189 | 82985 | D13907_g_at | NM_022395 | 0.002235026 | | | | | |
| 190 | 77807 | D13927_at | NM_053327 | | −0.008496145 | | | | |
| 190 | 77808 | D13927_g_at | NM_053327 | −0.001908254 | | | | | |
| 191 | 77810 | D13962_g_at | NM_017102 | −0.004090417 | | | | 0.003139048 | |
| 192 | 77811 | D13963_at | NM_022920 | | −0.048070463 | | | | |
| 193 | 77816 | D13985_g_at | NM_031719 | −0.001987018 | | 0.024037661 | | | |
| 193 | 81481 | rc_AI169005_at | NM_031719 | | | | | | 0.00985428 |
| 194 | 76448 | D14014_g_at | NM_171992 | 0.006148286 | | | | | |
| 195 | 77817 | D14029_at | NM_019236 | 0.001254016 | | | | | |
| 196 | 77819 | D14048_g_at | NM_057139 | −0.000966768 | | | | | |
| 197 | 82979 | D14076_at | NM_138538 | −0.010680186 | | | | | |
| 198 | 77828 | D14568_at | NM_017309 | 0.006605262 | | | | | |
| 199 | 82030 | D16302_at | NM_030861 | 0.010925422 | | | | | |
| 200 | 77839 | D16443_at | NM_012704 | −0.001676701 | 0.006211864 | | | | |
| 200 | 77840 | D16443_g_at | NM_012704 | | 0.060318033 | | | | |
| 201 | 77842 | D16817_g_at | NM_031040 | 0.000528128 | 0.031843979 | | | | |
| 202 | 82056 | D17614_at | NM_013053 | 0.012679842 | | | | | |
| 203 | 76462 | D21799_g_at | NM_017284 | 0.004978004 | | | | | |
| 204 | 77855 | D21869_s_at | NM_031715 | −0.003132416 | | | | | |
| 205 | 76463 | D25233UTR#1_at | D25233 | 0.001600213 | | | | | |
| 206 | 77861 | D26111_s_at | NM_173103 | −0.005564416 | | | | | |
| 207 | 77865 | D26393exon_s_at | D26393 | 0.009973904 | | | | | |
| 208 | 82419 | D26439_at | NM_017079 | 0.001243411 | | | | | |
| 209 | 77881 | D28111_g_at | NM_012720 | | | | | −0.017318385 | |
| 210 | 77883 | D28512_at | NM_019122 | −0.002647772 | | −0.016693169 | | | |
| 211 | 76467 | D28754_s_at | NM_199501 | −0.007269151 | | | | | |
| 212 | 82751 | D29646_at | NM_013127 | −0.006755851 | | | | | |
| 213 | 82152 | D29683_at | NM_053596 | −0.011403734 | | | | | −0.014227333 |
| 214 | 77893 | D29766cds#1_s_at | NM_012931 | −0.000856552 | 0.008057124 | −0.025576348 | | −0.007744507 | |
| 215 | 77896 | D30649mRNA_s_at | NM_019370 | 0.008440336 | | | | | |
| 216 | 82555 | D30735_at | NM_013222 | 0.004002481 | | | | | |
| 217 | 82531 | D37920_at | NM_017136 | | | | −0.021102309 | | |
| 218 | 79730 | D37934_at | D37934 | −0.001086162 | | 0.014576578 | | | |
| 219 | 77914 | D38036_at | D38036 | | | | | −0.00436745 | |
| 220 | 77916 | D38061exon_s_at | NM_057105 | 0.009027092 | | | | | |
| 220 | 78466 | S56937_s_at | NM_057105 | −0.010166739 | | | | | |
| 221 | 77918 | D38063exon_at | NM_175846 | 0.004146659 | | | −0.079159195 | | |
| 222 | 77921 | D38067exon_at | D38067 | 0.001421149 | | | | | |
| 223 | 82871 | D38468_s_at | NM_013016 | 0.005293822 | | | | | |
| 224 | 77926 | D38494_at | NM_017292 | −0.003429219 | | | | | |
| 225 | 77929 | D42116_s_at | NM_001005330 | | −0.026427587 | | | | |
| 225 | 82202 | L36532_s_at | NM_001005330 | 0.016035281 | | | | | |
| 226 | 77931 | D42148_at | NM_057100 | | | | | 0.014581256 | |
| 227 | 77938 | D45247_at | D45247 | 0.00024457 | | | | | |
| 227 | 77939 | D45247_g_at | D45247 | −0.005193357 | | | | | |
| 228 | 77945 | D45254_g_at | NM_022598 | −0.004456726 | | | | | |
| 229 | 79794 | D45413_at | D45413 | −0.004736458 | | | | | |
| 230 | 77948 | D45862_g_at | NM_013076 | −0.001306147 | | 0.008026127 | | | |
| 231 | 82654 | D49434_at | D49434 | 0.008700115 | | | | | |
| 232 | 80069 | D50093_s_at | NM_012631 | 0.009290906 | | | | | |
| 233 | 81357 | rc_AI044488_at | NM_017126 | 0.009068835 | | | | | |
| 233 | 82653 | D50436_at | NM_017126 | 0.007602032 | | 0.004478737 | | | |
| 234 | 82514 | D50694_at | NM_033236 | −0.001414045 | | | 0.030202167 | | |
| 235 | 82516 | D50695_at | NM_057122 | | | | | | 0.029237364 |
| 236 | 82515 | D50696_at | NM_057123 | 0.00183357 | | | −0.012550288 | | |
| 237 | 76786 | D63673_at | NM_057125 | | | | | | −0.032436238 |
| 238 | 76837 | D63761_g_at | NM_024153 | 0.004599496 | | | | | |
| 239 | 82703 | D63772_at | NM_013032 | 0.004117885 | | | | | |
| 240 | 80296 | D64046_g_at | NM_022185 | −0.01577066 | | | | | |
| 241 | 82225 | D64050_at | NM_053594 | 0.009049812 | | | | | |
| 242 | 77970 | D64085_g_at | NM_022211 | −0.005364785 | | | | | 0.013693781 |
| 243 | 80129 | D82868_at | NM_019326 | | | | | −0.012600727 | |
| 244 | 82508 | D83349_at | NM_019161 | −0.002097485 | | | | | |
| 245 | 79979 | D83598_at | NM_013040 | 0.000731827 | | | | | |
| 246 | 77984 | D84485_at | D84485 | −0.002943732 | | | | | |
| 247 | 82892 | D85189_at | NM_053623 | | −0.046112893 | | | | |
| 248 | 77988 | D85435_at | NM_134449 | 0.010695869 | | 0.051905957 | | | |
| 248 | 77989 | D85435_g_at | NM_134449 | 0.009386937 | | | | | |
| 249 | 76759 | D86215_at | NM_012985 | | | | | | −0.007449511 |
| 250 | 82830 | D86711_at | D86711 | | | | | | 0.021907979 |
| 251 | 77993 | D86745cds_s_at | NM_057133 | 0.009428658 | | | | | |
| 252 | 82643 | D87515_at | NM_031097 | 0.004228841 | | | | | |
| 253 | 80119 | D87748_at | NM_017179 | 0.001949845 | | | | | |
| 254 | 77996 | D87922exon_at | D87922 | 0.005995075 | | | | | |
| 255 | 77997 | D87991_at | NM_199081 | −0.00267491 | | | | | |
| 255 | 77998 | D87991_g_at | NM_199081 | | 0.027361048 | | | | |
| 256 | 82666 | D88666_at | NM_138882 | | 0.013429142 | | | | −0.010783958 |
| 257 | 80060 | D88751_at | NM_013100 | −0.00352483 | | | | | |
| 258 | 76489 | D89069_f_at | NM_019170 | −0.001933053 | | | | | |
| 258 | 82598 | X95986mRNA#1_f_at | NM_019170 | | | | 0.074714996 | | |
| 259 | 76490 | D89070cds_s_at | D89070 | 0.002332397 | | | | | |
| 260 | 82923 | D89983_at | NM_022585 | | | 0.035978876 | | | |
| 261 | 81175 | rc_AA946532_at | NM_012804 | 0.006521719 | | | | | −0.035885661 |
| 261 | 82757 | D90038_at | NM_012804 | −0.000948729 | | | | | |
| 262 | 78004 | D90258_s_at | NM_017280 | −0.006031789 | | | | | |
| 263 | 78005 | D90265_s_at | NM_017278 | | | | 0.035172715 | | |
| 264 | 78009 | D90404_g_at | NM_017097 | | | | | | −0.010860794 |
| 265 | 79398 | E00988mRNA_s_at | NM_031511 | 0.000299257 | | | | | |
| 266 | 76108 | rc_AI137856_s_at | NM_031576 | | | | | | 0.029517982 |
| 266 | 76549 | M10068mRNA_s_at | NM_031576 | 0.015160143 | | | | | −0.007663248 |
| 266 | 79394 | E01524cds_s_at | NM_031576 | | | | | | −0.012054547 |
| 267 | 79390 | E01884cds_s_at | NM_031512 | 0.005033183 | | | | | |
| 268 | 79388 | E02223cds_f_at | NM_012548 | | | −0.000905346 | | | |
| 269 | 76498 | E03344cds_s_at | NM_017234 | −0.009437422 | | | | | |
| 270 | 79380 | E03424cds_s_at | NM_024356 | | 0.045010987 | | | | |
| 271 | 79366 | E12275cds_s_at | NM_031049 | 0.003068308 | | | | | |
| 272 | 79363 | E12625cds_at | NM_080886 | 0.010305935 | | | | | 0.026668246 |
| 273 | 79358 | E13541cds_s_at | NM_019284 | −0.006413546 | | | | | |
| 274 | 79356 | E13573cds_s_at | NM_057130 | 0.005955648 | | | | | |
| 275 | 79354 | E13732cds_at | NM_020542 | | −0.038950845 | | | | |
| 276 | 76501 | J00728cds_f_at | J00728 | 0.002236485 | | | | | |
| 277 | 78012 | J00735_g_at | NM_012559 | | 0.011157745 | | | 0.008551993 | |
| 277 | 78973 | X05861exon#1-6_s_at | NM_012559 | | −0.036754803 | | | 0.005752955 | |
| 278 | 78017 | J00776cds_s_at | NM_207593 | −0.010772462 | | | | | |
| 279 | 78025 | J02596cds_g_at | NM_012501 | | 0.014397979 | | | | |
| 280 | 79531 | J02675_at | NM_013024 | 0.004383291 | | | | | |
| 281 | 76104 | J02679_s_at | NM_017000 | | | | 0.053294428 | | |
| 282 | 82783 | J02962_at | NM_031832 | 5.12E−05 | | | | | |
| 283 | 78027 | J02998_at | NM_031090 | −0.00552552 | | | | | |
| 284 | 78998 | X08056cds_s_at | NM_012793 | 0.000829584 | | | | | |
| 284 | 83017 | J03588_at | NM_012793 | 0.001062133 | | | | | |
| 285 | 82029 | J03819_at | NM_012672 | 0.001455781 | | | | | |
| 286 | 82766 | J04024_at | NM_017290 | | | | −0.068880859 | | |
| 287 | 82122 | J04197_i_at | NM_012621 | | | | −0.07241485 | | |
| 288 | 79491 | J04503_at | NM_017038 | | | | −0.019278311 | | |
| 289 | 81903 | J04532_at | NM_022507 | 0.003401504 | | | | | |
| 290 | 78277 | M25350_s_at | NM_017031 | | −0.03411663 | | | | |
| 290 | 82065 | J04563_at | NM_017031 | | −0.023095916 | | | | |
| 290 | 84558 | rc_AA799729_at | NM_017031 | 0.005367894 | | | | | 0.006983609 |
| 290 | 84559 | rc_AA799729_g_at | NM_017031 | | −0.003253038 | | | −0.011008583 | |
| 291 | 82978 | J04591_at | NM_012789 | 0.007971727 | | | | | |
| 292 | 80223 | J04731_at | NM_012970 | −0.001341426 | | | | | |
| 293 | 82069 | J04792_at | NM_012615 | 0.0023872 | | | | | |
| 294 | 78032 | J04807mRNA_i_at | NM_019130 | −0.00261233 | | | | | |
| 295 | 82769 | J04943_at | NM_012992 | −0.005066212 | −0.00627762 | | | | |
| 296 | 76874 | J05181_at | NM_012815 | −0.002111943 | | | | | |
| 297 | 76521 | J05425cds_s_at | NM_017202 | 0.010461081 | | | | | |
| 298 | 79529 | J05571_s_at | NM_134351 | 0.000970803 | | | | | 0.008348271 |
| 299 | 81901 | J05592_at | NM_022676 | 0.000839142 | | | | | |
| 299 | 81902 | J05592_g_at | NM_022676 | 0.00555484 | | | | | |
| 300 | 81324 | rc_AI013194_at | NM_020075 | 0.006064963 | | | | 0.018587509 | |
| 300 | 81643 | rc_AI177986_at | NM_020075 | | −0.056766708 | | | −0.004992924 | |
| 300 | 81949 | K01677_at | NM_020075 | | | | | | −0.001953965 |
| 301 | 80428 | K01933_at | NM_012582 | | 0.000397715 | | | | |
| 302 | 81866 | K02816_g_at | NM_001009618 | −0.002661556 | | | | | |
| 303 | 81837 | K03242_at | NM_017027 | −0.000240218 | | | | | |
| 304 | 78051 | K03468_s_at | NM_012604 | 0.00170629 | | 0.008542375 | | | |
| 305 | 79785 | L00124_at | NM_012553 | 0.002990055 | | | | | |
| 306 | 78053 | L00131cds_s_at | NM_012729 | −0.006465365 | | | | | |
| 307 | 79481 | L01507_at | NM_013008 | 0.00359275 | | | | | |
| 308 | 81912 | L01702_at | NM_012763 | 0.005971331 | | | | | |
| 309 | 78057 | L01793_at | NM_031043 | 0.008603779 | | | | | |
| 309 | 82733 | U96130_at | NM_031043 | | | | 0.021066936 | | |
| 310 | 80221 | L02315_at | L02315 | 0.008836332 | | | | | |
| 311 | 82997 | L02529_at | NM_021266 | 0.011252693 | | | | | |
| 312 | 76530 | L02926_s_at | NM_012854 | | 0.001244057 | | | | |
| 312 | 79984 | X60675_at | NM_012854 | 0.004160387 | | | | | |
| 313 | 76623 | L03294_at | NM_012598 | 0.006424179 | | | | | |
| 313 | 81788 | rc_AI237731_s_at | NM_012598 | 0.002677477 | | | | | |
| 314 | 81888 | L03382_at | NM_022707 | −0.003717055 | | | | | |
| 315 | 82146 | L05489_at | NM_012945 | | 0.05303571 | | | | |
| 316 | 79422 | L06804_at | L06804 | 0.003630961 | | | | | |
| 317 | 82020 | L07073_at | NM_133593 | 0.003719411 | 0.003569022 | 0.024922837 | | | |
| 318 | 82021 | L07074_at | NM_133305 | | | | 0.010158709 | | |
| 319 | 78074 | L07380_g_at | NM_012850 | 0.00465511 | | | | | |
| 320 | 78080 | L07402_f_at | L07402 | −0.00693583 | | | | | |
| 321 | 78082 | L07408_at | L07408 | 0.000632269 | | 0.004746168 | | | |
| 322 | 78084 | L07410_at | L07410 | 0.000248766 | | | | | |
| 323 | 78085 | L07925_at | NM_019250 | 0.003250862 | | | | | |
| 323 | 78086 | L07925_g_at | NM_019250 | 6.30E−05 | | | | | |
| 324 | 78087 | L08228exon#22_s_at | NM_017010 | | −0.004027202 | | | | |
| 324 | 80281 | U11418_s_at | NM_017010 | | | | | | −0.03565821 |
| 325 | 78093 | L08495cds_s_at | NM_021841 | 0.004305005 | | | | | |
| 326 | 78094 | L08496cds_s_at | NM_017289 | −0.002125725 | | | | | |
| 327 | 82058 | L11319_at | NM_031723 | 0.005031974 | | | | | |
| 328 | 79571 | L11694_at | NM_017033 | 0.000470079 | | | | | |
| 328 | 84381 | rc_AA894296_at | NM_017033 | 0.007742428 | | | | | 0.02919039 |
| 329 | 82641 | L12025_at | NM_017076 | 0.003421032 | | | | 0.001804291 | |
| 330 | 82726 | L12383_at | NM_024151 | 0.000799434 | | | | | |
| 331 | 78109 | L13039_s_at | NM_019905 | | 0.014035502 | | | 0.018380722 | |
| 332 | 78122 | L13407_i_at | NM_012519 | −0.000384315 | | | | | |
| 333 | 78124 | L13619_at | NM_022392 | −0.005737044 | | | | | |
| 334 | 78129 | L14003UTR#1_f_at | NM_012723 | 0.004526098 | | | | | |
| 335 | 78133 | L14462_at | NM_019220 | −0.010517811 | | | | | |
| 336 | 78136 | L14937cds#1_s_at | NM_133559 | | | | | | −0.042665795 |
| 336 | 78137 | L14937cds#2_s_at | NM_133559 | | 0.029715806 | | | | |
| 337 | 81165 | rc_AA946044_s_at | NM_030857 | 0.004024346 | | | | | |
| 337 | 81831 | L14951_at | NM_030857 | 0.00087006 | | | | | |
| 338 | 82053 | L16532_at | NM_012809 | | | | | | −0.000656276 |
| 339 | 79985 | L17138_at | NM_017265 | −0.001135088 | | | | | |
| 340 | 76217 | rc_AA893328_at | NM_172008 | 0.009539109 | | | | | |
| 340 | 76535 | L18889_at | NM_172008 | −0.001032166 | | | | | |
| 341 | 81921 | L19698_at | NM_031093 | | 0.033887292 | | | | |
| 342 | 80288 | L20468_at | NM_138511 | −0.006032297 | | | | | |
| 343 | 76875 | M32167_at | NM_031836 | 0.000924681 | | | | | |
| 343 | 78149 | L20913_s_at | NM_031836 | −0.004815023 | | | | | |
| 344 | 82209 | L22760_at | NM_019185 | 0.0057379 | | | | | |
| 344 | 84783 | rc_AA800786_at | NM_019185 | 0.004902681 | | | 0.08498884 | | |
| 345 | 81900 | L22761_at | NM_144730 | −0.002122984 | | | | | |
| 346 | 81867 | L24051_at | NM_053820 | 0.009775965 | | | | | |
| 347 | 79745 | L24389_at | L24389 | | | | −0.026223661 | | |
| 348 | 79563 | L24776_at | NM_057208 | 0.005222633 | | 0.018976002 | | | |
| 349 | 76634 | L24896_s_at | NM_017165 | −0.007555899 | | | | | |
| 350 | 79650 | L24897_s_at | L24897 | −0.006293948 | | | | | |
| 351 | 82256 | L24907_at | NM_134468 | 0.005242402 | | | | | |
| 352 | 82405 | L25785_at | L25785 | | | | | 0.012336024 | |
| 353 | 79890 | L26293_at | L26293 | −0.007339693 | | | | | |
| 354 | 79592 | L27075_at | NM_016987 | | | | | 0.014514282 | |
| 355 | 80226 | L27663_at | L27663 | −0.008002433 | | | | | |
| 356 | 82033 | L29281_at | NM_019335 | | | | 0.042019624 | | |
| 357 | 78173 | L29573_s_at | NM_031343 | 0.001193588 | | | | | |
| 358 | 81857 | L31619_g_at | NM_012832 | 0.007103125 | | | | | |
| 359 | 78177 | L31622_at | NM_019297 | −0.001749584 | −0.024669267 | | | | |
| 360 | 76093 | rc_AI070295_at | NM_024127 | 0.000885106 | | | | | |
| 360 | 76094 | rc_AI070295_g_at | NM_024127 | | −0.064273516 | | | | |
| 360 | 76542 | L32591mRNA_g_at | NM_024127 | | | −0.000430949 | | | |
| 361 | 78178 | L34074cds_at | NM_021860 | −0.0017903 | | | | | |
| 362 | 82025 | L34262_at | NM_022502 | 0.014541734 | | | | | |
| 363 | 78179 | L34542cds_at | NM_021859 | 0.001470242 | | | | | |
| 364 | 79631 | L36029_at | NM_024380 | −0.001139034 | | | | | |
| 365 | 79632 | L36030_at | NM_053831 | 0.003994899 | | | | | |
| 366 | 78181 | L36664_s_at | NM_012544 | | | | | −0.003813978 | |
| 367 | 76686 | L38437_at | NM_019223 | 0.003816558 | | | | | |
| 368 | 82063 | L39991_at | L39991 | | | | 0.00529995 | | |
| 369 | 78187 | L41684mRNA_at | NM_031819 | 0.010728986 | | | | | |
| 370 | 78193 | L47281_at | L47281 | 0.00268469 | | | | | |
| 371 | 76548 | L48209_s_at | L48209 | | | | | | 0.001270648 |
| 372 | 79441 | M10094_g_at | NM_001008830 | 0.008925061 | | | | | |
| 373 | 81871 | M11266_at | NM_013078 | 0.000743579 | | | | | |
| 374 | 76552 | M11942_s_at | NM_024351 | 0.01167287 | 0.049874433 | | | | |
| 375 | 83025 | M12156_at | NM_017248 | 0.003607981 | | | | | |
| 375 | 84619 | rc_AA799893_g_at | NM_017248 | −0.000443798 | | | | | −0.037062853 |
| 376 | 78205 | M12335expandedCDS_s_at | M12335 | | 0.029831802 | | | | |
| 377 | 80420 | M12579_at | NM_012767 | 0.000742569 | | 0.014093324 | | | |
| 378 | 81895 | M13750_at | NM_017036 | 0.003686236 | | | | | |
| 379 | 83014 | M13979_at | NM_138827 | | | | | 0.006219435 | |
| 380 | 81869 | M14656_at | NM_012881 | 0.009896762 | | | | 0.039938819 | |
| 381 | 76117 | rc_AA945573_f_at | NM_017158 | −0.002369789 | | | | | |
| 381 | 78216 | M14775_s_at | NM_017158 | | 0.002590986 | | | | |
| 382 | 82685 | M15327_at | NM_019286 | | | | −0.015095148 | | |
| 383 | 80435 | M15481_g_at | NM_178866 | 0.007674963 | | | | | |
| 384 | 78224 | M15528mRNA_at | M15528 | 0.005698674 | | | | | |
| 385 | 80267 | M15882_at | NM_031974 | −0.002076495 | | | | | |
| 386 | 78226 | M16406cds_s_at | NM_080773 | −0.001914192 | | | | | |
| 387 | 80086 | M16409_at | M16409 | 0.001451213 | | | | | |
| 388 | 79464 | M16410_at | NM_019162 | 0.001871955 | | | | | |
| 389 | 78228 | M17523mRNA_at | M17523 | −0.001862629 | | | | | |
| 390 | 76560 | M19533mRNA_i_at | NM_017101 | | | | | | 0.003408297 |
| 391 | 78244 | M19648cds_f_at | NM_001005382 | −0.002902663 | | | | | |
| 391 | 78259 | M22922cds_f_at | NM_001005382 | −0.004460334 | | | | | |
| 392 | 82995 | M19651_at | NM_012953 | | −0.04343168 | | | | |
| 393 | 78248 | M20722_s_at | M20722 | | | | | | −0.018938282 |
| 394 | 78249 | M20724_s_at | M20724 | 0.001214809 | | 0.001991975 | | −0.017566514 | |
| 395 | 78252 | M21770_at | NM_012503 | 0.001075282 | | | | | |
| 396 | 79690 | M22253_at | NM_030875 | 0.010407473 | | | | | |
| 397 | 78261 | M22993cds_s_at | NM_023103 | −0.001482466 | | | | | |
| 398 | 78262 | M23566exon_s_at | NM_012488 | 0.006115052 | | | | | |
| 399 | 79040 | X16044cds_s_at | NM_017040 | | −0.014649604 | | | | |
| 399 | 81898 | M23591mRNA#2_at | NM_017040 | | | | | | −0.016761673 |
| 400 | 78263 | M23643cds_s_at | NM_013046 | | 0.013268116 | | | | |
| 400 | 78307 | M36317_s_at | NM_013046 | 0.005947591 | | | | | 0.065269712 |
| 401 | 81881 | M23697_at | NM_013151 | 0.014271808 | | | | | |
| 402 | 82017 | M24067_at | NM_012620 | | | 0.001433834 | | | |
| 403 | 76565 | M24239cds#2_f_at | M24239 | −0.000612887 | | | | | |
| 404 | 81820 | M25073_at | NM_031012 | | | | | 0.020356238 | |
| 405 | 78276 | M25348_s_at | NM_013101 | 0.014193492 | | | | | |
| 406 | 79667 | M25804_g_at | NM_145775 | 0.00414945 | | | | | |
| 407 | 78279 | M25823_s_at | NM_138507 | | | | | 0.000820311 | |
| 408 | 76660 | M26125_at | NM_012844 | 0.000404377 | | | 0.045635855 | | |
| 409 | 76569 | M26127_s_at | NM_012541 | 0.002250061 | | | | | |
| 410 | 78324 | M61211cds_s_at | NM_012978 | −0.002445643 | | | | | |
| 410 | 79423 | M26199_s_at | NM_012978 | 0.005926155 | | | | 4.94E−05 | |
| 410 | 81781 | rc_AI236945_s_at | NM_012978 | 0.00443601 | | | | | |
| 411 | 79522 | M26534_f_at | M26534 | 0.004788314 | | | | | |
| 412 | 79457 | M26643_at | NM_013178 | 0.007145732 | | | | | |
| 413 | 82027 | M26686_at | M26686 | | | | | | 0.009182848 |
| 414 | 79451 | M27223_at | NM_019266 | | | | 0.056357808 | | 0.017503796 |
| 415 | 78285 | M27434_s_at | NM_147214 | 0.001265902 | | | | 0.007784002 | |
| 416 | 76685 | M27466_at | NM_019360 | 0.004903149 | | | 0.031115866 | | |
| 417 | 81803 | M29014_at | NM_017071 | 0.003028581 | | | | | |
| 418 | 81965 | M29293_at | NM_031117 | 0.000212838 | | | | | |
| 419 | 81947 | M29358_at | NM_017160 | | −0.001685792 | | | | |
| 420 | 81828 | M30689_at | NM_139257 | −0.001264109 | | | | | |
| 421 | 78296 | M31229mRNA_s_at | NM_133285 | −0.004863567 | | | | | |
| 422 | 81826 | M32016_at | NM_017068 | −0.00211238 | | | | | |
| 423 | 79474 | M33025_s_at | NM_031975 | | | | 4.33E−02 | | |
| 423 | 82271 | X16481_r_at | NM_031975 | 0.000530526 | | | | | |
| 424 | 81915 | M33962_g_at | NM_012637 | −0.002857687 | | | | | |
| 425 | 81969 | M35270complete_seq_i_at | NM_030656 | 0.000578948 | | | | | |
| 426 | 79525 | M35297_at | NM_153722 | −0.004910426 | | | | | |
| 427 | 78308 | M36418_s_at | NM_031608 | | 0.017242095 | | | | |
| 428 | 78312 | M36804_s_at | NM_001007597 | | −0.008279444 | | | 0.003564577 | |
| 429 | 78315 | M37568cds_at | M37568 | | | 0.028938418 | | | |
| 430 | 76583 | M38566mRNA_s_at | NM_178847 | 0.004986016 | | | | | |
| 431 | 82043 | M38759_at | NM_017016 | 0.015394833 | | | | | |
| 432 | 78319 | M55015cds_s_at | NM_012749 | −0.009732145 | | | | 0.014444572 | |
| 433 | 80414 | M57507_at | NM_012770 | | | | −0.039883117 | | |
| 434 | 82839 | M57664_g_at | NM_012529 | −0.012989424 | | | | | |
| 435 | 79560 | M57705_g_at | NM_019353 | −0.006396456 | | | | | |
| 436 | 81907 | M58758_g_at | NM_031604 | 0.003673209 | | | | | |
| 437 | 82095 | M59861_at | NM_022547 | 0.003455477 | | | | | |
| 438 | 76805 | M60322_at | NM_012498 | 0.004907209 | | | | | |
| 439 | 82045 | M60655_at | NM_016991 | 0.003723799 | | | | 0.013255805 | |
| 440 | 81141 | rc_AA944156_s_at | NM_017259 | −0.000188199 | | | | | |
| 440 | 82679 | M60921_at | NM_017259 | | −0.028876949 | | | −0.016598719 | |
| 440 | 82680 | M60921_g_at | NM_017259 | −0.007645031 | | | | | |
| 441 | 78325 | M61725_s_at | M61725 | 0.000882348 | | | | | |
| 442 | 76661 | M61875_s_at | NM_012924 | | −0.018846295 | | | | |
| 443 | 76591 | M62388_at | NM_031138 | −0.000373815 | | | | | |
| 444 | 81860 | M62891_at | NM_001010964 | 0.007559816 | | | | | |
| 445 | 81878 | M63006_at | NM_016989 | 0.003212739 | | | | | |
| 446 | 76654 | M63122_at | NM_013091 | −0.002401936 | | | | | |
| 447 | 79660 | M64299_at | NM_017155 | | | | | 0.012083657 | |
| 448 | 79622 | M64488_at | NM_012665 | −0.003928866 | | | | | |
| 449 | 79518 | M64793_at | M64793 | 0.002512867 | | | | | |
| 450 | 79798 | M64795_i_at | M64795 | −0.002619945 | | | | | |
| 451 | 79431 | M64862_at | NM_022667 | −0.012564105 | | | | | |
| 452 | 80431 | M64867_at | NM_017254 | 0.001065375 | | | | | |
| 453 | 82760 | M64986_g_at | NM_012963 | 0.013595304 | | | | | |
| 454 | 79554 | M65253_at | NM_133514 | −0.009185481 | | | | | |
| 455 | 78964 | X02322Poly_A_Site#1_s_at | NM_031577 | | | | | | −0.021754019 |
| 455 | 83008 | M73486_at | NM_031577 | −0.010238344 | | | | | |
| 456 | 81833 | M73714_at | NM_031731 | −0.00083521 | | | | | |
| 457 | 79454 | M74494_g_at | NM_012504 | 0.006625775 | | | 0.006133089 | | |
| 458 | 76598 | M74776_at | M74776 | −0.007349804 | | | | | |
| 459 | 81816 | M75148_at | M7518 | | | | | | 0.015970565 |
| 460 | 76602 | M76535cds_g_at | NM_019280 | 0.011466828 | | | | | |
| 461 | 76603 | M76704_s_at | NM_012861 | | | | 0.018622993 | | |
| 462 | 82775 | M77245_at | NM_017277 | 0.000192261 | | | | | |
| 463 | 82689 | M80550_at | NM_031007 | | −0.01693447 | | | | 0.015692905 |
| 464 | 76605 | M80784_s_at | NM_017256 | 0.003682445 | | | | | |
| 465 | 78354 | M81183Exon_UTR_g_at | M81183 | 0.001069896 | | | | | |
| 466 | 83033 | M81687_at | NM_013082 | 0.004948827 | | | | | |
| 467 | 76606 | M81766_at | NM_206849 | 0.007246221 | | | | | |
| 468 | 82099 | M84210_at | NM_053997 | −0.001443935 | | | | | |
| 469 | 80423 | M84361_at | NM_023981 | | | | | | 0.046694996 |
| 470 | 78367 | M86341_at | NM_183325 | | | | | −0.000263836 | |
| 471 | 76283 | rc_AA998683_g_at | NM_031970 | | 0.080966026 | | | 0.008440323 | |
| 471 | 76285 | rc_AI176658_s_at | NM_031970 | 0.003271431 | 0.03594857 | 0.008967663 | | 0.010410886 | |
| 471 | 76607 | M86389cds_s_at | NM_031970 | 0.007356954 | 0.036650097 | 0.016860416 | | 0.012919391 | |
| 472 | 79498 | M86514_at | M86514 | 0.0128694 | | | | | |
| 473 | 82762 | M86564_at | NM_021740 | 0.002126229 | | | | | |
| 474 | 76864 | M86758_at | NM_012883 | 0.008626611 | | | | | |
| 475 | 80222 | M87634_at | NM_012560 | −0.002211767 | | | | | |
| 476 | 78372 | M87786_at | M87786 | | | | 0.025757791 | | |
| 477 | 79425 | M88111_at | NM_053996 | −0.005093163 | | | | | |
| 478 | 78375 | M88488exon_at | M88488 | 0.002576042 | 0.027571293 | | | −0.009256581 | |
| 479 | 78376 | M89646_at | NM_031112 | | | | | | 0.04432628 |
| 480 | 78382 | M89953cds_at | NM_012852 | −0.001446736 | | 0.017715612 | | | |
| 481 | 79424 | M91234_f_at | M91234 | −0.001836591 | | | | | |
| 482 | 82415 | M91466_at | NM_017161 | | | | | | 0.024724113 |
| 483 | 78390 | M91595exon_s_at | NM_013122 | 0.005043394 | | | | | |
| 484 | 81988 | M92074_at | NM_017144 | −0.007452651 | | | | | |
| 485 | 78397 | M93257_s_at | NM_012531 | 0.002275195 | | | | | |
| 486 | 81337 | rc_AI030089_at | NM_022869 | 0.001106822 | | | | | |
| 486 | 81862 | M94287_at | NM_022869 | −0.010023387 | | | | | |
| 487 | 76849 | M94548_at | NM_019623 | 0.011447026 | | | | | |
| 488 | 78404 | M95791cds_f_at | M95791 | 0.000689205 | | | | | |
| 489 | 82552 | M96548_at | NM_052798 | 0.005370035 | | | | | |
| 490 | 78410 | M98819mRNA_s_at | NM_138876 | −0.008225413 | 0.04697558 | | | | |
| 491 | 78411 | M98826mRNA_at | M98826 | −0.000864781 | | | | | |
| 492 | 78412 | M98826mRNA_g_at | NM_031573 | 0.002273096 | | 0.007116274 | | | |
| 493 | 78414 | M99222_s_at | NM_012918 | −0.008179853 | | | | | |
| 494 | 83628 | rc_AA684919_at | AA684919 | | | | −0.027181343 | | −0.022852566 |
| 495 | 84403 | rc_AA799323_at | AA799323 | | | | | −0.008434908 | |
| 496 | 84404 | rc_AA799328_at | AA799328 | | | | | | −0.002271833 |
| 497 | 84405 | rc_AA799330_at | NM_001007634 | −0.00555613 | −0.014015419 | | | 0.008136828 | |
| 498 | 84416 | rc_AA799412_g_at | NM_001008511 | 0.003376791 | | | | | |
| 499 | 84420 | rc_AA799427_at | AA799427 | | | | | | 0.006244692 |
| 500 | 84426 | rc_AA799442_g_at | AA799442 | 0.008094791 | | | | | |
| 501 | 84440 | rc_AA799471_at | AA799471 | | | | −0.020136264 | | |
| 502 | 84445 | rc_AA799478_at | NM_022700 | 0.00380064 | | | | | |
| 503 | 76270 | rc_AA799479_g_at | AA799479 | −0.002450226 | | | | | |
| 504 | 84446 | rc_AA799480_at | AA799480 | −0.005630191 | | | | | |
| 505 | 84450 | rc_AA799488_at | AA799488 | −0.003487773 | | | | | |
| 506 | 84453 | rc_AA799495_at | AA799495 | 0.009715729 | | | 0.041752342 | | |
| 507 | 81065 | rc_AA799498_at | NM_031545 | 0.011334154 | | | | | |
| 508 | 84474 | rc_AA799534_at | AA799534 | 0.001167996 | | | | | |
| 509 | 84482 | rc_AA799544_at | AA799544 | −0.003650325 | | | | | |
| 510 | 84488 | rc_AA799554_at | AA799554 | | | | 0.003036535 | | |
| 511 | 84491 | rc_AA799566_g_at | AA799566 | 0.004284934 | | | | | |
| 512 | 84521 | rc_AA799637_at | AA799637 | −0.002952182 | | | | | |
| 513 | 84523 | rc_AA799641_at | NM_001008312 | 0.000526882 | | | | | −0.010327135 |
| 513 | 84524 | rc_AA799641_g_at | NM_001008312 | 0.01026144 | | | | | |
| 514 | 84529 | rc_AA799656_at | AA799656 | 0.000150322 | | | | | |
| 514 | 84530 | rc_AA799656_g_at | AA799656 | 0.000865355 | | | | | |
| 515 | 84556 | rc_AA799724_g_at | AA799724 | 0.005517782 | | | | | |
| 516 | 84576 | rc_AA799766_at | AA799766 | −0.010907393 | | | | | |
| 517 | 84577 | rc_AA799771_at | AA799771 | −0.009461511 | | | | | |
| 518 | 84578 | rc_AA799773_at | AA799773 | | | | | 0.005751445 | |
| 518 | 84579 | rc_AA799773_g_at | AA799773 | 0.005942549 | | 0.01634096 | | 0.004848447 | |
| 519 | 84585 | rc_AA799791_at | NM_184049 | 0.005509131 | | | | | 0.008957274 |
| 520 | 84593 | rc_AA799812_at | AA799812 | 0.003971831 | | | | | |
| 521 | 84603 | rc_AA799847_at | AA799847 | 0.014095003 | | 0.055169906 | | | |
| 522 | 84613 | rc_AA799887_s_at | NM_031331 | −0.004358308 | | | | | |
| 523 | 84630 | rc_AA799992_g_at | AA799992 | 0.000352441 | | | | | |
| 524 | 84638 | rc_AA800015_at | AA800015 | | | | −0.049503595 | | |
| 525 | 84652 | rc_AA800053_at | NM_001007618 | −0.006263749 | | | | | |
| 526 | 84663 | rc_AA800175_at | AA800175 | 0.003056539 | | | | | |
| 527 | 84685 | rc_AA800222_at | AA800222 | 0.004499702 | | | | | |
| 528 | 84696 | rc_AA800272_at | AA800272 | −0.003694561 | | | | | |
| 529 | 76706 | U53922_at | NM_022934 | | 0.005855112 | | | 0.005170846 | |
| 529 | 84719 | rc_AA800551_at | NM_022934 | | −0.053507545 | | | | |
| 530 | 84723 | rc_AA800572_at | AA800572 | −0.004247268 | | 0.041515002 | | | −0.0124693 |
| 531 | 84738 | rc_AA800651_s_at | AA800651 | | | | | 0.005697304 | |
| 532 | 84740 | rc_AA800665_at | NM_023955 | −0.000370521 | | | | | |
| 533 | 84745 | rc_AA800678_g_at | AA800678 | | −0.063346114 | | | | |
| 534 | 84747 | rc_AA800680_at | AA800680 | −0.002893909 | | | | | |
| 535 | 84749 | rc_AA800686_at | AA800686 | −0.008590838 | | | −0.063422676 | | |
| 536 | 84761 | rc_AA800719_at | AA800719 | 0.003958271 | | | | | |
| 537 | 84773 | rc_AA800750_f_at | AA800750 | 0.010765312 | | | | | |
| 538 | 84782 | rc_AA800784_at | NM_031327 | 0.001361422 | | | | | |
| 539 | 84799 | rc_AA800849_f_at | AA800849 | 0.007093074 | | | | | |
| 540 | 84804 | rc_AA800853_at | NM_001002830 | −0.001029726 | | | | 0.009232067 | |
| 541 | 84816 | rc_AA800912_g_at | NM_001001504 | 0.004810884 | | | | | |
| 542 | 81086 | rc_AA801286_at | NM_032057 | | | | | | −0.008449475 |
| 543 | 81489 | rc_AA817846_at | NM_053995 | −0.000910395 | | | | | |
| 544 | 81490 | rc_AA817887_at | NM_022511 | | | | −0.019688951 | | |
| 545 | 81511 | rc_AA818025_g_at | AA818025 | 0.002705228 | | | | | |
| 546 | 81524 | rc_AA818114_at | NM_017355 | | | | | | 0.011449245 |
| 547 | 81534 | rc_AA818144_at | NM_017096 | 0.003166608 | | | | | |
| 548 | 81600 | rc_AA818970_s_at | NM_017333 | 0.007046339 | | | | | |
| 549 | 81601 | rc_AA818982_at | NM_012887 | 0.001960938 | | | | | |
| 549 | 81602 | rc_AA818982_g_at | NM_012887 | 0.006347934 | | | | | |
| 550 | 81090 | rc_AA849036_at | NM_017090 | −0.00148607 | | | | | |
| 551 | 81092 | rc_AA849648_at | NM_053330 | 0.009772183 | | | | | |
| 552 | 81098 | rc_AA850669_at | NM_053891 | −0.000846356 | | | | | |
| 552 | 82494 | U50707_at | NM_053891 | | | | | | 0.00555806 |
| 553 | 81100 | rc_AA850756_at | NM_012857 | −0.003189568 | | | | | |
| 554 | 76272 | rc_AA851403_at | AA851403 | −0.001284935 | | | | | |
| 554 | 76273 | rc_AA851403_g_at | AA851403 | 0.003818363 | | | | | |
| 555 | 81797 | rc_AA858520_at | NM_012561 | −0.002367469 | | | | −0.009044169 | |
| 556 | 83046 | rc_AA858586_at | AA858586 | | | | | | 0.034230693 |
| 557 | 83050 | rc_AA858600_at | AA858600 | −0.00803417 | | | | | |
| 558 | 83052 | rc_AA858603_g_at | AA858603 | | | | | | −0.038308353 |
| 559 | 78660 | S82383_s_at | NM_173111 | | −0.007422005 | | | 0.008821859 | |
| 559 | 81795 | rc_AA859305_s_at | NM_173111 | | | | | 0.020387205 | |
| 560 | 76323 | rc_AA859372_s_at | AA859372 | −0.008167318 | | | 0.05285992 | | |
| 561 | 83066 | rc_AA859473_at | NM_053555 | 0.002735623 | | | | | |
| 562 | 83069 | rc_AA859483_at | AA859483 | −8.50E−05 | | | | | |
| 563 | 83082 | rc_AA859533_at | AA859533 | | | | −0.047762669 | | |
| 564 | 83094 | rc_AA859581_at | NM_138518 | | | | | | 0.061714657 |
| 565 | 83099 | rc_AA859597_at | AA859597 | | | | | | 0.005064774 |
| 566 | 77049 | rc_AA859648_at | AA859648 | | −0.019052977 | | | | |
| 567 | 83115 | rc_AA859663_at | AA859663 | 0.007430781 | | | | | |
| 568 | 83122 | rc_AA859680_at | AA859680 | 0.00931698 | | | | | |
| 569 | 83134 | rc_AA859718_at | AA859718 | | | | 1.64E−05 | | −0.007710244 |
| 570 | 83135 | rc_AA859719_at | AA859719 | 0.00016051 | | | | | |
| 571 | 83138 | rc_AA859740_at | AA859740 | | | | | | 0.001655408 |
| 572 | 83140 | rc_AA859750_at | AA859750 | | | | | | 0.021363096 |
| 573 | 83144 | rc_AA859760_at | AA859760 | −0.000952225 | | | | | |
| 574 | 83150 | rc_AA859804_at | AA859804 | 0.006016185 | | | | | |
| 575 | 83151 | rc_AA859805_at | NM_001012125 | 0.005341759 | | | | | |
| 576 | 83152 | rc_AA859806_at | NM_024161 | 0.005028894 | | | | | |
| 577 | 83154 | rc_AA859827_at | AA859827 | | 0.031544488 | | | 0.006662761 | |
| 578 | 81002 | rc_AA859830_s_at | NM_017087 | | | | | 0.031669772 | |
| 579 | 78820 | U57050_g_at | NM_031978 | 0.001912498 | | | | | |
| 579 | 83165 | rc_AA859869_s_at | NM_031978 | 0.003672405 | | | 0.001819666 | | |
| 580 | 77050 | rc_AA900380_at | NM_130426 | −0.002210573 | | | | | |
| 580 | 83172 | rc_AA859889_at | NM_130426 | | | | | | −0.002564839 |
| 581 | 83185 | rc_AA859922_at | AA859922 | 0.005289153 | | | 0.030315386 | | |
| 582 | 83199 | rc_AA859954_at | NM_138839 | −0.002480264 | | | | | |
| 583 | 83210 | rc_AA859983_at | AA859983 | −0.001803564 | | | | | |
| 584 | 83214 | rc_AA859994_at | AA859994 | | | | | | 0.060637375 |
| 585 | 83215 | rc_AA859996_at | AA859996 | | | | −0.020661856 | | |
| 586 | 83222 | rc_AA860024_at | AA860024 | −0.01071322 | | | | | |
| 587 | 81155 | rc_AA945321_at | NM_134326 | | −0.006286767 | | | | |
| 587 | 83234 | rc_AA860062_g_at | NM_134326 | | 0.041784117 | | | 0.02116456 | |
| 587 | 83240 | rc_AA866237_s_at | NM_134326 | 0.01035651 | | 0.030025172 | | | |
| 588 | 83235 | rc_AA866221_at | AA866221 | | | | | | 0.021857116 |
| 589 | 83250 | rc_AA866272_at | AA866272 | 0.001100005 | | | | | −0.04309076 |
| 590 | 83265 | rc_AA866362_at | AA866362 | −0.008781529 | | | | | |
| 591 | 83274 | rc_AA866414_at | NM_012651 | 0.017450818 | | | | | |
| 592 | 83277 | rc_AA866426_at | AA866426 | | | | | | −0.002404722 |
| 593 | 83281 | rc_AA866439_g_at | AA866439 | 0.010987448 | | | | | |
| 594 | 83283 | rc_AA866444_s_at | AA866444 | | | | 0.100440702 | | −0.015328696 |
| 595 | 83302 | rc_AA874803_g_at | AA874803 | | | | | 0.010381065 | |
| 596 | 83305 | rc_AA874815_at | AA874815 | | | | | | 0.007037474 |
| 597 | 83308 | rc_AA874832_at | AA874832 | 0.006270037 | | | | | |
| 598 | 83322 | rc_AA874874_at | NM_017270 | 0.0057985 | | | | | |
| 599 | 83346 | rc_AA874952_at | AA874952 | | | | 0.008067541 | | |
| 600 | 83354 | rc_AA874995_at | NM_001007703 | 0.002595813 | | 0.049089357 | | | |
| 601 | 83355 | rc_AA874999_at | AA874999 | 0.005158087 | | | | | |
| 602 | 83357 | rc_AA875002_at | AA875002 | 0.007862894 | | | | | |
| 603 | 83359 | rc_AA875010_at | NM_053589 | 0.001225273 | | | | | |
| 604 | 83360 | rc_AA875017_at | AA875017 | | | | | | 0.003767403 |
| 605 | 83374 | rc_AA875042_at | AA875042 | | | | | | −0.003395042 |
| 606 | 83385 | rc_AA875089_at | NM_053295 | 0.007609667 | | 0.061475675 | | | |
| 607 | 83388 | rc_AA875098_at | AA875098 | 0.00073985 | | | | | |
| 608 | 83392 | rc_AA875121_at | NM_012866 | | | 0.023945817 | | | |
| 609 | 83397 | rc_AA875126_g_at | AA875126 | | | 0.023132493 | | | |
| 610 | 83413 | rc_AA875165_at | AA875165 | | | | −0.069786007 | | |
| 611 | 83415 | rc_AA875171_at | AA875171 | 0.003580111 | | | | | |
| 611 | 83416 | rc_AA875171_g_at | AA875171 | 0.002270274 | | 0.03038693 | | | |
| 612 | 83418 | rc_AA875182_at | AA875182 | | | | 0.006078842 | | |
| 613 | 83423 | rc_AA875197_at | AA875197 | | | | | | 0.030036996 |
| 614 | 83424 | rc_AA875198_at | NM_001008386 | | | | | | −0.005616964 |
| 615 | 83428 | rc_AA875205_g_at | AA875205 | −0.000528627 | | | | | |
| 616 | 83429 | rc_AA875206_at | NM_053747 | −0.003130729 | | | | | |
| 617 | 83443 | rc_AA875255_at | AA875255 | | | | | | −0.002837261 |
| 618 | 83446 | rc_AA875263_at | AA875263 | 0.008967186 | | | | | |
| 619 | 83450 | rc_AA875275_at | AA875275 | | | | | | 0.03898176 |
| 620 | 76248 | rc_AA875327_at | NM_001006957 | −0.004153801 | | | | | |
| 621 | 83459 | rc_AA875348_at | AA875348 | | | | | | 0.029240504 |
| 622 | 83463 | rc_AA875405_at | NM_001012091 | 0.012075055 | | | | | |
| 623 | 83472 | rc_AA875428_at | AA875428 | | 0.009396528 | | | | |
| 624 | 83476 | rc_AA875444_at | AA875444 | | | | | | −0.003541399 |
| 625 | 83480 | rc_AA875468_at | AA875468 | | | | | | 0.003171944 |
| 626 | 81401 | rc_AI070967_g_at | NM_012903 | 0.002751123 | | | | | |
| 626 | 83483 | rc_AA875495_at | NM_012903 | 0.003680805 | | | | | |
| 626 | 84218 | rc_AA893496_at | NM_012903 | | | | 0.018984788 | | |
| 627 | 83486 | rc_AA875506_at | NM_053824 | | | | | | 0.002840699 |
| 628 | 83505 | rc_AA875598_at | AA875598 | 0.000830058 | | | | | |
| 629 | 83509 | rc_AA875617_at | NM_031083 | | | | | | −0.008089917 |
| 630 | 83516 | rc_AA875641_at | AA875641 | | | | | | 0.001881294 |
| 631 | 83522 | rc_AA875665_g_at | NM_001008694 | 0.003456358 | | | | | |
| 632 | 83635 | rc_AA891032_at | AA891032 | −0.002539816 | | | | | |
| 633 | 83642 | rc_AA891058_at | AA891058 | | | | | | −0.022152715 |
| 634 | 83644 | rc_AA891069_at | AA891069 | −0.00538359 | | | | | |
| 635 | 83649 | rc_AA891140_at | AA891140 | | | | | | −0.018521384 |
| 635 | 83650 | rc_AA891140_g_at | AA891140 | 0.002586929 | | | | | |
| 636 | 83655 | rc_AA891196_at | AA891196 | | | | 0.100944415 | | |
| 637 | 83660 | rc_AA891220_at | NM_177928 | 0.001611575 | | | −0.009705684 | | |
| 638 | 83661 | rc_AA891221_at | AA891221 | 0.007721937 | | | | | |
| 639 | 78837 | U63923_at | NM_031614 | 0.000688251 | | | | | |
| 639 | 83671 | rc_AA891286_at | NM_031614 | −0.010211869 | | | | | |
| 640 | 83681 | rc_AA891314_at | AA891314 | 0.007136435 | | | | | |
| 641 | 83684 | rc_AA891356_at | AA891356 | 0.006773782 | | | | | |
| 642 | 83685 | rc_AA891362_at | NM_057186 | | | | | | 0.013554353 |
| 643 | 83555 | rc_H31665_at | NM_080902 | | | | | | −0.028862975 |
| 643 | 83688 | rc_AA891422_at | NM_080902 | −0.007932954 | | | | | |
| 644 | 83689 | rc_AA891423_at | AA891423 | | | | −0.059021696 | | |
| 645 | 83698 | rc_AA891476_at | AA891476 | 0.009388522 | | | | | |
| 646 | 83702 | rc_AA891524_at | NM_001008367 | | | | | 0.011566687 | |
| 647 | 83705 | rc_AA891542_at | AA891542 | 0.001444456 | 0.03878479 | | | 0.003342183 | |
| 648 | 83708 | rc_AA891553_at | NM_001004283 | −0.006514491 | | | | | |
| 649 | 83716 | rc_AA891588_at | AA891588 | −0.003510287 | | | | | |
| 650 | 83718 | rc_AA891591_at | NM_031356 | | | | | | −0.016209811 |
| 651 | 83720 | rc_AA891596_at | AA891596 | 0.000863069 | | | | | |
| 652 | 83722 | rc_AA891614_at | NM_139332 | 0.002372024 | | | | | |
| 653 | 83723 | rc_AA891631_at | AA891631 | 0.010199871 | | | | | |
| 654 | 83729 | rc_AA891666_g_at | NM_001007804 | | | | | | −0.000187834 |
| 655 | 83743 | rc_AA891713_at | NM_173340 | | | | | −0.007428801 | |
| 656 | 83747 | rc_AA891719_at | AA891719 | −0.00443289 | | | | | |
| 657 | 83748 | rc_AA891721_at | NM_001009647 | 0.006110493 | | | | | −0.012200106 |
| 658 | 83749 | rc_AA891724_at | NM_198765 | −0.002022493 | | | −0.008623252 | | |
| 659 | 83752 | rc_AA891727_g_at | AA891727 | | | | | | −0.043856629 |
| 660 | 83757 | rc_AA891737_at | AA891737 | −0.004544375 | | | −0.047028324 | | |
| 661 | 83761 | rc_AA891746_at | AA891746 | 0.005867454 | | | | | |
| 662 | 81025 | rc_AA891764_s_at | NM_030827 | −0.001832133 | | | | | |
| 663 | 83781 | rc_AA891810_at | AA891810 | 0.003866891 | | | | | |
| 663 | 83782 | rc_AA891810_g_at | AA891810 | 0.008360481 | | | | | |
| 664 | 83797 | rc_AA891838_at | AA891838 | | −0.023837508 | | | | |
| 665 | 83800 | rc_AA891842_g_at | AA891842 | | 0.031931754 | | | | |
| 666 | 83803 | rc_AA891851_at | AA891851 | −0.001124354 | | | | | |
| 667 | 83813 | rc_AA891877_at | AA891877 | 0.008255255 | | | | | |
| 668 | 83815 | rc_AA891880_g_at | NM_022948 | −0.001837238 | | | | | 0.017260857 |
| 669 | 83822 | rc_AA891903_at | AA891903 | −0.001324754 | | | | | |
| 670 | 83824 | rc_AA891914_at | NM_001005383 | | | | −0.059143418 | | |
| 671 | 83825 | rc_AA891916_at | NM_032615 | −0.00626898 | | | | | |
| 671 | 83826 | rc_AA891916_g_at | NM_032615 | 0.011123591 | | | | | |
| 672 | 83827 | rc_AA891917_at | AA891917 | 0.002901743 | | | | | |
| 673 | 83838 | rc_AA891940_at | AA891940 | | −0.047915831 | | | | |
| 674 | 83839 | rc_AA891943_at | AA891943 | −0.001238999 | | | | | |
| 675 | 83846 | rc_AA891969_at | AA891969 | | −0.035329818 | | | | |
| 676 | 83851 | rc_AA892006_g_at | AA892006 | 0.002379297 | | | | | |
| 677 | 83855 | rc_AA892012_g_at | NM_013177 | −0.000992537 | | | 0.028538151 | | |
| 678 | 83857 | rc_AA892014_r_at | NM_139101 | | | | 0.030134899 | | |
| 679 | 83861 | rc_AA892042_at | AA892042 | | | | | | 0.020338447 |
| 680 | 83862 | rc_AA892049_at | AA892049 | | | | 0.040333594 | | |
| 681 | 83871 | rc_AA892112_g_at | AA892112 | | | | | −0.009478954 | |
| 682 | 83886 | rc_AA892204_at | AA892204 | | | | −0.000132542 | | |
| 683 | 83888 | rc_AA892228_at | AA892228 | | −0.023967283 | | | | |
| 684 | 76221 | rc_AA892248_at | AA892248 | | | | | | −0.01169609 |
| 684 | 76222 | rc_AA892248_g_at | AA892248 | 0.005168346 | | | | | −0.001978019 |
| 685 | 83895 | rc_AA892258_at | NM_053524 | | | | 0.012000131 | | |
| 686 | 83912 | rc_AA892298_at | NM_175707 | −0.004909686 | | | | | |
| 687 | 83917 | rc_AA892303_g_at | NM_001009666 | | −0.003309766 | | | 0.029387265 | |
| 688 | 83919 | rc_AA892305_at | NM_001007707 | −0.006348242 | | | | | |
| 689 | 83922 | rc_AA892310_at | AA892310 | 0.000779846 | | | | | |
| 690 | 83928 | rc_AA892318_g_at | AA892318 | 0.007176704 | | | | | |
| 691 | 83947 | rc_AA892378_g_at | AA892378 | 0.002486153 | | | | | |
| 692 | 83948 | rc_AA892380_at | AA892380 | −0.001520705 | | | −0.067903079 | | |
| 693 | 83958 | rc_AA892400_at | NM_198736 | −0.001099869 | | | | | |
| 694 | 83965 | rc_AA892462_at | AA892462 | | | | 0.003798135 | | 0.019270565 |
| 695 | 83969 | rc_AA892470_at | AA892470 | | −0.014962594 | | | | |
| 696 | 83977 | rc_AA892498_at | NM_001005547 | −0.010718758 | | | | | |
| 697 | 83985 | rc_AA892520_at | AA892520 | −0.003283709 | | | | | |
| 697 | 83986 | rc_AA892520_g_at | AA892520 | 0.000213305 | | | | | |
| 698 | 83988 | rc_AA892526_at | AA892526 | 0.01533824 | | | | | |
| 699 | 76243 | rc_AA892532_at | NM_001004442 | | −0.052536596 | | | | |
| 700 | 83999 | rc_AA892551_i_at | AA892551 | −0.005179648 | | | | | |
| 701 | 84004 | rc_AA892557_at | NM_001007802 | | | | | 0.006785172 | |
| 702 | 84014 | rc_AA892578_at | AA892578 | 0.007516131 | 0.056262537 | | | | |
| 703 | 84015 | rc_AA892582_s_at | AA892582 | | 0.029592314 | | | | |
| 704 | 84016 | rc_AA892593_at | AA892593 | | | | | | −0.000309005 |
| 705 | 84017 | rc_AA892598_at | NM_175580 | −0.000688959 | | | | | |
| 705 | 84018 | rc_AA892598_g_at | NM_175580 | | −0.006337728 | | | 0.001587735 | |
| 706 | 84022 | rc_AA892630_at | AA892630 | −0.00012429 | | | | | |
| 707 | 84025 | rc_AA892635_g_at | AA892635 | 0.001931834 | | | | | |
| 708 | 84031 | rc_AA892647_at | AA892647 | 0.002876995 | | | | | |
| 709 | 81037 | rc_AA892775_at | NM_012771 | | −0.035311125 | | | | |
| 710 | 84052 | rc_AA892779_at | AA892779 | 0.00783691 | | | | | |
| 711 | 84069 | rc_AA892820_at | AA892820 | | | −0.013211487 | | −0.004118393 | |
| 712 | 84072 | rc_AA892828_at | NM_001007620 | 0.011228277 | | | | | |
| 713 | 84076 | rc_AA892832_at | NM_134382 | 0.003898561 | | 0.049805322 | | | |
| 714 | 84079 | rc_AA892843_at | NM_001007637 | −0.001478317 | | | | | |
| 715 | 84083 | rc_AA892851_g_at | NM_001008521 | −0.00263455 | | | | | |
| 716 | 84085 | rc_AA892855_at | NM_001009690 | | | | | | 0.020121134 |
| 717 | 84089 | rc_AA892860_g_at | AA892860 | 0.002618366 | | | | | |
| 718 | 84090 | rc_AA892861_at | AA892861 | | | | −0.008973022 | | |
| 719 | 84106 | rc_AA892940_g_at | AA892940 | 0.001289925 | | | | | |
| 720 | 84115 | rc_AA892987_at | AA892987 | −0.007682307 | | | | | |
| 721 | 84118 | rc_AA892997_at | AA892997 | −0.006734893 | | | | | |
| 722 | 84128 | rc_AA893035_s_at | NM_134330 | | | | | | −0.0072966 |
| 723 | 84132 | rc_AA893043_at | AA893043 | | | | −0.050210056 | | |
| 724 | 84153 | rc_AA893172_at | AA893172 | | | | 0.09671519 | | |
| 725 | 84158 | rc_AA893184_at | AA893184 | 0.004214224 | | | | | |
| 726 | 84181 | rc_AA893237_at | AA893237 | 0.000479633 | | | | | |
| 727 | 84182 | rc_AA893239_at | NM_053493 | | | | | −0.009150149 | |
| 728 | 84183 | rc_AA893242_at | NM_012820 | | | | 0.012680237 | | |
| 728 | 84184 | rc_AA893242_g_at | NM_012820 | −0.0036044 | | | | | |
| 729 | 84185 | rc_AA893244_at | AA893244 | 0.008792292 | | | | | |
| 730 | 84186 | rc_AA893246_at | NM_199386 | | | | −0.031042485 | | |
| 731 | 84192 | rc_AA893280_at | NM_001007144 | | 0.027992204 | | | −0.006965129 | |
| 732 | 84216 | rc_AA893493_i_at | AA893493 | | | | | | 0.028944591 |
| 733 | 84224 | rc_AA893534_at | AA893534 | | | 0.056477818 | | | |
| 734 | 84230 | rc_AA893584_at | AA893584 | 0.009607751 | | | | | |
| 735 | 84232 | rc_AA893592_at | AA893592 | 0.003299129 | | | | | |
| 736 | 84250 | rc_AA893659_at | AA893659 | | | | | | 0.028308157 |
| 737 | 84279 | rc_AA893770_g_at | NM_139104 | 0.00429907 | | | | | |
| 738 | 84281 | rc_AA893781_at | AA893781 | 0.006968054 | | | | | 0.019372323 |
| 739 | 84287 | rc_AA893821_at | NM_134398 | | | | | | −0.011738643 |
| 740 | 84291 | rc_AA893857_at | AA893857 | | | | 0.01900966 | | |
| 741 | 84313 | rc_AA893984_at | AA893984 | | | | 0.070007923 | | |
| 742 | 84316 | rc_AA894004_at | AA894004 | | | | | 0.018239648 | |
| 743 | 84317 | rc_AA894008_at | AA894008 | −0.001489864 | | | | | |
| 744 | 84320 | rc_AA894016_at | AA894016 | | | | −0.022995815 | | |
| 745 | 84322 | rc_AA894027_at | AA894027 | | | | −0.081102578 | | |
| 746 | 84326 | rc_AA894045_at | NM_181822 | | −0.005842006 | | | | |
| 747 | 84338 | rc_AA894092_at | AA894092 | | 0.046224771 | | | | |
| 748 | 81056 | rc_AA894174_at | NM_001009668 | −0.004303737 | | | | | |
| 749 | 84360 | rc_AA894200_g_at | AA894200 | 0.003290009 | | | | | |
| 750 | 84374 | rc_AA894259_at | AA894259 | 0.003438482 | | | | | |
| 751 | 84380 | rc_AA894292_at | AA894292 | 0.005215236 | | | | | |
| 752 | 84383 | rc_AA894304_at | AA894304 | −0.005040496 | | | | | |
| 753 | 81059 | rc_AA894317_s_at | NM_032083 | 0.012697681 | | | | | |
| 754 | 84387 | rc_AA894318_at | AA894318 | 0.001315192 | | | | | |
| 755 | 84393 | rc_AA894345_at | AA894345 | 0.006273233 | | | | | |
| 756 | 81110 | rc_AA899106_at | NM_022267 | 0.004268189 | | | | | |
| 757 | 81116 | rc_AA900503_at | NM_019147 | | | | 0.042029105 | | −0.013994777 |
| 758 | 81126 | rc_AA900900_s_at | NM_032067 | | | | 0.091797057 | | |
| 759 | 77051 | rc_AA925300_at | AA925300 | | | | | −0.006025421 | |
| 760 | 81191 | rc_AA925752_at | NM_031561 | 0.010204397 | | | | | |
| 761 | 77054 | rc_AA926129_at | AA926129 | −0.006140817 | | | | | |
| 762 | 76254 | rc_AA926149_g_at | NM_012520 | 0.010403853 | | | | | |
| 763 | 81152 | rc_AA945169_at | NM_012681 | 0.005191478 | | | | | |
| 764 | 81154 | rc_AA945171_at | AA945171 | −0.000289178 | | | | | |
| 765 | 76346 | rc_AA945704_at | AA945704 | | −0.05522422 | | | | |
| 766 | 81162 | rc_AA945737_at | NM_022205 | 0.001072872 | | | | | |
| 767 | 76312 | rc_AA946040_at | AA946040 | | | | | | 0.014251159 |
| 768 | 81166 | rc_AA946108_at | AA946108 | 0.009364262 | | | | | |
| 769 | 81167 | rc_AA946251_at | NM_030829 | 0.000246211 | | | | | |
| 770 | 81172 | rc_AA946439_at | AA946439 | | | | 0.05623229 | | |
| 771 | 81226 | rc_AA957930_s_at | NM_017212 | | | | | | −0.019280678 |
| 772 | 81227 | rc_AA957961_at | NM_054006 | 0.000104188 | | | | | −0.021364913 |
| 772 | 81395 | rc_AI070521_s_at | NM_054006 | | | | 0.030489236 | | |
| 772 | 82424 | X52311_at | NM_054006 | | 0.014426865 | | | | |
| 773 | 76318 | rc_AA963449_s_at | NM_012941 | | | | −0.12358211 | | |
| 773 | 76319 | rc_AA997614_s_at | NM_012941 | −0.00294488 | | | | | |
| 773 | 76902 | U17697_s_at | NM_012941 | −0.001227975 | | | | 0.005734316 | |
| 774 | 81235 | rc_AA964530_at | NM_024150 | 0.003010634 | | | | | |
| 775 | 81238 | rc_AA965119_at | NM_031534 | 0.011144083 | | | | | |
| 776 | 76343 | rc_AI007820_s_at | NM_001004082 | −0.005946223 | | | | | |
| 776 | 76344 | rc_AI008074_s_at | NM_001004082 | 0.002051131 | | 0.030953844 | | | |
| 777 | 81262 | rc_AI007824_g_at | AI007824 | | | | | | −0.013277269 |
| 778 | 81263 | rc_AI007835_at | NM_053343 | 0.001639722 | | | | | |
| 779 | 76186 | rc_AI009141_at | AI009141 | | | | | | −0.015490749 |
| 780 | 76276 | rc_AI009390_at | AI009390 | −0.010502044 | | | | | |
| 781 | 81286 | rc_AI009605_at | NM_013216 | 0.002906278 | | | | | 0.00890716 |
| 782 | 81292 | rc_AI010083_at | NM_057114 | | | | 0.022898815 | | |
| 783 | 81295 | rc_AI010453_at | NM_022519 | | | | | 0.00458851 | |
| 784 | 76191 | rc_AI011706_at | AI011706 | | | | | | 0.024339502 |
| 785 | 81309 | rc_AI012595_at | NM_017039 | | | | −0.086817828 | | |
| 786 | 81325 | rc_AI013389_at | NM_012521 | 0.008698172 | | | | | |
| 787 | 81326 | rc_AI013472_at | NM_138905 | | | | | 0.013890327 | |
| 788 | 81329 | rc_AI013795_at | NM_022713 | 0.00805401 | | | | | |
| 789 | 76170 | rc_AI013834_s_at | NM_024392 | −0.017962317 | | | | | |
| 790 | 81318 | rc_AI014135_at | AI014135 | | 0.032116415 | | | | |
| 791 | 81320 | rc_AI014163_at | NM_019242 | | −0.020785919 | | | −0.008578385 | |
| 792 | 81333 | rc_AI029183_s_at | NM_012567 | 0.003737449 | | | | | |
| 793 | 81334 | rc_AI029279_at | NM_053868 | 0.001160078 | | | | | |
| 794 | 81342 | rc_AI031019_at | NM_172029 | −0.001381499 | | | | | |
| 795 | 81359 | rc_AI044517_at | NM_017215 | 0.000787246 | | | | | |
| 795 | 82215 | U15098_at | NM_017215 | 0.001213597 | | | | | |
| 796 | 81361 | rc_AI044716_at | NM_153735 | −0.007078477 | | | | | |
| 797 | 81362 | rc_AI044739_at | NM_133423 | 0.007738837 | | | | | |
| 798 | 81366 | rc_AI044985_g_at | NM_019241 | 0.006849151 | | | | | |
| 799 | 81392 | rc_AI070142_at | NM_017030 | 0.004317143 | | | | | |
| 800 | 81396 | rc_AI070577_at | NM_012817 | 0.005615894 | | 0.032602293 | | | |
| 801 | 81398 | rc_AI070783_at | NM_017313 | 0.003905441 | | | | | |
| 802 | 81403 | rc_AI071104_at | NM_012774 | 0.005502534 | | | | | |
| 803 | 81405 | rc_AI071299_at | NM_031135 | | | | 0.046623766 | | |
| 804 | 81412 | rc_AI071866_s_at | AI071866 | 0.009475712 | | 0.046925673 | | | −0.009066898 |
| 805 | 81426 | rc_AI073232 s_at | NM_019246 | 0.001003873 | | | | | |
| 806 | 81471 | rc_AI101320_at | AI101320 | | | | 0.047475109 | | |
| 807 | 81494 | rc_AI102044_at | AI102044 | 0.000251044 | | | | 0.02357011 | 0.005666544 |
| 808 | 81498 | rc_AI102205_s_at | NM_024000 | | | | | | −0.018765764 |
| 809 | 81513 | rc_AI103671_at | NM_053311 | | | | | | 0.00371935 |
| 810 | 81517 | rc_AI103911_at | NM_001008888 | 0.002842338 | | | | | |
| 811 | 81518 | rc_AI103957_at | NM_013087 | −0.004321481 | | | | | |
| 812 | 76313 | rc_AI104035_s_at | AI104035 | −0.003752014 | | | | | |
| 813 | 76204 | rc_AI104077_at | AI104077 | 0.001700796 | | | | | |
| 814 | 81527 | rc_AI104399_at | NM_022922 | | −0.035280823 | | | | |
| 815 | 81529 | rc_AI104513_at | NM_145783 | 0.002605906 | | | | | |
| 816 | 81536 | rc_AI104781_at | NM_017260 | −0.005911732 | | | | | |
| 817 | 81539 | rc_AI105348_i_at | NM_017147 | | | | | | −0.010353994 |
| 818 | 81429 | rc_AI112173_at | NM_013113 | 0.000267698 | | | | | |
| 819 | 81436 | rc_AI136396_at | NM_172034 | 0.00526251 | | | | | |
| 820 | 76228 | rc_AI136977_at | AI136977 | 0.006379977 | | | | | |
| 821 | 81447 | rc_AI137583_at | NM_013060 | 0.004202351 | | | | | |
| 822 | 81451 | rc_AI138143_at | NM_012796 | 0.015097915 | | | | | |
| 823 | 81459 | rc_AI145367_at | NM_053874 | 0.004850133 | | | | | |
| 824 | 81465 | rc_AI145680_s_at | NM_012716 | 0.004836307 | | | | | |
| 825 | 76347 | rc_AI169265_at | AI169265 | 0.002504574 | | | | | |
| 826 | 81483 | rc_AI169327_at | NM_053819 | 0.001143893 | 0.082290651 | | | | |
| 826 | 81484 | rc_AI169327_g_at | NM_053819 | 0.002720675 | 0.065911479 | | | | |
| 827 | 81544 | rc_AI169612_at | NM_053365 | | | | | | 0.022227731 |
| 828 | 81548 | rc_AI169756_s_at | AI169756 | | −0.003421449 | | | | |
| 829 | 76133 | rc_AI170379_at | AI170379 | 0.004379275 | 0.066801371 | | | −0.000900617 | |
| 830 | 81557 | rc_AI170411_s_at | NM_053591 | 0.00627782 | | | | | 0.016760481 |
| 831 | 81559 | rc_AI170608_at | NM_031616 | | | | | | 0.040525773 |
| 832 | 76288 | rc_AI170685_at | NM_032079 | −0.002727749 | | | | | |
| 832 | 76289 | rc_AI170685_g_at | NM_032079 | | | | −0.049022863 | | |
| 833 | 79331 | Z18878cds_at | NM_053994 | 0.000707401 | | | | | |
| 833 | 81563 | rc_AI170911_at | NM_053994 | 0.010606506 | | | | | |
| 834 | 81565 | rc_AI171067_at | NM_012600 | 0.007144394 | | | | | |
| 835 | 81577 | rc_AI171844_at | NM_139099 | | | | | | 0.006661896 |
| 836 | 81584 | rc_AI172064_at | NM_019904 | | | | | | 0.025308372 |
| 837 | 76357 | rc_AI172452_at | AI172452 | −0.00386007 | | −0.003677218 | | | 0.000913233 |
| 838 | 81595 | rc_AI175486_at | AI175486 | 0.002137807 | | | | | 0.00509212 |
| 839 | 81606 | rc_AI175935_at | NM_173101 | 0.002963474 | | | | | |
| 840 | 81611 | rc_AI176351_s_at | NM_031137 | | | | −0.012286134 | | |
| 840 | 81644 | rc_AI178007_at | NM_031137 | | | | 0.080288041 | | |
| 841 | 81613 | rc_AI176460_s_at | AI176460 | 0.001372121 | | | | | |
| 842 | 81614 | rc_AI176461_s_at | NM_017211 | −0.000662681 | | | | 0.010532147 | |
| 842 | 82500 | U08136_at | NM_017211 | | −0.004474987 | | | | |
| 843 | 81620 | rc_AI176595_s_at | NM_013156 | 0.005616753 | | | −0.041266536 | | |
| 844 | 81624 | rc_AI176710_at | NM_031628 | | 0.006435494 | −0.006665472 | | | |
| 845 | 76293 | rc_AI176726_at | AI176726 | 0.003732553 | | | | | |
| 846 | 81629 | rc_AI177052_at | AI177052 | 0.000926078 | | | | | |
| 847 | 81632 | rc_AI177161_at | NM_031789 | 0.005655247 | | | 0.028011179 | | |
| 847 | 81633 | rc_AI177161_g_at | NM_031789 | 0.001587968 | | | 0.05377377 | | |
| 848 | 81637 | rc_AI177503_at | NM_053985 | −0.000648001 | | | | 0.023714496 | |
| 849 | 81654 | rc_AI179012_s_at | NM_031144 | | | | | | −0.01031327 |
| 850 | 81660 | rc_AI179610_at | NM_012580 | 0.013127491 | | | | | |
| 851 | 81663 | rc_AI180013_at | NM_033351 | 7.34E−05 | | | | | |
| 852 | 81668 | rc_AI180410_i_at | NM_020079 | −0.000353802 | | | | | |
| 853 | 81671 | rc_AI180424_at | NM_013011 | | | | | | −0.02397297 |
| 854 | 81672 | rc_AI180442_at | NM_031840 | 0.000186921 | | | | | |
| 855 | 81678 | rc_AI228247_at | NM_013106 | | | | | | 0.008006737 |
| 856 | 81696 | rc_AI230211_s_at | NM_031739 | 0.013679582 | | | | | |
| 857 | 81700 | rc_AI230354_at | NM_022538 | 0.00503907 | | | | | |
| 858 | 81705 | rc_AI230712_at | NM_012999 | 0.002504384 | | | | | |
| 859 | 81706 | rc_AI230748_at | NM_053867 | | | | | | 0.001683624 |
| 860 | 81722 | rc_AI231445_at | AI231445 | −0.004891239 | | | | | −0.03965627 |
| 861 | 81726 | rc_AI231807_at | NM_022500 | | | | 0.074168501 | | |
| 862 | 81729 | rc_AI232078_at | NM_021587 | 0.009757599 | | | | | |
| 863 | 81731 | rc_AI232096_at | NM_031672 | 0.001437376 | | | | | |
| 864 | 81732 | rc_AI232194_at | NM_032084 | −0.010896328 | | | | | |
| 865 | 81733 | rc_AI232256_at | NM_030586 | −0.002797414 | | | | | |
| 866 | 81734 | rc_AI232268_at | AI232268 | 0.009207342 | | 0.017884374 | | | |
| 867 | 81755 | rc_AI234351_at | AI234351 | 0.006400552 | | | | | |
| 868 | 81759 | rc_AI234915_at | AI234915 | −0.000397042 | | | | | |
| 869 | 81778 | rc_AI236484_at | NM_198132 | 0.002831548 | | | | | |
| 870 | 76224 | rc_AI236601_at | NM_001011901 | −0.000742497 | 0.008878339 | | | −0.001643118 | |
| 871 | 81784 | rc_AI237535_s_at | AI237535 | | 0.006122441 | | | | |
| 872 | 81786 | rc_AI237592_at | AI237592 | | | | 0.030795454 | | |
| 873 | 81789 | rc_AI237825_at | NM_012533 | | | | | | 0.023602921 |
| 874 | 80438 | rc_AI638939_at | AI638939 | | 0.020116022 | | | | |
| 875 | 80439 | rc_AI638940_at | AI638940 | | | | | | −0.048857677 |
| 876 | 80450 | rc_AI638951_at | AI638951 | 0.003051321 | | | | | |
| 877 | 80451 | rc_AI638952_at | AI638952 | 0.005887171 | | | | | |
| 878 | 80465 | rc_AI638969_at | NM_133605 | −0.006517828 | | −0.021015805 | | | |
| 879 | 80489 | rc_AI638996_at | AI638996 | | | | | | 0.004854767 |
| 880 | 80497 | rc_AI639007_at | AI639007 | | | | | | 0.047538864 |
| 881 | 80523 | rc_AI639035_at | AI639035 | 0.00601714 | | | | | |
| 882 | 80545 | rc_AI639058_s_at | AI639058 | 0.002771825 | | | | 0.014402715 | |
| 883 | 80546 | rc_AI639060_at | AI639060 | 0.001611435 | | | | 0.023445156 | 0.010820573 |
| 884 | 80551 | rc_AI639065_at | AI639065 | −0.001875807 | | | | | |
| 885 | 80556 | rc_AI639071_at | AI639071 | 0.006338227 | | | | | |
| 886 | 80589 | rc_AI639107_at | AI639107 | 0.003370958 | | | | | |
| 887 | 80596 | rc_AI639115_at | NM_198743 | −0.012363288 | | −0.015403562 | | | |
| 888 | 80610 | rc_AI639132_s_at | AI639132 | | | | | 0.010690543 | |
| 889 | 80629 | rc_AI639152_at | AI639152 | 0.001944355 | | | | | |
| 890 | 80651 | rc_AI639175_at | AI639175 | 0.004664467 | | | | | |
| 891 | 80655 | rc_AI639179_at | AI639179 | 0.005282396 | | | | | |
| 892 | 80656 | rc_AI639181_at | AI639181 | 0.002275071 | | | | | |
| 893 | 80664 | rc_AI639189_at | AI639189 | | | | | 0.011695832 | |
| 894 | 80665 | rc_AI639190_at | AI639190 | 0.008799213 | | | | | |
| 895 | 80667 | rc_AI639192_at | AI639192 | | 0.040979431 | | | | |
| 896 | 80684 | rc_AI639209_at | AI639209 | −0.003084262 | | | | | |
| 897 | 80685 | rc_AI639210_at | AI639210 | 0.000994128 | | | | | |
| 898 | 80686 | rc_AI639212_at | NM_017298 | −0.005518264 | | | | | |
| 899 | 80691 | rc_AI639217_at | AI639217 | −0.003102176 | | | | | |
| 900 | 80692 | rc_AI639218_at | NM_001009268 | −0.00244123 | | | | | |
| 901 | 80715 | rc_AI639246_at | AI639246 | 0.001874346 | | | | | |
| 902 | 80723 | rc_AI639252_at | AI639252 | 0.00641721 | | 0.012032542 | | | |
| 903 | 80728 | rc_AI639256_at | NM_181376 | −0.000455993 | | | | | |
| 904 | 80733 | rc_AI639263_at | AI639263 | 0.003146426 | | | | | |
| 905 | 80734 | rc_AI639264_at | AI639264 | 0.003823948 | | | | | |
| 906 | 80755 | rc_AI639293_at | AI639293 | 0.004385016 | | 0.034895235 | | | |
| 907 | 80788 | rc_AI639324_g_at | AI639324 | 0.003628253 | | | | | |
| 908 | 80792 | rc_AI639328_at | AI639328 | 0.006796872 | | | | | |
| 909 | 80793 | rc_AI639329_at | AI639329 | 0.001601309 | | | | | |
| 910 | 80805 | rc_AI639343_at | AI639343 | 0.004818431 | | 0.032507172 | | | |
| 911 | 80831 | rc_AI639370_at | NM_001007725 | −0.000304289 | | | | | |
| 912 | 80857 | rc_AI639400_at | AI639400 | 0.000736162 | | | | | |
| 913 | 80861 | rc_AI639404_at | AI639404 | −0.001657501 | | | | | |
| 914 | 80862 | rc_AI639405_at | NM_138831 | −0.005652122 | | | | | |
| 915 | 80868 | rc_AI639411_at | NM_207614 | −0.003396882 | | | | | |
| 916 | 80870 | rc_AI639412_at | AI639412 | | | | | | 0.016763799 |
| 917 | 80888 | rc_AI639432_at | AI639432 | 0.012029347 | | | | | |
| 918 | 80018 | U38253_g_at | NM_133609 | −0.001559076 | | | | | |
| 918 | 80897 | rc_AI639441_at | NM_133609 | −0.002825786 | | | | | |
| 919 | 80901 | rc_AI639444_g_at | NM_057191 | −0.007027413 | | | | | |
| 920 | 80920 | rc_AI639461_at | AI639461 | 0.00542247 | | | | | |
| 921 | 80927 | rc_AI639467_at | AI639467 | | | | | | 0.022117874 |
| 922 | 80937 | rc_AI639476_s_at | AI639476 | 0.009529284 | | | | | |
| 923 | 80954 | rc_AI639495_at | AI639495 | −0.005609949 | | | | | |
| 924 | 80957 | rc_AI639498_i_at | AI639498 | | | | 0.001033469 | | |
| 925 | 80964 | rc_AI639505_at | AI639505 | 0.007385825 | | | | −0.005060053 | |
| 926 | 80975 | rc_AI639518_at | AI639518 | | | | | | 0.010928341 |
| 927 | 80998 | rc_AI639536_at | AI639536 | | | | | 0.010130934 | |
| 928 | 83529 | rc_H31232_at | NM_053472 | 0.002770692 | | | | | |
| 929 | 83544 | rc_H31535_at | H31535 | 0.002603161 | | | | | |
| 930 | 83545 | rc_H31550_at | H31550 | −0.005723925 | | | | | |
| 931 | 83546 | rc_H31554_at | H31554 | | | | | | −0.006893113 |
| 932 | 83549 | rc_H31597_at | NM_153294 | 0.003983892 | | | | | |
| 933 | 83556 | rc_H31692_at | NM_021597 | 0.010503007 | | | | | |
| 934 | 83572 | rc_H31887_at | H31887 | 0.006445313 | | | | | |
| 935 | 83573 | rc_H31897_at | H31897 | | | | | −0.014377454 | |
| 936 | 83578 | rc_H31964_at | H31964 | −0.006022209 | | | | | |
| 937 | 83581 | rc_H31990_at | H31990 | | | | −0.012878864 | | |
| 938 | 83587 | rc_H33084_at | H33084 | | | | | | −0.034356545 |
| 939 | 83593 | rc_H33149_at | H33149 | 0.009481038 | | | | | |
| 940 | 83611 | rc_H33528_at | H33528 | | | | | 0.001233866 | |
| 941 | 83614 | rc_H33566_at | H33566 | −5.19E−05 | | | | | |
| 942 | 83615 | rc_H33577_at | H33577 | | | | | | 0.021443999 |
| 943 | 83621 | rc_H33660_at | H33660 | | | | | 0.009762389 | |
| 944 | 78424 | S42358_s_at | NM_024372 | −0.001337475 | | | | | |
| 945 | 78426 | S43408_g_at | NM_013143 | −0.000533128 | | | | | |
| 946 | 78428 | S45663_at | NM_138549 | −0.004496789 | | | | | |
| 947 | 78435 | S46798cds#1_s_at | S46798 | | | | | | 0.012078104 |
| 948 | 78445 | S50461_s_at | NM_031034 | 0.001515439 | | | | | |
| 949 | 78452 | S53987_at | S53987 | −0.003226474 | | | | | |
| 950 | 78457 | S55427_s_at | NM_017037 | 0.005155973 | | | | | |
| 951 | 78460 | S56464mRNA_at | S56464 | −0.000220313 | | | | | |
| 951 | 78461 | S56464mRNA_g_at | S56464 | | | 0.034149288 | | | |
| 952 | 78470 | S58528_at | S58528 | −0.005722592 | | | | | |
| 953 | 78478 | S60054_s_at | NM_012642 | −0.005221152 | | 0.00080536 | | | |
| 954 | 78481 | S61868_at | NM_012649 | | −0.01753467 | | | −0.000568077 | |
| 954 | 78482 | S61868_g_at | NM_012649 | | −0.045297045 | | | | |
| 955 | 78485 | S61960_s_at | S61960 | | −0.01732857 | | | | |
| 956 | 78486 | S61973_at | NM_153308 | | | | 0.047112313 | | |
| 957 | 78489 | S62096_s_at | NM_031654 | −0.002277267 | | | | | |
| 958 | 78495 | S63233_g_at | NM_053290 | 0.009447919 | | | | | |
| 959 | 78505 | S65091_at | S65091 | | 0.022077176 | | | | |
| 960 | 78509 | S65555_at | NM_017305 | 0.00043068 | | | | | |
| 960 | 78510 | S65555_g_at | NM_017305 | 0.007007605 | | | | | |
| 961 | 78511 | S66024_at | NM_017334 | | 0.036155252 | | | 0.003518114 | |
| 961 | 78512 | S66024_g_at | NM_017334 | | −0.003171065 | −0.029872289 | | | |
| 962 | 78513 | S66184_s_at | NM_017061 | | | | | | −0.001866186 |
| 962 | 78601 | S77494_s_at | NM_017061 | | | | | | 0.000538632 |
| 963 | 78516 | S67435_s_at | NM_022852 | −0.003893561 | | | | | |
| 964 | 78521 | S67900_s_at | NM_080477 | −0.001954245 | | | | | |
| 965 | 78529 | S68809_s_at | NM_001007636 | −0.002675493 | | | | | |
| 966 | 78532 | S69160_s_at | NM_013047 | | | | | | −0.010803526 |
| 967 | 76994 | X78848cds_f_at | NM_031509 | | | | 0.03339981 | | |
| 967 | 78555 | S72505_f_at | NM_031509 | | | | 0.057415439 | | |
| 968 | 78557 | S72594_s_at | NM_021989 | | | | | | −0.024250187 |
| 969 | 78563 | S73424_s_at | NM_031051 | | | | | | 0.015474456 |
| 970 | 78569 | S74257_g_at | S74257 | | | | | | 0.044897399 |
| 971 | 78572 | S74393_s_at | NM_013001 | | | | | | −0.016408692 |
| 972 | 78574 | S74572_g_at | NM_033096 | −0.000442205 | | | | | |
| 973 | 78595 | S76466_at | NM_199230 | 0.00607669 | | | | | |
| 974 | 78602 | S77528cds_s_at | NM_024125 | 0.005201937 | | | | 0.000839527 | |
| 974 | 82411 | X60769mRNA_at | NM_024125 | | | | | −0.011509724 | |
| 975 | 78603 | S77532_at | S77532 | | | | | | −0.010610521 |
| 976 | 78604 | S77556_s_at | NM_013137 | 0.006078599 | | | | | |
| 977 | 78606 | S77858_s_at | S77858 | −0.000897916 | | | | | |
| 978 | 78616 | S78556_at | S78556 | −0.016894378 | | | | | |
| 979 | 78623 | S79304_s_at | S79304 | | | | | | 0.006510978 |
| 980 | 78629 | S79730_s_at | NM_013007 | −0.000666526 | | | | | |
| 981 | 78634 | S79820_g_at | S79820 | 0.002942637 | | | | | |
| 982 | 78638 | S80118_s_at | S80118 | 0.010652168 | | | | | |
| 983 | 78639 | S80127_s_at | NM_017044 | | | 0.037048854 | | | |
| 984 | 78642 | S80379_s_at | S80379 | 0.000302726 | | | | 0.001558153 | |
| 985 | 78644 | S80456_s_at | NM_012613 | −0.016219148 | | | | | |
| 986 | 78653 | S81497_s_at | NM_012732 | −0.007131862 | | | | | |
| 987 | 78670 | S83320_g_at | S83320 | 0.005616642 | 0.08309864 | | | | |
| 988 | 76872 | S83436_i_at | NM_181371 | 0.002778085 | | | | | |
| 989 | 78672 | S85184_at | S85184 | 0.004603081 | | | | | |
| 989 | 78673 | S85184_g_at | S85184 | 0.004912998 | | | | | |
| 990 | 78679 | S94371_at | S94371 | −0.0001861 | | | | | |
| 991 | 79818 | U02316_s_at | NM_031588 | | 0.001211732 | | | | |
| 992 | 82149 | U02522_at | NM_022588 | | | | | | 0.009394401 |
| 993 | 79758 | U03120_at | NM_013033 | −0.003674588 | | | | | |
| 994 | 79594 | U03414_s_at | NM_053573 | 0.005432627 | | | | | |
| 995 | 78694 | U03763cds_at | NM_017174 | −0.000273801 | | | | | |
| 996 | 79330 | Z15158mRNA_at | NM_013086 | −0.003755423 | | | | | |
| 996 | 79773 | U04835_at | NM_013086 | | 0.018673417 | | | | |
| 997 | 79725 | U04998_at | NM_013080 | 0.004643796 | | | | | |
| 998 | 76888 | U05014_at | NM_053857 | 0.00924054 | | | | | |
| 999 | 82060 | U05675_at | NM_020071 | | −0.039616532 | | | | |
| 1000 | 76715 | U06099_at | NM_017169 | 0.004932077 | | | | | |
| 1001 | 78701 | U06752_at | U06752 | 9.45E−05 | | | | | |
| 1002 | 82002 | U07683_at | NM_019276 | | | | | 0.020594385 | |
| 1003 | 79624 | U08256_at | NM_024379 | | | | | −0.014336002 | |
| 1004 | 82439 | U09228_at | NM_053369 | −0.008696813 | | | | | |
| 1005 | 79921 | U09229_at | U09229 | 0.003545238 | | | | | |
| 1006 | 78709 | U09401_s_at | U09401 | −0.004653404 | | 0.013006011 | | 0.00588018 | |
| 1007 | 82163 | U10188_at | NM_017100 | 0.006941681 | | | | | |
| 1008 | 79623 | U11031_at | NM_019329 | −0.001196407 | | | | | |
| 1009 | 82032 | U11760_at | NM_053864 | | | | −0.057973359 | | |
| 1010 | 79754 | U12184_at | NM_030831 | −0.009037864 | | | | | |
| 1011 | 79888 | U12514_at | NM_012982 | −0.004588803 | | | | | |
| 1012 | 82031 | U14007_at | NM_012825 | | | | −0.009184246 | | |
| 1013 | 82176 | U14746_at | NM_052801 | −0.003625967 | | | | | 0.010518562 |
| 1014 | 79659 | U17253_at | NM_022856 | −0.014157237 | | | | −0.008668048 | |
| 1015 | 76900 | U17260_s_at | NM_053853 | −0.004001843 | | | | | |
| 1016 | 79070 | X52817cds_s_at | NM_053865 | 0.000150922 | | | | | |
| 1016 | 82662 | U17604_at | NM_053865 | | | 0.023581173 | | | |
| 1017 | 82094 | U18771_at | NM_133580 | −0.006859875 | | | | | |
| 1018 | 76903 | U18982_s_at | NM_012954 | 0.006160741 | | | | | |
| 1019 | 82090 | U19614_at | NM_145092 | | | | | 0.026964762 | |
| 1020 | 78731 | U20110cds_at | NM_053325 | | | | | | −0.005958764 |
| 1021 | 79688 | U20181_at | NM_022863 | −0.000843318 | | | | | |
| 1022 | 79684 | U20283_at | NM_031126 | −0.003665817 | | | | | |
| 1023 | 76904 | U20643mRNA#2_f_at | NM_012495 | 0.00321188 | | | | | |
| 1024 | 82039 | U20796_at | NM_147210 | 0.012998993 | | | | | |
| 1025 | 78735 | U21719mRNA_s_at | U21719 | −0.004254885 | 0.008550674 | | | | |
| 1026 | 79694 | U22414_at | NM_013025 | | | | | −0.021191482 | |
| 1027 | 78739 | U23146cds_s_at | NM_057103 | 0.000175287 | 0.009241013 | | | | |
| 1027 | 78779 | U41453_at | NM_057103 | −0.003614717 | | −0.01708408 | | | −0.00122922 |
| 1028 | 82134 | U23407_at | NM_017244 | 0.002854806 | | | | | |
| 1029 | 76905 | U23438_at | NM_022199 | 0.011386763 | | | | | |
| 1030 | 82207 | U23769_at | NM_017365 | | | | | | 0.007986864 |
| 1031 | 79787 | U25137_at | NM_017064 | 0.000132489 | | | | | |
| 1032 | 78740 | U25148_at | U25148 | 0.003699688 | | | | 0.010203011 | |
| 1033 | 76731 | X05137_at | NM_012610 | 0.002392573 | | | | | |
| 1033 | 76906 | U25650_f_at | NM_012610 | 0.001053396 | | | | | |
| 1034 | 82148 | U26033_at | NM_031987 | 0.013001781 | −0.005573322 | 0.053074611 | | | |
| 1035 | 82480 | U26397_at | NM_031002 | 0.001663902 | | | | | |
| 1036 | 82848 | U30381_at | NM_031615 | −0.001718099 | | | | | |
| 1037 | 82167 | U30831_at | NM_138849 | 0.007111894 | | | | | |
| 1038 | 78749 | U31160mRNA_s_at | NM_147211 | −0.005279436 | | | −0.055561453 | | |
| 1039 | 82136 | U31777_at | NM_017228 | 0.003179515 | | | | | |
| 1040 | 78759 | U33441mRNA_s_at | NM_133513 | | | | | | 0.033915519 |
| 1041 | 79726 | U35774_at | NM_017253 | | | | | −0.011429399 | |
| 1042 | 82130 | U35775_g_at | NM_031552 | | −0.019857079 | | | | |
| 1043 | 78762 | U36444cds#1_g_at | NM_031077 | −0.001239407 | | | | | |
| 1044 | 78765 | U36771_g_at | NM_017274 | 0.005616688 | | | | | |
| 1044 | 78767 | U36773_g_at | NM_017274 | −0.000866549 | | | | | |
| 1045 | 76691 | U38376_s_at | NM_133551 | 0.007027698 | | | | | |
| 1046 | 82601 | U38481_at | NM_013022 | 0.000562126 | | | | | |
| 1047 | 78773 | U39571_at | U39571 | 0.01246326 | | | 0.077073259 | | |
| 1048 | 78775 | U39608_at | U39608 | −0.000190387 | | | | | |
| 1049 | 82520 | U39875_at | NM_024139 | 0.006814146 | | | | | |
| 1050 | 78778 | U40819_at | NM_019142 | 0.003559137 | | 0.005167997 | | | |
| 1051 | 76916 | U40836mRNA_s_at | NM_012786 | | | | | | 0.019786105 |
| 1052 | 82715 | U41845_at | NM_012991 | 0.007890622 | | | | | |
| 1053 | 76765 | U41853_at | NM_138867 | 0.012213922 | | | | | |
| 1054 | 82236 | U43534_at | NM_012894 | −0.004440377 | | | | | |
| 1055 | 78781 | U47014_at | U47014 | −0.007702328 | | | | | |
| 1056 | 80045 | U47110_at | NM_022184 | 0.009724092 | | | | | |
| 1057 | 76919 | U47313_at | U47313 | −0.000494993 | | | | | |
| 1058 | 76921 | U47315_s_at | NM_133561 | 0.002430067 | | | | | |
| 1059 | 76101 | U48220_at | NM_138515 | 0.008478977 | | | | | |
| 1060 | 76754 | U48596_at | NM_053887 | | | | | | −0.02535369 |
| 1061 | 82488 | U49099_at | U49099 | 0.006301452 | | | | | |
| 1062 | 76767 | U49930_at | NM_012922 | 0.006587665 | | | | | |
| 1063 | 80029 | U50412_at | NM_013005 | −0.006307345 | | | | | |
| 1064 | 78801 | U52034cds_i_at | NM_022175 | | | | 0.005195837 | | |
| 1065 | 78807 | U52950_g_at | U52950 | | | | | 0.017935712 | −0.005814227 |
| 1066 | 82243 | U53184_at | U53184 | | 0.02906887 | | | −0.003404813 | |
| 1067 | 82483 | U53211_at | NM_012892 | 0.002403536 | | | | | |
| 1068 | 78810 | U53486mRNA_s_at | NM_030999 | −0.001357776 | | | | | |
| 1069 | 82264 | U53513_at | U53513 | −0.000644721 | | | | | |
| 1070 | 82267 | U53706_at | NM_031062 | 0.008457699 | | | | | |
| 1071 | 78811 | U53873cds_at | U53873 | 0.00459739 | | 0.023344089 | | | |
| 1072 | 78812 | U53927_at | NM_022619 | 0.002880911 | | | | | |
| 1073 | 80031 | U55836_at | NM_031089 | −0.002679597 | | | | | |
| 1074 | 82684 | U55938_at | NM_013029 | 0.003786277 | | | | | |
| 1075 | 79994 | U56815_at | NM_031665 | −0.001724168 | | | | | |
| 1076 | 82484 | U56839_at | NM_017255 | −0.005564924 | | | | | |
| 1077 | 79952 | U57049_at | U57049 | 0.001389865 | | | | | |
| 1078 | 82524 | U57062_at | NM_134332 | | | | | | 0.007824695 |
| 1079 | 79982 | U57063_at | NM_153466 | | −0.012194139 | | | | |
| 1080 | 82577 | U59241_at | NM_013044 | | | | −0.025098723 | | |
| 1081 | 79961 | U59672_at | NM_024394 | 8.61E−05 | | −0.00879096 | | | |
| 1082 | 78834 | U62635_s_at | NM_022529 | | | | | | −0.010588194 |
| 1083 | 80063 | U62667_at | NM_031123 | −0.004933976 | | | | | |
| 1084 | 76779 | U62940_at | NM_024487 | 0.001351786 | | | | | |
| 1085 | 78836 | U63318_s_at | NM_139090 | −0.00209692 | | | | | |
| 1086 | 76927 | U64451_at | NM_013084 | −0.005934691 | | | | | |
| 1087 | 78838 | U64689_at | NM_053600 | −0.000929849 | | | | | |
| 1088 | 78839 | U64705cds_i_at | NM_001008335 | | | | −0.004614501 | | |
| 1089 | 82644 | U65656_at | NM_031054 | | | | −0.049020993 | | |
| 1090 | 78843 | U66292_at | NM_021677 | −0.00020404 | | 0.003409962 | | 0.009127421 | |
| 1091 | 78844 | U66293_at | U66293 | 0.007807538 | | | | | |
| 1092 | 78845 | U66298_at | NM_013107 | −0.005825195 | | | | | |
| 1093 | 80068 | U66322_at | NM_138863 | | | | −0.067823809 | | |
| 1094 | 80054 | U66478_at | NM_013130 | 0.002558296 | | | | | |
| 1095 | 80074 | U67082_at | NM_019620 | 0.006478937 | | | | | |
| 1096 | 78848 | U67207_s_at | NM_012596 | −0.000177436 | | | | | |
| 1097 | 76789 | U69272_at | NM_013129 | 0.003988226 | | | | | |
| 1097 | 76790 | U69272_g_at | NM_013129 | 0.005737444 | | | | | |
| 1098 | 80122 | U69279_at | NM_053903 | 0.002938354 | | | | | |
| 1099 | 82847 | U70050_at | U70050 | 0.008357922 | | | | | |
| 1100 | 76931 | U71089cds_at | NM_019310 | −0.000796698 | | | | | |
| 1101 | 82581 | U72660_at | NM_012867 | | −0.011364006 | | | | |
| 1102 | 80319 | U72741_at | NM_012977 | 0.005034962 | | | | | |
| 1102 | 80320 | U72741_g_at | NM_012977 | | | | −0.000528661 | | |
| 1103 | 80181 | U72995_at | NM_053585 | −0.00259883 | | | | | |
| 1104 | 78872 | U75412cds_at | U75412 | | | | | 0.014749423 | |
| 1105 | 76936 | U75899mRNA_at | NM_130431 | | | | | | 0.048034537 |
| 1106 | 82650 | U75916_at | U75916 | | | | 0.050010261 | | |
| 1107 | 82651 | U75917_at | NM_022952 | | | | 0.047012933 | | |
| 1108 | 78873 | U75920_at | NM_138509 | −0.002439169 | | | | | |
| 1109 | 78874 | U75921UTR#1_at | U75921 | | | | | 0.008321447 | |
| 1110 | 78875 | U75923cds_at | U75923 | 0.004677647 | | | | | |
| 1111 | 80287 | U77583_at | NM_053615 | 0.010314532 | | | | | |
| 1112 | 78894 | U77829mRNA_i_at | U77829 | −0.000711711 | | | | | |
| 1113 | 82606 | U77918_at | NM_031595 | | | | −0.041646417 | | |
| 1114 | 78895 | U77931_at | NM_147136 | 0.012664645 | | | | | |
| 1115 | 78902 | U79568_s_at | NM_133289 | −0.001213537 | | | | | |
| 1116 | 78903 | U79661_at | NM_022203 | 0.010440387 | | 0.046066235 | | | |
| 1117 | 76822 | U80054_at | NM_031053 | 0.004864799 | | | | | |
| 1118 | 78905 | U81035_s_at | NM_053909 | −0.002670014 | | | | | |
| 1118 | 78906 | U81036_s_at | NM_053909 | −0.008232535 | | | | | |
| 1119 | 78907 | U81186_at | NM_032066 | | | | 0.146208724 | | 0.042869392 |
| 1120 | 82945 | U82591_at | NM_133525 | | | | | | −0.011108871 |
| 1121 | 82913 | U83230_at | NM_013217 | −0.009024953 | | | | | |
| 1122 | 78914 | U83666_at | U83666 | 0.005410489 | | | | | |
| 1123 | 78916 | U83880UTR#1_at | NM_012736 | 0.00584112 | | | | | |
| 1124 | 82640 | U83896_at | NM_053911 | −0.002391643 | | | | | |
| 1125 | 78919 | U84727_at | NM_022398 | −0.001254714 | | | | | |
| 1126 | 78921 | U85513_at | NM_031666 | −0.000355632 | | | | | |
| 1127 | 78922 | U87305_at | NM_022206 | 0.012616272 | | | | | |
| 1128 | 78923 | U87306_at | NM_022207 | −0.002893647 | | | | | |
| 1129 | 82660 | U87983_at | NM_012964 | | | | | | −0.008506524 |
| 1130 | 78933 | U90261UTR#1_g_at | NM_053360 | | | | | 0.016533905 | |
| 1131 | 78936 | U90725_s_at | NM_172039 | | | | | 0.027645792 | |
| 1132 | 78938 | U90829_g_at | NM_032072 | | | | −0.004520698 | | −0.034862989 |
| 1133 | 78942 | U92081mRNA_s_at | NM_019358 | 0.002315315 | | | | | |
| 1134 | 80203 | U92564_g_at | NM_053583 | 0.007525417 | | | | | |
| 1135 | 80300 | U92803_at | NM_078621 | | | | | | −0.034725037 |
| 1136 | 78945 | U93052_s_at | NM_031828 | −0.00443885 | | | | | |
| 1137 | 80188 | U94330_at | NM_012870 | | | | | 0.000178218 | |
| 1138 | 82933 | U94904_s_at | NM_173143 | −0.002352342 | | | | | |
| 1139 | 78949 | U95001UTR#1_s_at | NM_053598 | −0.007983856 | | | | | |
| 1140 | 80253 | U95178_s_at | NM_024159 | | | | | 0.014214131 | |
| 1141 | 82570 | X02361_at | NM_012493 | 0.000524006 | | | | | |
| 1142 | 76948 | X02904cds_s_at | NM_138974 | 0.006002078 | | | | | |
| 1143 | 82357 | X02918_at | NM_012998 | −0.000927548 | | | | | |
| 1144 | 78968 | X03914mergedCDS_UTR_at | X03914 | 0.010902466 | | | | | |
| 1145 | 82436 | X04070_at | NM_017251 | 0.012008931 | | | | | |
| 1146 | 82384 | X05300_at | NM_013067 | −0.0035316 | | | | | |
| 1147 | 78974 | X06150cds_at | NM_017084 | | | | | | 0.020312412 |
| 1148 | 76736 | X06827_at | NM_013168 | 0.007975354 | | | | | |
| 1149 | 78981 | X06832cds#2_s_at | NM_021655 | 0.006901494 | | 0.036723844 | | | |
| 1150 | 78982 | X06889cds_at | NM_013018 | 0.006109385 | | | | | |
| 1151 | 82354 | X06916_at | NM_012618 | | | | | 0.025351832 | |
| 1152 | 78984 | X06984cds_s_at | NM_012497 | | | 0.007419575 | | | |
| 1153 | 78986 | X07259cds_s_at | NM_153307 | −0.001729941 | | −4.78E−05 | | | |
| 1154 | 79012 | X13905cds_g_at | NM_001008370 | | | | 0.012453977 | | |
| 1155 | 76958 | X13933_s_at | NM_031969 | −0.006289804 | | | | | |
| 1156 | 79025 | X14848cds#12_at | X14848 | 0.008799836 | | | | | |
| 1157 | 82350 | X15679_at | NM_173301 | | | | | | −0.002819969 |
| 1158 | 76963 | X15705cds_at | NM_021863 | | | | | 0.029887704 | |
| 1159 | 76722 | X16072_at | NM_012937 | | | | | | −0.043514095 |
| 1160 | 79044 | X16623cds_s_at | X16623 | | | | | | −0.021202041 |
| 1161 | 79048 | X17053cds_s_at | NM_031530 | | 0.016501536 | | | 0.011364152 | |
| 1161 | 79049 | X17053mRNA_s_at | NM_031530 | | 0.026013864 | | | 0.011488311 | |
| 1162 | 79051 | X17607cds_s_at | NM_012492 | | | | −0.051721624 | | |
| 1163 | 79060 | X51706cds_at | NM_001007598 | | | | | | −0.003264854 |
| 1164 | 79063 | X51781cds_s_at | NM_013023 | −7.16E−05 | | | | | |
| 1165 | 76971 | X53428cds_s_at | NM_032080 | 0.00087413 | 0.003048868 | | | | |
| 1166 | 79080 | X53504cds_g_at | X53504 | −0.00210435 | | | | | |
| 1167 | 82282 | X53773_at | NM_031008 | −0.003053957 | | | | | |
| 1168 | 82301 | X54510_at | NM_053602 | | | | | | −0.034426426 |
| 1169 | 82312 | X54531mRNA_at | NM_080689 | −0.00083552 | | | | | |
| 1170 | 79087 | X54617mRNA_s_at | X54617 | | | 0.041023635 | | | |
| 1171 | 82349 | X54640_at | NM_012783 | −0.002433691 | | | | | |
| 1172 | 76974 | X54686cds_at | NM_021836 | | −0.026251747 | | | | |
| 1173 | 76750 | X54793_at | NM_022229 | | | | | | −0.010197175 |
| 1174 | 79088 | X54798cds_at | X54798 | 0.00778292 | | | | | |
| 1175 | 76748 | X55286_at | NM_013134 | −0.008323502 | | | | | |
| 1176 | 82332 | X55660_at | NM_019331 | −0.008532897 | | | | | |
| 1177 | 79929 | X55955_at | NM_012742 | −0.011108524 | | | | | |
| 1178 | 79090 | X55986mRNA_s_at | NM_017281 | −0.000706926 | | | | | |
| 1179 | 79095 | X56448mRNA_at | X56448 | −0.003310692 | | −0.011361545 | | | |
| 1180 | 82430 | X56546_at | NM_013103 | | | | | 0.002075257 | |
| 1181 | 79104 | X57432cds_s_at | NM_031838 | | 0.007322836 | | | | |
| 1182 | 79113 | X58465mRNA_at | X58465 | 0.00388684 | | | | | 0.007679724 |
| 1183 | 79115 | X58550mRNA_s_at | NM_012490 | | | | | | −0.007446424 |
| 1184 | 82525 | X58828_at | NM_053990 | | −0.049266346 | | | | |
| 1185 | 82361 | X58865mRNA_at | NM_013190 | −0.003032462 | | | | | |
| 1186 | 79118 | X59375mRNA_at | X59375 | | | | 0.036894425 | | |
| 1187 | 79123 | X59737mRNA_at | X59737 | 0.002756449 | | 0.011792466 | | 0.015190929 | |
| 1187 | 79124 | X59737mRNA_g_at | X59737 | | | | | 0.016403436 | |
| 1188 | 79127 | X59949cds_s_at | NM_052799 | −0.000372749 | | | | | |
| 1189 | 82393 | X60212_i_at | NM_201415 | 0.021341138 | | 0.100114084 | | | 0.028033565 |
| 1190 | 79132 | X60469mRNA_s_at | NM_080478 | −0.00791677 | | | | | |
| 1191 | 79133 | X60651mRNA_s_at | NM_013026 | −0.002738856 | | | | | |
| 1192 | 79145 | X62145cds_at | X62145 | −0.004481564 | | | | | |
| 1193 | 79149 | X62166cds_s_at | NM_198753 | 0.002173674 | | | | 0.014305863 | |
| 1194 | 79153 | X62325cds_f_at | X62325 | 0.005071277 | | | | | |
| 1195 | 79166 | X62950mRNA_f_at | X62950 | | 0.028975395 | | | | |
| 1196 | 79167 | X62951mRNA_s_at | X62951 | | | | | | 0.030303936 |
| 1197 | 79170 | X63369cds_at | NM_133290 | | | | | −0.0097977 | |
| 1198 | 79824 | X66974_at | NM_053988 | −0.007896364 | | | | | |
| 1199 | 82287 | X67859_at | NM_031119 | 0.012675573 | | | | | |
| 1200 | 79893 | X68191_at | NM_019268 | −0.002615368 | | | | | |
| 1201 | 76730 | X68199_at | NM_053986 | 0.006044538 | | | | | |
| 1202 | 79182 | X70667cds_at | X70667 | −0.003534526 | | | | | 0.00219665 |
| 1203 | 82425 | X71898_at | NM_134352 | −0.000757385 | 0.032590608 | | | 0.008341811 | |
| 1204 | 76720 | X72757_g_at | X72757 | | | | | | 0.001179565 |
| 1205 | 82327 | X73371_at | NM_175756 | 0.00095486 | | | | | |
| 1206 | 79185 | X73411mRNA_s_at | NM_130738 | 0.00540037 | | | | | |
| 1207 | 79186 | X74227cds_at | NM_019312 | | | | −0.038627469 | | |
| 1208 | 82545 | X74549_at | NM_024382 | 0.014304221 | | | | | |
| 1209 | 76990 | X74565cds_at | NM_022516 | 0.004744347 | | | | | |
| 1209 | 76991 | X74565cds_g_at | NM_022516 | 0.003468802 | | | | | |
| 1210 | 82406 | X74593_at | NM_017052 | 0.015370819 | | | | | |
| 1211 | 79189 | X74833cds_at | NM_012528 | 0.006777929 | | | | | −0.003344072 |
| 1212 | 79190 | X74834cds_s_at | NM_019145 | | | | | −0.000576866 | |
| 1213 | 79192 | X74836cds_s_at | NM_017194 | | | | | 0.008639081 | |
| 1214 | 79193 | X74978exon_at | X74978 | 0.000169182 | | | | | |
| 1215 | 82414 | X75856_at | X75856 | | | | | | −0.007123435 |
| 1216 | 79196 | X76452cds_s_at | NM_001005871 | −0.006065695 | | −0.006405555 | | | |
| 1217 | 79198 | X76489cds_at | X76489 | | | | | | 0.021661652 |
| 1217 | 79199 | X76489cds_g_at | X76489 | | | | | | 0.021113575 |
| 1218 | 80285 | X76724_at | NM_017304 | 0.00291018 | | | | | |
| 1219 | 82466 | X76985_at | NM_031655 | 0.016127877 | | | | | |
| 1220 | 76791 | X77209_at | NM_212546 | −0.000365968 | | | | | |
| 1221 | 80024 | X77818_at | X77818 | −0.002030349 | | | | | |
| 1222 | 82548 | X78606_at | NM_053978 | −0.002047578 | | | | | |
| 1223 | 76995 | X79081mRNA_f_at | NM_019184 | 0.001404971 | | | | | |
| 1224 | 82210 | X81193_at | NM_057144 | 0.008892347 | | | | 0.007812858 | |
| 1225 | 82331 | X82152_at | NM_080698 | 0.007546666 | | 0.000310029 | | | |
| 1226 | 82554 | X82396_at | NM_022597 | 0.003039038 | | | | | |
| 1227 | 82392 | X82445_at | NM_017271 | −0.003055175 | | | | | |
| 1228 | 82223 | X83399_at | NM_053974 | −2.02E−05 | | | | | |
| 1229 | 79218 | X83585cds_s_at | NM_031602 | | | | | | 0.02914792 |
| 1230 | 79221 | X83735exon#3_at | NM_001008889 | −0.001827724 | | 0.004172236 | | | |
| 1231 | 79993 | X84210complete_seq_s_at | NM_012988 | 0.004611015 | | | | | |
| 1232 | 79223 | X86178mRNA_at | X86178 | −0.000221159 | | | | | |
| 1233 | 80097 | X86561cds#2_at | NM_001008724 | −0.004551591 | | | | | |
| 1234 | 79228 | X89695cds_at | NM_214821 | −0.00189784 | | | | | |
| 1235 | 79229 | X89696cds_at | NM_001000363 | | | 0.012176268 | | | −0.019588878 |
| 1236 | 79239 | X89705cds_at | NM_001000502 | −0.003291795 | | | | | |
| 1237 | 82504 | X91234_at | NM_024391 | 0.00725178 | | | | | |
| 1238 | 82239 | X92069_at | NM_080780 | | −0.000829165 | | | | |
| 1239 | 82558 | X92097_at | NM_031722 | 0.009346506 | | | | | |
| 1240 | 79250 | X93219cds_s_at | NM_012896 | 0.003979532 | | | | | |
| 1241 | 79988 | X95188_at | NM_053339 | 0.002113037 | | | | | |
| 1242 | 79256 | X95990exon_s_at | NM_053619 | 0.00754288 | | | 0.028457038 | | |
| 1243 | 79257 | X96437mRNA_at | NM_212505 | −0.007439251 | −0.055662435 | | | | |
| 1244 | 77005 | X98490cds_at | NM_021582 | | | | −0.030509847 | | |
| 1245 | 79264 | X98564cds_at | NM_021697 | 0.001156352 | | | | | |
| 1246 | 79265 | X98746cds_at | NM_134329 | −0.0043652 | | | | | |
| 1247 | 77006 | Y00156cds_f_at | NM_153314 | 0.003308465 | | | | | |
| 1248 | 77007 | Y00396mRNA_at | NM_012603 | 9.11E−05 | | | | | |
| 1248 | 77008 | Y00396mRNA_g_at | NM_012603 | | −0.008270231 | | | | |
| 1249 | 79894 | Y00766_at | NM_013119 | −0.0026598 | | | | | |
| 1250 | 79281 | Y08138_at | NM_022696 | | −0.071388115 | | | | −0.007189925 |
| 1251 | 79288 | Y09453cds_at | NM_019255 | −0.000589954 | | | | | |
| 1252 | 79291 | Y10823cds_s_at | NM_053762 | | | | | 0.000334329 | |
| 1253 | 79294 | Y11489cds_at | Y11489 | −0.000597271 | | | | −6.42E−05 | |
| 1254 | 82731 | Y12635_at | NM_057213 | −0.001011873 | | | | | |
| 1255 | 82853 | Y13714_at | NM_012656 | 0.00452094 | | | | | |
| 1256 | 76378 | Y15068_at | NM_138911 | 0.000684766 | | | | | |
| 1257 | 79309 | Y17048_g_at | NM_133529 | −0.00700948 | | | | | |
| 1258 | 79319 | Z11932cds_g_at | NM_019136 | 0.000524803 | | | | | 0.008499624 |
| 1259 | 79325 | Z14118cds_at | Z14118 | −0.002399383 | | | | | |
| 1259 | 79326 | Z14118cds_g_at | Z14118 | −0.002434108 | | | | | |
| 1260 | 79327 | Z14119cds_at | NM_031525 | 0.005727558 | | | | | |
| 1261 | 82221 | Z36980_at | NM_024131 | 0.000297484 | | | | | |
| 1262 | 79349 | Z83757mRNA_at | NM_017094 | −0.002064143 | | | | | |
| 1263 | 79351 | Z83869cds_at | NM_021699 | −0.00068321 | | | | 0.00782114 | −0.017420592 |

TABLE 3

| | | | | | | | | Vascu- | |
|---|---|---|---|---|---|---|---|---|---|
| | Dose | Time | Route of | | Andrenergic | | Myocardial | lature | Heart |
| Agent | (mg/kg) | (hrs) | Administration | General | Agonist | Arrhythmia | Necrosis | Agent | Failure |
| Acyclovir | 100 | 6, 24 | Intraperitoneal | −1 | −1 | −1 | −1 | −1 | −1 |
| Adriamycin | 12 | 6, 24 | Intravenous | 1 | −1 | −1 | 1 | −1 | 1 |
| AmphotercinB | 2.5 | 6, 24 | Intravenous | 1 | −1 | 1 | −1 | −1 | Not Used |
| BICompound | 50 | 6, 24 | Oral gavage | 1 | Not Used | Not Used | Not Used | Not Used | Not Used |
| Carboplatin | 50 | 6, 24 | Intravenous | −1 | −1 | −1 | −1 | −1 | −1 |
| CCl4 | 1000 | 6, 24 | Oral gavage | −1 | −1 | −1 | −1 | −1 | −1 |
| Cisplatin | 5 | 6, 24 | Intravenous | 1 | −1 | 1 | 1 | −1 | −1 |
| Clenbuterol | 4 | 6, 24 | Subcutaneous | 1 | 1 | 1 | 1 | 1 | Not Used |
| Cyclophosphamide | 200 | 6, 24 | Intraperitoneal | 1 | −1 | −1 | −1 | −1 | 1 |
| Dantrolene | 2000 | 6 | Oral gavage | 1 | −1 | 1 | −1 | Not Used | 1 |
| Dopamine | 4 | 6, 24 | Subcutaneous | 1 | 1 | 1 | −1 | −1 | Not Used |
| Epinephrine | 1 | 6, 24 | Subcutaneous | 1 | 1 | 1 | 1 | −1 | −1 |
| Epirubicin | 12 | 6, 24 | Intravenous | 1 | −1 | 1 | −1 | −1 | 1 |
| Famotidine | 500 | 6, 24 | Intraperitoneal | 1 | −1 | 1 | −1 | −1 | −1 |
| Hydralazine | 25 | 6, 24 | Intraperitoneal | 1 | −1 | 1 | 1 | 1 | −1 |
| Ifosfamide | 100 | 6, 24 | Intraperitoneal | 1 | −1 | 1 | −1 | −1 | 1 |
| Imatinib | 200 | 6, 24 | Oral gavage | 1 | Not Used | 1 | Not Used | Not Used | 1 |
| Isoproterenol | 0.5 | 6, 24 | Subcutaneous | 1 | 1 | 1 | 1 | Not Used | Not Used |
| Minoxidil | 120 mg/L | 6, 24 | Drinking water | 1 | −1 | 1 | 1 | 1 | 1 |
| Monocrotaline | 105 | 6, 24 | Subcutaneous | 1 | Not Used | −1 | −1 | Not Used | Not Used |
| Norephinephrine | 0.5 | 6, 24 | Subcutaneous | 1 | 1 | 1 | 1 | Not Used | −1 |
| Paroxetine | 75 | 6, 24 | Oral gavage | 1 | Not Used | 1 | −1 | Not Used | Not Used |
| Pentamidine | 30 | 6, 24 | Intraperitoneal | 1 | Not Used | 1 | −1 | Not Used | −1 |
| PfizerCompound | 1000 | 6, 24 | Oral gavage | 1 | Not Used | Not Used | Not Used | Not Used | Not Used |
| Phenylpropanolamine | 64 | 6, 24 | Intraperitoneal | 1 | 1 | 1 | 1 | Not Used | −1 |
| Rosiglitazone | 180 | 6, 24 | Oral gavage | 1 | Not Used | Not Used | Not Used | Not Used | 1 |
| Temozolomide | 5 | 6, 24 | Oral gavage | −1 | Not Used | −1 | −1 | Not Used | −1 |

TABLE 4

| SEQ ID | GLGC | GenBank Acc or | | |
|---|---|---|---|---|
| NO. | ID No. | RefSeq ID No. | Gene Name | Pathway Name |
| 1 | 77058 | A03913 | | |
| 2 | 77023 | AA684963 | similar to binding protein | |
| 3 | 77024 | AA685112 | NADH dehydrogenase (ubiquinone) | (Oxidative phosphorylation, Ubiquinone |
| | | | Fe—S protein 8 (predicted) | biosynthesis) |
| 4 | 77026 | AA685376 | | |
| 5 | 77028 | NM_001012197 | tumor rejection antigen gp96 | |
| | | | (predicted) | |
| 6 | 77029 | NM_001005550 | NADH dehydrogenase (ubiquinone) | (Oxidative phosphorylation, Ubiquinone |
| | | | Fe—S protein 1, 75 kDa | biosynthesis) |
| 7 | 77030 | NM_001009672 | SMT3 suppressor of mif two 3 | (Basic Mechanisms of SUMOylation, Regulation |
| | | | homolog 1 (yeast) (predicted) | of transcriptional activity by PML, Role of Parkin |
| | | | | in the Ubiquitin-Proteasomal Pathway, |
| | | | | SUMOylation as a mechanism to modulate CtBP- |
| | | | | dependent gene responses, Sumoylation by |
| | | | | RanBP2 Regulates Transcriptional Repression) |
| 8 | 77032 | AA686870 | | |
| 9 | 82173 | NM_031091 | RAB3B, member RAS oncogene | Tight junction |
| | | | family | |
| 9 | 82174 | NM_031091 | RAB3B, member RAS oncogene | Tight junction |
| | | | family | |
| 10 | 82240 | NM_031763 | platelet-activating factor | Glycerophospholipid metabolism |
| | | | acetylhydrolase, isoform Ib, alpha | |
| | | | subunit 45 kDa | |
| 11 | 77033 | AA819943 | | |
| 12 | 77036 | AA848421 | uracil-DNA glycosylase (predicted) | |
| 13 | 77037 | NM_001009542 | similar to programmed cell death 10 | |
| 14 | 76161 | NM_212504 | heat shock 70 kD protein 1B | MAPK signaling pathway |
| 15 | 77039 | AA875509 | transformed mouse 3T3 cell double | (ATM Signaling Pathway, CTCF: First Multivalent |
| | | | minute 2 (predicted) | Nuclear Factor, Cell Cycle: G2/M Checkpoint, |
| | | | | Cell cycle, Control of of p53 Subcellular |
| | | | | Localization, HIV-I Nef: negative effector of Fas |
| | | | | and TNF, Hypoxia and p53 in the Cardiovascular |
| | | | | system, Puma and Noxa Proteins Mediate p53- |
| | | | | and Drug-induced Apoptotic Responses, |
| | | | | Sumoylation by RanBP2 Regulates |
| | | | | Transcriptional Repression, Tumor Suppressor |
| | | | | Arf Inhibits Ribosomal Biogenesis, p53 Signaling |
| | | | | Pathway) |
| 16 | 80380 | NM_022607 | MIPP65 protein | (Oxidative phosphorylation, Ubiquinone |
| | | | | biosynthesis) |
| 17 | 80197 | NM_017217 | solute carrier family 7 (cationic | |
| | | | amino acid transporter, y+ system), | |
| | | | member 3 | |
| 18 | 82747 | NM_134403 | ankyrin repeat and BTB (POZ) | |
| | | | domain containing 2 | |
| 19 | 80386 | NM_053509 | zona pellucida glycoprotein 1 | |
| 20 | 77074 | NM_032075 | growth hormone secretagogue | Neuroactive ligand-receptor interaction |
| | | | receptor | |
| 21 | 77078 | NM_021867 | fibroblast growth factor 16 | (MAPK signaling pathway, Regulation of actin |
| | | | | cytoskeleton) |
| 22 | 77080 | NM_019349 | serine/threonine kinase 2 | |
| 23 | 77082 | NM_133553 | UDP-Gal:betaGlcNAc beta 1,3- | |
| | | | galactosyltransferase, polypeptide 4 | |
| 24 | 82743 | NM_022706 | GABA(A) receptor-associated | |
| | | | protein like 2 | |
| 25 | 80343 | NM_031707 | homer homolog 1 (Drosophila) | |
| 26 | 77083 | AB003753 | | |
| 27 | 77099 | AB005541 | | |
| 28 | 82881 | NM_031635 | fucosyltransferase 2 (secretor status | (Blood group glycolipid biosynthesis-lactoseries, |
| | | | included) | Blood group glycolipid biosynthesis- |
| | | | | neolactoseries, Globoside metabolism) |
| 29 | 82878 | NM_019351 | translocator of inner mitochondrial | TIM23 Translocase |
| | | | membrane 17a (yeast) | |
| 30 | 82879 | NM_019352 | translocase of inner mitochondrial | (Bile acid biosynthesis, Butanoate metabolism, |
| | | | membrane 23 homolog (yeast) | Fructose and mannose metabolism, Galactose |
| | | | | metabolism, Glycerophospholipid metabolism, |
| | | | | Glycine, serine and threonine metabolism, Lysine |
| | | | | degradation, Nucleotide sugars metabolism, |
| | | | | TIM23 Translocase) |
| 31 | 77104 | NM_057135 | 6-phosphofructo-2-kinase/fructose- | Fructose and mannose metabolism |
| | | | 2,6-biphosphatase 3 | |
| 32 | 77105 | AB006802 | | |
| 33 | 77107 | NM_147207 | ischemia related factor vof-16 | |
| 34 | 77111 | NM_053309 | homer homolog 2 (Drosophila) | |
| 35 | 82920 | NM_031753 | activated leukocyte cell adhesion | |
| | | | molecule | |
| 36 | 77135 | NM_147135 | SH3-binding kinase | |
| 37 | 77141 | NM_019273 | potassium large conductance | |
| | | | calcium-activated channel, subfamily | |
| | | | M, beta member 1 | |
| 38 | 77155 | NM_173102 | tubulin, beta 5 | |
| 39 | 80155 | AB012233 | nuclear factor I/C | |
| 40 | 80157 | AB012234 | nuclear factor I/X | |
| 41 | 77161 | NM_024362 | aryl hydrocarbon receptor nuclear | Circadian Rhythms |
| | | | translocator-like | |
| 42 | 77162 | NM_053607 | acyl-CoA synthetase long-chain | Fatty acid metabolism |
| | | | family member 5 | |
| 43 | 77171 | NM_013030 | solute carrier family 34 (sodium | |
| | | | phosphate), member 1 | |
| 44 | 77172 | AB013454 | similar to Ac2-210 | |
| 43 | 77173 | NM_013030 | solute carrier family 34 (sodium | |
| | | | phosphate), member 1 | |
| 45 | 77178 | NM_022505 | Rhesus blood group CE and D | |
| 45 | 77179 | NM_022505 | Rhesus blood group CE and D | |
| 46 | 77190 | NM_145778 | tubulin, gamma 1 | |
| 47 | 77200 | NM_031330 | heterogeneous nuclear | (Alanine and aspartate metabolism, Arginine and |
| | | | ribonucleoprotein A/B | proline metabolism, Urea cycle and metabolism |
| | | | | of amino groups) |
| 48 | 77202 | AB017044 | | |
| 25 | 77204 | NM_031707 | homer homolog 1 (Drosophila) | |
| 49 | 77206 | NM_022953 | slit homolog 1 (Drosophila) | |
| 50 | 77215 | NM_031335 | polymerase II | (Purine metabolism, Pyrimidine metabolism, RNA |
| | | | | polymerase) |
| 51 | 77228 | AB020504 | PMF32 protein | |
| 52 | 77235 | NM_139091 | nucleoporin like 1 | Nuclear Pore Complex |
| 53 | 77239 | NM_022208 | general transcription factor 2a, 1 | (Basal transcription factors, CARM1 and |
| | | | | Regulation of the Estrogen Receptor, Chromatin |
| | | | | Remodeling by hSWI/SNF ATP-dependent |
| | | | | Complexes, Nuclear receptors coordinate the |
| | | | | activities of chromatin remodeling complexes and |
| | | | | coactivators to facilitate initiation of transcription |
| | | | | in carcinoma cells, Preinitiation Complex |
| | | | | Assembly of RNA Polymerase II at the TATA box) |
| 54 | 76391 | NM_022698 | bcl2-associated death promoter | (AKT Signaling Pathway, Amyotrophic lateral |
| | | | | sclerosis (ALS), Apoptosis, Apoptotic Signaling in |
| | | | | Response to DNA Damage, Ceramide Signaling |
| | | | | Pathway, Focal adhesion, IL-2 Receptor Beta |
| | | | | Chain in T cell Activation, Inhibition of Cellular |
| | | | | Proliferation by Gleevec, Multiple antiapoptotic |
| | | | | pathways from IGF-1R signaling lead to BAD |
| | | | | phosphorylation, Phosphoinositides and their |
| | | | | downstream targets., Ras Signaling Pathway, |
| | | | | Regulation of BAD phosphorylation, Regulation of |
| | | | | Insulin and IGF Signaling by IRS Proteins, Role |
| | | | | of nicotinic acetylcholine receptors in the |
| | | | | regulation of apoptosis, Trefoil Factors Initiate |
| | | | | Mucosal Healing) |
| 55 | 77247 | NM_030996 | opioid receptor, sigma 1 | |
| 56 | 82857 | NM_030863 | moesin | Regulation of actin cytoskeleton |
| 57 | 77252 | AF008912 | | |
| 58 | 77253 | NM_012583 | hypoxanthine guanine | Purine metabolism |
| | | | phosphoribosyl transferase | |
| 59 | 82942 | NM_053611 | nuclear protein 1 | |
| 41 | 82819 | NM_024362 | aryl hydrocarbon receptor nuclear | Circadian Rhythms |
| | | | translocator-like | |
| 60 | 80323 | NM_173315 | putative pheromone receptor (Go- | |
| | | | VN2) | |
| 61 | 80326 | NM_173130 | putative pheromone receptor (Go- | |
| | | | VN5) | |
| 62 | 77270 | NM_057136 | Epsin 1 | (Endocytotic role of NDK, Phosphins and |
| | | | | Dynamin, Stimulation of Synaptic Vesicle |
| | | | | Recycling by Nitrous Oxide) |
| 63 | 77271 | NM_019151 | growth differentiation factor 8 | |
| 64 | 77275 | NM_031768 | integrin, alpha E, epithelial- | Regulation of actin cytoskeleton |
| | | | associated | |
| 65 | 77276 | NM_053655 | dynamin 1-like | |
| 65 | 77277 | NM_053655 | dynamin 1-like | |
| 66 | 82918 | NM_030987 | guanine nucleotide binding protein, | (Activation of Csk by cAMP-dependent Protein |
| | | | beta 1 | Kinase Inhibits Signaling through the T Cell |
| | | | | Receptor, Activation of cAMP-dependent protein |
| | | | | kinase, PKA, Aspirin Blocks Signaling Pathway |
| | | | | Involved in Platelet Activation, Attenuation of |
| | | | | GPCR Signaling, Bioactive Peptide Induced |
| | | | | Signaling Pathway, Blood Pressure and RGS-2, |
| | | | | CCR3 signaling in Eosinophils, CXCR4 Signaling |
| | | | | Pathway, ChREBP regulation by carbohydrates |
| | | | | and cAMP, Corticosteroids and cardioprotection, |
| | | | | Ephrins and Ephs: forward and reverse signalling, |
| | | | | Erk1/Erk2 Mapk Signaling pathway, Formation of |
| | | | | Phagosomes and Recruitment of Endoplasmic |
| | | | | Reticulum, G-Protein Signaling Through Tubby |
| | | | | Proteins How Progesterone Initiates the Oocyte |
| | | | | Maturation, Ion Channels and Their Functional |
| | | | | Role in Vascular Endothelium, PKC-catalyzed |
| | | | | phosphorylation of inhibitory phosphoprotein of |
| | | | | myosin phosphatase, Phospholipids as signalling |
| | | | | intermediaries, Role of beta-arrestins in the |
| | | | | activation and targeting of MAP kinases, Roles of |
| | | | | beta-arrestin-dependent Recruitment of Src |
| | | | | Kinases in GPCR Signaling, Signaling Pathway |
| | | | | from G-Protein Families, The Role of Slit-Robo |
| | | | | Pathway in Axon Guidance, Thrombin signaling |
| | | | | and protease-activated receptors, beta-arrestins |
| | | | | in GPCR Desensitization, fMLP induced |
| | | | | chemokine gene expression in HMC-1 cells) |
| 67 | 76834 | NM_080397 | carbohydrate sulfotransferase 10 | |
| 68 | 77284 | NM_031771 | thrombomodulin | Complement and coagulation cascades |
| 69 | 82949 | NM_133591 | rabphilin 3A-like (without C2 | |
| | | | domains) | |
| 70 | 77286 | AF022952 | vascular endothelial growth factor B | |
| 71 | 77287 | NM_012551 | early growth response 1 | (Phosphorylation of MEK1 by cdk5/p35 down |
| | | | | regulates the MAP kinase pathway, Regulation of |
| | | | | Steroidogenesis by Orphan NuclearReceptor SF- |
| | | | | 1) |
| 72 | 77289 | AF023621 | sortilin 1 | GGA1 Adaptor Complex Pathway |
| 73 | 77291 | NM_022522 | caspase 2 | (Caspase Cascade in Apoptosis, HIV-I Nef: |
| | | | | negative effector of Fas and TNF, MAPK |
| | | | | signaling pathway, TNF/Stress Related Signaling, |
| | | | | TNFR1 Signaling Pathway) |
| 74 | 80347 | NM_031777 | upstream transcription factor 1 | |
| 75 | 80348 | AF026529 | stathmin-like 4 | |
| 76 | 77293 | AF027188 | similar to RIKEN cDNA 4933424N09 | |
| | | | (predicted) | |
| 77 | 77295 | NM_024353 | phospholipase C, beta 4 | (Calcium signaling pathway, Inositol phosphate |
| | | | | metabolism, Phosphatidylinositol signaling |
| | | | | system, Wnt signaling pathway) |
| 78 | 77298 | AF028784 | | |
| 79 | 77300 | NM_001000583 | olfactory receptor 857 (predicted) | |
| 80 | 77309 | NM_053662 | cyclin L1 | |
| 81 | 77312 | NM_134455 | chemokine (C—X3—C motif) ligand 1 | Cytokine-cytokine receptor interaction |
| 82 | 82963 | NM_019277 | SEC15-like 1 (S. cerevisiae) | |
| 83 | 82964 | NM_139043 | exocyst complex component 8 | |
| 84 | 77331 | NM_206850 | olfactory receptor 1696 | |
| 85 | 77336 | NM_022939 | syntaxin 12 | |
| 86 | 77339 | AF035952 | kinesin family member 6 | |
| 87 | 77349 | NM_031787 | homeodomain interacting protein | Phosphatidylinositol signaling system |
| | | | kinase 3 | |
| 88 | 82705 | NM_138922 | C-terminal PDZ domain ligand of | |
| | | | neuronal nitric oxide synthase | |
| 89 | 82946 | NM_131913 | X-prolyl aminopeptidase | |
| | | | (aminopeptidase P) 1, soluble | |
| 90 | 80379 | NM_053553 | synaptogyrin 2 | |
| 91 | 77354 | AF039308 | glutaminyl-peptide cyclotransferase | |
| | | | (glutaminyl cyclase) | |
| 92 | 82951 | NM_022951 | protein phosphatase 1, regulatory | |
| | | | subunit 10 | |
| 93 | 80384 | AF041107 | GTPase activating RANGAP | |
| | | | domain-like 1 | |
| 94 | 77374 | NM_171996 | 2-4-dienoyl-Coenzyme A reductase | (Purine metabolism, Pyrimidine metabolism) |
| | | | 2, peroxisomal | |
| 95 | 82965 | NM_022509 | survival of motor neuron 1, telomeric | U1snRNP Export and Import |
| 96 | 77380 | NM_031353 | voltage-dependent anion channel 1 | (Calcium signaling pathway, Mitochondrial |
| | | | | Permeability Transition Pore Complex) |
| 96 | 77381 | NM_031353 | voltage-dependent anion channel 1 | (Calcium signaling pathway, Mitochondrial |
| | | | | Permeability Transition Pore Complex) |
| 97 | 77385 | AF050214 | | |
| 98 | 77389 | NM_031593 | synaptic vesicle glycoprotein 2c | ECM-receptor interaction |
| 99 | 77395 | NM_013132 | annexin A5 | |
| 100 | 77406 | NM_181092 | synaptic Ras GTPase activating | |
| | | | protein 1 homolog (rat) | |
| 101 | 80160 | AF053987 | putative pheromone receptor V2R1 | |
| 102 | 80164 | NM_173318 | putative pheromone receptor (Go- | |
| | | | VN4) | |
| 103 | 77414 | AF055667 | | |
| 104 | 77420 | NM_022394 | scaffold attachment factor B | |
| 104 | 77421 | NM_022394 | scaffold attachment factor B | |
| 105 | 80256 | NM_031744 | solute carrier family 16, member 8 | |
| 106 | 82798 | NM_053557 | heterogeneous nuclear | |
| | | | ribonucleoprotein methyltransferase- | |
| | | | like 3 (S. cerevisiae) | |
| 98 | 77428 | NM_031593 | synaptic vesicle glycoprotein 2c | ECM-receptor interaction |
| 107 | 77438 | NM_053575 | calcineurin binding protein 1 | (Control of skeletal myogenesis by HDAC & |
| | | | | calcium/calmodulin-dependent kinase (CaMK), |
| | | | | Role of MEF2D in T-cell Apoptosis) |
| 108 | 77441 | NM_053561 | nucleosome assembly protein 1-like 1 | Nucleosome Organization |
| 109 | 77445 | NM_145091 | pyruvate dehydrogenase | |
| | | | phosphatase isoenzyme 2 | |
| 110 | 77450 | NM_022925 | protein tyrosine phosphatase, | |
| | | | receptor type, Q | |
| 111 | 82806 | NM_053563 | nuclear RNA helicase, DECD variant | |
| | | | of DEAD box family | |
| 112 | 77453 | NM_017318 | protein tyrosine kinase 2 beta | (Angiotensin II mediated activation of JNK |
| | | | | Pathway via Pyk2 dependent signaling, Bioactive |
| | | | | Peptide Induced Signaling Pathway, CXCR4 |
| | | | | Signaling Pathway, Calcium Signaling by HBx of |
| | | | | Hepatitis B virus, Calcium signaling pathway, IL-7 |
| | | | | Signal Transduction, Ion Channel and Phorbal |
| | | | | Esters Signaling Pathway, Links between Pyk2 |
| | | | | and Map Kinases, Pertussis toxin-insensitive |
| | | | | CCR5 Signaling in Macrophage, Ras- |
| | | | | Independent pathway in NK cell-mediated |
| | | | | cytotoxicity, Role of nicotinic acetylcholine |
| | | | | receptors in the regulation of apoptosis, Signaling |
| | | | | of Hepatocyte Growth Factor Receptor, Thrombin |
| | | | | signaling and protease-activated receptors) |
| 113 | 77456 | NM_024163 | brain-enriched guanylate kinase- | |
| | | | associated | |
| 114 | 77460 | NM_031756 | gamma-glutamyl carboxylase | Purine metabolism |
| 115 | 77473 | NM_024483 | adrenergic receptor, alpha 1d | Calcium signaling pathway |
| 116 | 77480 | AF072892 | chondroitin sulfate proteoglycan 2 | |
| 117 | 77481 | NM_022692 | RAB5A, member RAS oncogene | (Formation of Phagosomes and Recruitment of |
| | | | family | Endoplasmic Reticulum Phosphoinositides and |
| | | | | their downstream targets., Rab GTPases Mark |
| | | | | Targets In The Endocytotic Machinery, The role |
| | | | | of FYVE-finger proteins in vesicle transport) |
| 118 | 77483 | NM_133610 | potassium voltage-gated channel, | |
| | | | subfamily H (eag-related), member 5 | |
| 119 | 77491 | NM_134406 | phosphofurin acidic cluster sorting | |
| | | | protein 1 | |
| 119 | 77493 | NM_134406 | phosphofurin acidic cluster sorting | |
| | | | protein 1 | |
| 120 | 77494 | NM_031623 | growth factor receptor bound protein | |
| | | | 14 | |
| 121 | 77496 | NM_057204 | protein tyrosine phosphatase, non- | |
| | | | receptor type 23 | |
| 121 | 77497 | NM_057204 | protein tyrosine phosphatase, non- | |
| | | | receptor type 23 | |
| 122 | 77507 | NM_173293 | olfactory receptor 59 | |
| 123 | 77518 | NM_019211 | RAS guanyl releasing protein 1 | MAPK signaling pathway |
| 124 | 77530 | NM_019289 | actin related protein 2/3 complex, | (How Progesterone Initiates the Oocyte |
| | | | subunit 1B | Maturation, How does salmonella hijack a cell, |
| | | | | Listeria-Induced Signal Transduction, Regulation |
| | | | | of actin cytoskeleton, Rho cell motility signaling |
| | | | | pathway, Role of PI3K subunit p85 in regulation |
| | | | | of Actin Organization and Cell Migration, Role of |
| | | | | syndapins in vesicle trafficking, Y branching of |
| | | | | actin filaments) |
| 124 | 77531 | NM_019289 | actin related protein 2/3 complex, | (How Progesterone Initiates the Oocyte |
| | | | subunit 1B | Maturation, How does salmonella hijack a cell, |
| | | | | Listeria-Induced Signal Transduction, Regulation |
| | | | | of actin cytoskeleton, Rho cell motility signaling |
| | | | | pathway, Role of PI3K subunit p85 in regulation |
| | | | | of Actin Organization and Cell Migration, Role of |
| | | | | syndapins in vesicle trafficking, Y branching of |
| | | | | actin filaments) |
| 125 | 77537 | NM_053666 | delta-like 3 (Drosophila) | Notch signaling pathway |
| 126 | 77546 | NM_019290 | B-cell translocation gene 3 | |
| 127 | 77548 | NM_031749 | glucosidase 1 | N-Glycan biosynthesis |
| 128 | 77551 | NM_031597 | potassium voltage-gated channel, | |
| | | | subfamily Q, member 3 | |
| 129 | 77565 | NM_138535 | glutamate receptor interacting | |
| | | | protein 2 | |
| 130 | 77572 | NM_031816 | retinoblastoma binding protein 7 | |
| 131 | 77576 | NM_001000504 | olfactory receptor 37 | |
| 132 | 77577 | NM_001000507 | olfactory receptor 337 | |
| 133 | 77582 | NM_001006598 | olfactory receptor 1362 | |
| 134 | 77583 | NM_001000724 | olfactory receptor 1468 | |
| 135 | 77591 | NM_001000979 | olfactory receptor 1370 (predicted) | |
| 136 | 77595 | AF091580 | carboxypeptidase X 1 (M14 family) | |
| | | | (predicted) | |
| 137 | 77603 | NM_053665 | A kinase (PRKA) anchor protein 1 | |
| 25 | 77607 | NM_031707 | homer homolog 1 (Drosophila) | |
| 138 | 77625 | NM_022690 | ubiquitin-conjugating enzyme E2G 1 | (Parkinson's disease, Role of Parkin in the |
| | | | (UBC7 homolog, C. elegans) | Ubiquitin-Proteasomal Pathway, Ubiquitin |
| | | | | mediated proteolysis) |
| 139 | 77643 | NM_171990 | 3′(2′),5′-bisphosphate nucleotidase | |
| 140 | 80387 | NM_021997 | cytoplasmic linker 2 | |
| 141 | 77645 | NM_031514 | Janus kinase 2 | (Bioactive Peptide Induced Signaling Pathway, |
| | | | | Chaperones modulate interferon Signaling |
| | | | | Pathway, EPO Signaling Pathway, Erythropoietin |
| | | | | mediated neuroprotection through NE-kB, |
| | | | | Evasion of Innate Immunity by Protozoan |
| | | | | Parasites, Growth Hormone Signaling Pathway, |
| | | | | ICAM-1 in signal transduction, IFN gamma |
| | | | | signaling pathway, IL 3 signaling pathway, IL 6 |
| | | | | signaling pathway, IL12 and Stat4 Dependent |
| | | | | Signaling Pathway in Th1 Development, IL22 |
| | | | | Soluble Receptor Signaling Pathway, Inhibition of |
| | | | | Cellular Proliferation by Gleevec, Jak-STAT |
| | | | | signaling pathway, NO2-dependent IL 12 |
| | | | | Pathway in NK cells, Regulation of Inducible |
| | | | | Nitric Oxide Synthase by Interleukin-17, |
| | | | | Regulation of Insulin and IGF Signaling by IRS |
| | | | | Proteins, Stat3 Signaling Pathway, TPO Signaling |
| | | | | Pathway) |
| 142 | 77648 | NM_053715 | solute carrier family 5 (inositol | |
| | | | transporters), member 3 | |
| 143 | 77649 | NM_053301 | hemochromatosis | |
| 144 | 77651 | NM_013004 | phosphate regulating gene with | |
| | | | homologies to endopeptidases on | |
| | | | the X chromosome | |
| 145 | 76426 | AJ002942 | retinoic acid receptor, beta | |
| 146 | 77660 | NM_053349 | SRY-box containing gene 11 | |
| 147 | 77666 | NM_134452 | collagen, type V, alpha 1 | |
| 148 | 77670 | NM_021842 | endosulfine alpha | |
| 149 | 76430 | AJ006070 | recombination activating gene 1 | |
| 150 | 77676 | NM_024154 | amiloride-sensitive cation channel 2, | |
| | | | neuronal | |
| 151 | 77684 | NM_057143 | fertility protein SP22 | Parkinson's disease |
| 151 | 77685 | NM_057143 | fertility protein SP22 | Parkinson's disease |
| 152 | 77686 | NM_133567 | centaurin, alpha 1 | |
| 153 | 77690 | NM_145095 | potassium voltage-gated channel, | |
| | | | subfamily H, member 8 | |
| 154 | 77699 | NM_021838 | nitric oxide synthase 3, endothelial | (Actions of Nitric Oxide in the Heart, Arginine and |
| | | | cell | proline metabolism, Caveolin Based Estrogen |
| | | | | Signalling, Caveolin-Assisted eNOS Activation, |
| | | | | Corticosteroids and cardioprotection, Hypoxia- |
| | | | | Inducible Factor in the Cardiovascular System, |
| | | | | Ion Channels and Their Functional Role in |
| | | | | Vascular Endothelium, VEGF, Hypoxia, and |
| | | | | Angiogenesis) |
| 154 | 77700 | NM_021838 | nitric oxide synthase 3, endothelial | (Actions of Nitric Oxide in the Heart, Arginine and |
| | | | cell | proline metabolism, Caveolin Based Estrogen |
| | | | | Signalling, Caveolin-Assisted eNOS Activation, |
| | | | | Corticosteroids and cardioprotection, Hypoxia- |
| | | | | Inducible Factor in the Cardiovascular System, |
| | | | | Ion Channels and Their Functional Role in |
| | | | | Vascular Endothelium, VEGF, Hypoxia, and |
| | | | | Angiogenesis) |
| 155 | 77704 | NM_053480 | polymerase (DNA directed), alpha 2 | (DNA polymerase, Purine metabolism, Pyrimidine |
| | | | | metabolism) |
| 156 | 77708 | NM_031700 | claudin 3 | Tight junction |
| 157 | 77709 | AJ011811 | claudin 7 | Tight junction |
| 158 | 77713 | NM_020306 | a disintegrin and metalloproteinase | (Notch signaling pathway, Presenilin action in |
| | | | domain 17 (tumor necrosis factor, | Notch and Wnt signaling, Proteolysis and |
| | | | alpha, converting enzyme) | Signaling Pathway of Notch, g-Secretase |
| | | | | mediated ErbB4 Signaling Pathway) |
| 158 | 77714 | NM_020306 | a disintegrin and metalloproteinase | (Notch signaling pathway, Presenilin action in |
| | | | domain 17 (tumor necrosis factor, | Notch and Wnt signaling, Proteolysis and |
| | | | alpha, converting enzyme) | Signaling Pathway of Notch, g-Secretase |
| | | | | mediated ErbB4 Signaling Pathway) |
| 158 | 77715 | NM_020306 | a disintegrin and metalloproteinase | (Notch signaling pathway, Presenilin action in |
| | | | domain 17 (tumor necrosis factor, | Notch and Wnt signaling, Proteolysis and |
| | | | alpha, converting enzyme) | Signaling Pathway of Notch, g-Secretase |
| | | | | mediated ErbB4 Signaling Pathway) |
| 159 | 76433 | AJ223083 | retinoid X receptor gamma | |
| 160 | 77725 | NM_133418 | solute carrier family 25 | |
| | | | (mitochondrial carrier; dicarboxylate | |
| | | | transporter), member 10 | |
| 161 | 76815 | NM_053487 | peroxisomal biogenesis factor 11A | |
| 162 | 77728 | D00092 | similar to Dihydrolipoamide | |
| | | | acetyltransferase component of | |
| | | | pyruvate dehydrogenase complex | |
| | | | (E2) (PDC-E2) (70 kDa | |
| | | | mitochondrial autoantigen of primary | |
| | | | biliary cirrhosis) (PBC) | |
| 163 | 76780 | NM_057197 | 2,4-dienoyl CoA reductase 1, | |
| | | | mitochondrial | |
| 163 | 76781 | NM_057197 | 2,4-dienoyl CoA reductase 1, | |
| | | | mitochondrial | |
| 164 | 79889 | NM_012701 | adrenergic receptor, beta 1 | (Calcium signaling pathway, Neuroactive ligand- |
| | | | | receptor interaction) |
| 165 | 77732 | NM_138877 | diaphorase 1 | Aminosugars metabolism |
| 166 | 76441 | D00688 | monoamine oxidase A | (Arginine and proline metabolism, Glycine, serine |
| | | | | and threonine metabolism, Histidine metabolism, |
| | | | | Phenylalanine metabolism, Tryptophan |
| | | | | metabolism, Tyrosine metabolism) |
| 167 | 79598 | NM_017306 | dodecenoyl-coenzyme A delta | Fatty acid metabolism |
| | | | isomerase | |
| 168 | 81799 | NM_031531 | Serine protease inhibitor | |
| 169 | 77738 | NM_032617 | RAB11B, member RAS oncogene | |
| | | | family | |
| 170 | 82782 | NM_031095 | renin binding protein | Aminosugars metabolism |
| 171 | 82255 | NM_017127 | choline kinase alpha | (Glycerophospholipid metabolism, Glycine, serine |
| | | | | and threonine metabolism) |
| 172 | 77749 | NM_031044 | histamine N-methyltransferase | Histidine metabolism |
| 173 | 77751 | NM_017237 | ubiquitin carboxy-terminal hydrolase | Parkinson's disease |
| | | | L1 | |
| 174 | 77759 | NM_017283 | proteasome (prosome, macropain) | Proteasome |
| | | | subunit, alpha type 6 | |
| 175 | 77765 | D10853 | phosphoribosyl pyrophosphate | (Glutamate metabolism, Purine metabolism) |
| | | | amidotransferase | |
| 176 | 77769 | NM_053586 | cytochrome c oxidase subunit Vb | Oxidative phosphorylation |
| 177 | 77771 | NM_030845 | chemokine (C—X—C motif) ligand 1 | |
| 178 | 77781 | NM_057102 | solute carrier family 25 | Calcium signaling pathway |
| | | | (mitochondrial carrier; adenine | |
| | | | nucleotide translocator), member 5 | |
| 179 | 77785 | D12978 | POU domain, class 2, transcription | |
| | | | factor 1 | |
| 180 | 77786 | NM_019383 | ATP synthase, H+ transporting, | (ATP synthesis, Oxidative phosphorylation) |
| | | | mitochondrial F0 complex, subunit d | |
| 181 | 77787 | NM_012915 | ATPase inhibitor | |
| 182 | 77790 | NM_133556 | ATP synthase, H+ transporting, | (ATP synthesis, Oxidative phosphorylation) |
| | | | mitochondrial F0 complex, subunit c | |
| | | | (subunit 9), isoform 2 | |
| 183 | 77796 | NM_031563 | nuclease sensitive element binding | Transcriptional activation of dbpb from mRNA |
| | | | protein 1 | |
| 184 | 82850 | NM_024349 | adenylate kinase 1 | Purine metabolism |
| 185 | 77798 | NM_024360 | hairy and enhancer of split 1 | Notch signaling pathway |
| | | | (Drosophila) | |
| 186 | 82035 | NM_012764 | GATA binding protein 1 | Hemoglobin''s Chaperone |
| 187 | 77800 | D13556 | | |
| 188 | 77801 | NM_001008280 | similar to ribosome-binding protein | |
| | | | p34 —rat (predicted) | |
| 189 | 82985 | NM_022395 | peptidase (mitochondrial processing) | |
| | | | beta | |
| 190 | 77807 | NM_053327 | chloride channel K1 | |
| 190 | 77808 | NM_053327 | chloride channel K1 | |
| 191 | 77810 | NM_017102 | solute carrier family 2 (facilitated | Vitamin C in the Brain |
| | | | glucose transporter), member 3 | |
| 192 | 77811 | NM_022920 | glutamate receptor, metabotropic 6 | Neuroactive ligand-receptor interaction |
| 193 | 77816 | NM_031719 | chloride channel, nucleotide- | |
| | | | sensitive, 1A | |
| 194 | 76448 | NM_171992 | cyclin D1 | (BTG family proteins and cell cycle regulation, |
| | | | | CARM1 and Regulation of the Estrogen |
| | | | | Receptor, Cell Cycle: G1/S Check Point, Cell |
| | | | | cycle, Cyclins and Cell Cycle Regulation, Focal |
| | | | | adhesion, Inactivation of Gsk3 by AKT causes |
| | | | | accumulation of b-catenin in Alveolar |
| | | | | Macrophages, Influence of Ras and Rho proteins |
| | | | | on G1 to S Transition, Jak-STAT signaling |
| | | | | pathway, Migration Inhibitory Factor (MIF) in |
| | | | | Signalling and Cell Cycle Progression, Oncogene |
| | | | | Inactivation of p21 Tumor Suppressor, Signaling |
| | | | | Network Regulating Bone Morphogenesis, WNT |
| | | | | Signaling Pathway, Wnt signaling pathway, p53 |
| | | | | Signaling Pathway) |
| 195 | 77817 | NM_019236 | hairy and enhancer of split 2 | |
| | | | (Drosophila) | |
| 196 | 77819 | NM_057139 | heterogeneous nuclear | |
| | | | ribonucleoprotein U | |
| 197 | 82979 | NM_138538 | testicular dynamin | |
| 198 | 77828 | NM_017309 | protein phospatase 3, regulatory | (Apoptosis, MAPK signaling pathway, Wnt |
| | | | subunit B, alpha isoform, type 1 | signaling pathway) |
| 199 | 82030 | NM_030861 | Alpha-1,3-mannosyl-glycoprotein 2- | N-Glycan biosynthesis |
| | | | beta-N- | |
| | | | acetylglucosaminyltransferase | |
| 200 | 77839 | NM_012704 | prostaglandin E receptor 3 (subtype | (Calcium signaling pathway, Neuroactive ligand- |
| | | | EP3) | receptor interaction) |
| 200 | 77840 | NM_012704 | prostaglandin E receptor 3 (subtype | (Calcium signaling pathway, Neuroactive ligand- |
| | | | EP3) | receptor interaction) |
| 201 | 77842 | NM_031040 | glutamate receptor, metabotropic 7 | Neuroactive ligand-receptor interaction |
| 202 | 82056 | NM_013053 | tyrosine 3- | (Cell Cycle: G2/M Checkpoint, Chaperone —Raf |
| | | | monooxygenase/tryptophan 5- | Molecular Interactions) |
| | | | monooxygenase activation protein, | |
| | | | theta polypeptide | |
| 203 | 76462 | NM_017284 | proteasome (prosome, macropain) | Proteasome |
| | | | subunit, beta type 2 | |
| 204 | 77855 | NM_031715 | phosphofructokinase, muscle | (Fructose and mannose metabolism, Galactose |
| | | | | metabolism, Glycolysis/Gluconeogenesis, |
| | | | | Pentose phosphate pathway) |
| 205 | 76463 | D25233 | retinoblastoma 1 | (BTG family proteins and cell cycle regulation, |
| | | | | Cell Cycle: G1/S Check Point, Cell cycle, |
| | | | | Chaperones modulate interferon Signaling |
| | | | | Pathway, Cyclin E Destruction Pathway, Cyclins |
| | | | | and Cell Cycle Regulation, E2F1 Destruction |
| | | | | Pathway, FAS signaling pathway (CD95), HIV-I |
| | | | | Nef: negative effector of Fas and TNF, Human |
| | | | | Cytomegalovirus and Map Kinase Pathways, |
| | | | | Influence of Ras and Rho proteins on G1 to S |
| | | | | Transition, Mechanism of Gene Regulation by |
| | | | | Peroxisome Proliferators via PPARa(alpha), |
| | | | | Migration Inhibitory Factor (MIF) in Signalling and |
| | | | | Cell Cycle Progression, Overview of telomerase |
| | | | | RNA component gene hTerc Transcriptional |
| | | | | Regulation, RB Tumor Suppressor/Checkpoint |
| | | | | Signaling in response to DNA damage, |
| | | | | Regulation of p27 Phosphorylation during Cell |
| | | | | Cycle Progression, Regulation of transcriptional |
| | | | | activity by PML, TNFR1 Signaling Pathway, |
| | | | | Telomeres, Telomerase, Cellular Aging, and |
| | | | | Immortality, Tumor Suppressor Arf Inhibits |
| | | | | Ribosomal Biogenesis, p53 Signaling Pathway) |
| 206 | 77861 | NM_173103 | chloride channel Kb | |
| 207 | 77865 | D26393 | | |
| 208 | 82419 | NM_017079 | CD1d1 antigen | |
| 209 | 77881 | NM_012720 | myelin-associated oligodendrocytic | |
| | | | basic protein | |
| 210 | 77883 | NM_019122 | synaptotagmin 3 | |
| 211 | 76467 | NM_199501 | cyclin dependent kinase 2 | (Cell cycle, Phosphatidylinositol signaling system) |
| 212 | 82751 | NM_013127 | CD38 antigen | (Calcium signaling pathway, Nicotinate and |
| | | | | nicotinamide metabolism) |
| 213 | 82152 | NM_053596 | endothelin converting enzyme 1 | |
| 214 | 77893 | NM_012931 | breast cancer anti-estrogen | (CXCR4 Signaling Pathway, Cell to Cell Adhesion |
| | | | resistance 1 | Signaling, Focal adhesion, Integrin Signaling |
| | | | | Pathway, Links between Pyk2 and Map Kinases, |
| | | | | PTEN dependent cell cycle arrest and apoptosis, |
| | | | | Regulation of actin cytoskeleton, Signaling |
| | | | | Network Regulating Bone Morphogenesis) |
| 215 | 77896 | NM_019370 | ectonucleotide | (Nicotinate and nicotinamide metabolism, |
| | | | pyrophosphatase/phosphodiesterase 3 | Pantothenate and CoA biosynthesis, Purine |
| | | | | metabolism, Starch and sucrose metabolism) |
| 216 | 82555 | NM_013222 | growth factor, erv1-like | |
| 217 | 82531 | NM_017136 | squalene epoxidase | Biosynthesis of steroids |
| 218 | 79730 | D37934 | | |
| 219 | 77914 | D38036 | | |
| 220 | 77916 | NM_057105 | UDP glycosyltransferase 1 family, | (Androgen and estrogen metabolism, Pentose |
| | | | polypeptide A6 | and glucuronate interconversions, Porphyrin and |
| | | | | chlorophyll metabolism, Starch and sucrose |
| | | | | metabolism) |
| 221 | 77918 | NM_175846 | UDP glycosyltransferase 1 family, | (Androgen and estrogen metabolism, Pentose |
| | | | polypeptide A8 | and glucuronate interconversions, Porphyrin and |
| | | | | chlorophyll metabolism, Starch and sucrose |
| | | | | metabolism) |
| 222 | 77921 | D38067 | | |
| 223 | 82871 | NM_013016 | protein tyrosine phosphatase, non- | |
| | | | receptor type substrate 1 | |
| 224 | 77926 | NM_017292 | gamma-aminobutyric acid A | Neuroactive ligand-receptor interaction |
| | | | receptor, rho 2 | |
| 225 | 77929 | NM_001005330 | complement receptor related protein | Complement and coagulation cascades |
| 226 | 77931 | NM_057100 | growth arrest specific 6 | |
| 227 | 77938 | D45247 | proteasome (prosome, macropain) | Proteasome |
| | | | subunit, beta type 5 | |
| 227 | 77939 | D45247 | proteasome (prosome, macropain) | Proteasome |
| | | | subunit, beta type 5 | |
| 228 | 77945 | NM_022598 | cellular nucleic acid binding protein 1 | |
| 229 | 79794 | D45413 | protein tyrosine phosphatase, | |
| | | | receptor type, H | |
| 230 | 77948 | NM_013076 | leptin | (Cytokine-cytokine receptor interaction, Jak-STAT |
| | | | | signaling pathway, Neuroactive ligand-receptor |
| | | | | interaction, Reversal of Insulin Resistance by |
| | | | | Leptin) |
| 231 | 82654 | D49434 | arylsulfatase B | Glycosaminoglycan degradation |
| 232 | 80069 | NM_012631 | prion protein | Prion disease |
| 233 | 82653 | NM_017126 | ferredoxin 1 | |
| 234 | 82514 | NM_033236 | proteasome (prosome, macropain) | Proteasome |
| | | | 26S subunit, ATPase 2 | |
| 235 | 82516 | NM_057122 | proteasome (prosome, macropain) | Proteasome |
| | | | 26S subunit, ATPase, 4 | |
| 236 | 82515 | NM_057123 | peptidase (prosome, macropain) | Proteasome |
| | | | 26S subunit, ATPase 1 | |
| 237 | 76786 | NM_057125 | peroxisomal biogenesis factor 6 | Peroxisome Biogenesis and Matrix Protein Import |
| 238 | 76837 | NM_024153 | ferredoxin reductase | |
| 239 | 82703 | NM_013032 | solute carrier family 1 | |
| | | | (neuronal/epithelial high affinity | |
| | | | glutamate transporter, system Xag), | |
| | | | member 1 | |
| 240 | 80296 | NM_022185 | phosphatidylinositol 3-kinase, | (Apoptosis, Focal adhesion, Jak-STAT signaling |
| | | | regulatory subunit, polypeptide 2 | pathway, Phosphatidylinositol signaling system, |
| | | | | Regulation of actin cytoskeleton, Toll-like receptor |
| | | | | signaling pathway) |
| 241 | 82225 | NM_053594 | protein tyrosine phosphatase, | (Erk1/Erk2 Mapk Signaling pathway, MAPK |
| | | | receptor type, R | signaling pathway) |
| 242 | 77970 | NM_022211 | fibroblast growth factor 5 | (MAPK signaling pathway, Regulation of actin |
| | | | | cytoskeleton) |
| 243 | 80129 | NM_019326 | neurogenic differentiation 2 | |
| 244 | 82508 | NM_019161 | cadherin 22 | |
| 245 | 79979 | NM_013040 | ATP-binding cassette, sub-family C | |
| | | | (CFTR/MRP), member 9 | |
| 246 | 77984 | D84485 | ADP-ribosylhydrolase like 2 | |
| | | | (predicted) | |
| 247 | 82892 | NM_053623 | acyl-CoA synthetase long-chain | Fatty acid metabolism |
| | | | family member 4 | |
| 248 | 77988 | NM_134449 | protein kinase C, delta binding | |
| | | | protein | |
| 248 | 77989 | NM_134449 | protein kinase C, delta binding | |
| | | | protein | |
| 249 | 76759 | NM_012985 | NADH dehydrogenase (ubiquinone) | (Oxidative phosphorylation, Ubiquinone |
| | | | 1 alpha subcomplex 5 | biosynthesis) |
| 250 | 82830 | D86711 | similar to mKIAA1748 protein | |
| 251 | 77993 | NM_057133 | nuclear receptor subfamily 0, group | (FXR and LXR Regulation of Cholesterol |
| | | | B, member 2 | Metabolism, Nuclear Receptors in Lipid |
| | | | | Metabolism and Toxicity, Roles of Nuclear |
| | | | | Receptors in the Regulation of Bile Acid |
| | | | | Metabolism and Cholesterol Homeostasis, The |
| | | | | Liver X Receptor as a Key Regulator of |
| | | | | Cholesterol and Lipid Metabolism, The Nuclear |
| | | | | Pregnane X Receptor as a Generalized Sensor of |
| | | | | Hydrophobic Toxins, The Role of Farnesoid X |
| | | | | Receptor (FXR) in Regulation of Bile Acid |
| | | | | Homeostasis) |
| 252 | 82643 | NM_031097 | arginyl aminopeptidase | |
| | | | (aminopeptidase B) | |
| 253 | 80119 | NM_017179 | Unc4.1 homeobox (C. elegans) | |
| 254 | 77996 | D87922 | | |
| 255 | 77997 | NM_199081 | galactose transporter | |
| 255 | 77998 | NM_199081 | galactose transporter | |
| 256 | 82666 | NM_138882 | phosphatidylserine-specific | |
| | | | phospholipase A1 | |
| 257 | 80060 | NM_013100 | prostaglandin E receptor 1 | (Calcium signaling pathway, Eicosanoid |
| | | | | Metabolism, Neuroactive ligand-receptor |
| | | | | interaction, Phospholipase C-epsilon pathway) |
| 258 | 76489 | NM_019170 | carbonyl reductase 1 | Prostaglandin and leukotriene metabolism |
| 259 | 76490 | D89070 | | |
| 260 | 82923 | NM_022585 | ornithine decarboxylase antizyme | |
| | | | inhibitor | |
| 261 | 82757 | NM_012804 | ATP-binding cassette, sub-family D | |
| | | | (ALD), member 3 | |
| 262 | 78004 | NM_017280 | proteasome (prosome, macropain) | Proteasome |
| | | | subunit, alpha type 3 | |
| 263 | 78005 | NM_017278 | proteasome (prosome, macropain) | Proteasome |
| | | | subunit, alpha type 1 | |
| 264 | 78009 | NM_017097 | cathepsin C | |
| 265 | 79398 | NM_031511 | insulin-like growth factor 2 | |
| 266 | 79394 | NM_031576 | P450 (cytochrome) oxidoreductase | |
| 267 | 79390 | NM_031512 | interleukin 1 beta | (Apoptosis, Cytokine-cytokine receptor |
| | | | | interaction, IL 5 Signaling Pathway, |
| | | | | Immunotherapeutic Approaches to Alzheimer''s |
| | | | | Disease, MAPK signaling pathway, Matrix |
| | | | | Metalloproteinase 1 in Cell Signaling, Msp/Ron |
| | | | | Receptor Signaling Pathway, NFkB activation by |
| | | | | Nontypeable Hemophilus influenzae, PPAR |
| | | | | Gamma in Inflammation Control, Role of |
| | | | | Peroxisome Proliferator-Activated Receptor in |
| | | | | Inflammation, Signal transduction through IL1R, |
| | | | | The Liver X Receptor as a Key Regulator of |
| | | | | Cholesterol and Lipid Metabolism, The Role of |
| | | | | Farnesoid X Receptor (FXR) in Regulation of Bile |
| | | | | Acid Homeostasis, Toll-like receptor signaling |
| | | | | pathway) |
| 268 | 79388 | NM_012548 | endothelin 1 | (Hypoxia-Inducible Factor in the Cardiovascular |
| | | | | System, NFAT and Hypertrophy of the heart |
| | | | | (Transcription in the broken heart), Neuroactive |
| | | | | ligand-receptor interaction, PPAR Gamma in |
| | | | | Inflammation Control, Role of EGF Receptor |
| | | | | Transactivation by GPCRs in Cardiac |
| | | | | Hypertrophy, Role of Peroxisome Proliferator- |
| | | | | Activated Receptor in Inflammation) |
| 269 | 76498 | NM_017234 | peroxisomal membrane protein 3 | Peroxisome Biogenesis and Matrix Protein Import |
| 270 | 79380 | NM_024356 | GTP cyclohydrolase 1 | Folate biosynthesis |
| 271 | 79366 | NM_031049 | lanosterol synthase | Biosynthesis of steroids |
| 272 | 79363 | NM_080886 | sterol-C4-methyl oxidase-like | |
| 273 | 79358 | NM_019284 | chondroitin sulfate proteoglycan 5 | |
| 274 | 79356 | NM_057130 | BH3 interacting (with BCL2 family) | |
| | | | domain, apoptosis agonist | |
| 275 | 79354 | NM_020542 | macrophage inflammatory protein-1 | (Cytokine-cytokine receptor interaction, Selective |
| | | | alpha receptor gene | expression of chemokine receptors during T-cell |
| | | | | polarization) |
| 276 | 76501 | J00728 | | |
| 277 | 78012 | NM_012559 | fibrinogen, gamma polypeptide | (Acute Myocardial Infarction, C-Reactive Protein, |
| | | | | Apo-B in Lipid Metabolism and Cardiovascular |
| | | | | Disease, Complement and coagulation cascades, |
| | | | | Extrinsic Prothrombin Activation Pathway, |
| | | | | Fibrinolysis Pathway, Intrinsic Prothrombin |
| | | | | Activation Pathway, Platelet thrombus formation |
| | | | | at injury sites, Role of Peroxisome Proliferator- |
| | | | | Activated Receptor in Inflammation) |
| 278 | 78017 | NM_207593 | prostatic steroid-binding protein C2 | |
| 279 | 78025 | NM_012501 | apolipoprotein C-III | |
| 280 | 79531 | NM_013024 | spermine binding protein | |
| 281 | 76104 | NM_017000 | NAD(P)H dehydrogenase, quinone 1 | (Biosynthesis of steroids, Hypoxia and p53 in the |
| | | | | Cardiovascular system) |
| 282 | 82783 | NM_031832 | lectin, galactose binding, soluble 3 | |
| 283 | 78027 | NM_031090 | RAB1, member RAS oncogene | Rab GTPases Mark Targets In The Endocytotic |
| | | | family | Machinery |
| 284 | 83017 | NM_012793 | guanidinoacetate methyltransferase | (Arginine and proline metabolism, Glycine, serine |
| | | | | and threonine metabolism, Urea cycle and |
| | | | | metabolism of amino groups) |
| 285 | 82029 | NM_012672 | thyroid hormone receptor beta | (Map Kinase Inactivation of SMRT Corepressor, |
| | | | | Neuroactive ligand-receptor interaction) |
| 286 | 82766 | NM_017290 | ATPase, Ca++ transporting, cardiac | Calcium signaling pathway |
| | | | muscle, slow twitch 2 | |
| 287 | 82122 | NM_012621 | 6-phosphofructo-2-kinase/fructose- | Fructose and mannose metabolism |
| | | | 2,6-biphosphatase 1 | |
| 288 | 79491 | NM_017038 | protein phosphatase 1A, magnesium | MAPK signaling pathway |
| | | | dependent, alpha isoform | |
| 289 | 81903 | NM_022507 | protein kinase C, zeta | (CD44-Mediated Signaling, Insulin Stimulates |
| | | | | Glucose Transport, Phosphatidylinositol signaling |
| | | | | system, Phosphoinositides and their downstream |
| | | | | targets., Regulation of Insulin and IGF Signaling |
| | | | | by IRS Proteins, Rho-family Proteins in Cell |
| | | | | Adhesion and Cancer, Tight junction) |
| 290 | 82065 | NM_017031 | phosphodiesterase 4B | Purine metabolism |
| 291 | 82978 | NM_012789 | dipeptidylpeptidase 4 | |
| 292 | 80223 | NM_012970 | potassium voltage-gated channel, | |
| | | | shaker-related subfamily, member 2 | |
| 293 | 82069 | NM_012615 | ornithine decarboxylase 1 | (Arginine and proline metabolism, Urea cycle and |
| | | | | metabolism of amino groups) |
| 294 | 78032 | NM_019130 | insulin 2 | (Control of skeletal myogenesis by HDAC & |
| | | | | calcium/calmodulin-dependent kinase (CaMK), |
| | | | | Dentatorubropallidoluysian atrophy (DRPLA), |
| | | | | Growth Hormone Signaling Pathway, Insulin |
| | | | | Signaling Pathway, Insulin Stimulates Glucose |
| | | | | Transport, Mechanism of Gene Regulation by |
| | | | | Peroxisome Proliferators via PPARa(alpha), |
| | | | | Regulation of Insulin and IGF Signaling by IRS |
| | | | | Proteins, Regulation of actin cytoskeleton, Role of |
| | | | | HNF Transcription Factors in Diabetes, The Liver |
| | | | | X Receptor as a Key Regulator of Cholesterol |
| | | | | and Lipid Metabolism) |
| 295 | 82769 | NM_012992 | nucleophosmin 1 | |
| 296 | 76874 | NM_012815 | glutamate-cysteine ligase, catalytic | (Glutamate metabolism, Glutathione metabolism) |
| | | | subunit | |
| 297 | 76521 | NM_017202 | cytochrome c oxidase subunit IV | Oxidative phosphorylation |
| | | | isoform 1 | |
| 298 | 79529 | NM_134351 | methionine adenosyltransferase II, | (Methionine metabolism, Selenoamino acid |
| | | | alpha | metabolism) |
| 299 | 81901 | NM_022676 | protein phosphatase 1, regulatory | |
| | | | (inhibitor) subunit 1A | |
| 299 | 81902 | NM_022676 | protein phosphatase 1, regulatory | |
| | | | (inhibitor) subunit 1A | |
| 300 | 81949 | NM_020075 | eukaryotic translation initiation factor 5 | (Eukaryotic protein translation, Regulation of |
| | | | | eIF2) |
| 301 | 80428 | NM_012582 | haptoglobin | |
| 302 | 81866 | NM_001009618 | RNA polymerase II transcriptional | |
| | | | coactivator | |
| 303 | 81837 | NM_017027 | myelin protein zero | |
| 304 | 78051 | NM_012604 | myosin, heavy polypeptide 3, | |
| | | | skeletal muscle, embryonic | |
| 305 | 79785 | NM_012553 | elastase 2 | Proepithelin Conversion to Epithelin and Wound |
| | | | | Repair Control |
| 306 | 78053 | NM_012729 | protease, serine, 2 | |
| 307 | 79481 | NM_013008 | POU domain, class 1, transcription | |
| | | | factor 1 | |
| 308 | 81912 | NM_012763 | protein tyrosine phosphatase, | Activation of Src by Protein-tyrosine phosphatase |
| | | | receptor type, A | alpha |
| 309 | 78057 | NM_031043 | glycogenin 1 | |
| 310 | 80221 | L02315 | calcium channel, voltage-dependent, | |
| | | | beta 4 subunit | |
| 311 | 82997 | NM_021266 | frizzled homolog 1 (Drosophila) | (ALK in cardiac myocytes, Inactivation of Gsk3 by |
| | | | | AKT causes accumulation of b-catenin in Alveolar |
| | | | | Macrophages, Multi-step Regulation of |
| | | | | Transcription by Pitx2, Presenilin action in Notch |
| | | | | and Wnt signaling, Rho-family Proteins in Cell |
| | | | | Adhesion and Cancer, Segmentation Clock, WNT |
| | | | | Signaling Pathway, Wnt signaling pathway, |
| | | | | Wnt/LRP6 Signalling) |
| 312 | 76530 | NM_012854 | interleukin 10 | (Adaptive Immune Response to Cancer Cells, |
| | | | | Antigen Dependent B Cell Activation, Cytokine |
| | | | | Network, Cytokine-cytokine receptor interaction, |
| | | | | Cytokines and Inflammatory Response, Dendritic |
| | | | | cells in regulating TH1 and TH2 Development, IL- |
| | | | | 10 Anti-inflammatory Signaling Pathway, |
| | | | | Immunotherapeutic Approaches to Alzheimer''s |
| | | | | Disease, Jak-STAT signaling pathway, PPAR |
| | | | | Gamma in Inflammation Control) |
| 313 | 76623 | NM_012598 | lipoprotein lipase | (Alzheimer's disease, C-Reactive Protein, Apo-B |
| | | | | in Lipid Metabolism and Cardiovascular Disease, |
| | | | | Glycerolipid metabolism, Low-density lipoprotein |
| | | | | (LDL) pathway during atherogenesis, Mechanism |
| | | | | of Gene Regulation by Peroxisome Proliferators |
| | | | | via PPARa(alpha), Role of PPAR-gamma |
| | | | | Coactivators in Obesity and Thermogenesis, |
| | | | | Roles of Nuclear Receptors in the Regulation of |
| | | | | Bile Acid Metabolism and Cholesterol |
| | | | | Homeostasis, Visceral Fat Deposits and the |
| | | | | Metabolic Syndrome) |
| 314 | 81888 | NM_022707 | phospholamban | Calcium signaling pathway |
| 315 | 82146 | NM_012945 | diphtheria toxin receptor | NFAT and Hypertrophy of the heart (Transcription |
| | | | | in the broken heart) |
| 316 | 79422 | L06804 | LIM homeobox protein 2 (predicted) | |
| 317 | 82020 | NM_133593 | adaptor-related protein complex 3, | |
| | | | mu 1 subunit | |
| 318 | 82021 | NM_133305 | adaptor-related protein complex 3, | |
| | | | mu 2 subunit | |
| 319 | 78074 | NM_012850 | growth hormone releasing hormone | Neuroactive ligand-receptor interaction |
| | | | receptor | |
| 320 | 78080 | L07402 | | |
| 321 | 78082 | L07408 | | |
| 322 | 78084 | L07410 | | |
| 323 | 78085 | NM_019250 | ral guanine nucleotide dissociation | Ras Signaling Pathway |
| | | | stimulator | |
| 323 | 78086 | NM_019250 | ral guanine nucleotide dissociation | Ras Signaling Pathway |
| | | | stimulator | |
| 324 | 78087 | NM_017010 | glutamate receptor, ionotropic, N- | (Calcium signaling pathway, Erythropoietin |
| | | | methyl D-aspartate 1 | mediated neuroprotection through NF-kB, |
| | | | | Ethanol-Induced Signal Transduction, LIN |
| | | | | Transport Complex, Neuregulin Receptor |
| | | | | Signaling, Neuroactive ligand-receptor |
| | | | | interaction, Nitric Oxide Signaling Pathway, |
| | | | | Stimulation of Synaptic Vesicle Recycling by |
| | | | | Nitrous Oxide, Synaptic Proteins at the Synaptic |
| | | | | Junction) |
| 325 | 78093 | NM_021841 | gamma-aminobutyric acid A | (Cardiac Protection Against ROS, Gamma- |
| | | | receptor, alpha 6 | aminobutyric Acid Receptor Life Cycle, |
| | | | | Neuroactive ligand-receptor interaction) |
| 326 | 78094 | NM_017289 | gamma-aminobutyric acid A | Neuroactive ligand-receptor interaction |
| | | | receptor, delta | |
| 327 | 82058 | NM_031723 | signal peptidase complex 18 kD | Protein export |
| 328 | 79571 | NM_017033 | phosphoglucomutase 1 | (Galactose metabolism, Glycolysis/ |
| | | | | Gluconeogenesis, Pentose phosphate pathway, |
| | | | | Starch and sucrose metabolism) |
| 329 | 82641 | NM_017076 | poliovirus receptor | CD155-Assisted Signal Transduction |
| 330 | 82726 | NM_024151 | ADP-ribosylation factor 4 | |
| 331 | 78109 | NM_019905 | annexin A2 | Caveolin-Assisted Cholesterol Transport |
| 332 | 78122 | NM_012519 | calcium/calmodulin-dependent | (Bioactive Peptide Induced Signaling Pathway, |
| | | | protein kinase II, delta | CD44-Mediated Signaling, Ca++/Calmodulin- |
| | | | | dependent Protein Kinase Activation, Calcium |
| | | | | signaling pathway, Meiotic Arrest in Oogenesis, |
| | | | | Regulation of PGC-1a, Stathmin and breast |
| | | | | cancer resistance to antimicrotubule agents, |
| | | | | Transcription factor CREB and its extracellular |
| | | | | signals, Wnt signaling pathway) |
| 333 | 78124 | NM_022392 | insulin induced gene 1 | |
| 334 | 78129 | NM_012723 | polymeric immunoglobulin receptor | |
| 335 | 78133 | NM_019220 | amino-terminal enhancer of split | |
| 336 | 78136 | NM_133559 | proprotein convertase subtilisin/kexin | |
| | | | type 4 | |
| 336 | 78137 | NM_133559 | proprotein convertase subtilisin/kexin | |
| | | | type 4 | |
| 337 | 81831 | NM_030857 | Yamaguchi sarcoma viral (v-yes-1) | (BCR Signaling Pathway, Calcium signaling |
| | | | oncogene homolog | pathway, Eph Kinases and ephrins support |
| | | | | platelet aggregation, Fc Epsilon Receptor I |
| | | | | Signaling in Mast Cells, Phosphoinositides and |
| | | | | their downstream targets., Platelet thrombus |
| | | | | formation at injury sites, Ub/Proteosome System |
| | | | | in EBV-associated Malignancies) |
| 338 | 82053 | NM_012809 | cyclic nucleotide phosphodiesterase 1 | |
| 14 | 76534 | NM_212504 | heat shock 70 kD protein 1B | MAPK signaling pathway |
| 339 | 79985 | NM_017265 | hydroxy-delta-5-steroid | (Androgen and estrogen metabolism, C21-Steroid |
| | | | dehydrogenase, 3 beta- and steroid | hormone metabolism) |
| | | | delta-isomerase 1 | |
| 340 | 76535 | NM_172008 | calnexin | |
| 341 | 81921 | NM_031093 | v-ral simian leukemia viral oncogene | (Migfilin-Mediated Actin Remodeling, Ras |
| | | | homolog A (ras related) | Signaling Pathway) |
| 342 | 80288 | NM_138511 | glypican 2 (cerebroglycan) | |
| 343 | 78149 | NM_031836 | vascular endothelial growth factor A | (Actions of Nitric Oxide in the Heart, Focal |
| | | | | adhesion, Hypoxia-Inducible Factor in the |
| | | | | Cardiovascular System, PIGF: A Key Player in |
| | | | | Pathological Angiogenesis, The Liver X Receptor |
| | | | | as a Key Regulator of Cholesterol and Lipid |
| | | | | Metabolism, VEGF, Hypoxia, and Angiogenesis) |
| 344 | 82209 | NM_019185 | GATA binding protein 6 | |
| 345 | 81900 | NM_144730 | GATA binding protein 4 | (ALK in cardiac myocytes, Hop Pathway in |
| | | | | Cardiac Development, NFAT and Hypertrophy of |
| | | | | the heart (Transcription in the broken heart), |
| | | | | Regulation of Steroidogenesis by Orphan |
| | | | | NuclearReceptor SF-1) |
| 346 | 81867 | NM_053820 | early B-cell factor 1 | |
| 347 | 79745 | L24389 | | |
| 348 | 79563 | NM_057208 | tropomyosin 3, gamma | |
| 349 | 76634 | NM_017165 | glutathione peroxidase 4 | Glutathione metabolism |
| 350 | 79650 | L24897 | myosin, heavy polypeptide 13, | |
| | | | skeletal muscle | |
| 351 | 82256 | NM_134468 | calcium/calmodulin-dependent | |
| | | | protein kinase I | |
| 352 | 82405 | L25785 | transforming growth factor beta 1 | |
| | | | induced transcript 4 | |
| 353 | 79890 | L26293 | | |
| 354 | 79592 | NM_016987 | ATP citrate lyase | (Citrate cycle (TCA cycle), Shuttle for transfer of |
| | | | | acetyl groups from mitochondria to the cytosol) |
| 355 | 80226 | L27663 | POU domain, class 3, transcription | |
| | | | factor 2 | |
| 356 | 82033 | NM_019335 | Protein kinase, interferon-inducible | (Inositol phosphate metabolism, Nicotinate and |
| | | | double stranded RNA dependent | nicotinamide metabolism, Phosphatidylinositol |
| | | | | signaling system) |
| 357 | 78173 | NM_031343 | solute carrier family 6 | |
| | | | (neurotransmitter transporter, | |
| | | | noradrenalin), member 2 | |
| 358 | 81857 | NM_012832 | cholinergic receptor, nicotinic, alpha | Calcium signaling pathway |
| | | | polypeptide 7 | |
| 359 | 78177 | NM_019297 | cholinergic receptor, nicotinic, beta | |
| | | | polypeptide 2 (neuronal) | |
| 360 | 76542 | NM_024127 | growth arrest and DNA-damage- | (ATM Signaling Pathway, Cell Cycle: G2/M |
| | | | inducible 45 alpha | Checkpoint, Cell cycle, Hypoxia and p53 in the |
| | | | | Cardiovascular system, MAPK signaling pathway, |
| | | | | p53 Signaling Pathway) |
| 361 | 78178 | NM_021860 | olfactory receptor 1654 (predicted) | |
| 362 | 82025 | NM_022502 | palmitoyl-protein thioesterase | |
| 363 | 78179 | NM_021859 | megakaryocyte-associated tyrosine | Regulation of actin cytoskeleton |
| | | | kinase | |
| 364 | 79631 | NM_024380 | guanylyl cyclase 2e | Purine metabolism |
| 365 | 79632 | NM_053831 | guanylate cyclase 2f | Purine metabolism |
| 225 | 82202 | NM_001005330 | complement receptor related protein | Complement and coagulation cascades |
| 366 | 78181 | NM_012544 | angiotensin 1 converting enzyme | Angiotensin-converting enzyme 2 regulates heart |
| | | | | function |
| 367 | 76686 | NM_019223 | NADH dehydrogenase (ubiquinone) | (Oxidative phosphorylation, Ubiquinone |
| | | | Fe—S protein 6 | biosynthesis) |
| 368 | 82063 | L39991 | nucleoporin 98 | |
| 369 | 78187 | NM_031819 | fat tumor suppressor homolog | |
| | | | (Drosophila) | |
| 370 | 78193 | L47281 | collagen, type IV, alpha 3 | ECM-receptor interaction |
| 371 | 76548 | L48209 | cytochrome c oxidase, subunit VIIIa | Oxidative phosphorylation |
| 266 | 76549 | NM_031576 | P450 (cytochrome) oxidoreductase | |
| 372 | 79441 | NM_001008830 | RT1 class I, A3 | |
| 373 | 81871 | NM_013078 | ornithine transcarbamylase | (Arginine and proline metabolism, Urea cycle and |
| | | | | metabolism of amino groups) |
| 374 | 76552 | NM_024351 | heat shock protein 8 | MAPK signaling pathway |
| 375 | 83025 | NM_017248 | heterogeneous nuclear | |
| | | | ribonucleoprotein A1 | |
| 376 | 78205 | M12335 | | |
| 377 | 80420 | NM_012767 | gonadotropin-releasing hormone 1 | Neuroactive ligand-receptor interaction |
| 378 | 81895 | NM_017036 | prolactin-like protein A | |
| 379 | 83014 | NM_138827 | solute carrier family 2 (facilitated | Vitamin C in the Brain |
| | | | glucose transporter), member 1 | |
| 380 | 81869 | NM_012881 | secreted phosphoprotein 1 | (ECM-receptor interaction, Focal adhesion) |
| 381 | 78216 | NM_017158 | cytochrome P450, family 2, | (Fatty acid metabolism, Tryptophan metabolism) |
| | | | subfamily c, polypeptide 7 | |
| 382 | 82685 | NM_019286 | alcohol dehydrogenase 1 | (Bile acid biosynthesis, Fatty acid metabolism, |
| | | | | Glycerolipid metabolism, Glycolysis/ |
| | | | | Gluconeogenesis, Tyrosine metabolism) |
| 383 | 80435 | NM_178866 | insulin-like growth factor 1 | (Control of skeletal myogenesis by HDAC & |
| | | | | calcium/calmodulin-dependent kinase (CaMK), |
| | | | | Erythrocyte Differentiation Pathway, Ghrelin: |
| | | | | Regulation of Food Intake and Energy |
| | | | | Homeostasis, IGF-1 Signaling Pathway, NFAT |
| | | | | and Hypertrophy of the heart (Transcription in the |
| | | | | broken heart), Regulation of BAD |
| | | | | phosphorylation, Regulation of Insulin and IGF |
| | | | | Signaling by IRS Proteins, Skeletal muscle |
| | | | | hypertrophy is regulated via AKT/mTOR pathway, |
| | | | | The IGF-1 Receptor and Longevity) |
| 384 | 78224 | M15528 | | |
| 385 | 80267 | NM_031974 | clathrin, light polypeptide (Lca) | (Enthoprotin interactions at the trans-Golgi |
| | | | | network, GGA1 Adaptor Complex Pathway, |
| | | | | Huntington's disease, Stimulation of Synaptic |
| | | | | Vesicle Recycling by Nitrous Oxide) |
| 386 | 78226 | NM_080773 | cholinergic receptor, muscarinic 1 | (Actions of Nitric Oxide in the Heart, Agrin in |
| | | | | Postsynaptic Differentiation, Blockade of |
| | | | | Neurotransmitter Relase by Botulinum Toxin, |
| | | | | Calcium signaling pathway, G-Protein Signaling |
| | | | | Through Tubby Proteins, Neuroactive ligand- |
| | | | | receptor interaction, Regulation of actin |
| | | | | cytoskeleton) |
| 387 | 80086 | M16409 | cholinergic receptor, muscarinic 4 | |
| 388 | 79464 | NM_019162 | tachykinin 2 | Neuroactive ligand-receptor interaction |
| 389 | 78228 | M17523 | | |
| 390 | 76560 | NM_017101 | peptidylprolyl isomerase A | (Caveolin-Assisted Cholesterol Transport, IL-2 |
| | | | | Receptor Beta Chain in T cell Activation) |
| 391 | 78244 | NM_001005382 | glandular kallikrein 12, | |
| | | | submandibular/renal | |
| 392 | 82995 | NM_012953 | fos-like antigen 1 | (Bone Remodelling, Wnt signaling pathway) |
| 393 | 78248 | M20722 | proline-rich protein | |
| 394 | 78249 | M20724 | proline-rich protein | |
| 395 | 78252 | NM_012503 | asialoglycoprotein receptor 1 | |
| 396 | 79690 | NM_030875 | sodium channel, voltage-gated, type | Opiate-Induced Signal Transduction |
| | | | 1, alpha polypeptide | |
| 391 | 78259 | NM_001005382 | glandular kallikrein 12, | |
| | | | submandibular/renal | |
| 397 | 78261 | NM_023103 | Murinoglobulin 1 homolog (mouse) | |
| 398 | 78262 | NM_012488 | alpha-2-macroglobulin | (Alzheimer's disease, Complement and |
| | | | | coagulation cascades) |
| 399 | 81898 | NM_017040 | protein phosphatase 2a, catalytic | (TGF-beta signaling pathway, Tight junction, Wnt |
| | | | subunit, beta isoform | signaling pathway) |
| 400 | 78263 | NM_013046 | thyrotropin releasing hormone | Neuroactive ligand-receptor interaction |
| 401 | 81881 | NM_013151 | plasminogen activator, tissue | (Acute Myocardial Infarction, Complement and |
| | | | | coagulation cascades, Fibrinolysis Pathway, |
| | | | | Platelet Amyloid Precursor Protein Pathway) |
| 402 | 82017 | NM_012620 | serine (or cysteine) proteinase | (Activation of Plasminogen-Activator Inhibitor |
| | | | inhibitor, clade E, member 1 | (PIA-1) by NFkB, Complement and coagulation |
| | | | | cascades, Fibrinolysis Pathway, Platelet Amyloid |
| | | | | Precursor Protein Pathway) |
| 403 | 76565 | M24239 | | |
| 404 | 81820 | NM_031012 | alanyl (membrane) aminopeptidase | (Dendritic cells in regulating TH1 and TH2 |
| | | | | Development, Glutathione metabolism, SARS |
| | | | | Coronavirus Protease) |
| 405 | 78276 | NM_013101 | phosphodiesterase 4A | Purine metabolism |
| 290 | 78277 | NM_017031 | phosphodiesterase 4B | Purine metabolism |
| 406 | 79667 | NM_145775 | nuclear receptor subfamily 1, group | Circadian rhythm |
| | | | D, member 1 | |
| 407 | 78279 | NM_138507 | protein tyrosine phosphatase, | (Activation of Csk by cAMP-dependent Protein |
| | | | receptor type, C | Kinase Inhibits Signaling through the T Cell |
| | | | | Receptor, B Lymphocyte Cell Surface Molecules, |
| | | | | Lck and Fyn tyrosine kinases in initiation of TCR |
| | | | | Activation, T Cytotoxic Cell Surface Molecules, T |
| | | | | Helper Cell Surface Molecules) |
| 408 | 76660 | NM_012844 | epoxide hydrolase 1 | |
| 409 | 76569 | NM_012541 | cytochrome P450, family 1, | (Fatty acid metabolism, Mechanism of |
| | | | subfamily a, polypeptide 2 | Acetaminophen Activity and Toxicity, Nuclear |
| | | | | Receptors in Lipid Metabolism and Toxicity, |
| | | | | Tryptophan metabolism) |
| 410 | 79423 | NM_012978 | luteinizing | (Calcium signaling pathway, Neuroactive ligand- |
| | | | hormone/choriogonadotropin | receptor interaction) |
| | | | receptor | |
| 411 | 79522 | M26534 | | |
| 412 | 79457 | NM_013178 | sodium channel, voltage-gated, type | |
| | | | IV, alpha polypeptide | |
| 413 | 82027 | M26686 | | |
| 414 | 79451 | NM_019266 | sodium channel, voltage-gated, type | |
| | | | 8, alpha polypeptide | |
| 415 | 78285 | NM_147214 | alpha-2u globulin PGCL1 | |
| 416 | 76685 | NM_019360 | cytochrome c oxidase, subunit VIc | Oxidative phosphorylation |
| 417 | 81803 | NM_017071 | insulin receptor | (Adherens junction, Control of skeletal |
| | | | | myogenesis by HDAC & calcium/calmodulin- |
| | | | | dependent kinase (CaMK), |
| | | | | Dentatorubropallidoluysian atrophy (DRPLA), |
| | | | | Growth Hormone Signaling Pathway, Insulin |
| | | | | Signaling Pathway, Insulin Stimulates Glucose |
| | | | | Transport, Regulation of Insulin and IGF |
| | | | | Signaling by IRS Proteins, Role of HNF |
| | | | | Transcription Factors in Diabetes) |
| 418 | 81965 | NM_031117 | small nuclear ribonucleoprotein N | (Spliceosomal Assembly, U1snRNP Export and |
| | | | | Import) |
| 419 | 81947 | NM_017160 | ribosomal protein S6 | (Ribosome, Skeletal muscle hypertrophy is |
| | | | | regulated via AKT/mTOR pathway, mTOR |
| | | | | Signaling Pathway) |
| 420 | 81828 | NM_139257 | lymphocyte antigen 6 complex, locus B | |
| 421 | 78296 | NM_133285 | H1 histone family, member 4 | |
| 422 | 81826 | NM_017068 | lysosomal membrane glycoprotein 2 | |
| 343 | 76875 | NM_031836 | vascular endothelial growth factor A | (Actions of Nitric Oxide in the Heart, Focal |
| | | | | adhesion, Hypoxia-Inducible Factor in the |
| | | | | Cardiovascular System, PIGF: A Key Player in |
| | | | | Pathological Angiogenesis, The Liver X Receptor |
| | | | | as a Key Regulator of Cholesterol and Lipid |
| | | | | Metabolism, VEGF, Hypoxia, and Angiogenesis) |
| 423 | 79474 | NM_031975 | parathymosin | |
| 424 | 81915 | NM_012637 | protein tyrosine phosphatase, non- | Adherens junction |
| | | | receptor type 1 | |
| 425 | 81969 | NM_030656 | alanine-glyoxylate aminotransferase | (Alanine and aspartate metabolism, Glycine, |
| | | | | serine and threonine metabolism) |
| 426 | 79525 | NM_153722 | MAS-related GPR, member F | |
| 400 | 78307 | NM_013046 | thyrotropin releasing hormone | Neuroactive ligand-receptor interaction |
| 427 | 78308 | NM_031608 | glutamate receptor, ionotropic, | Neuroactive ligand-receptor interaction |
| | | | AMPA1 (alpha 1) | |
| 428 | 78312 | NM_001007597 | follicle-stimulating hormone subunit | (Neuroactive ligand-receptor interaction, |
| | | | beta | Regulation of Spermatogenesis by CREM) |
| 429 | 78315 | M37568 | | |
| 430 | 76583 | NM_178847 | cytochrome P450, family 27, | Bile acid biosynthesis |
| | | | subfamily a, polypeptide 1 | |
| 431 | 82043 | NM_017016 | histidine decarboxylase | Histidine metabolism |
| 432 | 78319 | NM_012749 | nucleolin | SARS Coronavirus Protease |
| 433 | 80414 | NM_012770 | guanylate cyclase 1, soluble, beta 2 | Purine metabolism |
| 434 | 82839 | NM_012529 | creatine kinase, brain | (Arginine and proline metabolism, Urea cycle and |
| | | | | metabolism of amino groups) |
| 435 | 79560 | NM_019353 | thyroid peroxidase | (Cytokine-cytokine receptor interaction, Jak-STAT |
| | | | | signaling pathway, Tyrosine metabolism) |
| 436 | 81907 | NM_031604 | ATPase, H+ transporting, lysosomal | (ATP synthesis, Oxidative phosphorylation) |
| | | | V0 subunit a isoform 1 | |
| 437 | 82095 | NM_022547 | formyltetrahydrofolate | One carbon pool by folate |
| | | | dehydrogenase | |
| 438 | 76805 | NM_012498 | Aldehyde reductase 1 (low Km | (Fructose and mannose metabolism, Galactose |
| | | | aldose reductase) (5.8 kb PstI | metabolism, Glycerolipid metabolism, Pentose |
| | | | fragment, probably the functional | and glucuronate interconversions, Pyruvate |
| | | | gene) | metabolism) |
| 439 | 82045 | NM_016991 | adrenergic receptor, alpha 1b | (Calcium signaling pathway, Neuroactive ligand- |
| | | | | receptor interaction, Phospholipase C d1 in |
| | | | | phospholipid associated cell signaling) |
| 440 | 82679 | NM_017259 | B-cell translocation gene 2, anti- | |
| | | | proliferative | |
| 440 | 82680 | NM_017259 | B-cell translocation gene 2, anti- | |
| | | | proliferative | |
| 410 | 78324 | NM_012978 | luteinizing | (Calcium signaling pathway, Neuroactive ligand- |
| | | | hormone/choriogonadotropin | receptor interaction) |
| | | | receptor | |
| 441 | 78325 | M61725 | upstream binding transcription | |
| | | | factor, RNA polymerase I | |
| 442 | 76661 | NM_012924 | CD44 antigen | (Adhesion Molecules on Lymphocyte, CD44 |
| | | | | Signaling Pathway, CD44-Mediated Signaling, |
| | | | | Caveolin-Assisted eNOS Activation, ECM- |
| | | | | receptor interaction, Monocyte and its Surface |
| | | | | Molecules, Neutrophil and Its Surface Molecules) |
| 443 | 76591 | NM_031138 | ubiquitin conjugating enzyme | Ubiquitin mediated proteolysis |
| 444 | 81860 | NM_001010964 | killer cell lectin-like receptor | |
| | | | subfamily B member 1A | |
| 445 | 81878 | NM_016989 | adenylate cyclase activating | Neuroactive ligand-receptor interaction |
| | | | polypeptide 1 | |
| 446 | 76654 | NM_013091 | tumor necrosis factor receptor | (Acetylation and Deacetylation of RelA in The |
| | | | superfamily, member 1a | Nucleus, Apoptosis, Ceramide Signaling |
| | | | | Pathway, Chaperones modulate interferon |
| | | | | Signaling Pathway, Cytokine-cytokine receptor |
| | | | | interaction, HIV-I Nef: negative effector of Fas |
| | | | | and TNF, Keratinocyte Differentiation, MAPK |
| | | | | signaling pathway, NF-kB Signaling Pathway, |
| | | | | Regulation of Inducible Nitric Oxide Synthase by |
| | | | | Interleukin-17, Regulation of Insulin and IGF |
| | | | | Signaling by IRS Proteins, Regulation of |
| | | | | transcriptional activity by PML, SODD/TNFR1 |
| | | | | Signaling Pathway, TNF/Stress Related |
| | | | | Signaling, TNFR1 Signaling Pathway) |
| 447 | 79660 | NM_017155 | adenosine A1 receptor | Neuroactive ligand-receptor interaction |
| 448 | 79622 | NM_012665 | synaptotagmin 2 | |
| 449 | 79518 | M64793 | | |
| 450 | 79798 | M64795 | | |
| 451 | 79431 | NM_022667 | solute carrier organic anion | |
| | | | transporter family, member 2a1 | |
| 452 | 80431 | NM_017254 | 5-hydroxytryptamine (serotonin) | (Calcium signaling pathway, Chlamydia-Induced |
| | | | receptor 2A | Signal Transduction, Neuroactive ligand-receptor |
| | | | | interaction) |
| 453 | 82760 | NM_012963 | high mobility group box 1 | |
| 454 | 79554 | NM_133514 | matrix metallopeptidase 10 | |
| 455 | 83008 | NM_031577 | growth hormone releasing hormone | Neuroactive ligand-receptor interaction |
| 456 | 81833 | NM_031731 | aldehyde dehydrogenase family 3, | (Arginine and proline metabolism, Bile acid |
| | | | subfamily A2 | biosynthesis, Butanoate metabolism, Fatty acid |
| | | | | metabolism, Glycerolipid metabolism, Glycolysis/ |
| | | | | Gluconeogenesis, Histidine metabolism, Lysine |
| | | | | degradation, Propanoate metabolism, Pyruvate |
| | | | | metabolism, Tryptophan metabolism, Valine, |
| | | | | leucine and isoleucine degradation, beta-Alanine |
| | | | | metabolism) |
| 156 | 81868 | NM_031700 | claudin 3 | Tight junction |
| 457 | 79454 | NM_012504 | ATPase, Na+/K+ transporting, alpha | |
| | | | 1 polypeptide | |
| 458 | 76598 | M74776 | | |
| 459 | 81816 | M75148 | kinesin 2 | (Rhodopsin Processing and Transport, Trafficking |
| | | | | of MAPK Signaling Modules by Kinesin I) |
| 460 | 76602 | NM_019280 | gap junction membrane channel | |
| | | | protein alpha 5 | |
| 461 | 76603 | NM_012861 | O-6-methylguanine-DNA | |
| | | | methyltransferase | |
| 462 | 82775 | NM_017277 | adaptor protein complex AP-1, beta | Enthoprotin interactions at the trans-Golgi |
| | | | 1 subunit | network |
| 463 | 82689 | NM_031007 | adenylate cyclase 2 | (Calcium signaling pathway, Opiate-Induced |
| | | | | Signal Transduction, Purine metabolism) |
| 464 | 76605 | NM_017256 | transforming growth factor, beta | (ALK in cardiac myocytes, CTCF: First Multivalent |
| | | | receptor 3 | Nuclear Factor, Role of Tob in T-cell activation) |
| 465 | 78354 | M81183 | | |
| 466 | 83033 | NM_013082 | syndecan 2 | |
| 467 | 76606 | NM_206849 | retinoid X receptor beta | |
| 468 | 82099 | NM_053997 | potassium voltage gated channel, | |
| | | | Shaw-related subfamily, member 3 | |
| 469 | 80423 | NM_023981 | colony stimulating factor 1 | (Cytokine-cytokine receptor interaction, Cytokines |
| | | | (macrophage) | and Inflammatory Response, Low-density |
| | | | | lipoprotein (LDL) pathway during atherogenesis, |
| | | | | METS affect on Macrophage Differentiation, |
| | | | | Msp/Ron Receptor Signaling Pathway, |
| | | | | Regulation of hematopoiesis by cytokines) |
| 470 | 78367 | NM_183325 | ADP-ribosylarginine hydrolase | |
| 471 | 76607 | NM_031970 | heat shock 27 kDa protein 1 | (Downregulated of MTA-3 in ER-negative Breast |
| | | | | Tumors, MAPK signaling pathway, Stress |
| | | | | Induction of HSP Regulation, p38 MAPK |
| | | | | Signaling Pathway) |
| 472 | 79498 | M86514 | proline-rich protein | |
| 473 | 82762 | NM_021740 | prothymosin alpha | |
| 474 | 76864 | NM_012883 | sulfotransferase, estrogen preferring | Androgen and estrogen metabolism |
| 475 | 80222 | NM_012560 | forkhead box G1 | |
| 476 | 78372 | M87786 | | |
| 477 | 79425 | NM_053996 | solute carrier family 6 | |
| | | | (neurotransmitter transporter, L- | |
| | | | proline), member 7 | |
| 478 | 78375 | M88488 | | |
| 479 | 78376 | NM_031112 | ribosomal protein S24 | Ribosome |
| 480 | 78382 | NM_012852 | 5-hydroxytryptamine (serotonin) | Neuroactive ligand-receptor interaction |
| | | | receptor 1D | |
| 481 | 79424 | M91234 | | |
| 482 | 82415 | NM_017161 | adenosine A2B receptor | (Calcium signaling pathway, Neuroactive ligand- |
| | | | | receptor interaction) |
| 483 | 78390 | NM_013122 | insulin-like growth factor binding | |
| | | | protein 2 | |
| 484 | 81988 | NM_017144 | troponin I, cardiac | |
| 485 | 78397 | NM_012531 | catechol-O-methyltransferase | Tyrosine metabolism |
| 486 | 81862 | NM_022869 | nucleolar and coiled-body | |
| | | | phosphoprotein 1 | |
| 487 | 76849 | NM_019623 | cytochrome P450, family 4, | (Fatty acid metabolism, Prostaglandin and |
| | | | subfamily F, polypeptide 2 | leukotriene metabolism, Tryptophan metabolism) |
| 488 | 78404 | M95791 | | |
| 489 | 82552 | NM_052798 | zinc finger protein 354A | |
| 490 | 78410 | NM_138876 | adenosine monophosphate | Purine metabolism |
| | | | deaminase 1 (isoform M) | |
| 491 | 78411 | M98826 | | |
| 492 | 78412 | NM_031573 | phosphorylase kinase gamma 1 | Calcium signaling pathway |
| 493 | 78414 | NM_012918 | calcium channel, voltage-dependent, | Calcium signaling pathway |
| | | | P/Q type, alpha 1A subunit | |
| 494 | 83628 | AA684919 | | |
| 495 | 84403 | AA799323 | pleckstrin (predicted) | |
| 496 | 84404 | AA799328 | | |
| 497 | 84405 | NM_001007634 | pelota homolog | |
| 498 | 84416 | NM_001008511 | estrogen related receptor, alpha | Regulation of PGC-1a |
| 499 | 84420 | AA799427 | similar to RIKEN cDNA 1110018J18 | |
| | | | (predicted) | |
| 500 | 84426 | AA799442 | hypothetical LOC293114 (predicted) | |
| 501 | 84440 | AA799471 | | |
| 502 | 84445 | NM_022700 | ADP-ribosylation factor-like 3 | |
| 503 | 76270 | AA799479 | NADH dehydrogenase (ubiquinone) | (Oxidative phosphorylation, Ubiquinone |
| | | | Fe—S protein 8 (predicted) | biosynthesis) |
| 504 | 84446 | AA799480 | | |
| 505 | 84450 | AA799488 | similar to D330021B20 protein | |
| | | | (predicted) | |
| 506 | 84453 | AA799495 | | |
| 507 | 81065 | NM_031545 | natriuretic peptide precursor type B | |
| 508 | 84474 | AA799534 | | |
| 509 | 84482 | AA799544 | ladinin (predicted) | |
| 510 | 84488 | AA799554 | | |
| 511 | 84491 | AA799566 | incisor protein | |
| 512 | 84521 | AA799637 | LIM and senescent cell antigen-like | |
| | | | domains 1 (predicted) | |
| 513 | 84523 | NM_001008312 | tumor differentially expressed 1 | |
| | | | (predicted) | |
| 513 | 84524 | NM_001008312 | tumor differentially expressed 1 | |
| | | | (predicted) | |
| 514 | 84529 | AA799656 | mitochondrial ribosomal protein S31 | |
| | | | (predicted) | |
| 514 | 84530 | AA799656 | mitochondrial ribosomal protein S31 | |
| | | | (predicted) | |
| 515 | 84556 | AA799724 | similar to RNA polymerase 1-3 | |
| 290 | 84558 | NM_017031 | phosphodiesterase 4B | Purine metabolism |
| 290 | 84559 | NM_017031 | phosphodiesterase 4B | Purine metabolism |
| 516 | 84576 | AA799766 | similar to Jtv1-pending protein | |
| 517 | 84577 | AA799771 | | |
| 518 | 84578 | AA799773 | filamin C, gamma (actin binding | |
| | | | protein 280) (predicted) | |
| 518 | 84579 | AA799773 | filamin C, gamma (actin binding | |
| | | | protein 280) (predicted) | |
| 519 | 84585 | NM_184049 | serine/threonine kinase 25 (STE20 | |
| | | | homolog, yeast) | |
| 520 | 84593 | AA799812 | | |
| 521 | 84603 | AA799847 | | |
| 522 | 84613 | NM_031331 | proteasome (prosome, macropain) | Proteasome |
| | | | 26S subunit, non-ATPase, 4 | |
| 375 | 84619 | NM_017248 | heterogeneous nuclear | |
| | | | ribonucleoprotein A1 | |
| 523 | 84630 | AA799992 | similar to C11orf17 protein | |
| | | | (predicted) | |
| 524 | 84638 | AA800015 | TAF10 RNA polymerase II, TATA | |
| | | | box binding protein (TBP)- | |
| | | | associated factor (predicted) | |
| 525 | 84652 | NM_001007618 | ring finger and CHY zinc finger | |
| | | | domain containing 1 | |
| 526 | 84663 | AA800175 | protein (peptidyl-prolyl cis/trans | How Progesterone Initiates the Oocyte |
| | | | isomerase) NIMA-interacting 1 | Maturation |
| | | | (predicted) | |
| 527 | 84685 | AA800222 | autocrine motility factor receptor | Ubiquitin mediated proteolysis |
| | | | (predicted) | |
| 528 | 84696 | AA800272 | mitochondrial ribosomal protein L3 | |
| | | | (predicted) | |
| 529 | 84719 | NM_022934 | DnaJ-like protein | |
| 530 | 84723 | AA800572 | sprouty homolog 1 (Drosophila) | Sprouty regulation of tyrosine kinase signals |
| | | | (predicted) | |
| 531 | 84738 | AA800651 | protein phosphatase 2, regulatory | |
| | | | subunit B (B56), alpha isoform | |
| | | | (predicted) | |
| 532 | 84740 | NM_023955 | secretory carrier membrane protein 2 | |
| 533 | 84745 | AA800678 | similar to AW046014 protein | Toll-like receptor signaling pathway |
| 534 | 84747 | AA800680 | Notch-regulated ankyrin repeat | |
| | | | protein (predicted) | |
| 535 | 84749 | AA800686 | | |
| 536 | 84761 | AA800719 | | |
| 537 | 84773 | AA800750 | | |
| 538 | 84782 | NM_031327 | cysteine rich protein 61 | |
| 344 | 84783 | NM_019185 | GATA binding protein 6 | |
| 539 | 84799 | AA800849 | | |
| 540 | 84804 | NM_001002830 | RAS-like family 11 member B | |
| 541 | 84816 | NM_001001504 | general transcription factor II I repeat | |
| | | | domain-containing 1 | |
| 542 | 81086 | NM_032057 | Inositol (myo)-1(or 4)- | (Inositol phosphate metabolism, |
| | | | monophosphatase 1 | Phosphatidylinositol signaling system) |
| 543 | 81489 | NM_053995 | 3-hydroxybutyrate dehydrogenase | (Butanoate metabolism, Synthesis and |
| | | | (heart, mitochondrial) | degradation of ketone bodies) |
| 544 | 81490 | NM_022511 | profilin 1 | (Erk and PI-3 Kinase Are Necessary for Collagen |
| | | | | Binding in Corneal Epithelia, Listeria-Induced |
| | | | | Signal Transduction, Regulation of actin |
| | | | | cytoskeleton, Rho cell motility signaling pathway) |
| 545 | 81511 | AA818025 | | |
| 546 | 81524 | NM_017355 | RAB4B, member RAS oncogene | |
| | | | family | |
| 547 | 81534 | NM_017096 | C-reactive protein, petaxin related | |
| 14 | 76160 | NM_212504 | heat shock 70 kD protein 1B | MAPK signaling pathway |
| 548 | 81600 | NM_017333 | endothelin receptor type B | (Calcium signaling pathway, Neuroactive ligand- |
| | | | | receptor interaction, Role of EGF Receptor |
| | | | | Transactivation by GPCRs in Cardiac |
| | | | | Hypertrophy) |
| 549 | 81601 | NM_012887 | thymopoietin | |
| 549 | 81602 | NM_012887 | thymopoietin | |
| 550 | 81090 | NM_017090 | guanylate cyclase 1, soluble, alpha 3 | Purine metabolism |
| 551 | 81092 | NM_053330 | ribosomal protein L21 | Ribosome |
| 552 | 81098 | NM_053891 | cyclin-dependent kinase 5, | (Deregulation of CDK5 in Alzheimers Disease, |
| | | | regulatory subunit 1 (p35) | Lissencephaly gene (LIS1) in neuronal migration |
| | | | | and development, Phosphorylation of MEK1 by |
| | | | | cdk5/p35 down regulates the MAP kinase |
| | | | | pathway, Rac 1 cell motility signaling pathway, |
| | | | | Reelin Signaling Pathway, Regulation of ck1/cdk5 |
| | | | | by type 1 glutamate receptors) |
| 553 | 81100 | NM_012857 | lysosomal membrane glycoprotein 1 | |
| 554 | 76272 | AA851403 | NADH dehydrogenase (ubiquinone) | (Oxidative phosphorylation, Ubiquinone |
| | | | 1 beta subcomplex 8 (predicted) | biosynthesis) |
| 554 | 76273 | AA851403 | NADH dehydrogenase (ubiquinone) | (Oxidative phosphorylation, Ubiquinone |
| | | | 1 beta subcomplex 8 (predicted) | biosynthesis) |
| 555 | 81797 | NM_012561 | follistatin | TGF-beta signaling pathway |
| 556 | 83046 | AA858586 | | |
| 557 | 83050 | AA858600 | leucine-zipper-like transcriptional | |
| | | | regulator, 1 (predicted) | |
| 558 | 83052 | AA858603 | similar to hypothetical protein | |
| | | | MGC20700 (predicted) | |
| 559 | 81795 | NM_173111 | tropomyosin isoform 6 | |
| 560 | 76323 | AA859372 | | |
| 561 | 83066 | NM_053555 | vesicle-associated membrane | |
| | | | protein 5 | |
| 562 | 83069 | AA859483 | | |
| 563 | 83082 | AA859533 | tubby like protein 1 (predicted) | Rhodopsin Processing and Transport |
| 564 | 83094 | NM_138518 | late gestation lung protein 1 | |
| 565 | 83099 | AA859597 | | |
| 566 | 77049 | AA859648 | DnaJ (Hsp40) homolog, subfamily B, | (Glucocorticoid Receptor-Hsp90 Heterocomplex, |
| | | | member 1 (predicted) | Rhodopsin Processing and Transport) |
| 567 | 83115 | AA859663 | similar to RIKEN cDNA 4933406J04 | |
| 568 | 83122 | AA859680 | similar to CG2662-PA (predicted) | |
| 569 | 83134 | AA859718 | similar to RIKEN cDNA A230063L24 | |
| | | | gene | |
| 570 | 83135 | AA859719 | mitochondrial ribosomal protein S14 | |
| | | | (predicted) | |
| 571 | 83138 | AA859740 | | |
| 572 | 83140 | AA859750 | | |
| 147 | 83143 | NM_134452 | collagen, type V, alpha 1 | |
| 573 | 83144 | AA859760 | | |
| 574 | 83150 | AA859804 | eukaryotic translation initiation factor | |
| | | | 4E like 3 (predicted) | |
| 575 | 83151 | NM_001012125 | lysyl oxidase-like 1 (predicted) | |
| 576 | 83152 | NM_024161 | cysteine string protein | |
| 577 | 83154 | AA859827 | uridine-cytidine kinase 2 | Pyrimidine metabolism |
| 578 | 81002 | NM_017087 | biglycan | Small Leucine-rich Proteoglycan (SLRP) |
| | | | | molecules |
| 579 | 83165 | NM_031978 | proteasome (prosome, macropain) | Proteasome |
| | | | 26S subunit, non-ATPase, 1 | |
| 580 | 83172 | NM_130426 | tumor necrosis factor receptor | (Acetylation and Deacetylation of RelA in The |
| | | | superfamily, member 1b | Nucleus, Butanoate metabolism, Chaperones |
| | | | | modulate interferon Signaling Pathway, Cytokine- |
| | | | | cytokine receptor interaction, Fatty acid |
| | | | | biosynthesis (path 2), Fatty acid metabolism, HIV- |
| | | | | I Nef: negative effector of Fas and TNF, |
| | | | | Keratinocyte Differentiation, Lysine degradation, |
| | | | | Mechanism of Gene Regulation by Peroxisome |
| | | | | Proliferators via PPARa(alpha), Multi-Drug |
| | | | | Resistance Factors, NE-kB Signaling Pathway, |
| | | | | Nuclear Receptors in Lipid Metabolism and |
| | | | | Toxicity, Propanoate metabolism, Regulation of |
| | | | | Inducible Nitric Oxide Synthase by Interleukin-17, |
| | | | | Regulation of Insulin and IGF Signaling by IRS |
| | | | | Proteins, Regulation of transcriptional activity by |
| | | | | PML, SODD/TNFR1 Signaling Pathway, TNFR2 |
| | | | | Signaling Pathway, Tryptophan metabolism, |
| | | | | Valine, leucine and isoleucine degradation, beta- |
| | | | | Alanine metabolism) |
| 581 | 83185 | AA859922 | similar to SRD5A2L (predicted) | |
| 582 | 83199 | NM_138839 | vacuole Membrane Protein 1 | |
| 583 | 83210 | AA859983 | cyclin M4 (predicted) | |
| 584 | 83214 | AA859994 | | |
| 585 | 83215 | AA859996 | | |
| 586 | 83222 | AA860024 | eukaryotic translation elongation | |
| | | | factor 1 gamma | |
| 38 | 83224 | NM_173102 | tubulin, beta 5 | |
| 587 | 83234 | NM_134326 | albumin | |
| 588 | 83235 | AA866221 | | |
| 587 | 83240 | NM_134326 | albumin | |
| 589 | 83250 | AA866272 | apoptosis, caspase activation | |
| | | | inhibitor (predicted) | |
| 590 | 83265 | AA866362 | zinc finger protein 580 (predicted) | |
| 591 | 83274 | NM_012651 | solute carrier family 4, member 1 | (Band 3-based macrocomplex in the erythrocyte |
| | | | | membrane, Membrane-cytoskeleton connections |
| | | | | in erythrocytes) |
| 592 | 83277 | AA866426 | | |
| 593 | 83281 | AA866439 | | |
| 594 | 83283 | AA866444 | Rho GTPase activating protein 10 | |
| | | | (predicted) | |
| 87 | 83287 | NM_031787 | homeodomain interacting protein | Phosphatidylinositol signaling system |
| | | | kinase 3 | |
| 595 | 83302 | AA874803 | | |
| 596 | 83305 | AA874815 | | |
| 597 | 83308 | AA874832 | anaphase-promoting complex | |
| | | | subunit 5 (predicted) | |
| 598 | 83322 | NM_017270 | alcohol dehydrogenase 4 (class II), | (Bile acid biosynthesis, Fatty acid metabolism, |
| | | | pi polypeptide | Glycerolipid metabolism, Glycolysis/ |
| | | | | Gluconeogenesis, Tyrosine metabolism) |
| 599 | 83346 | AA874952 | zinc finger protein 262 (predicted) | |
| 600 | 83354 | NM_001007703 | similar to BC002216 protein | |
| 601 | 83355 | AA874999 | Sec61 beta subunit (predicted) | |
| 602 | 83357 | AA875002 | leucine-rich repeat-containing 8 | |
| | | | (predicted) | |
| 603 | 83359 | NM_053589 | RAB14, member RAS oncogene | |
| | | | family | |
| 604 | 83360 | AA875017 | | |
| 605 | 83374 | AA875042 | LOC361774 (predicted) | |
| 606 | 83385 | NM_053295 | calpastatin | |
| 607 | 83388 | AA875098 | FK506 binding protein 11 (predicted) | |
| 608 | 83392 | NM_012866 | nuclear transcription factor-Y gamma | Overview of telomerase RNA component gene |
| | | | | hTerc Transcriptional Regulation |
| 609 | 83397 | AA875126 | | |
| 610 | 83413 | AA875165 | similar to Gamma-tubulin complex | |
| | | | component 4 (GCP-4) (hGCP4) | |
| | | | (h76p) (Hgrip76) | |
| 611 | 83415 | AA875171 | N-acetylglucosamine-1- | |
| | | | phosphotransferase, gamma subunit | |
| | | | (predicted) | |
| 611 | 83416 | AA875171 | N-acetylglucosamine-1- | |
| | | | phosphotransferase, gamma subunit | |
| | | | (predicted) | |
| 612 | 83418 | AA875182 | zinc finger protein 523 (predicted) | |
| 613 | 83423 | AA875197 | similar to 3300001A09Rik protein | |
| | | | (predicted) | |
| 614 | 83424 | NM_001008386 | notch1-induced protein | |
| 615 | 83428 | AA875205 | eukaryotic translation initiation factor | |
| | | | 3, subunit 9 (eta) (predicted) | |
| 616 | 83429 | NM_053747 | ubiquilin 1 | Gamma-aminobutyric Acid Receptor Life Cycle |
| 617 | 83443 | AA875255 | | |
| 618 | 83446 | AA875263 | microspherule protein 1 (predicted) | |
| 619 | 83450 | AA875275 | | |
| 620 | 76248 | NM_001006957 | eukaryotic translation initiation factor | |
| | | | 4H | |
| 621 | 83459 | AA875348 | | |
| 622 | 83463 | NM_001012091 | forkhead-like 18 (Drosophila) | |
| | | | (predicted) | |
| 623 | 83472 | AA875428 | similar to RIKEN cDNA 9130410M22 | |
| 624 | 83476 | AA875444 | dihydropyrimidinase-like 2 | (Pantothenate and CoA biosynthesis, Pyrimidine |
| | | | | metabolism, beta-Alanine metabolism) |
| 625 | 83480 | AA875468 | | |
| 626 | 83483 | NM_012903 | acidic (leucine-rich) nuclear | Granzyme A mediated Apoptosis Pathway |
| | | | phosphoprotein 32 family, member A | |
| 627 | 83486 | NM_053824 | casein kinase II, alpha 1 polypeptide | (Adherens junction, EGF Signaling Pathway, |
| | | | | EPO Signaling Pathway, IGF-1 Signaling |
| | | | | Pathway, IL 2 signaling pathway, IL 6 signaling |
| | | | | pathway, Insulin Signaling Pathway, |
| | | | | Lissencephaly gene (LIS1) in neuronal migration |
| | | | | and development, Nerve growth factor pathway |
| | | | | (NGF), PDGF Signaling Pathway, |
| | | | | Phosphatidylinositol signaling system, TPO |
| | | | | Signaling Pathway, Tight junction, WNT Signaling |
| | | | | Pathway, Wnt signaling pathway) |
| 628 | 83505 | AA875598 | cullin 2 (predicted) | |
| 629 | 83509 | NM_031083 | phosphatidylinositol 4-kinase, | (Inositol phosphate metabolism, |
| | | | catalytic, beta polypeptide | Phosphatidylinositol signaling system) |
| 630 | 83516 | AA875641 | | |
| 631 | 83522 | NM_001008694 | reticulocalbin 3, EF-hand calcium | |
| | | | binding domain | |
| 632 | 83635 | AA891032 | | |
| 52 | 81017 | NM_139091 | nucleoporin like 1 | Nuclear Pore Complex |
| 633 | 83642 | AA891058 | | |
| 634 | 83644 | AA891069 | serine/arginine-rich protein specific | |
| | | | kinase 2 (predicted) | |
| 635 | 83649 | AA891140 | similar to CDNA sequence | |
| | | | BC027246 (predicted) | |
| 635 | 83650 | AA891140 | similar to CDNA sequence | |
| | | | BC027246 (predicted) | |
| 636 | 83655 | AA891196 | similar to Butyrate-induced transcript 1 | |
| 637 | 83660 | NM_177928 | pre-B-cell colony enhancing factor 1 | Nicotinate and nicotinamide metabolism |
| 638 | 83661 | AA891221 | similar to RIKEN cDNA 0610007P06 | |
| 639 | 83671 | NM_031614 | thioredoxin reductase 1 | Pyrimidine metabolism |
| 640 | 83681 | AA891314 | poly(rC) binding protein 4 (predicted) | |
| 641 | 83684 | AA891356 | hypothetical LOC304650 (predicted) | |
| 642 | 83685 | NM_057186 | L-3-hydroxyacyl-Coenzyme A | (Butanoate metabolism, Fatty acid biosynthesis |
| | | | dehydrogenase, short chain | (path 2), Fatty acid metabolism, Lysine |
| | | | | degradation, Tryptophan metabolism, Valine, |
| | | | | leucine and isoleucine degradation) |
| 643 | 83688 | NM_080902 | hypoxia induced gene 1 | |
| 644 | 83689 | AA891423 | similar to hypothetical protein | |
| | | | FLJ12118 (predicted) | |
| 645 | 83698 | AA891476 | | |
| 646 | 83702 | NM_001008367 | similar to RIKEN cDNA 0610042E07 | |
| | | | (predicted) | |
| 647 | 83705 | AA891542 | DnaJ (Hsp40) homolog, subfamily B, | |
| | | | member 5 (predicted) | |
| 648 | 83708 | NM_001004283 | eukaryotic translation initiation factor | |
| | | | 3 subunit 7 | |
| 649 | 83716 | AA891588 | similar to hypothetical protein | |
| | | | E130310N06 | |
| 650 | 83718 | NM_031356 | programmed cell death 8 | (Apoptosis, Ceramide Signaling Pathway, |
| | | | | Opposing roles of AIF in Apoptosis and Cell |
| | | | | Survival, Role of Mitochondria in Apoptotic |
| | | | | Signaling) |
| 651 | 83720 | AA891596 | | |
| 652 | 83722 | NM_139332 | two pore channel 1 | |
| 653 | 83723 | AA891631 | | |
| 654 | 83729 | NM_001007804 | similar to EAP30 subunit of ELL | |
| | | | complex (predicted) | |
| 655 | 83743 | NM_173340 | ribosomal protein L13A | Ribosome |
| 656 | 83747 | AA891719 | ectonucleotide | |
| | | | pyrophosphatase/phosphodiesterase | |
| | | | 6 (predicted) | |
| 657 | 83748 | NM_001009647 | mitochondrial ribosomal protein L16 | |
| 658 | 83749 | NM_198765 | bicaudal D homolog 2 (Drosophila) | |
| 659 | 83752 | AA891727 | similar to RIKEN cDNA 2900010M23 | |
| | | | (predicted) | |
| 660 | 83757 | AA891737 | | |
| 661 | 83761 | AA891746 | endothelial differentiation-related | |
| | | | factor 1 (predicted) | |
| 662 | 81025 | NM_030827 | low density lipoprotein receptor- | |
| | | | related protein 2 | |
| 663 | 83781 | AA891810 | | |
| 663 | 83782 | AA891810 | | |
| 664 | 83797 | AA891838 | similar to ribosomal protein P0-like | |
| | | | protein; 60S acidic ribosomal protein | |
| | | | PO; ribosomal protein, large, P0-like | |
| | | | (predicted) | |
| 665 | 83800 | AA891842 | tumor necrosis factor receptor | |
| | | | superfamily, member 21 (predicted) | |
| 666 | 83803 | AA891851 | | |
| 667 | 83813 | AA891877 | | |
| 668 | 83815 | NM_022948 | sideroflexin 3 | |
| 669 | 83822 | AA891903 | | |
| 670 | 83824 | NM_001005383 | aminoacylase 1 | Urea cycle and metabolism of amino groups |
| 671 | 83825 | NM_032615 | membrane interacting protein of | |
| | | | RGS16 | |
| 671 | 83826 | NM_032615 | membrane interacting protein of | |
| | | | RGS16 | |
| 672 | 83827 | AA891917 | capping protein (actin filament) | (Listeria-Induced Signal Transduction, |
| | | | muscle Z-line, alpha 1 (predicted) | Microtubule-Organelle Interactions) |
| 673 | 83838 | AA891940 | similar to mKIAA1631 protein | |
| 674 | 83839 | AA891943 | | |
| 675 | 83846 | AA891969 | similar to small unique nuclear | Translin in Cell Signaling |
| | | | receptor co-repressor | |
| 676 | 83851 | AA892006 | ATPase, H+ transporting, V1 subunit | (ATP synthesis, Oxidative phosphorylation) |
| | | | A, isoform 1 (predicted) | |
| 677 | 83855 | NM_013177 | glutamate oxaloacetate | (Alanine and aspartate metabolism, Arginine and |
| | | | transaminase 2 | proline metabolism, Cysteine metabolism, |
| | | | | Glutamate metabolism, Phenylalanine |
| | | | | metabolism, Phenylalanine, tyrosine and |
| | | | | tryptophan biosynthesis, Tyrosine metabolism) |
| 678 | 83857 | NM_139101 | potassium channel regulator 1 | (Acetylation and Deacetylation of RelA in The |
| | | | | Nucleus, Adherens junction, BTG family proteins |
| | | | | and cell cycle regulation, CARM1 and Regulation |
| | | | | of the Estrogen Receptor, Calcium signaling |
| | | | | pathway, Cell Cycle: G1/S Check Point, Cell |
| | | | | cycle, Chaperones modulate interferon Signaling |
| | | | | Pathway, Cyclins and Cell Cycle Regulation, |
| | | | | Cytokine-cytokine receptor interaction, DNA |
| | | | | polymerase, ECM-receptor interaction, Focal |
| | | | | adhesion, Fructose and mannose metabolism, |
| | | | | Glycerophospholipid metabolism, HIV-I Nef: |
| | | | | negative effector of Fas and TNF, Inactivation of |
| | | | | Gsk3 by AKT causes accumulation of b-catenin in |
| | | | | Alveolar Macrophages, Influence of Ras and Rho |
| | | | | proteins on G1 to S Transition, Jak-STAT |
| | | | | signaling pathway, Keratinocyte Differentiation, |
| | | | | MAPK signaling pathway, Membrane- |
| | | | | cytoskeleton connections in erythrocytes, |
| | | | | Migration Inhibitory Factor (MIF) in Signalling and |
| | | | | Cell Cycle Progression, N-Glycan biosynthesis, |
| | | | | NF-kB Signaling Pathway, Neuroactive ligand- |
| | | | | receptor interaction, Oncogene Inactivation of |
| | | | | p21 Tumor Suppressor, Phosphatidylinositol |
| | | | | signaling system, Regulation of Inducible Nitric |
| | | | | Oxide Synthase by Interleukin-17, Regulation of |
| | | | | Insulin and IGF Signaling by IRS Proteins, |
| | | | | Regulation of actin cytoskeleton, Regulation of |
| | | | | transcriptional activity by PML, SODD/TNFR1 |
| | | | | Signaling Pathway, Signaling Network Regulating |
| | | | | Bone Morphogenesis, TGF-beta signaling |
| | | | | pathway, TNFR2 Signaling Pathway, Tight |
| | | | | junction, WNT Signaling Pathway, Wnt signaling |
| | | | | pathway, p53 Signaling Pathway) |
| 679 | 83861 | AA892042 | DEAD (Asp-Glu-Ala-Asp) box | |
| | | | polypeptide 3, X-linked | |
| 680 | 83862 | AA892049 | histone 1, H4j (predicted) | |
| 681 | 83871 | AA892112 | proline dehydrogenase (oxidase) 2 | |
| | | | (predicted) | |
| 682 | 83886 | AA892204 | | |
| 683 | 83888 | AA892228 | CUG triplet repeat, RNA binding | |
| | | | protein 1 (predicted) | |
| 684 | 76221 | AA892248 | | |
| 684 | 76222 | AA892248 | | |
| 685 | 83895 | NM_053524 | NADPH oxidase 4 | |
| 686 | 83912 | NM_175707 | peptidylprolyl isomerase | |
| | | | (cyclophilin)-like 3 | |
| 687 | 83917 | NM_001009666 | dynein, axonmal, light chain 4 | |
| | | | (predicted) | |
| 688 | 83919 | NM_001007707 | brain protein 16 | |
| 689 | 83922 | AA892310 | adaptor-related protein complex AP- | Enthoprotin interactions at the trans-Golgi |
| | | | 1, mu subunit 1 (predicted) | network |
| 690 | 83928 | AA892318 | ADP-ribosylation factor-like 6 | |
| | | | interacting protein 4 (predicted) | |
| 691 | 83947 | AA892378 | tetratricopeptide repeat domain 11 | |
| | | | (predicted) | |
| 692 | 83948 | AA892380 | serine palmitoyltransferase, long | |
| | | | chain base subunit 1 (predicted) | |
| 693 | 83958 | NM_198736 | potassium channel tetramerisation | |
| | | | domain containing 13 | |
| 694 | 83965 | AA892462 | | |
| 695 | 83969 | AA892470 | H2A histone family, member V | |
| | | | (predicted) | |
| 696 | 83977 | NM_001005547 | transmembrane 4 superfamily | |
| | | | member 8 | |
| 697 | 83985 | AA892520 | vesicle amine transport protein 1 | |
| | | | homolog (T californica) (predicted) | |
| 697 | 83986 | AA892520 | vesicle amine transport protein 1 | |
| | | | homolog (T californica) (predicted) | |
| 698 | 83988 | AA892526 | | |
| 699 | 76243 | NM_001004442 | thioredoxin domain containing 7 | (ATP synthesis, Emerin Assited Gene Regulation, |
| | | | | Oxidative phosphorylation) |
| 700 | 83999 | AA892551 | | |
| 701 | 84004 | NM_001007802 | chemokine-like factor super family 6 | |
| | | | (predicted) | |
| 702 | 84014 | AA892578 | similar to RIKEN cDNA 6330406I15 | |
| | | | (predicted) | |
| 703 | 84015 | AA892582 | | |
| 704 | 84016 | AA892593 | | |
| 705 | 84017 | NM_175580 | guanine nucleotide binding protein- | |
| | | | like 3 (nucleolar) | |
| 705 | 84018 | NM_175580 | guanine nucleotide binding protein- | |
| | | | like 3 (nucleolar) | |
| 706 | 84022 | AA892630 | ADP-ribosyltransferase (NAD+, | |
| | | | poly(ADP-ribose) polymerase)-like 2 | |
| | | | (predicted) | |
| 707 | 84025 | AA892635 | | |
| 708 | 84031 | AA892647 | histone 1, H4h (predicted) | |
| 709 | 81037 | NM_012771 | lysozyme | |
| 710 | 84052 | AA892779 | similar to RIKEN cDNA 3110052N05 | |
| | | | (predicted) | |
| 711 | 84069 | AA892820 | filamin, beta (predicted) | |
| 712 | 84072 | NM_001007620 | pyruvate dehydrogenase (lipoamide) | (Butanoate metabolism, Glycolysis/ |
| | | | beta | Gluconeogenesis, Pyruvate metabolism) |
| 713 | 84076 | NM_134382 | ELOVL family member 5, elongation | |
| | | | of long chain fatty acids (yeast) | |
| 714 | 84079 | NM_001007637 | mitochondrial ribosomal protein L24 | |
| 715 | 84083 | NM_001008521 | protein tyrosine kinase 9 (predicted) | |
| 716 | 84085 | NM_001009690 | rod outer segment membrane | |
| | | | protein 1 (predicted) | |
| 717 | 84089 | AA892860 | Son of sevenless homolog 1 | (Angiotensin II mediated activation of JNK |
| | | | (Drosophila) (predicted) | Pathway via Pyk2 dependent signaling, BCR |
| | | | | Signaling Pathway, Bioactive Peptide Induced |
| | | | | Signaling Pathway, Calcium Signaling by HBx of |
| | | | | Hepatitis B virus, EGF Signaling Pathway, EPO |
| | | | | Signaling Pathway, Erk1/Erk2 Mapk Signaling |
| | | | | pathway, Fc Epsilon Receptor I Signaling in Mast |
| | | | | Cells, Growth Hormone Signaling Pathway, IGF-1 |
| | | | | Signaling Pathway, IL 2 signaling pathway, IL 3 |
| | | | | signaling pathway, IL 6 signaling pathway, IL-2 |
| | | | | Receptor Beta Chain in T cell Activation, |
| | | | | Inhibition of Cellular Proliferation by Gleevec, |
| | | | | Insulin Signaling Pathway, Integrin Signaling |
| | | | | Pathway, Links between Pyk2 and Map Kinases, |
| | | | | Multiple antiapoptotic pathways from IGF-1R |
| | | | | signaling lead to BAD phosphorylation, Nerve |
| | | | | growth factor pathway (NGF), PDGF Signaling |
| | | | | Pathway, PTEN dependent cell cycle arrest and |
| | | | | apoptosis, Role of ERBB2 in Signal Transduction |
| | | | | and Oncology, Signaling of Hepatocyte Growth |
| | | | | Factor Receptor, Sprouty regulation of tyrosine |
| | | | | kinase signals, T Cell Receptor Signaling |
| | | | | Pathway, TPO Signaling Pathway, Transcription |
| | | | | factor CREB and its extracellular signals, Trefoil |
| | | | | Factors Initiate Mucosal Healing, Trka Receptor |
| | | | | Signaling Pathway) |
| 718 | 84090 | AA892861 | similar to RIKEN cDNA 2610528J11 | |
| | | | (predicted) | |
| 719 | 84106 | AA892940 | similar to 116 kDa U5 small nuclear | |
| | | | ribonucleoprotein component (U5 | |
| | | | snRNP-specific protein, 116 kDa) | |
| | | | (U5-116 kDa) | |
| 720 | 84115 | AA892987 | TATA box binding protein (Tbp)- | |
| | | | associated factor, RNA polymerase | |
| | | | I, C (predicted) | |
| 721 | 84118 | AA892997 | | |
| 722 | 84128 | NM_134330 | kidney expressed gene 1 | |
| 723 | 84132 | AA893043 | | |
| 724 | 84153 | AA893172 | | |
| 725 | 84158 | AA893184 | similar to Ac1164 | |
| 726 | 84181 | AA893237 | hypothetical LOC300441 | |
| 727 | 84182 | NM_053493 | 2-hydroxyphytanoyl-Coenzyme A | |
| | | | lyase | |
| 728 | 84183 | NM_012820 | acyl-CoA synthetase long-chain | Fatty acid metabolism |
| | | | family member 1 | |
| 728 | 84184 | NM_012820 | acyl-CoA synthetase long-chain | Fatty acid metabolism |
| | | | family member 1 | |
| 729 | 84185 | AA893244 | 3′-phosphoadenosine 5′- | (Purine metabolism, Selenoamino acid |
| | | | phosphosulfate synthase 2 | metabolism) |
| | | | (predicted) | |
| 730 | 84186 | NM_199386 | ATPase, H+ transporting, lysosomal | (ATP synthesis, Oxidative phosphorylation) |
| | | | 34 kDa, V1 subunit D | |
| 731 | 84192 | NM_001007144 | adipose differentiation-related | |
| | | | protein | |
| 340 | 76217 | NM_172008 | calnexin | |
| 732 | 84216 | AA893493 | | |
| 626 | 84218 | NM_012903 | acidic (leucine-rich) nuclear | Granzyme A mediated Apoptosis Pathway |
| | | | phosphoprotein 32 family, member A | |
| 733 | 84224 | AA893534 | | |
| 734 | 84230 | AA893584 | biogenesis of lysosome-related | |
| | | | organelles complex-1, subunit 2 | |
| | | | (predicted) | |
| 735 | 84232 | AA893592 | reticulocalbin (predicted) | |
| 736 | 84250 | AA893659 | similar to RIKEN cDNA 4933433P14 | |
| | | | gene (predicted) | |
| 737 | 84279 | NM_139104 | EGF-like domain 7 | |
| 738 | 84281 | AA893781 | centaurin, beta 1 (predicted) | |
| 739 | 84287 | NM_134398 | p34 protein | |
| 740 | 84291 | AA893857 | | |
| 741 | 84313 | AA893984 | cytoplasmic FMR1 interacting | Regulation of actin cytoskeleton |
| | | | protein 2 (predicted) | |
| 742 | 84316 | AA894004 | capping protein (actin filament), | |
| | | | gelsolin-like (predicted) | |
| 743 | 84317 | AA894008 | similar to erythrocyte membrane | (CASK Protein Interactions, Enthoprotin |
| | | | protein band 4.1 (elliptocytosis 1, | interactions at the trans-Golgi network, |
| | | | RH-linked) isoform 1 | Membrane-cytoskeleton connections in |
| | | | | erythrocytes, Tight junction) |
| 744 | 84320 | AA894016 | RNA-binding region (RNP1, RRM) | |
| | | | containing 2 (predicted) | |
| 745 | 84322 | PA894027 | | |
| 746 | 84326 | NM_181822 | natural cytotoxicity triggering | |
| | | | receptor 3 | |
| 747 | 84338 | AA894092 | periostin, osteoblast specific factor | |
| | | | (predicted) | |
| 748 | 81056 | NM_001009668 | electron transferring flavoprotein, | |
| | | | alpha polypeptide | |
| 749 | 84360 | AA894200 | myosin, light polypeptide 6, alkali, | Focal adhesion |
| | | | smooth muscle and non-muscle | |
| | | | (predicted) | |
| 750 | 84374 | AA894259 | similar to RIKEN cDNA 2010110M21 | |
| 751 | 84380 | AA894292 | adenylate cyclase 7 | Purine metabolism |
| 328 | 84381 | NM_017033 | phosphoglucomutase 1 | (Galactose metabolism, Glycolysis/ |
| | | | | Gluconeogenesis, Pentose phosphate pathway, |
| | | | | Starch and sucrose metabolism) |
| 752 | 84383 | AA894304 | | |
| 753 | 81059 | NM_032083 | chimerin (chimaerin) 1 | |
| 754 | 84387 | AA894318 | similar to RIKEN cDNA B230312A22 | |
| | | | (predicted) | |
| 755 | 84393 | AA894345 | phosphoprotein enriched in | |
| | | | astrocytes 15 (predicted) | |
| 756 | 81110 | NM_022267 | cyclin D2 | |
| 580 | 77050 | NM_130426 | tumor necrosis factor receptor | (Acetylation and Deacetylation of RelA in The |
| | | | superfamily, member 1b | Nucleus, Chaperones modulate interferon |
| | | | | Signaling Pathway, Cytokine-cytokine receptor |
| | | | | interaction, HIV-I Nef: negative effector of Fas |
| | | | | and TNF, Keratinocyte Differentiation, NF-kB |
| | | | | Signaling Pathway, Regulation of Inducible Nitric |
| | | | | Oxide Synthase by Interleukin-17, Regulation of |
| | | | | Insulin and IGF Signaling by IRS Proteins, |
| | | | | Regulation of transcriptional activity by PML, |
| | | | | SODD/TNFR1 Signaling Pathway, TNFR2 |
| | | | | Signaling Pathway) |
| 757 | 81116 | NM_019147 | jagged 1 | (Notch signaling pathway, Phagocyte superoxide- |
| | | | | generating NADPH oxidase, Phosphoinositides |
| | | | | and their downstream targets.) |
| 758 | 81126 | NM_032067 | ralA binding protein 1 | (Rac 1 cell motility signaling pathway, Ras |
| | | | | Signaling Pathway) |
| 759 | 77051 | AA925300 | mitogen activated protein kinase | (MAPKinase Signaling Pathway, |
| | | | kinase kinase 3 (predicted) | Phosphatidylinositol signaling system) |
| 760 | 81191 | NM_031561 | cd36 antigen | ECM-receptor interaction |
| 761 | 77054 | AA926129 | schlafen 2 (predicted) | |
| 762 | 76254 | NM_012520 | catalase | (Amyotrophic lateral sclerosis (ALS), Methane |
| | | | | metabolism, The IGF-1 Receptor and Longevity, |
| | | | | Tryptophan metabolism) |
| 440 | 81141 | NM_017259 | B-cell translocation gene 2, anti- | |
| | | | proliferative | |
| 763 | 81152 | NM_012681 | transthyretin | |
| 764 | 81154 | AA945171 | apolipoprotein C-IV | |
| 587 | 81155 | NM_134326 | albumin | |
| 381 | 76117 | NM_017158 | cytochrome P450, family 2, | (Fatty acid metabolism, Tryptophan metabolism) |
| | | | subfamily c, polypeptide 7 | |
| 765 | 76346 | AA945704 | DnaJ (Hsp40) homolog, subfamily B, | (Glucocorticoid Receptor-Hsp90 Heterocomplex, |
| | | | member 1 (predicted) | Rhodopsin Processing and Transport) |
| 766 | 81162 | NM_022205 | chemokine (C—X—C motif) receptor 4 | (CXCR4 Signaling Pathway, Cytokine-cytokine |
| | | | | receptor interaction, Ephrins and Ephs: forward |
| | | | | and reverse signalling, HIV-1 defeats host- |
| | | | | mediated resistance by CEM15, Pertussis toxin- |
| | | | | insensitive CCR5 Signaling in Macrophage, |
| | | | | Selective expression of chemokine receptors |
| | | | | during T-cell polarization, The Role of Slit-Robo |
| | | | | Pathway in Axon Guidance, The Role of the |
| | | | | Cxcr4 Chemokine Receptor and pVHL Tumor |
| | | | | Suppressor in Metastasis) |
| 767 | 76312 | AA946040 | | |
| 337 | 81165 | NM_030857 | Yamaguchi sarcoma viral (v-yes-1) | (BCR Signaling Pathway, Calcium signaling |
| | | | oncogene homolog | pathway, Eph Kinase and ephrins support |
| | | | | platelet aggregation, Fc Epsilon Receptor I |
| | | | | Signaling in Mast Cells, Phosphoinositides and |
| | | | | their downstream targets., Platelet thrombus |
| | | | | formation at injury sites, Ub/Proteosome System |
| | | | | in EBV-associated Malignancies) |
| 768 | 81166 | AA946108 | laminin, alpha 3 | (Agrin in Postsynaptic Differentiation, |
| | | | | Hemidesmosome Assembly, Matrix |
| | | | | Metalloproteinase 1 in Cell Signaling, |
| | | | | Neuromuscular Junction Protiens) |
| 769 | 81167 | NM_030829 | G protein-coupled receptor kinase 5 | (Inositol phosphate metabolism, Nicotinate and |
| | | | | nicotinamide metabolism, Phosphatidylinositol |
| | | | | signaling system) |
| 770 | 81172 | AA946439 | | |
| 261 | 81175 | NM_012804 | ATP-binding cassette, sub-family D | |
| | | | (ALD), member 3 | |
| 771 | 81226 | NM_017212 | microtubule-associated protein tau | (Alzheimer's disease, MAPK signaling pathway) |
| 772 | 81227 | NM_054006 | unr protein | |
| 773 | 76318 | NM_012941 | cytochrome P450, subfamily 51 | (Fatty acid metabolism, Tryptophan metabolism) |
| 774 | 81235 | NM_024150 | ADP-ribosylation factor 2 | |
| 775 | 81238 | NM_031534 | Wilms tumor 1 | (Chaperones modulate interferon Signaling |
| | | | | Pathway, Overview of telomerase protein |
| | | | | component gene hTert Transcriptional |
| | | | | Regulation, Regulation of Steroidogenesis by |
| | | | | Orphan NuclearReceptor SF-1) |
| 773 | 76319 | NM_012941 | cytochrome P450, subfamily 51 | (Fatty acid metabolism, Tryptophan metabolism) |
| 471 | 76283 | NM_031970 | heat shock 27 kDa protein 1 | (Downregulated of MTA-3 in ER-negative Breast |
| | | | | Tumors, MAPK signaling pathway, Stress |
| | | | | Induction of HSP Regulation, p38 MAPK |
| | | | | Signaling Pathway) |
| 776 | 76343 | NM_001004082 | heat shock 90 kDa protein 1, beta | |
| 777 | 81262 | AI007824 | | |
| 778 | 81263 | NM_053343 | double cortin and | Phosphatidylinositol signaling system |
| | | | calcium/calmodulin-dependent | |
| | | | protein kinase-like 1 | |
| 776 | 76344 | NM_001004082 | heat shock 90 kDa protein 1, beta | |
| 779 | 76186 | AI009141 | | |
| 780 | 76276 | AI009390 | NADH dehydrogenase (ubiquinone) | (Oxidative phosphorylation, Ubiquinone |
| | | | Fe—S protein 5b, 15 kDa (NADH- | biosynthesis) |
| | | | coenzyme Q reductase) (predicted) | |
| 781 | 81286 | NM_013216 | Ras homolog enriched in brain | |
| 782 | 81292 | NM_057114 | peroxiredoxin 1 | (Methane metabolism, Phenylalanine |
| | | | | metabolism, Prostaglandin and leukotriene |
| | | | | metabolism) |
| 783 | 81295 | NM_022519 | serine (or cysteine) proteinase | Complement and coagulation cascades |
| | | | inhibitor, clade A (alpha-1 | |
| | | | antiproteinase, antitrypsin), member 1 | |
| 784 | 76191 | AI011706 | splicing factor, arginine/serine-rich 3 | |
| | | | (SRp20) (predicted) | |
| 785 | 81309 | NM_017039 | protein phosphatase 2a, catalytic | (AKAP95 role in mitosis and chromosome |
| | | | subunit, alpha isoform | dynamics, AKT Signaling Pathway, Anion |
| | | | | Transport in Heart, Aurora B Kinases in |
| | | | | Regulation of Cell Division, CTCF: First |
| | | | | Multivalent Nuclear Factor, ChREBP regulation |
| | | | | by carbohydrates and cAMP, Constitutive |
| | | | | Androstane Receptor in Regulation of Xenobiotic |
| | | | | Metabolism, Deregulation of CDK5 in Alzheimers |
| | | | | Disease, Erk1/Erk2 Mapk Signaling pathway, |
| | | | | Inactivation of Gsk3 by AKT causes accumulation |
| | | | | of b-catenin in Alveolar Macrophages, Inhibition |
| | | | | of Protein Synthesis by AMP Kinase, |
| | | | | Keratinocyte Differentiation, Protein Kinase A at |
| | | | | the Centrosome, Regulation of ck1/cdk5 by type |
| | | | | 1 glutamate receptors, Skeletal muscle |
| | | | | hypertrophy is regulated via AKT/mTOR pathway, |
| | | | | TGF-beta signaling pathway, Telomeres, |
| | | | | Telomerase, Cellular Aging, and Immortality, |
| | | | | Tight junction, WNT Signaling Pathway, Wnt |
| | | | | signaling pathway, mTOR Signaling Pathway) |
| 300 | 81324 | NM_020075 | eukaryotic translation initiation factor 5 | (Eukaryotic protein translation, Regulation of |
| | | | | eIF2) |
| 786 | 81325 | NM_012521 | calbindin 3, (vitamin D-dependent | |
| | | | calcium binding protein) | |
| 787 | 81326 | NM_138905 | ER transmembrane protein Dri 42 | (Glycerolipid metabolism, Glycerophospholipid |
| | | | | metabolism, Glycosphingolipid metabolism) |
| 788 | 81329 | NM_022713 | transglutaminase 4 (prostate) | |
| 789 | 76170 | NM_024392 | hydroxysteroid (17-beta) | (Androgen and estrogen metabolism, Butanoate |
| | | | dehydrogenase 4 | metabolism, Fatty acid biosynthesis (path 2), |
| | | | | Fatty acid metabolism, Lysine degradation, |
| | | | | Mechanism of Gene Regulation by Peroxisome |
| | | | | Proliferators via PPARa(alpha), Tryptophan |
| | | | | metabolism, Valine, leucine and isoleucine |
| | | | | degradation) |
| 790 | 81318 | AI014135 | | |
| 791 | 81320 | NM_019242 | interferon-related developmental | |
| | | | regulator 1 | |
| 792 | 81333 | NM_012567 | gap junction membrane channel | |
| | | | protein alpha 1 | |
| 793 | 81334 | NM_053868 | neuroligin 1 | |
| 486 | 81337 | NM_022869 | nucleolar and coiled-body | |
| | | | phosphoprotein 1 | |
| 794 | 81342 | NM_172029 | eukaryotic translation initiation factor | VEGF, Hypoxia, and Angiogenesis |
| | | | 2B, subunit 1 alpha | |
| 233 | 81357 | NM_017126 | ferredoxin 1 | |
| 795 | 81359 | NM_017215 | solute carrier family 1 (glial high | Amyotrophic lateral sclerosis (ALS) |
| | | | affinity glutamate transporter), | |
| | | | member 2 | |
| 796 | 81361 | NM_153735 | neuronal pentraxin 1 | |
| 797 | 81362 | NM_133423 | splicing factor YT521-B | |
| 798 | 81366 | NM_019241 | gap junction membrane channel | |
| | | | protein beta 5 | |
| 799 | 81392 | NM_017030 | propionyl coenzyme A carboxylase, | (Propanoate metabolism, Valine, leucine and |
| | | | beta polypeptide | isoleucine degradation) |
| 360 | 76093 | NM_024127 | growth arrest and DNA-damage- | (ATM Signaling Pathway, Cell Cycle: G2/M |
| | | | inducible 45 alpha | Checkpoint, Cell cycle, Hypoxia and p53 in the |
| | | | | Cardiovascular system, MAPK signaling pathway, |
| | | | | p53 Signaling Pathway) |
| 360 | 76094 | NM_024127 | growth arrest and DNA-damage- | (ATM Signaling Pathway, Cell Cycle: G2/M |
| | | | inducible 45 alpha | Checkpoint, Cell cycle, Hypoxia and p53 in the |
| | | | | Cardiovascular system, MAPK signaling pathway, |
| | | | | p53 Signaling Pathway) |
| 772 | 81395 | NM_054006 | unr protein | |
| 800 | 81396 | NM_012817 | insulin-like growth factor binding | |
| | | | protein 5 | |
| 801 | 81398 | NM_017313 | RAB3A interacting protein | |
| 626 | 81401 | NM_012903 | acidic (leucine-rich) nuclear | Granzyme A mediated Apoptosis Pathway |
| | | | phosphoprotein 32 family, member A | |
| 802 | 81403 | NM_012774 | glypican 3 | |
| 803 | 81405 | NM_031135 | TGFB inducible early growth | |
| | | | response | |
| 804 | 81412 | AI071866 | | |
| 805 | 81426 | NM_019246 | proprotein convertase subtilisin/kexin | |
| | | | type 7 | |
| 806 | 81471 | AI101320 | jagged 2 | Notch signaling pathway |
| 807 | 81494 | AI102044 | | |
| 808 | 81498 | NM_024000 | vesicle-associated calmodulin- | |
| | | | binding protein | |
| 809 | 81513 | NM_053311 | ATPase, Ca++ transporting, plasma | Calcium signaling pathway |
| | | | membrane 1 | |
| 810 | 81517 | NM_001008888 | ubiquinol-cytochrome c reductase, | Oxidative phosphorylation |
| | | | Rieske iron-sulfur polypeptide 1 | |
| 811 | 81518 | NM_013087 | CD 81 antigen | |
| 812 | 76313 | AI104035 | | |
| 813 | 76204 | AI104077 | | |
| 814 | 81527 | NM_022922 | triosephosphate isomerase 1 | (Fructose and mannose metabolism, Glycolysis/ |
| | | | | Gluconeogenesis, Glycolysis Pathway, Inositol |
| | | | | metabolism) |
| 815 | 81529 | NM_145783 | cytochrome c oxidase, subunit Va | Oxidative phosphorylation |
| 47 | 81530 | NM_031330 | heterogeneous nuclear | |
| | | | ribonucleoprotein A/B | |
| 816 | 81536 | NM_017260 | arachidonate 5-lipoxygenase | |
| | | | activating protein | |
| 817 | 81539 | NM_017147 | cofilin 1 | Regulation of actin cytoskeleton |
| 818 | 81429 | NM_013113 | ATPase, Na+/K+ transporting, beta 1 | |
| | | | polypeptide | |
| 819 | 81436 | NM_172034 | farnesyltransferase, CAAX box, beta | |
| 820 | 76228 | AI136977 | FK506 binding protein 4 | Caveolin-Assisted Cholesterol Transport |
| 821 | 81447 | NM_013060 | Inhibitor of DNA binding 2, dominant | TGF-beta signaling pathway |
| | | | negative helix-loop-helix protein | |
| 266 | 76108 | NM_031576 | P450 (cytochrome) oxidoreductase | |
| 822 | 81451 | NM_012796 | glutathione S-transferase, theta 2 | Glutathione metabolism |
| 823 | 81459 | NM_053874 | CAP, adenylate cyclase-associated | |
| | | | protein, 2 (yeast) | |
| 824 | 81465 | NM_012716 | solute carrier family 16 | |
| | | | (monocarboxylic acid transporters), | |
| | | | member 1 | |
| 193 | 81481 | NM_031719 | chloride channel, nucleotide- | |
| | | | sensitive, 1A | |
| 825 | 76347 | AI169265 | | |
| 826 | 81483 | NM_053819 | tissue inhibitor of metalloproteinase 1 | Inhibition of Matrix Metalloproteinases 1 |
| 826 | 81484 | NM_053819 | tissue inhibitor of metalloproteinase 1 | Inhibition of Matrix Metalloproteinases |
| 827 | 81544 | NM_053365 | fatty acid binding protein 4, | |
| | | | adipocyte | |
| 828 | 81548 | AI169756 | | |
| 829 | 76133 | AI170379 | | |
| 830 | 81557 | NM_053591 | dipeptidase 1 (renal) | |
| 831 | 81559 | NM_031616 | zinc finger protein 265 | |
| 832 | 76288 | NM_032079 | DnaJ (Hsp40) homolog, subfamily A, | (Dystrobrevin Complex, Proteins in |
| | | | member 2 | Neuromuscular Disease, Stimulation of Synaptic |
| | | | | Vesicle Recycling by Nitrous Oxide) |
| 832 | 76289 | NM_032079 | DnaJ (Hsp40) homolog, subfamily A, | (Dystrobrevin Complex, Proteins in |
| | | | member 2 | Neuromuscular Disease, Stimulation of Synaptic |
| | | | | Vesicle Recycling by Nitrous Oxide) |
| 833 | 81563 | NM_053994 | pyruvate dehydrogenase E1 alpha 2 | (Butanoate metabolism, Glycolysis/ |
| | | | | Gluconeogenesis, Pyruvate metabolism) |
| 834 | 81565 | NM_012600 | malic enzyme 1 | (Mechanism of Gene Regulation by Peroxisome |
| | | | | Proliferators via PPARa(alpha), Pyruvate |
| | | | | metabolism, Shuttle for transfer of acetyl groups |
| | | | | from mitochondria to the cytosol) |
| 835 | 81577 | NM_139099 | ATP synthase, H+ transporting, | (ATP synthesis, Oxidative phosphorylation) |
| | | | mitochondrial F1 complex, epsilon | |
| | | | subunit | |
| 836 | 81584 | NM_019904 | lectin, galactose binding, soluble 1 | |
| 837 | 76357 | AI172452 | cytochrome c oxidase subunit VIIa | Oxidative phosphorylation |
| | | | polypeptide 2 like (predicted) | |
| 838 | 81595 | AI175486 | hypothetical LOC298661 | |
| 839 | 81606 | NM_173101 | myosin IE | |
| 840 | 81611 | NM_031137 | tripeptidyl peptidase II | |
| 841 | 81613 | AI176460 | | |
| 842 | 81614 | NM_017211 | golgi apparatus protein 1 | |
| 843 | 81620 | NM_013156 | cathepsin L | |
| 471 | 76285 | NM_031970 | heat shock 27 kDa protein 1 | (Downregulated of MTA-3 in ER-negative Breast |
| | | | | Tumors, MAPK signaling pathway, Stress |
| | | | | Induction of HSP Regulation, p38 MAPK |
| | | | | Signaling Pathway) |
| 844 | 81624 | NM_031628 | nuclear receptor subfamily 4, group | |
| | | | A, member 3 | |
| 845 | 76293 | AI176726 | | |
| 846 | 81629 | AI177052 | nucleoporin 153 kD | |
| 847 | 81632 | NM_031789 | nuclear factor, erythroid derived 2, | (Oxidative Stress Induced Gene Expression Via |
| | | | like 2 | Nrf2, Prion Pathway, Prion disease) |
| 847 | 81633 | NM_031789 | nuclear factor, erythroid derived 2, | (Oxidative Stress Induced Gene Expression Via |
| | | | like 2 | Nrf2, Prion Pathway, Prion disease) |
| 848 | 81637 | NM_053985 | H3 histone, family 3B | |
| 300 | 81643 | NM_020075 | eukaryotic translation initiation factor 5 | (Eukaryotic protein translation, Regulation of |
| | | | | eIF2) |
| 840 | 81644 | NM_031137 | tripeptidyl peptidase II | |
| 849 | 81654 | NM_031144 | actin, beta | (Adherens junction, Focal adhesion, Regulation |
| | | | | of actin cytoskeleton, SWI/SNF Activator |
| | | | | Complex, Tight junction). |
| 850 | 81660 | NM_012580 | heme oxygenase (decycling) 1 | (IL-10 Anti-inflammatory Signaling Pathway, |
| | | | | Porphyrin and chlorophyll metabolism) |
| 851 | 81663 | NM_033351 | Fc receptor, IgG, alpha chain | |
| | | | transporter | |
| 852 | 81668 | NM_020079 | prolactin-like protein C 1 | |
| 853 | 81671 | NM_013011 | tyrosine 3- | |
| | | | monooxygenase/tryptophan 5- | |
| | | | monooxygenase activation protein, | |
| | | | zeta polypeptide | |
| 854 | 81672 | NM_031840 | farensyl diphosphate synthase | Biosynthesis of steroids |
| 855 | 81678 | NM_013106 | guanine nucleotide binding protein, | Tight junction |
| | | | alpha inhibiting 3 | |
| 165 | 76114 | NM_138877 | diaphorase 1 | Aminosugars metabolism |
| 856 | 81696 | NM_031739 | potassium voltage gated channel, | |
| | | | Shal-related family, member 3 | |
| 857 | 81700 | NM_022538 | phosphatidic acid phosphatase 2a | (Glycerolipid metabolism, Glycerophospholipid |
| | | | | metabolism, Glycosphingolipid metabolism) |
| 858 | 81705 | NM_012999 | Subtilisin-like endoprotease | |
| 859 | 81706 | NM_053867 | tumor protein, translationally- | |
| | | | controlled 1 | |
| 860 | 81722 | AI231445 | | |
| 861 | 81726 | NM_022500 | ferritin light chain 1 | |
| 862 | 81729 | NM_021587 | latent transforming growth factor | |
| | | | beta binding protein 1 | |
| 863 | 81731 | NM_031672 | solute carrier family 15 (H+/peptide | |
| | | | transporter), member 2 | |
| 864 | 81732 | NM_032084 | chimerin (chimaerin) 2 | |
| 865 | 81733 | NM_030586 | cytochrome b5, outer mitochondrial | |
| | | | membrane isoform | |
| 866 | 81734 | AI232268 | low density lipoprotein receptor- | |
| | | | related protein associated protein 1 | |
| 867 | 81755 | AI234351 | similar to immunoglobulin light chain | |
| | | | variable region | |
| 868 | 81759 | AI234915 | WD repeat domain 5B (predicted) | |
| 869 | 81778 | NM_198132 | heterogeneous nuclear | |
| | | | ribonucleoprotein A3 | |
| 870 | 76224 | NM_001011901 | heat shock protein 105 (predicted) | |
| 410 | 81781 | NM_012978 | luteinizing | (Calcium signaling pathway, Neuroactive ligand- |
| | | | hormone/choriogonadotropin | receptor interaction) |
| | | | receptor | |
| 871 | 81784 | AI237535 | LPS-induced TNF-alpha factor | |
| 872 | 81786 | AI237592 | progestin induced protein | |
| 313 | 81788 | NM_012598 | lipoprotein lipase | (Alzheimer's disease, C-Reactive Protein, Apo-B |
| | | | | in Lipid Metabolism and Cardiovascular Disease, |
| | | | | Glycerolipid metabolism, Low-density lipoprotein |
| | | | | (LDL) pathway during atherogenesis, Mechanism |
| | | | | of Gene Regulation by Peroxisome Proliferators |
| | | | | via PPARa(alpha), Role of PPAR-gamma |
| | | | | Coactivators in Obesity and Thermogenesis, |
| | | | | Roles of Nuclear Receptors in the Regulation of |
| | | | | Bile Acid Metabolism and Cholesterol |
| | | | | Homeostasis, Visceral Fat Deposits and the |
| | | | | Metabolic Syndrome) |
| 873 | 81789 | NM_012533 | carboxypeptidase B1 | |
| 874 | 80438 | AI638939 | bobby sox homolog (Drosophila) | |
| | | | (predicted) | |
| 875 | 80439 | AI638940 | | |
| 876 | 80450 | AI638951 | | |
| 877 | 80451 | AI638952 | | |
| 878 | 80465 | NM_133605 | calcium/calmodulin-dependent | (Bioactive Peptide Induced Signaling Pathway, |
| | | | protein kinase II gamma | CD44-Mediated Signaling, Ca++/Calmodulin- |
| | | | | dependent Protein Kinase Activation, Calcium |
| | | | | signaling pathway, Meiotic Arrest in Oogenesis, |
| | | | | Regulation of PGC-1a, Stathmin and breast |
| | | | | cancer resistance to antimicrotubule agents, |
| | | | | Transcription factor CREB and its extracellular |
| | | | | signals, Wnt signaling pathway) |
| 879 | 80489 | AI638996 | | |
| 880 | 80497 | AI639007 | | |
| 881 | 80523 | AI639035 | | |
| 882 | 80545 | AI639058 | transmembrane, prostate androgen | |
| | | | induced RNA (predicted) | |
| 883 | 80546 | AI639060 | | |
| 884 | 80551 | AI639065 | | |
| 885 | 80556 | AI639071 | | |
| 886 | 80589 | AI639107 | ADP-ribosylation factor-like 11 | |
| | | | (predicted) | |
| 887 | 80596 | NM_198743 | BWK-1 | |
| 888 | 80610 | AI639132 | similar to RIKEN cDNA 6720467C03 | |
| | | | (predicted) | |
| 889 | 80629 | AI639152 | similar to Alpha-1-antitrypsin-related | |
| | | | protein precursor (predicted) | |
| 890 | 80651 | AI639175 | | |
| 891 | 80655 | AI639179 | | |
| 892 | 80656 | AI639181 | | |
| 893 | 80664 | AI639189 | similar to nuclear protein with a | |
| | | | coiled coil-4 domain of bilaterial | |
| | | | origin like (3L720) | |
| 894 | 80665 | AI639190 | | |
| 895 | 80667 | AI639192 | | |
| 896 | 80684 | AI639209 | | |
| 897 | 80685 | AI639210 | doublesex and mab-3 related | |
| | | | transcription factor like family A1 | |
| | | | (predicted) | |
| 898 | 80686 | NM_017298 | calcium channel, voltage-dependent, | Calcium signaling pathway |
| | | | L type, alpha 1D subunit | |
| 89 | 80689 | NM_131913 | X-prolyl aminopeptidase | |
| | | | (aminopeptidase P) 1, soluble | |
| 899 | 80691 | AI639217 | | |
| 900 | 80692 | NM_001009268 | ARP2 actin-related protein 2 | (How Progesterone Initiates the Oocyte |
| | | | homolog (yeast) (predicted) | Maturation, How does salmonella hijack a cell, |
| | | | | Listeria-Induced Signal Transduction, Rho cell |
| | | | | motility signaling pathway, Role of PI3K subunit |
| | | | | p85 in regulation of Actin Organization and Cell |
| | | | | Migration, Role of syndapins in vesicle trafficking, |
| | | | | Y branching of actin filaments) |
| 901 | 80715 | AI639246 | extra cellular link domain-containing | |
| | | | 1 (predicted) | |
| 902 | 80723 | AI639252 | | |
| 903 | 80728 | NM_181376 | spermatogenesis associated, serine- | |
| | | | rich 1 | |
| 904 | 80733 | AI639263 | | |
| 905 | 80734 | AI639264 | | |
| 906 | 80755 | AI639293 | | |
| 907 | 80788 | AI639324 | F-box protein 38 (predicted) | |
| 908 | 80792 | AI639328 | | |
| 909 | 80793 | AI639329 | WD repeat domain 37 (predicted) | |
| 910 | 80805 | AI639343 | | |
| 911 | 80831 | NM_001007725 | intercellular adhesion molecule 2 | |
| 912 | 80857 | AI639400 | | |
| 913 | 80861 | AI639404 | | |
| 914 | 80862 | NM_138831 | solute carrier family 16 | |
| | | | (monocarboxylic acid transporters), | |
| | | | member 10 | |
| 915 | 80868 | NM_207614 | P55 | |
| 916 | 80870 | AI639412 | similar to asporin precursor | |
| 917 | 80888 | AI639432 | | |
| 918 | 80897 | NM_133609 | eukaryotic translation initiation factor | VEGF, Hypoxia, and Angiogenesis |
| | | | 2B, subunit 3 gamma | |
| 919 | 80901 | NM_057191 | kelch repeat and BTB (POZ) domain | |
| | | | containing 10 | |
| 920 | 80920 | AI639461 | | |
| 921 | 80927 | AI639467 | synaptotagmin 1 | |
| 922 | 80937 | AI639476 | squamous cell carcinoma antigen | |
| | | | recognized by T-cells 3 (predicted) | |
| 923 | 80954 | AI639495 | | |
| 924 | 80957 | AI639498 | | |
| 925 | 80964 | AI639505 | | |
| 926 | 80975 | AI639518 | polymerase (RNA) II (DNA directed) | (Purine metabolism, Pyrimidine metabolism, RNA |
| | | | polypeptide H (predicted) | polymerase) |
| 927 | 80998 | AI639536 | | |
| 928 | 83529 | NM_053472 | cytochrome c oxidase subunit IV | Oxidative phosphorylation |
| | | | isoform 2 | |
| 929 | 83544 | H31535 | similar to RIKEN cDNA 2610511E22 | |
| | | | (predicted) | |
| 930 | 83545 | H31550 | | |
| 931 | 83546 | H31554 | | |
| 932 | 83549 | NM_153294 | prepro-Neuropeptide W polypeptide | Neuroactive ligand-receptor interaction |
| 643 | 83555 | NM_080902 | hypoxia induced gene 1 | |
| 933 | 83556 | NM_021597 | GERp95 | Dicer Pathway |
| 934 | 83572 | H31887 | similar to RIKEN cDNA 1700037H04 | |
| | | | (predicted) | |
| 935 | 83573 | H31897 | | |
| 936 | 83578 | H31964 | | |
| 937 | 83581 | H31990 | | |
| 938 | 83587 | H33084 | | |
| 939 | 83593 | H33149 | similar to RIKEN cDNA 1810047C23 | |
| | | | (predicted) | |
| 940 | 83611 | H33528 | | |
| 941 | 83614 | H33566 | | |
| 942 | 83615 | H33577 | | |
| 943 | 83621 | H33660 | similar to chromosome 14 open | |
| | | | reading frame 50 (predicted) | |
| 944 | 78424 | NM_024372 | solute carrier family 6 | |
| | | | (neurotransmitter transporter, | |
| | | | GABA), member 11 | |
| 945 | 78426 | NM_013143 | meprin 1 alpha | |
| 946 | 78428 | NM_138549 | glycoprotein, synaptic 2 | |
| 947 | 78435 | S46798 | | |
| 948 | 78445 | NM_031034 | guanine nucleotide binding protein, | (MAPK signaling pathway, PKC-catalyzed |
| | | | alpha 12 | phosphorylation of inhibitory phosphoprotein of |
| | | | | myosin phosphatase, Regulation of actin |
| | | | | cytoskeleton, Rho-Selective Guanine Exchange |
| | | | | Factor AKAP13 Mediates Stress Fiber Formation, |
| | | | | Thrombin signaling and protease-activated |
| | | | | receptors) |
| 949 | 78452 | S53987 | | |
| 950 | 78457 | NM_017037 | peripheral myelin protein 22 | |
| 951 | 78460 | S56464 | | |
| 951 | 78461 | S56464 | | |
| 220 | 78466 | NM_057105 | UDP glycosyltransferase 1 family, | (Androgen and estrogen metabolism, Pentose |
| | | | polypeptide A6 | and glucuronate interconversions, Porphyrin and |
| | | | | chlorophyll metabolism, Starch and sucrose |
| | | | | metabolism) |
| 952 | 78470 | S58528 | | |
| 953 | 78478 | NM_012642 | renin 1 | Angiotensin-converting enzyme 2 regulates heart |
| | | | | function |
| 954 | 78481 | NM_012649 | syndecan 4 | ECM-receptor interaction |
| 954 | 78482 | NM_012649 | syndecan 4 | ECM-receptor interaction |
| 955 | 78485 | S61960 | cysteine conjugate-beta lyase | Tryptophan metabolism |
| | | | (predicted) | |
| 956 | 78486 | NM_153308 | glutamate receptor, ionotropic, N- | |
| | | | methyl D-asparate-associated | |
| | | | protein 1 (glutamate binding) | |
| 957 | 78489 | NM_031654 | Rab geranylgeranyl transferase, a | |
| | | | subunit | |
| 958 | 78495 | NM_053290 | phosphoglycerate mutase 1 | Glycolysis/Gluconeogenesis |
| 959 | 78505 | S65091 | protein phosphatase 1, regulatory | |
| | | | subunit 1C | |
| 960 | 78509 | NM_017305 | glutamate cysteine ligase, modifier | (Glutamate metabolism, Glutathione metabolism) |
| | | | subunit | |
| 960 | 78510 | NM_017305 | glutamate cysteine ligase, modifier | (Glutamate metabolism, Glutathione metabolism) |
| | | | subunit | |
| 961 | 78511 | NM_017334 | cAMP responsive element modulator | |
| 961 | 78512 | NM_017334 | cAMP responsive element modulator | |
| 962 | 78513 | NM_017061 | lysyl oxidase | |
| 963 | 78516 | NM_022852 | pancreatic and duodenal homeobox | |
| | | | gene 1 | |
| 964 | 78521 | NM_080477 | 6-phosphofructo-2-kinase/fructose- | Fructose and mannose metabolism |
| | | | 2,6-biphosphatase 2 | |
| 965 | 78529 | NM_001007636 | S100 calcium binding protein A1 | |
| 966 | 78532 | NM_013047 | thyrotropin releasing hormone | (Calcium signaling pathway, Neuroactive ligand- |
| | | | receptor | receptor interaction) |
| 967 | 78555 | NM_031509 | glutathione S-transferase A5 | Glutathione metabolism |
| 968 | 78557 | NM_021989 | tissue inhibitor of metalloproteinase 2 | Inhibition of Matrix Metalloproteinases |
| 969 | 78563 | NM_031051 | macrophage migration inhibitory | (Phenylalanine metabolism, Tyrosine |
| | | | factor | metabolism) |
| 970 | 78569 | S74257 | | |
| 971 | 78572 | NM_013001 | paired box gene 6 | |
| 972 | 78574 | NM_033096 | protein phosphatase 1B, magnesium | MAPK signaling pathway |
| | | | dependent, beta isoform | |
| 973 | 78595 | NM_199230 | activin A receptor, type 1B | (Adherens junction, Cytokine-cytokine receptor |
| | | | | interaction, MAPK signaling pathway, |
| | | | | Phosphatidylinositol signaling system, TGF-beta |
| | | | | signaling pathway) |
| 962 | 78601 | NM_017061 | lysyl oxidase | |
| 974 | 78602 | NM_024125 | CCAAT/enhancer binding protein | IL 6 signaling pathway |
| | | | (C/EBP), beta | |
| 975 | 78603 | S77532 | | |
| 976 | 78604 | NM_013137 | discoidin domain receptor family, | |
| | | | member 1 | |
| 977 | 78606 | S77858 | | |
| 978 | 78616 | S78556 | | |
| 979 | 78623 | S79304 | | |
| 980 | 78629 | NM_013007 | prepronociceptin | |
| 981 | 78634 | S79820 | | |
| 982 | 78638 | S80118 | | |
| 983 | 78639 | NM_017044 | parathyroid hormone | Neuroactive ligand-receptor interaction |
| 984 | 78642 | S80379 | | |
| 985 | 78644 | NM_012613 | natriuretic peptide receptor 1 | Purine metabolism |
| 986 | 78653 | NM_012732 | lipase A, lysosomal acid | (Bile acid biosynthesis, Glycerolipid metabolism) |
| 559 | 78660 | NM_173111 | tropomyosin isoform 6 | |
| 987 | 78670 | S83320 | | |
| 988 | 76872 | NM_181371 | glutathione S-transferase, | Glutathione metabolism |
| | | | mitochondrial | |
| 989 | 78672 | S85184 | | |
| 989 | 78673 | S85184 | | |
| 990 | 78679 | S94371 | | |
| 991 | 79818 | NM_031588 | neuregulin 1 | (Agrin in Postsynaptic Differentiation, Neuregulin |
| | | | | Receptor Signaling, Neuroregulin receptor |
| | | | | degradation protein-1 Controls ErbB3 receptor |
| | | | | recycling) |
| 992 | 82149 | NM_022588 | metastasis associated 1 | Downregulated of MTA-3 in ER-negative Breast |
| | | | | Tumors |
| 993 | 79758 | NM_013033 | solute carrier family 5 | |
| | | | (sodium/glucose cotransporter), | |
| | | | member 1 | |
| 994 | 79594 | NM_053573 | olfactomedin 1 | |
| 995 | 78694 | NM_017174 | phospholipase A2, group V | (Glycerophospholipid metabolism, MAPK |
| | | | | signaling pathway, Prostaglandin and leukotriene |
| | | | | metabolism) |
| 996 | 79773 | NM_013086 | cAMP responsive element modulator | |
| 997 | 79725 | NM_013080 | protein tyrosine phosphatase, | |
| | | | receptor-type, Z polypeptide 1 | |
| 998 | 76888 | NM_053857 | eukaryotic translation initiation factor | (Inhibition of Protein Synthesis by AMP Kinase, |
| | | | 4E binding protein-1 | Regulation of eIF4e and p70 S6 Kinase, Skeletal |
| | | | | muscle hypertrophy is regulated via AKT/mTOR |
| | | | | pathway, mTOR Signaling Pathway) |
| 999 | 82060 | NM_020071 | fibrinogen, B beta polypeptide | (Acute Myocardial Infarction, C-Reactive Protein, |
| | | | | Apo-B in Lipid Metabolism and Cardiovascular |
| | | | | Disease, Complement and coagulation cascades, |
| | | | | Extrinsic Prothrombin Activation Pathway, |
| | | | | Fibrinolysis Pathway, Intrinsic Prothrombin |
| | | | | Activation Pathway, Platelet thrombus formation |
| | | | | at injury sites, Role of Peroxisome Proliferator- |
| | | | | Activated Receptor in Inflammation) |
| 1000 | 76715 | NM_017169 | peroxiredoxin 2 | (Methane metabolism, Phenylalanine |
| | | | | metabolism, Prostaglandin and leukotriene |
| | | | | metabolism) |
| 1001 | 78701 | U06752 | mucin 4 | |
| 1002 | 82002 | NM_019276 | UDP-glucuronosyltransferase 8 | |
| 842 | 82500 | NM_017211 | golgi apparatus protein 1 | |
| 1003 | 79624 | NM_024379 | glutamate receptor, ionotropic, delta 2 | Neuroactive ligand-receptor interaction |
| 1004 | 82439 | NM_053369 | transcription factor 4 | |
| 1005 | 79921 | U09229 | cut-like 1 (Drosophila) | |
| 1006 | 78709 | U09401 | tenascin C | |
| 1007 | 82163 | NM_017100 | polo-like kinase 1 (Drosophila) | (Cell Cycle: G2/M Checkpoint, Cell cycle, |
| | | | | Phosphatidylinositol signaling system, |
| | | | | Ubiquitylation in the Control of Cell Cycle) |
| 1008 | 79623 | NM_019329 | contactin 3 | |
| 324 | 80281 | NM_017010 | glutamate receptor, ionotropic, N- | (Calcium signaling pathway, Erythropoietin |
| | | | methyl D-aspartate 1 | mediated neuroprotection through NF-kB, |
| | | | | Ethanol-Induced Signal Transduction, LIN |
| | | | | Transport Complex, Neuregulin Receptor |
| | | | | Signaling, Neuroactive ligand-receptor |
| | | | | interaction, Nitric Oxide Signaling Pathway, |
| | | | | Stimulation of Synaptic Vesicle Recycling by |
| | | | | Nitrous Oxide, Synaptic Proteins at the Synaptic |
| | | | | Junction) |
| 1009 | 82032 | NM_053864 | valosin-containing protein | |
| 1010 | 79754 | NM_030831 | G-protein coupled receptor 12 | |
| 1011 | 79888 | NM_012982 | msh homeo box homolog 2 | |
| | | | (Drosophila) | |
| 141 | 78719 | NM_031514 | Janus kinase 2 | (Bioactive Peptide Induced Signaling Pathway, |
| | | | | Chaperones modulate interferon Signaling |
| | | | | Pathway, EPO Signaling Pathway, Erythropoietin |
| | | | | mediated neuroprotection through NF-kB, |
| | | | | Evasion of Innate Immunity by Protozoan |
| | | | | Parasites, Growth Hormone Signaling Pathway, |
| | | | | ICAM-1 in signal transduction, IFN gamma |
| | | | | signaling pathway, IL 3 signaling pathway, IL 6 |
| | | | | signaling pathway, IL12 and Stat4 Dependent |
| | | | | Signaling Pathway in Th1 Development, IL22 |
| | | | | Soluble Receptor Signaling Pathway, Inhibition of |
| | | | | Cellular Proliferation by Gleevec, Jak-STAT |
| | | | | signaling pathway, NO2-dependent IL 12 |
| | | | | Pathway in NK cells, Regulation of Inducible |
| | | | | Nitric Oxide Synthase by Interleukin-17, |
| | | | | Regulation of Insulin and IGF Signaling by IRS |
| | | | | Proteins, Stat3 Signaling Pathway, TPO Signaling |
| | | | | Pathway) |
| 1012 | 82031 | NM_012825 | aquaporin 4 | |
| 1013 | 82176 | NM_052801 | von Hippel-Lindau syndrome | (Cullin-based ligases: von Hippel-Lindau |
| | | | homolog | Complex, Hypoxia-Inducible Factor in the |
| | | | | Cardiovascular System, The Role of the Cxcr4 |
| | | | | Chemokine Receptor and pVHL Tumor |
| | | | | Suppressor in Metastasis, VEGF, Hypoxia, and |
| | | | | Angiogenesis) |
| 795 | 82215 | NM_017215 | solute carrier family 1 (glial high | Amytotrophic lateral sclerosis (ALS) |
| | | | affinity glutamate transporter), | |
| | | | member 2 | |
| 1014 | 79659 | NM_022856 | Ngfi-A binding protein 1 | |
| 1015 | 76900 | NM_053865 | N-acetyltransferase 1 (arylamine N- | |
| | | | acetyltransferase) | |
| 1016 | 82662 | NM_053865 | reticulon 1 | |
| 773 | 76902 | NM_012941 | cytochrome P450, subfamily 51 | (Fatty acid metabolism, Tryptophan metabolism) |
| 1017 | 82094 | NM_133580 | RAB26, member RAS oncogene | |
| | | | family | |
| 1018 | 76903 | NM_012954 | fos-like antigen 2 | |
| 1019 | 82090 | NM_145092 | lamina-associated polypeptide 1B | |
| 1020 | 78731 | NM_053325 | synaptotagmin 8 | |
| 1021 | 79688 | NM_022863 | iron responsive element binding | |
| | | | protein 2 | |
| 1022 | 79684 | NM_031126 | syntaxin binding protein 2 | |
| 1023 | 76904 | NM_012495 | aldolase A | (Downregulated of MTA-3 in ER-negative Breast |
| | | | | Tumors, Fructose and mannose metabolism, |
| | | | | Glycolysis/Gluconeogenesis, Inositol |
| | | | | metabolism, Pentose phosphate pathway) |
| 1024 | 82039 | NM_147210 | nuclear receptor subfamily 1, group | |
| | | | D, member 2 | |
| 1025 | 78735 | U21719 | DEAD (Asp-Glu-Ala-Asp) box | |
| | | | polypeptide 21b (predicted) | |
| 1026 | 79694 | NM_013025 | chemokine (C-C motif) ligand 3 | (Cytokine-cytokine receptor interaction, |
| | | | | Erythrocyte Differentiation Pathway, Selective |
| | | | | expression of chemokine receptors during T-cell |
| | | | | polarization) |
| 1027 | 78739 | NM_057103 | A kinase (PRKA) anchor protein | |
| | | | (gravin) 12 | |
| 1028 | 82134 | NM_017244 | cellular retinoic acid binding protein 2 | |
| 1029 | 76905 | NM_022199 | dual specificity phosphatase 4 | MAPK signaling pathway |
| 1030 | 82207 | NM_017365 | PDZ and LIM domain 1 (elfin) | |
| 1031 | 79787 | NM_017064 | signal transducer and activator of | (Bioactive Peptide Induced Signaling Pathway, |
| | | | transcription 5A | EGF Signaling Pathway, EPO Signaling Pathway, |
| | | | | Growth Hormone Signaling Pathway, IL 2 |
| | | | | signaling pathway, IL 3 signaling pathway, IL-10 |
| | | | | Anti-inflammatory Signaling Pathway, IL-2 |
| | | | | Receptor Beta Chain in T cell Activation, IL-7 |
| | | | | Signal Transduction, IL22 Soluble Receptor |
| | | | | Signaling Pathway, Inhibition of Cellular |
| | | | | Proliferation by Gleevec, Jak-STAT signaling |
| | | | | pathway, Mechanism of Gene Regulation by |
| | | | | Peroxisome Proliferators via PPARa(alpha), |
| | | | | PDGF Signaling Pathway, TPO Signaling |
| | | | | Pathway) |
| 1032 | 78740 | U25148 | myosin IA | |
| 1033 | 76906 | NM_012610 | nerve growth factor receptor (TNFR | (Erk1/Erk2 Mapk Signaling pathway, Nerve |
| | | | superfamily, member 16) | growth factor pathway (NGF), Nogo-A Signal |
| | | | | Transduction Pathway and Axon Regeneration, |
| | | | | Phosphorylation of MEK1 by cdk5/p35 down |
| | | | | regulates the MAP kinase pathway) |
| 1034 | 82148 | NM_031987 | carnitine O-octanoyltransferase | (Butanoate metabolism, Glycerophospholipid |
| | | | | metabolism, Histidine metabolism, Lysine |
| | | | | degradation, Phenylalanine metabolism, Tyrosine |
| | | | | metabolism, Valine, leucine and isoleucine |
| | | | | degradation) |
| 1035 | 82480 | NM_031002 | inositol polyphosphate-4- | (Inositol phosphate metabolism, |
| | | | phosphatase, type 1 | Phosphatidylinositol signaling system) |
| 1036 | 82848 | NM_031615 | zinc finger protein 148 | |
| 1037 | 82167 | NM_138849 | B/K protein | |
| 1038 | 78749 | NM_147211 | SH3 domain binding protein CR16 | |
| 1039 | 82136 | NM_017228 | dentatorubral pallidoluysian atrophy | Dentatorubropallidoluysian atrophy (DRPLA) |
| 1040 | 78759 | NM_133513 | mucin 10, submandibular gland | |
| | | | salivary mucin | |
| 1041 | 79726 | NM_017253 | branched chain aminotransferase 1, | (Pantothenate and CoA biosynthesis, Valine, |
| | | | cytosolic | leucine and isoleucine degradation) |
| 1042 | 82130 | NM_031552 | adducin 3 (gamma) | |
| 1043 | 78762 | NM_031077 | PCTAIRE-motif protein kinase 1 | Phosphatidylinositol signaling system |
| 1044 | 78765 | NM_017274 | glycerol-3-phosphate | (Glycerolipid metabolism, Glycerophospholipid |
| | | | acyltransferase, mitochondrial | metabolism) |
| 1044 | 78767 | NM_017274 | glycerol-3-phosphate | (Glycerolipid metabolism, Glycerophospholipid |
| | | | acyltransferase, mitochondrial | metabolism) |
| 918 | 80018 | NM_133609 | eukaryotic translation initiation factor | VEGF, Hypoxia, and Angiogenesis |
| | | | 2B, subunit 3 gamma | |
| 1045 | 76691 | NM_133551 | phospholipase A2, group IVA | (Aspirin Blocks Signaling Pathway Involved in |
| | | | (cytosolic, calcium-dependent) | Platelet Activation, Eicosanoid Metabolism, Fc |
| | | | | Epsilon Receptor I Signaling in Mast Cells, |
| | | | | Glycerophospholipid metabolism, Prostaglandin |
| | | | | and leukotriene metabolism, p38 MAPK Signaling |
| | | | | Pathway) |
| 1046 | 82601 | NM_013022 | Rho-associated coiled-coil forming | (CCR3 signaling in Eosinophils, Focal adhesion, |
| | | | kinase 2 | Nogo-A Signal Transduction Pathway and Axon |
| | | | | Regeneration, Phosphatidylinositol signaling |
| | | | | system, Regulation of actin cytoskeleton, Rho- |
| | | | | family Proteins in Cell Adhesion and Cancer, |
| | | | | TGF-beta signaling pathway, The Role of the |
| | | | | Cxcr4 Chemokine Receptor and pVHL Tumor |
| | | | | Suppressor in Metastasis, Writ signaling |
| | | | | pathway) |
| 1047 | 78773 | U39571 | | |
| 1048 | 78775 | U39608 | | |
| 1049 | 82520 | NM_024139 | calcium binding protein p22 | Association of Signaling Intermediates With |
| | | | | Sodium-Hydrogen Exchanger NHE1 |
| 1050 | 78778 | NM_019142 | protein kinase, AMP-activated, alpha | (ChREBP regulation by carbohydrates and |
| | | | 1 catalytic subunit | cAMP, Inhibition of Protein Synthesis by AMP |
| | | | | Kinase, Inositol phosphate metabolism, |
| | | | | Nicotinate and nicotinamide metabolism, |
| | | | | Phosphatidylinositol signaling system, Reversal |
| | | | | of Insulin Resistance by Leptin) |
| 1051 | 76916 | NM_012786 | Cytochrom c oxidase subunit VIII-H | Oxidative phosphorylation |
| | | | (heart/muscle) | |
| 1027 | 78779 | NM_057103 | A kinase (PRKA) anchor protein | |
| | | | (gravin) 12 | |
| 1052 | 82715 | NM_012991 | nuclear pore associated protein | |
| 1053 | 76765 | NM_138867 | hypoxia up-regulated 1 | |
| 1054 | 82236 | NM_012894 | adenosine deaminase, RNA-specific, | |
| | | | B1 | |
| 1055 | 78781 | U47014 | proprotein convertase subtilisin/kexin | |
| | | | type 5 | |
| 1056 | 80045 | NM_022184 | calcium/calmodulin-dependent | (CASK Protein Interactions, LIN Transport |
| | | | serine protein kinase | Complex, PALS Membrane Complexes, |
| | | | | Regulation of Inward Rectifier Potassium |
| | | | | Channels, Tight junction) |
| 1057 | 76919 | U47313 | | |
| 1058 | 76921 | NM_133561 | brain protein 44-like | |
| 1059 | 76101 | NM_138515 | cytochrome P450, family 2, | (Fatty acid metabolism, Tryptophan metabolism) |
| | | | subfamily d, polypeptide 22 | |
| 1060 | 76754 | NM_053887 | mitogen activated protein kinase | (Angiotensin II mediated activation of JNK |
| | | | kinase kinase 1 | Pathway via Pyk2 dependent signaling, BCR |
| | | | | Signaling Pathway, CD40L Signaling Pathway, |
| | | | | Ceramide Signaling Pathway, EGF Signaling |
| | | | | Pathway, FAS signaling pathway (CD95), Fc |
| | | | | Epsilon Receptor I Signaling in Mast Cells, HIV-I |
| | | | | Nef: negative effector of Fas and TNF, Human |
| | | | | Cytomegalovirus and Map Kinase Pathways, |
| | | | | Inhibition of Cellular Proliferation by Gleevec, |
| | | | | Keratinocyte Differentiation, Links between Pyk2 |
| | | | | and Map Kinases, MAPK signaling pathway, |
| | | | | MAPKinase Signaling Pathway, Map Kinase |
| | | | | Inactivation of SMRT Corepressor, NF-kB |
| | | | | Signaling Pathway, Neuropeptides VIP and |
| | | | | PACAP inhibit the apoptosis of activated T cells, |
| | | | | PDGF Signaling Pathway, Phosphatidylinositol |
| | | | | signaling system, Rac 1 cell motility signaling |
| | | | | pathway, Role of MAL in Rho-Mediated Activation |
| | | | | of SRF, Signal transduction through IL1R, T Cell |
| | | | | Receptor Signaling Pathway, TNF/Stress Related |
| | | | | Signaling, TNFR1 Signaling Pathway, TNFR2 |
| | | | | Signaling Pathway, The 4-1BB-dependent |
| | | | | immune response, Toll-Like Receptor Pathway, |
| | | | | fMLP induced chemokine gene expression in |
| | | | | HMC-1 cells, p38 MAPK Signaling Pathway) |
| 1061 | 82488 | U49099 | | |
| 1062 | 76767 | NM_012922 | caspase 3 | (Alzheimer's disease, Apoptosis, Apoptotic DNA |
| | | | | fragmentation and tissue homeostasis, Apoptotic |
| | | | | Signaling in Response to DNA Damage, B Cell |
| | | | | Survival Pathway, Caspase Cascade in |
| | | | | Apoptosis, D4-GDI Signaling Pathway, |
| | | | | Dentatorubropallidoluysian atrophy (DRPLA), |
| | | | | FAS signaling pathway (CD95), HIV-I Nef: |
| | | | | negative effector of Fas and TNF, Huntington's |
| | | | | disease, Induction of apoptosis through DR3 and |
| | | | | DR4/5 Death Receptors, MAPK signaling |
| | | | | pathway, Role of Mitochondria in Apoptotic |
| | | | | Signaling, Stress Induction of HSP Regulation, |
| | | | | TNFR1 Signaling Pathway, TSP-1 Induced |
| | | | | Apoptosis in Microvascular Endothelial Cell) |
| 1063 | 80029 | NM_013005 | phosphatidylinositol 3-kinase, | (Apoptosis, Focal adhesion, Jak-STAT signaling |
| | | | regulatory subunit, polypeptide 1 | pathway, Phosphatidylinositol signaling system, |
| | | | | Regulation of actin cytoskeleton, Toll-like receptor |
| | | | | signaling pathway) |
| 552 | 82494 | NM_053891 | cyclin-dependent kinase 5, | (Deregulation of CDK5 in Alzheimers Disease, |
| | | | regulatory subunit 1 (p35) | Lissencephaly gene (LIS1) in neuronal migration |
| | | | | and development, Phosphorylation of MEK1 by |
| | | | | cdk5/p35 down regulates the MAP kinase |
| | | | | pathway, Rac 1 cell motility signaling pathway, |
| | | | | Reelin Signaling Pathway, Regulation of ck1/cdk5 |
| | | | | by type 1 glutamate receptors) |
| 1064 | 78801 | NM_022175 | placentae and embryos oncofetal | |
| | | | gene | |
| 1065 | 78807 | U52950 | microtubule-associated protein 1b | |
| 1066 | 82243 | U53184 | LPS-induced TNF-alpha factor | |
| 1067 | 82483 | NM_012892 | amiloride-sensitive cation channel 1, | |
| | | | neuronal (degenerin) | |
| 1068 | 78810 | NM_030999 | corticotropin releasing hormone | Neuroactive ligand-receptor interaction |
| | | | receptor 1 | |
| 1069 | 82264 | U53513 | glycine-, glutamate-, | |
| | | | thienylcyclohexylpiperidine-binding | |
| | | | protein | |
| 1070 | 82267 | NM_031062 | mevalonate (diphospho) | Biosynthesis of steroids |
| | | | decarboxylase | |
| 1071 | 78811 | U53873 | | |
| 529 | 76706 | NM_022934 | DnaJ-like protein | |
| 1072 | 78812 | NM_022619 | solute carrier family 7 (cationic | |
| | | | amino acid transporter, y+ system), | |
| | | | member 2 | |
| 1073 | 80031 | NM_031089 | parathyroid hormone receptor 2 | Neuroactive ligand-receptor interaction |
| 1074 | 82684 | NM_013029 | sialyltransferase 8 C | |
| 1075 | 79994 | NM_031665 | syntaxin 6 | |
| 1076 | 82484 | NM_017255 | purinergic receptor P2Y, G-protein | (Ion Channel and Phorbal Esters Signaling |
| | | | coupled 2 | Pathway, Neuroactive ligand-receptor interaction) |
| 1077 | 79952 | U57049 | 5,10-methylenetetrahydrofolate | One carbon pool by folate |
| | | | reductase | |
| 579 | 78820 | NM_031978 | proteasome (prosome, macropain) | Proteasome |
| | | | 26S subunit, non-ATPase, 1 | |
| 1078 | 82524 | NM_134332 | granzyme C | |
| 1079 | 79982 | NM_153466 | natural killer cell protease 7 | |
| 1080 | 82577 | NM_013044 | tropomodulin 1 | |
| 1081 | 79961 | NM_024394 | 5-hydroxytryptamine (serotonin) | Calcium signaling pathway |
| | | | receptor 3a | |
| 1082 | 78834 | NM_022529 | mitochondrial ribosomal protein L23 | |
| 1083 | 80063 | NM_031123 | stanniocalcin 1 | |
| 1084 | 76779 | NM_024487 | GrpE-like 1, mitochondrial | |
| 1085 | 78836 | NM_139090 | active receptor-like kinase 7 | (Adherens junction, MAPK signaling pathway, |
| | | | | Phosphatidylinositol signaling system, TGF-beta |
| | | | | signaling pathway) |
| 639 | 78837 | NM_031614 | thioredoxin reductase 1 | Pyrimidine metabolism |
| 1086 | 76927 | NM_013084 | acyl-Coenzyme A dehydrogenase, | Bile acid biosynthesis |
| | | | short/branched chain | |
| 1087 | 78838 | NM_053600 | fasciculation and elongation protein | |
| | | | zeta 2 (zygin II) | |
| 1088 | 78839 | NM_001008335 | eukaryotic translation initiation factor | (Eukaryotic protein translation, Internal Ribosome |
| | | | 4A2 (predicted) | Entry Pathway, Regulation of eIF4e and p70 S6 |
| | | | | Kinase, mTOR Signaling Pathway) |
| 1089 | 82644 | NM_031054 | matrix metallopeptidase 2 | (Inhibition of Matrix Metalloproteinases, Sexual |
| | | | | Differentiation of the Reproductive System) |
| 1090 | 78843 | NM_021677 | pregnancy-specific beta 1- | |
| | | | glycoprotein | |
| 1091 | 78844 | U66293 | pregnancy-specific beta 1- | |
| | | | glycoprotein | |
| 1092 | 78845 | NM_013107 | bone morphogenetic protein 6 | TGF-beta signaling pathway |
| 1093 | 80068 | NM_138863 | leukotriene B4 12- | |
| | | | hydroxydehydrogenase | |
| 1094 | 80054 | NM_013130 | SMAD, mothers against DPP | (ALK in cardiac myocytes, CTCF: First Multivalent |
| | | | homolog 1 (Drosophila) | Nuclear Factor, TGF-beta signaling pathway) |
| 1095 | 80074 | NM_019620 | zinc finger protein 386 (Kruppel-like) | |
| 1096 | 78848 | NM_012596 | leptin receptor | (Cytokine-cytokine receptor interaction, Jak-STAT |
| | | | | signaling pathway, Neuroactive ligand-receptor |
| | | | | interaction, Reversal of Insulin Resistance by |
| | | | | Leptin) |
| 112 | 76776 | NM_017318 | protein tyrosine kinase 2 beta | (Angiotensin II mediated activation of JNK |
| | | | | Pathway via Pyk2 dependent signaling, Bioactive |
| | | | | Peptide Induced Signaling Pathway, CXCR4 |
| | | | | Signaling Pathway, Calcium Signaling by HBx of |
| | | | | Hepatitis B virus, Calcium signaling pathway, IL-7 |
| | | | | Signal Transduction, Ion Channel and Phorbal |
| | | | | Esters Signaling Pathway, Links between Pyk2 |
| | | | | and Map Kinases, Pertussis toxin-insensitive |
| | | | | CCR5 Signaling in Macrophage, Ras- |
| | | | | Independent pathway in NK cell-mediated |
| | | | | cytotoxicity, Role of nicotinic acetylcholine |
| | | | | receptors in the regulation of apoptosis, Signaling |
| | | | | of Hepatocyte Growth Factor Receptor, Thrombin |
| | | | | signaling and protease-activated receptors) |
| 1097 | 76789 | NM_013129 | interleukin 15 | (Cytokine Network, Cytokine-cytokine receptor |
| | | | | interaction, Cytokines and Inflammatory |
| | | | | Response, Jak-STAT signaling pathway) |
| 1097 | 76790 | NM_013129 | interleukin 15 | (Cytokine Network, Cytokine-cytokine receptor |
| | | | | interaction, Cytokines and Inflammatory |
| | | | | Response, Jak-STAT signaling pathway) |
| 1098 | 80122 | NM_053903 | ephrin A5 | |
| 1099 | 82847 | U70050 | jagged 2 | Notch signaling pathway |
| 1100 | 76931 | NM_019310 | interleukin 8 receptor, alpha | Cytokine-cytokine receptor interaction |
| 1101 | 82581 | NM_012867 | ninjurin 1 | |
| 1102 | 80319 | NM_012977 | lectin, galactose binding, soluble 9 | |
| 1102 | 80320 | NM_012977 | lectin, galactose binding, soluble 9 | |
| 1103 | 80181 | NM_053585 | MAP-kinase activating death domain | TNFR1 Signaling Pathway |
| 71 | 78865 | NM_012551 | early growth response 1 | (Phosphorylation of MEK1 by cdk5/p35 down |
| | | | | regulates the MAP kinase pathway, Regulation of |
| | | | | Steroidogenesis by Orphan NuclearReceptor SF- |
| | | | | 1) |
| 1104 | 78872 | U75412 | | |
| 1105 | 76936 | NM_130431 | heat shock 27 kD protein 2 | (Downregulated of MTA-3 in ER-negative Breast |
| | | | | Tumors, MAPK signaling pathway, Stress |
| | | | | Induction of HSP Regulation, p38 MAPK |
| | | | | Signaling Pathway) |
| 1106 | 82650 | U75916 | tight junction protein 2 | Tight junction |
| 1107 | 82651 | NM_022952 | adaptor-related protein complex 2, | |
| | | | sigma 1 subunit | |
| 1108 | 78873 | NM_138509 | microtubule-associated protein, | |
| | | | RP/EB family, member 1 | |
| 1109 | 78874 | U75921 | | |
| 1110 | 78875 | U75923 | isoleucine-tRNA synthetase | Aminoacyl-tRNA biosynthesis |
| 1111 | 80287 | NM_053615 | casein kinase 1, alpha 1 | (Circadian rhythm, Deregulation of CDK5 in |
| | | | | Alzheimers Disease, Hypoxia and p53 in the |
| | | | | Cardiovascular system, NFAT and Hypertrophy of |
| | | | | the heart (Transcription in the broken heart), |
| | | | | WNT Signaling Pathway, Wnt signaling pathway) |
| 1112 | 78894 | U77829 | growth arrest specific 5 | |
| 1113 | 82606 | NM_031595 | proteasome (prosome, macropain) | Proteasome |
| | | | 26S subunit, ATPase 3 | |
| 1114 | 78895 | NM_147136 | rRNA promoter binding protein | |
| 1115 | 78902 | NM_133289 | sodium channel, voltage-gated, type | |
| | | | 9, alpha polypeptide | |
| 1116 | 78903 | NM_022203 | calcium-activated potassium channel | |
| | | | beta subunit | |
| 1117 | 76822 | NM_031053 | mutL homolog 1 (E. coli) | |
| 1118 | 78905 | NM_053909 | neurofascin | |
| 1118 | 78906 | NM_053909 | neurofascin | |
| 1119 | 78907 | NM_032066 | smooth muscle-specific 17 beta- | (Bile acid biosynthesis, Butanoate metabolism, |
| | | | hydroxysteroid dehydrogenase type 3 | Fructose and mannose metabolism, Galactose |
| | | | | metabolism, Glycerophospholipid metabolism, |
| | | | | Glycine, serine and threonine metabolism, Lysine |
| | | | | degradation, Nucleotide sugars metabolism, |
| | | | | TIM23 Translocase) |
| 1120 | 82945 | NM_133525 | chromosome 6 open reading frame | |
| | | | 108 | |
| 1121 | 82913 | NM_013217 | afadin | (Adherens junction, Nectin Intercellular Adhesion |
| | | | | Complexes, Tight junction) |
| 1122 | 78914 | U83666 | budding uninhibited by | (Cell cycle, Phosphatidylinositol signaling system) |
| | | | benzimidazoles 1 homolog, beta (S. cerevisiae) | |
| 1123 | 78916 | NM_012736 | glycerol-3-phosphate | (Electron Transport Reaction in Mitochondria, |
| | | | dehydrogenase 2 | Glycerophospholipid metabolism) |
| 1124 | 82640 | NM_053911 | pleckstrin homology, Sec7 and | ADP-Ribosylation Factor |
| | | | coiled/coil domains 2 | |
| 1125 | 78919 | NM_022398 | 2-oxoglutarate carrier | (Malate-aspartate shuttle, Shuttle for transfer of |
| | | | | acetyl groups from mitochondria to the cytosol) |
| 1126 | 78921 | NM_031666 | synaptotagmin X | |
| 1127 | 78922 | NM_022206 | unc-5 homolog A (C. elegans) | |
| 1128 | 78923 | NM_022207 | unc-5 homolog B (C. elegans) | |
| 1129 | 82660 | NM_012964 | hyaluronan mediated motility | |
| | | | receptor | |
| 66 | 78927 | NM_030987 | guanine nucleotide binding protein, | (Activation of Csk by cAMP-dependent Protein |
| | | | beta 1 | Kinase Inhibits Signaling through the T Cell |
| | | | | Receptor, Activation of cAMP-dependent protein |
| | | | | kinase, PKA, Aspirin Blocks Signaling Pathway |
| | | | | Involved in Platelet Activation, Attenuation of |
| | | | | GPCR Signaling, Bioactive Peptide Induced |
| | | | | Signaling Pathway, Blood Pressure and RGS-2, |
| | | | | CCR3 signaling in Eosinophils, CXCR4 Signaling |
| | | | | Pathway, ChREBP regulation by carbohydrates |
| | | | | and cAMP, Corticosteroids and cardioprotection, |
| | | | | Ephrins and Ephs: forward and reverse signalling, |
| | | | | Erk1/Erk2 Mapk Signaling pathway, Formation of |
| | | | | Phagosomes and Recruitment of Endoplasmic |
| | | | | Reticulum, G-Protein Signaling Through Tubby |
| | | | | Proteins, How Progesterone Initiates the Oocyte |
| | | | | Maturation, Ion Channels and Their Functional |
| | | | | Role in Vascular Endothelium, PKC-catalyzed |
| | | | | phosphorylation of inhibitory phosphoprotein of |
| | | | | myosin phosphatase, Phospholipids as signalling |
| | | | | intermediaries, Role of beta-arrestins in the |
| | | | | activation and targeting of MAP kinases, Roles of |
| | | | | beta-arrestin-dependent Recruitment of Src |
| | | | | Kinases in GPCR Signaling, Signaling Pathway |
| | | | | from G-Protein Families, The Role of Slit-Robo |
| | | | | Pathway in Axon Guidance, Thrombin signaling |
| | | | | and protease-activated receptors, beta-arrestins |
| | | | | in GPCR Desensitization, fMLP induced |
| | | | | chemokine gene expression in HMC-1 cells) |
| 1130 | 78933 | NM_053360 | SH3-domain kinase binding protein 1 | CBL mediated ligand-induced downregulation of |
| | | | | EGF receptors |
| 1131 | 78936 | NM_172039 | high density lipoprotein binding | |
| | | | protein | |
| 1132 | 78938 | NM_032072 | amyloid beta precursor protein | Alzheimer's disease |
| | | | binding protein 1 | |
| 1133 | 78942 | NM_019358 | glycoprotein 38 | |
| 1134 | 80203 | NM_053583 | Olf-1/EBF associated Zn finger | |
| | | | protein Roaz | |
| 1135 | 80300 | NM_078621 | chemokine binding protein 2 | |
| 1136 | 78945 | NM_031828 | potassium large conductance | |
| | | | calcium-activated channel, subfamily | |
| | | | M, alpha member 1 | |
| 1137 | 80188 | NM_012870 | tumor necrosis factor receptor | Cytokine-cytokine receptor interaction |
| | | | superfamily, member 11b | |
| | | | (osteoprotegerin) | |
| 1138 | 82933 | NM_173143 | abl-interactor 2 | Y branching of actin filaments |
| 1139 | 78949 | NM_053598 | nudix (nucleoside diphosphate linked | |
| | | | moiety X)-type motif 4 | |
| 1140 | 80253 | NM_024159 | disabled homolog 2 (Drosophila) | |
| 309 | 82733 | NM_031043 | glycogenin 1 | |
| 455 | 78964 | NM_031577 | growth hormone releasing hormone | Neuroactive ligand-receptor interaction |
| 1141 | 82570 | NM_012493 | alpha-fetoprotein | |
| 1142 | 76948 | NM_138974 | glutathione S-transferase, pi 2 | (Glutathione metabolism, Multi-Drug Resistance |
| | | | | Factors) |
| 1143 | 82357 | NM_012998 | prolyl 4-hydroxylase, beta | (Arginine and proline metabolism, Hypoxia- |
| | | | polypeptide | Inducible Factor in the Cardiovascular System, |
| | | | | Vitamin C in the Brain) |
| 1144 | 78968 | X03914 | | |
| 1145 | 82436 | NM_017251 | gap junction membrane channel | |
| | | | protein beta 1 | |
| 1033 | 76731 | NM_012610 | nerve growth factor receptor (TNFR | (Erk1/Erk2 Mapk Signaling pathway, Nerve |
| | | | superfamily, member 16) | growth factor pathway (NGF), Nogo-A Signal |
| | | | | Transduction Pathway and Axon Regeneration, |
| | | | | Phosphorylation of MEK1 by cdk5/p35 down |
| | | | | regulates the MAP kinase pathway) |
| 1146 | 82384 | NM_013067 | ribophorin I | N-Glycan biosynthesis |
| 277 | 78973 | NM_012559 | fibrinogen, gamma polypeptide | (Acute Myocardial Infarction, C-Reactive Protein, |
| | | | | Apo-B in Lipid Metabolism and Cardiovascular |
| | | | | Disease, Complement and coagulation cascades, |
| | | | | Extrinsic Prothrombin Activation Pathway, |
| | | | | Fibrinolysis Pathway, Intrinsic Prothrombin |
| | | | | Activation Pathway, Platelet thrombus formation |
| | | | | at injury sites, Role of Peroxisome Proliferator- |
| | | | | Activated Receptor in Inflammation) |
| 1147 | 78974 | NM_017084 | glycine N-methyltransferase | Glycine, serine and threonine metabolism |
| 1148 | 76736 | NM_013168 | hydroxymethylbilane synthase | (Hemoglobin''s Chaperone, Porphyrin and |
| | | | | chlorophyll metabolism) |
| 1149 | 78981 | NM_021655 | chromogranin A | |
| 1150 | 78982 | NM_013018 | RAB3A, member RAS oncogene | Rab GTPases Mark Targets In The Endocytotic |
| | | | family | Machinery |
| 1151 | 82354 | NM_012618 | S100 calcium-binding protein A4 | |
| 1152 | 78984 | NM_012497 | aldolase C, fructose-biphosphate | (Fructose and mannose metabolism, Glycolysis/ |
| | | | | Gluconeogenesis, Inositol metabolism, Pentose |
| | | | | phosphate pathway) |
| 1153 | 78986 | NM_153307 | cytochrome P450, family 4, | Fatty acid metabolism |
| | | | subfamily a, polypeptide 10 | |
| 284 | 78998 | NM_012793 | guanidinoacetate methyltransferase | (Arginine and proline metabolism, Glycine, serine |
| | | | | and threonine metabolism, Urea cycle and |
| | | | | metabolism of amino groups) |
| 1154 | 79012 | NM_001008370 | similar to Ras-related protein Rab- | |
| | | | 1B | |
| 1155 | 76958 | NM_031969 | calmodulin 1 | (Calcium signaling pathway, Huntington's |
| | | | | disease, Phosphatidylinositol signaling system) |
| 1156 | 79025 | X14848 | | |
| 1157 | 82350 | NM_173301 | preprotrypsinogen IV | |
| 1158 | 76963 | NM_021863 | heat shock protein 2 | |
| 399 | 79040 | NM_017040 | protein phosphatase 2a, catalytic | (TGF-beta signaling pathway, Tight junction, Wnt |
| | | | subunit, beta isoform | signaling pathway) |
| 1159 | 76722 | NM_012937 | crystallin, beta B2 | |
| 423 | 82271 | NM_031975 | parathymosin | |
| 1160 | 79044 | X16623 | | |
| 1161 | 79048 | NM_031530 | chemokine (C-C motif) ligand 2 | Cytokine-cytokine receptor interaction |
| 1161 | 79049 | NM_031530 | chemokine (C-C motif) ligand 2 | Cytokine-cytokine receptor interaction |
| 1162 | 79051 | NM_012492 | adrenergic receptor, beta 2 | (Calcium signaling pathway, Corticosteroids and |
| | | | | cardioprotection, Cystic fibrosis transmembrane |
| | | | | conductance regulator and beta 2 adrenergic |
| | | | | receptor pathway, Neuroactive ligand-receptor |
| | | | | interaction, Phospholipase C-epsilon pathway) |
| 1163 | 79060 | NM_001007598 | ribosomal protein L9 | |
| 1164 | 79063 | NM_013023 | retinal S-antigen | |
| 772 | 82424 | NM_054006 | unr protein | |
| 1016 | 79070 | NM_053865 | reticulon 1 | |
| 1165 | 76971 | NM_032080 | glycogen synthase kinase 3 beta | (ALK in cardiac myocytes, Alzheimer's disease, |
| | | | | Cell Cycle: G1/S Check Point, Cell cycle, |
| | | | | Deregulation of CDK5 in Alzheimers Disease, |
| | | | | Focal adhesion, Inactivation of Gsk3 by AKT |
| | | | | causes accumulation of b-catenin in Alveolar |
| | | | | Macrophages, Molecular Interactions of Integrin- |
| | | | | Linked Kinase, Multi-step Regulation of |
| | | | | Transcription by Pitx2, NFAT and Hypertrophy of |
| | | | | the heart (Transcription in the broken heart), |
| | | | | PAR/EMK/MARK, a Novel Family of Protein |
| | | | | KinasesThat Phosphorylate Microtubule- |
| | | | | associated Proteins, Phosphatidylinositol |
| | | | | signaling system, Phosphoinositides and their |
| | | | | downstream targets., Presenilin action in Notch |
| | | | | and Wnt signaling, Regulation of Insulin and IGF |
| | | | | Signaling by IRS Proteins, Regulation of eIF2, |
| | | | | Rho-family Proteins in Cell Adhesion and Cancer, |
| | | | | Segmentation Clock, Skeletal muscle hypertrophy |
| | | | | is regulated via AKT/mTOR pathway, Sonic |
| | | | | Hedgehog (Shh) Pathway, Transcriptional |
| | | | | Activation by Amyloid Precursor Protein, WNT |
| | | | | Signaling Pathway, Wnt signaling pathway) |
| 1166 | 79080 | X53504 | | |
| 1167 | 82282 | NM_031008 | adaptor protein complex AP-2, alpha | |
| | | | 2 subunit | |
| 1168 | 82301 | NM_053602 | ATP synthase, H+ transporting, | (ATP synthesis, Oxidative phosphorylation, |
| | | | mitochondrial F0 complex, subunit | Purine metabolism) |
| | | | F6 | |
| 1169 | 82312 | NM_080689 | dynamin 1 | (Endocytotic role of NDK, Phosphins and |
| | | | | Dynamin, Ephrins and Ephs: forward and reverse |
| | | | | signalling, Gamma-aminobutyric Acid Receptor |
| | | | | Life Cycle, Role of beta-arrestins in the activation |
| | | | | and targeting of MAP kinases, Role of syndapins |
| | | | | in vesicle trafficking, Roles of beta-arrestin- |
| | | | | dependent Recruitment of Src Kinases in GPCR |
| | | | | Signaling, Stimulation of Synaptic Vesicle |
| | | | | Recycling by Nitrous Oxide, beta-arrestins in |
| | | | | GPCR Desensitization) |
| 1170 | 79087 | X54617 | | |
| 1171 | 82349 | NM_012783 | basigin | |
| 1172 | 76974 | NM_021836 | Jun-B oncogene | GATA3 participate in activating the Th2 cytokine |
| | | | | genes expression |
| 1173 | 76750 | NM_022229 | heat shock protein 1 (chaperonin) | Prion disease |
| 1174 | 79088 | X54798 | | |
| 1175 | 76748 | NM_013134 | 3-hydroxy-3-methylglutaryl- | Biosynthesis of steroids |
| | | | Coenzyme A reductase | |
| 1176 | 82332 | NM_019331 | proprotein convertase subtilisin/kexin | |
| | | | type 3 | |
| 1177 | 79929 | NM_012742 | forkhead box A1 | |
| 1178 | 79090 | NM_017281 | proteasome (prosome, macropain) | Proteasome |
| | | | subunit, alpha type 4 | |
| 1179 | 79095 | X56448 | cell surface glycoprotein gp42 | |
| 1180 | 82430 | NM_013103 | transcription factor 2 | |
| 1181 | 79104 | NM_031838 | ribosomal protein S2 | Ribosome |
| 1182 | 79113 | X58465 | ribosomal protein S5 | |
| 1183 | 79115 | NM_012490 | acrosin | |
| 1184 | 82525 | NM_053990 | protein tyrosine phosphatase, non- | |
| | | | receptor type 2 | |
| 1185 | 82361 | NM_013190 | phosphofructokinase, liver, B-type | (Fructose and mannose metabolism, Galactose |
| | | | | metabolism, Glycolysis/Gluconeogenesis, |
| | | | | Glycolysis Pathway, Pentose phosphate pathway, |
| | | | | Phosphoinositides and their downstream targets.) |
| 1186 | 79118 | X59375 | | |
| 1187 | 79123 | X59737 | creatine kinase, mitochondrial 1, | (Arginine and proline metabolism, Mitochondrial |
| | | | ubiquitous | Permeability Transition Pore Complex, Urea cycle |
| | | | | and metabolism of amino groups) |
| 1187 | 79124 | X59737 | creatine kinase, mitochondrial 1, | (Arginine and proline metabolism, Mitochondrial |
| | | | ubiquitous | Permeability Transition Pore Complex, Urea cycle |
| | | | | and metabolism of amino groups) |
| 1188 | 79127 | NM_052799 | nitric oxide synthase 1, neuronal | Arginine and proline metabolism |
| 1189 | 82393 | NM_201415 | ribosomal protein L17 | |
| 1190 | 79132 | NM_080478 | amyloid beta (A4) precursor protein- | (Alzheimer's disease, Transcriptional Activation |
| | | | binding, family B, member 1 | by Amyloid Precursor Protein) |
| 1191 | 79133 | NM_013026 | syndecan 1 | (CASK Protein Interactions, ECM-receptor |
| | | | | interaction, Matrix Metalloproteinase 1 in Cell |
| | | | | Signaling) |
| 312 | 79984 | NM_012854 | interleukin 10 | (Adaptive Immune Response to Cancer Cells, |
| | | | | Antigen Dependent B Cell Activation, Cytokine |
| | | | | Network, Cytokine-cytokine receptor interaction, |
| | | | | Cytokines and Inflammatory Response, Dendritic |
| | | | | cells in regulating TH1 and TH2 Development, IL- |
| | | | | 10 Anti-inflammatory Signaling Pathway, |
| | | | | Immunotherapeutic Approaches to Alzheimer''s |
| | | | | Disease, Jak-STAT signaling pathway, PPAR |
| | | | | Gamma in Inflammation Control) |
| 974 | 82411 | NM_024125 | CCAAT/enhancer binding protein | IL 6 signaling pathway |
| | | | (C/EBP), beta | |
| 1192 | 79145 | X62145 | ribosomal protein L8 | |
| 1193 | 79149 | NM_198753 | ribosomal protein L3 | Ribosome |
| 1194 | 79153 | X62325 | | |
| 1195 | 79166 | X62950 | | |
| 1196 | 79167 | X62951 | | |
| 1197 | 79170 | NM_133290 | zinc finger protein 36 | |
| 1198 | 79824 | NM_053988 | calbindin 2 | |
| 1199 | 82287 | NM_031119 | Sjogren syndrome antigen B | (DNA Mismatch Repair, RNA polymerase III |
| | | | | transcription) |
| 1200 | 79893 | NM_019268 | solute carrier family 8 | Calcium signaling pathway |
| | | | (sodium/calcium exchanger), | |
| | | | member 1 | |
| 1201 | 76730 | NM_053986 | myosin Ib | |
| 1202 | 79182 | X70667 | melanocortin 3 receptor | |
| 1203 | 82425 | NM_134352 | plasminogen activator, urokinase | Complement and coagulation cascades |
| | | | receptor | |
| 1204 | 76720 | X72757 | | |
| 1205 | 82327 | NM_175756 | Fc receptor, IgG, low affinity IIb | B Lymphocyte Cell Surface Molecules |
| 1206 | 79185 | NM_130738 | SNRPN upstream reading frame | (Spliceosomal Assembly, U1snRNP Export and |
| | | | | Import) |
| 1207 | 79186 | NM_019312 | inositol 1,4,5-trisphosphate 3-kinase B | (Inositol phosphate metabolism, |
| | | | | Phosphatidylinositol signaling system) |
| 1208 | 82545 | NM_024382 | serine (or cysteine) proteinase | Complement and coagulation cascades |
| | | | inhibitor, clade D, member 1 | |
| 1209 | 76990 | NM_022516 | polypyrimidine tract binding protein 1 | |
| 1209 | 76991 | NM_022516 | polypyrimidine tract binding protein 1 | |
| 1210 | 82406 | NM_017052 | sorbitol dehydrogenase | Fructose and mannose metabolism |
| 1211 | 79189 | NM_012528 | cholinergic receptor, nicotinic, beta | (Neuromuscular Junction Protiens, Role of |
| | | | polypeptide 1 (muscle) | nicotinic acetylcholine receptors in the regulation |
| | | | | of apoptosis) |
| 1212 | 79190 | NM_019145 | cholinergic receptor, nicotinic, | (Neuromuscular Junction Protiens, Role of |
| | | | gamma polypeptide | nicotinic acetylcholine receptors in the regulation |
| | | | | of apoptosis) |
| 1213 | 79192 | NM_017194 | cholinergic receptor, nicotinic, | |
| | | | epsilon polypeptide | |
| 1214 | 79193 | X74978 | | |
| 1215 | 82414 | X75856 | testis enhanced gene transcript | |
| 1216 | 79196 | NM_001005871 | ATPase, Ca++ transporting, plasma | Calcium signaling pathway |
| | | | membrane 4 | |
| 1217 | 79198 | X76489 | CD9 antigen | |
| 1217 | 79199 | X76489 | CD9 antigen | |
| 1218 | 80285 | NM_017304 | potassium voltage-gated channel, | |
| | | | shaker-related subfamily, beta | |
| | | | member 2 | |
| 1219 | 82466 | NM_031655 | latexin | |
| 1220 | 76791 | NM_212546 | heat shock 70 kD protein 1-like | |
| 1221 | 80024 | X77818 | | |
| 1222 | 82548 | NM_053978 | RAB28, member RAS oncogene | |
| | | | family | |
| 967 | 76994 | NM_031509 | glutathione S-transferase A5 | Glutathione metabolism |
| 1223 | 76995 | NM_019184 | Cytochrome P450, subfamily IIC | (Fatty acid metabolism, Tryptophan metabolism) |
| | | | (mephenytoin 4-hydroxylase) | |
| 1224 | 82210 | NM_057144 | cysteine-rich protein 3 | |
| 1225 | 82331 | NM_080698 | fibromodulin | Small Leucine-rich Proteoglycan (SLRP) |
| | | | | molecules |
| 1226 | 82554 | NM_022597 | cathepsin B | |
| 1227 | 82392 | NM_017271 | nuclear distribution gene C homolog | |
| | | | (Aspergillus) | |
| 1228 | 82223 | NM_053974 | eukaryotic translation initiation factor | (Eukaryotic protein translation, Internal Ribosome |
| | | | 4E | Entry Pathway, NMD; Nonsense-Mediated mRNA |
| | | | | Decay, Regulation of eIF4e and p70 S6 Kinase, |
| | | | | SARS Coronavirus Protease, Skeletal muscle |
| | | | | hypertrophy is regulated via AKT/mTOR pathway, |
| | | | | mTOR Signaling Pathway) |
| 1229 | 79218 | NM_031602 | potassium inwardly-rectifying | |
| | | | channel, subfamily J, member 10 | |
| 1230 | 79221 | NM_001008889 | HIV-induced protein-7-like protease | |
| 1231 | 79993 | NM_012988 | nuclear factor I/A | |
| 1232 | 79223 | X86178 | | |
| 1233 | 80097 | NM_001008724 | fibrinogen, alpha polypeptide | (Acute Myocardial Infarction, C-Reactive Protein, |
| | | | | Apo-B in Lipid Metabolism and Cardiovascular |
| | | | | Disease, Extrinsic Prothrombin Activation |
| | | | | Pathway, Fibrinolysis Pathway, Intrinsic |
| | | | | Prothrombin Activation Pathway, Platelet |
| | | | | thrombus formation at injury sites, Role of |
| | | | | Peroxisome Proliferator-Activated Receptor in |
| | | | | Inflammation) |
| 1234 | 79228 | NM_214821 | olfactory receptor 1413 | |
| 1235 | 79229 | NM_001000363 | olfactory receptor 728 (predicted) | |
| 1236 | 79239 | NM_001000502 | olfactory receptor 1606 | |
| 1237 | 82504 | NM_024391 | hydroxysteroid (17-beta) | Androgen and estrogen metabolism |
| | | | dehydrogenase 2 | |
| 1238 | 82239 | NM_080780 | purinergic receptor P2X, ligand- | (Calcium signaling pathway, Neuroactive ligand- |
| | | | gated ion channel, 5 | receptor interaction) |
| 1239 | 82558 | NM_031722 | coated vesicle membrane protein | |
| 1240 | 79250 | NM_012896 | adenosine A3 receptor | Neuroactive ligand-receptor interaction |
| 1241 | 79988 | NM_053339 | acyl-Coenzyme A oxidase 3, | Fatty acid metabolism |
| | | | pristanoyl | |
| 258 | 82598 | NM_019170 | carbonyl reductase 1 | Prostaglandin and leukotriene metabolism |
| 1242 | 79256 | NM_053619 | complement component 5, receptor 1 | Complement and coagulation cascades, |
| | | | | Neuroactive ligand-receptor interaction) |
| 1243 | 79257 | NM_212505 | immediate early response 3 | |
| 1244 | 77005 | NM_021582 | replication protein A2 | |
| 1245 | 79264 | NM_021697 | potassium channel, subfamily V, | |
| | | | member 1 | |
| 1246 | 79265 | NM_134329 | alcohol dehydrogenase 7 (class IV), | (Bile acid biosynthesis, Fatty acid metabolism, |
| | | | mu or sigma polypeptide | Glycerolipid metabolism, Glycolysis/ |
| | | | | Gluconeogenesis, Tyrosine metabolism) |
| 1247 | 77006 | NM_153314 | UDP-glucuronosyltransferase 2B3 | (Androgen and estrogen metabolism, Pentose |
| | | | precursor, microsomal | and glucuronate interconversions, Porphyrin and |
| | | | | chlorophyll metabolism, Starch and sucrose |
| | | | | metabolism) |
| 1248 | 77007 | NM_012603 | myelocytomatosis viral oncogene | (CTCF: First Multivalent Nuclear Factor, |
| | | | homolog (avian) | Cadmium induces DNA synthesis and |
| | | | | proliferation in macrophages, Erk1/Erk2 Mapk |
| | | | | Signaling pathway, IL-2 Receptor Beta Chain in T |
| | | | | cell Activation, Inhibition of Cellular Proliferation |
| | | | | by Gleevec, Jak-STAT signaling pathway, MAPK |
| | | | | signaling pathway, MAPKinase Signaling |
| | | | | Pathway, Mechanism of Gene Regulation by |
| | | | | Peroxisome Proliferators via PPARa(alpha), |
| | | | | Neuropeptides VIP and PACAP inhibit the |
| | | | | apoptosis of activated T cells, Overview of |
| | | | | telomerase protein component gene hTert |
| | | | | Transcriptional Regulation, Role of EGF Receptor |
| | | | | Transactivation by GPCRs in Cardiac |
| | | | | Hypertrophy, TGF-beta signaling pathway, |
| | | | | Telomeres, Telomerase, Cellular Aging, and |
| | | | | Immortality, The Role Caveolin-1 in Tumour-cell |
| | | | | Invasion, Tumor Suppressor Arf Inhibits |
| | | | | Ribosomal Biogenesis, WNT Signaling Pathway, |
| | | | | Wnt signaling pathway, p38 MAPK Signaling |
| | | | | Pathway) |
| 1248 | 77008 | NM_012603 | myelocytomatosis viral oncogene | (CTCF: First Multivalent Nuclear Factor, |
| | | | homolog (avian) | Cadmium induces DNA synthesis and |
| | | | | proliferation in macrophages, Erk1/Erk2 Mapk |
| | | | | Signaling pathway, IL-2 Receptor Beta Chain in T |
| | | | | cell Activation, Inhibition of Cellular Proliferation |
| | | | | by Gleevec, Jak-STAT signaling pathway, MAPK |
| | | | | signaling pathway, MAPKinase Signaling |
| | | | | Pathway, Mechanism of Gene Regulation by |
| | | | | Peroxisome Proliferators via PPARa(alpha), |
| | | | | Neuropeptides VIP and PACAP inhibit the |
| | | | | apoptosis of activated T cells, Overview of |
| | | | | telomerase protein component gene hTert |
| | | | | Transcriptional Regulation, Role of EGF Receptor |
| | | | | Transactivation by GPCRs in Cardiac |
| | | | | Hypertrophy, TGF-beta signaling pathway, |
| | | | | Telomeres, Telomerase, Cellular Aging, and |
| | | | | Immortality, The Role Caveolin-1 in Tumour-cell |
| | | | | Invasion, Tumor Suppressor Arf Inhibits |
| | | | | Ribosomal Biogenesis, WNT Signaling Pathway, |
| | | | | Wnt signaling pathway, p38 MAPK Signaling |
| | | | | Pathway) |
| 1249 | 79894 | NM_013119 | sodium channel, voltage-gated, type | |
| | | | III, alpha polypeptide | |
| 1250 | 79281 | NM_022696 | heart and neural crest derivatives | |
| | | | expressed transcript 2 | |
| 1251 | 79288 | NM_019255 | calcium channel, voltage-dependent, | |
| | | | gamma subunit 1 | |
| 1252 | 79291 | NM_053762 | zona pellucida glycoprotein 3 | |
| 1253 | 79294 | Y11489 | a disintegrin and metalloprotease | |
| | | | domain 5 | |
| 1254 | 82731 | NM_057213 | ATPase, H+ transporting, V1 subunit | (ATP synthesis, Oxidative phosphorylation) |
| | | | B, isoform 2 | |
| 1255 | 82853 | NM_012656 | secreted acidic cysteine rich | |
| | | | glycoprotein | |
| 1256 | 76378 | NM_138911 | stress-induced-phosphoprotein 1 | Glucocorticoid Receptor-Hsp90 Heterocomplex |
| | | | (Hsp70/Hsp90-organizing protein) | |
| 1257 | 79309 | NM_133529 | calcium binding protein 1 | |
| 1258 | 79319 | NM_019136 | arginine vasopressin receptor 2 | Neuroactive ligand-receptor interaction |
| 1259 | 79325 | Z14118 | platelet derived growth factor | |
| | | | receptor, alpha polypeptide | |
| 1259 | 79326 | Z14118 | platelet derived growth factor | |
| | | | receptor, alpha polypeptide | |
| 1260 | 79327 | NM_031525 | platelet derived growth factor | (Calcium signaling pathway, Focal adhesion, |
| | | | receptor, beta polypeptide | MAPK signaling pathway, Regulation of actin |
| | | | | cytoskeleton) |
| 996 | 79330 | NM_013086 | cAMP responsive element modulator | |
| 833 | 79331 | NM_053994 | pyruvate dehydrogenase E1 alpha 2 | (Butanoate metabolism, Glycolysis/ |
| | | | | Gluconeogenesis, Pyruvate metabolism) |
| 14 | 79333 | NM_212504 | heat shock 70 kD protein 1B | MAPK signaling pathway |
| 1261 | 82221 | NM_024131 | D-dopachrome tautomerase | |
| 14 | 76766 | NM_212504 | heat shock 70 kD protein 1B | MAPK signaling pathway |
| 1262 | 79349 | NM_017094 | growth hormone receptor | (AKT Signaling Pathway, Cytokine-cytokine |
| | | | | receptor interaction, Growth Hormone Signaling |
| | | | | Pathway, Jak-STAT signaling pathway, |
| | | | | Neuroactive ligand-receptor interaction, |
| | | | | Regulation of eIF4e and p70 S6 Kinase, The IGF- |
| | | | | 1 Receptor and Longevity, Trefoil Factors Initiate |
| | | | | Mucosal Healing) |
| 1263 | 79351 | NM_021699 | serine/threonine kinase | (Inositol phosphate metabolism, Nicotinate and |
| | | | | nicotinamide metabolism, Phosphatidylinositol |
| | | | | signaling system) |

1. A method of predicting at least one toxic effect of a compound, comprising:
(a) detecting the level of expression in a tissue or cell sample exposed to the compound of ten or more genes from Table 1, 2 or 4; wherein differential expression of the genes in Table 1, 2 or 4 is indicative of at least one toxic effect.

2. (canceled)

3. A method of predicting the progression of a toxic effect of a compound, comprising:
(a) detecting the level of expression in a tissue or cell sample exposed to the compound of ten or more genes from Table 1, 2 or 4; wherein differential expression of the genes in Table 1, 2 or 4 is indicative of toxicity progression.

4. (canceled)

5. A method of predicting the cardiotoxicity of a compound, comprising:
(a) detecting the level of expression in a tissue or cell sample exposed to the compound of ten or more genes from Table 1, 2 or 4; wherein differential expression of the genes in Table 1, 2 or 4 is indicative of cardiotoxicity.

6. (canceled)

7. A method of identifying an agent that modulates the onset or progression of a toxic response, comprising:
(a) exposing an animal or a cell to the agent and a known toxin; and
(b) detecting the expression level of two or more genes from Table 1, 2 or 4; wherein differential expression of the genes in Table 1, 2 or 4 is indicative of toxicity.

8. A method of predicting the cellular pathways that a compound modulates in a cell, comprising:
(a) detecting the level of expression in a tissue or cell sample exposed to the compound of two or more genes from Table 1, 2 or 4; wherein differential expression of the genes in Table 1, 2 or 4 is associated with the modulation of at least one cellular pathway.

9. (canceled)

10. A method of predicting at least one toxic effect of a test compound, comprising:
(a) preparing a gene expression profile from a heart cell or tissue sample exposed to the test compound; and
(b) comparing the gene expression profile to a database comprising quantitative gene expression information for at least 10 genes, gene fragments of Table 1, 2 or 4 from a heart cell or tissue sample that has been exposed to at least one toxin and quantitative gene expression information for at least one gene, gene fragment of Table 1, 2 or 4 from a control heart cell or tissue sample exposed to the toxin excipient, thereby predicting at least one toxic effect of the test compound.

11. (canceled)

12. A method of predicting at least one toxic effect of a test agent comprising:
(a) providing nucleic acid hybridization data for a plurality of genes from at least one heart cell or tissue sample exposed to the test agent;
(b) converting the hybridization data from at least one gene corresponding to a gene or gene fragment of Table 1, 2 or 4 to a gene expression measure;
(c) generating a gene regulation score from the gene expression measure for said at least one gene;
(d) generating a sample prediction score for the agent; and
(e) comparing the sample prediction score to a cardiotoxicity reference prediction score, thereby predicting at least one toxic effect of the test agent.

13. (canceled)

14. A method of claim 12, wherein at least one cell or tissue sample is exposed to a test agent vehicle.

15. (canceled)

16. A method of claim 14, wherein the gene expression measure is a gene fold change value.

17. A method of claim 16, wherein the fold change value is calculated by a log scale linear additive model.

18. A method of claim 17, wherein the log scale linear additive model is a robust multi-array (RMA).

19. A method of claim 12, wherein the nucleic acid hybridization data has been screened by a quality control process that measures outlier data.

20. A method of claim 12, wherein step (c) comprises dimension reduction using Partial Least Squares (PLS).

21. A method of claim 12, wherein the sample prediction score is generated with a weighted index score for each gene.

22. A method of claim 21, wherein the weighted index score is a PLS score from Table 2.

23. A method of 12, wherein the sample prediction score for the agent is generated from the gene regulation score for said at least one gene.

24-26. (canceled)

27. A method of claim 12, wherein the toxicity reference prediction score is generated by a method comprising:
(a) providing nucleic acid hybridization data for a plurality of genes from at least one tissue or cell sample exposed to a cardiotoxin and at least one tissue or cell sample exposed to the toxin vehicle;
(b) converting the hybridization data from at least ten genes to fold change values;
(c) generating a gene regulation score from the fold change value for said at least ten genes; and
(d) generating a toxicity reference prediction score for the toxin.

28. A method of claim 27, wherein the cardiotoxin or non-cardiotoxin is selected from the group consisting of acyclovir, adriamycin, amphotericin B, BI compound, carboplatin, CCl4, cisplatin, clenbuterol, cyclophosphamide, dantrolene, dopamine, epinephrine, epirubicin, famotidine, hydralazine, ifosfamide, imatinib, isoproterenol, minoxidil, monocrotaline, norephinephrine, paroxetine, pentamidine, Pfizer compound, phenylpropanolamine, rosiglitazone, and temozolomide.

29-43. (canceled)

44. A method of providing a report comprising a prediction of at least one toxic effect of a test agent comprising:
(a) receiving nucleic acid hybridization data for a plurality of genes from at least one heart cell or tissue sample exposed to the test agent and at least one heart cell or tissue sample exposed to the test agent vehicle to a server via a remote link, wherein said plurality of genes is selected from the genes or gene fragments of Table 1, 2 or 4;
(b) converting the hybridization data from at least one gene to robust multi-array (RMA) fold change values;
(c) generating a gene regulation score from the RMA fold change value for said at least one gene;
(d) generating a sample prediction score for the agent;
(e) comparing the sample prediction score to a toxicity reference prediction score; and
(f) providing a report comprising information related to said at least one toxic effect.

45. (canceled)

46. A method of creating a toxicity model comprising:
(a) providing nucleic acid hybridization data for a plurality of genes from at least one heart cell or tissue sample exposed to a toxin;
(b) converting the hybridization data from at least one gene corresponding to a gene or gene fragment of Table 1, 2 or 4 to a gene expression measure;
(c) generating a gene regulation score from gene expression measure for said at least one gene;
(d) generating a toxicity reference prediction score for the toxin, thereby creating a toxicity model.

47-66. (canceled)

67. A computer system comprising:
(a) a computer readable medium comprising a toxicity model for predicting toxicity of a test agent, wherein the toxicity model is generated by a method of claim 46; and
(b) software that allows a user to predict at least one toxic effect of a test agent by comparing a sample prediction score to a toxicity reference prediction score in the toxicity model.

68. A computer system of claim 67, wherein the toxicity model comprises a model selected from Table 1, 2 or 4.

69-83. (canceled)

84. A computer readable medium comprising a data structure comprising at least one toxicity reference prediction score and software for accessing said data structure.

85-87. (canceled)

88. A solid support comprising at least two probes, wherein each of the probes comprises a sequence that specifically hybridizes to a gene or gene fragment in Table 1, 2 or 4.

89-98. (canceled)
